SEQUENCE LISTING (595 sequences)

SEQ ID NO: 1 - DNA, 36 bp, synthetic construct
  source 1..36: mol_type = other DNA
gtctcgaagccctatctaccctccctatctaaactc

SEQ ID NO: 2 - DNA, 36 bp, synthetic construct
  source 1..36: mol_type = other DNA
gctccactcaaccagactcccataccattctacagc

SEQ ID NO: 3 - DNA, 36 bp, synthetic construct
  source 1..36: mol_type = other DNA
agtccgctgcaatcctaatccgatcatacaacaccg

SEQ ID NO: 4 - DNA, 36 bp, synthetic construct
  source 1..36: mol_type = other DNA
tacctgccgatcccacacaccgttatcaccattctc

SEQ ID NO: 5 - DNA, 36 bp, synthetic construct
  source 1..36: mol_type = other DNA
ggaagttcaggtcggatatgggtagggttcagtgtc

SEQ ID NO: 6 - DNA, 36 bp, synthetic construct
  source 1..36: mol_type = other DNA
gtaaagtgccgtgtaccctctcccttcgacccttat

SEQ ID NO: 7 - DNA, 36 bp, synthetic construct
  source 1..36: mol_type = other DNA
ttcctcgagccttaaccgacctatctcaaacaccgc

SEQ ID NO: 8 - DNA, 36 bp, synthetic construct
  source 1..36: mol_type = other DNA
ggcagtcttacgctaacccaaccaatctacccaacg

SEQ ID NO: 9 - DNA, 36 bp, synthetic construct
  source 1..36: mol_type = other DNA
taatgctcggtccgcaacactacatacaaccaacgc

SEQ ID NO: 10 - DNA, 36 bp, synthetic construct
  source 1..36: mol_type = other DNA
ctgcccacttaaccgctctctgtcgttcatcaccat

SEQ ID NO: 11 - DNA, 36 bp, synthetic construct
  source 1..36: mol_type = other DNA
gagtaagagcccaaccaagcattccctaaacgagtc

SEQ ID NO: 12 - DNA, 36 bp, synthetic construct
  source 1..36: mol_type = other DNA
cctccgacataccataccgatttgactaaccgaacc

SEQ ID NO: 13 - DNA, 36 bp, synthetic construct
  source 1..36: mol_type = other DNA
ccaccgctctcatctaatcagaaacacgacgccaga

SEQ ID NO: 14 - DNA, 36 bp, synthetic construct
  source 1..36: mol_type = other DNA
ccatcacacgaccagttacggcaatctcaccacaag

SEQ ID NO: 15 - DNA, 36 bp, synthetic construct
  source 1..36: mol_type = other DNA
ctgtagccaccgatcaaagacgcaaccacctaatcc

SEQ ID NO: 16 - DNA, 36 bp, synthetic construct
  source 1..36: mol_type = other DNA
aagggaggtcggtaggattcggagacaaggttcagg

SEQ ID NO: 17 - DNA, 36 bp, synthetic construct
  source 1..36: mol_type = other DNA
atcccacgctagtcacacacccttctctatgccgtt

SEQ ID NO: 18 - DNA, 36 bp, synthetic construct
  source 1..36: mol_type = other DNA
cgggatagtgcaagtacggacagatagggtaagatc

SEQ ID NO: 19 - DNA, 36 bp, synthetic construct
  source 1..36: mol_type = other DNA
aaagggctgttcggttcatctacttatcggctgcag

SEQ ID NO: 20 - DNA, 36 bp, synthetic construct
  source 1..36: mol_type = other DNA
gggagtatggtgcgtcaggtccgtatcgatgtctta

SEQ ID NO: 21 - DNA, 36 bp, synthetic construct
  source 1..36: mol_type = other DNA
atccgactagctgcctaacacatgaacccactatgc

SEQ ID NO: 22 - DNA, 36 bp, synthetic construct
  source 1..36: mol_type = other DNA
cagaatcaacgagctccacgacacgcacaaccaaga

SEQ ID NO: 23 - DNA, 36 bp, synthetic construct
  source 1..36: mol_type = other DNA
ctatccactccgatcctaaactgcctgcgagcgaat

SEQ ID NO: 24 - DNA, 36 bp, synthetic construct
  source 1..36: mol_type = other DNA
gtgtgggtggtctacatatggttgcgagtcttggag

SEQ ID NO: 25 - DNA, 36 bp, synthetic construct
  source 1..36: mol_type = other DNA
ctccactcctgttcgacgacttaccaatccaaccgc

SEQ ID NO: 26 - DNA, 36 bp, synthetic construct
  source 1..36: mol_type = other DNA
tacctcttcgcaacaccactaccctcgaacaagtct

SEQ ID NO: 27 - DNA, 36 bp, synthetic construct
  source 1..36: mol_type = other DNA
gttgtattgggtgtcgcgaataggtagtggagatcg

SEQ ID NO: 28 - DNA, 36 bp, synthetic construct
  source 1..36: mol_type = other DNA
ggtgagatgcggtagaactacgtgttggaattgggc

SEQ ID NO: 29 - DNA, 36 bp, synthetic construct
  source 1..36: mol_type = other DNA
aacactctcttggtcccgtttactccgatttcaccg

SEQ ID NO: 30 - DNA, 36 bp, synthetic construct
  source 1..36: mol_type = other DNA
tagggaggagatagtcgaacacgagaggctggatga

SEQ ID NO: 31 - DNA, 36 bp, synthetic construct
  source 1..36: mol_type = other DNA
catcccttcctcgcatatgatcacgcacttccagag

SEQ ID NO: 32 - DNA, 36 bp, synthetic construct
  source 1..36: mol_type = other DNA
ccgaagatccaccgaacgcaacctaagacccttcca

SEQ ID NO: 33 - DNA, 36 bp, synthetic construct
  source 1..36: mol_type = other DNA
gattagggttgagctcgtgtcagtatcaggttcgtg

SEQ ID NO: 34 - DNA, 36 bp, synthetic construct
  source 1..36: mol_type = other DNA
agccatatcccacgccacttcggaacctagatttgc

SEQ ID NO: 35 - DNA, 36 bp, synthetic construct
  source 1..36: mol_type = other DNA
tctcgtgcccattccatcctgcatacttctcgaaac

SEQ ID NO: 36 - DNA, 36 bp, synthetic construct
  source 1..36: mol_type = other DNA
gctacgactatccatgcgactcaccaaccttctgca

SEQ ID NO: 37 - DNA, 36 bp, synthetic construct
  source 1..36: mol_type = other DNA
ccatatcgtgccatccagtacgcgaatccacaactt

SEQ ID NO: 38 - DNA, 36 bp, synthetic construct
  source 1..36: mol_type = other DNA
tgccaccgacctgtatcaacctagctgacgcgaatt

SEQ ID NO: 39 - DNA, 36 bp, synthetic construct
  source 1..36: mol_type = other DNA
agggtaggtgtcagcgaaggtctagaagcgtgcaga

SEQ ID NO: 40 - DNA, 36 bp, synthetic construct
  source 1..36: mol_type = other DNA
aacccttaccgcttctgtcatgccgtcctagctttg

SEQ ID NO: 41 - DNA, 36 bp, synthetic construct
  source 1..36: mol_type = other DNA
ctctagcacacgcgatagtaacaccacttccttcca

SEQ ID NO: 42 - DNA, 36 bp, synthetic construct
  source 1..36: mol_type = other DNA
cctgtctatacctcggcaagcttctcgtcgcatctg

SEQ ID NO: 43 - DNA, 36 bp, synthetic construct
  source 1..36: mol_type = other DNA
cttgctgccgtaccttgatcgaactctgtgccaatg

SEQ ID NO: 44 - DNA, 36 bp, synthetic construct
  source 1..36: mol_type = other DNA
gagctgcaataccaacccgacgaacaacacgaaaga

SEQ ID NO: 45 - DNA, 36 bp, synthetic construct
  source 1..36: mol_type = other DNA
gcatatcctcctagtccggttccatcctagatcacc

SEQ ID NO: 46 - DNA, 36 bp, synthetic construct
  source 1..36: mol_type = other DNA
ccctgcaacgaccgataacgcctactagtaaccacc

SEQ ID NO: 47 - DNA, 36 bp, synthetic construct
  source 1..36: mol_type = other DNA
gcgcagatccaactatcaacgacaaactcccaacac

SEQ ID NO: 48 - DNA, 36 bp, synthetic construct
  source 1..36: mol_type = other DNA
cctcagtaacagcacgctagaccacggacgactaac

SEQ ID NO: 49 - DNA, 36 bp, synthetic construct
  source 1..36: mol_type = other DNA
caacccaatgccacgtatgctactagaaccgcacct

SEQ ID NO: 50 - DNA, 36 bp, synthetic construct
  source 1..36: mol_type = other DNA
ctaaccacgacccgaacttatccgctagattccacc

SEQ ID NO: 51 - DNA, 36 bp, synthetic construct
  source 1..36: mol_type = other DNA
gggtgaataggaaggtcgatgtgatgagacacggga

SEQ ID NO: 52 - DNA, 36 bp, synthetic construct
  source 1..36: mol_type = other DNA
tatgcccttgagcgtacctagacaaaccggaaccac

SEQ ID NO: 53 - DNA, 36 bp, synthetic construct
  source 1..36: mol_type = other DNA
acatgtcagtcgtgggtgtgatctttagtgtgccag

SEQ ID NO: 54 - DNA, 36 bp, synthetic construct
  source 1..36: mol_type = other DNA
gccgtccagatcacattgaagcgactacaaatctcc

SEQ ID NO: 55 - DNA, 36 bp, synthetic construct
  source 1..36: mol_type = other DNA
gccctcatacgacgtatcgcaactacacaccttact

SEQ ID NO: 56 - DNA, 36 bp, synthetic construct
  source 1..36: mol_type = other DNA
tgtgggtattgagggtcgcgtgttgtttgcatcgta

SEQ ID NO: 57 - DNA, 36 bp, synthetic construct
  source 1..36: mol_type = other DNA
gtacacctagcgtaaggaccaatcaaactccaaccc

SEQ ID NO: 58 - DNA, 36 bp, synthetic construct
  source 1..36: mol_type = other DNA
aggcaccactcatcatgtcatcgctcagacttcaag

SEQ ID NO: 59 - DNA, 36 bp, synthetic construct
  source 1..36: mol_type = other DNA
gccgaaagagaacaccgtacccaccaacaagctctt

SEQ ID NO: 60 - DNA, 36 bp, synthetic construct
  source 1..36: mol_type = other DNA
gcgaaggtgcgaggatatggtttgcggttgtctggt

SEQ ID NO: 61 - DNA, 36 bp, synthetic construct
  source 1..36: mol_type = other DNA
aactagctcgtgtgggtaatcgtttgtggaggtgca

SEQ ID NO: 62 - DNA, 36 bp, synthetic construct
  source 1..36: mol_type = other DNA
agtgggtatggacgtcggaatgcacgggaagtttag

SEQ ID NO: 63 - DNA, 36 bp, synthetic construct
  source 1..36: mol_type = other DNA
gctgagtagtacgttcggttctgggtgcattgagta

SEQ ID NO: 64 - DNA, 36 bp, synthetic construct
  source 1..36: mol_type = other DNA
cacctcttccgttcagtctcgatcacagtggaatct

SEQ ID NO: 65 - DNA, 36 bp, synthetic construct
  source 1..36: mol_type = other DNA
accatcctagccttgcacctgttatcgttggactga

SEQ ID NO: 66 - DNA, 36 bp, synthetic construct
  source 1..36: mol_type = other DNA
gtatcatcggtggcagtagtcgtgtattcggtgtag

SEQ ID NO: 67 - DNA, 36 bp, synthetic construct
  source 1..36: mol_type = other DNA
agcgaggtatcacgacatctacacataagcggatcc

SEQ ID NO: 68 - DNA, 36 bp, synthetic construct
  source 1..36: mol_type = other DNA
gtggctgtcagataggctcggtaaagttgtaggagg

SEQ ID NO: 69 - DNA, 36 bp, synthetic construct
  source 1..36: mol_type = other DNA
gtgcgtacaggttatcgttccaaagccgtccattac

SEQ ID NO: 70 - DNA, 36 bp, synthetic construct
  source 1..36: mol_type = other DNA
ggagatgtcggacgtttgcagtgtatagacggtgtg

SEQ ID NO: 71 - DNA, 36 bp, synthetic construct
  source 1..36: mol_type = other DNA
gcccacttgatgattcctaccgatgttgccctgatc

SEQ ID NO: 72 - DNA, 36 bp, synthetic construct
  source 1..36: mol_type = other DNA
gcatctagtgtccatccagaatacgctcctccctga

SEQ ID NO: 73 - DNA, 36 bp, synthetic construct
  source 1..36: mol_type = other DNA
ccgtttcctgccctgttaaccctaccaagctacaag

SEQ ID NO: 74 - DNA, 36 bp, synthetic construct
  source 1..36: mol_type = other DNA
gctgcacgatctctacatgacgacggttctgagtta

SEQ ID NO: 75 - DNA, 36 bp, synthetic construct
  source 1..36: mol_type = other DNA
agtggagcggaagtttgggatagcgtgcataacaga

SEQ ID NO: 76 - DNA, 36 bp, synthetic construct
  source 1..36: mol_type = other DNA
acccatgctcttggtacggctctatacatgactctc

SEQ ID NO: 77 - DNA, 36 bp, synthetic construct
  source 1..36: mol_type = other DNA
gtccatgtacgtatcggtctagcacgggtctgagta

SEQ ID NO: 78 - DNA, 36 bp, synthetic construct
  source 1..36: mol_type = other DNA
cacgttaaccaaggcgagctattgagggagagtatg

SEQ ID NO: 79 - DNA, 36 bp, synthetic construct
  source 1..36: mol_type = other DNA
cacaaaccaagagcgcaacatcgtcgccacatacct

SEQ ID NO: 80 - DNA, 36 bp, synthetic construct
  source 1..36: mol_type = other DNA
aaagggagtggacgctagtgtgtcaaatcgatgagc

SEQ ID NO: 81 - DNA, 36 bp, synthetic construct
  source 1..36: mol_type = other DNA
gaaccgcaagcatccaaccggacaactactcatcat

SEQ ID NO: 82 - DNA, 36 bp, synthetic construct
  source 1..36: mol_type = other DNA
tcctcataacgctcgacttacatgtgacgcttgggt

SEQ ID NO: 83 - DNA, 36 bp, synthetic construct
  source 1..36: mol_type = other DNA
gccgactcaatactccacgctagccgaacaaatgac

SEQ ID NO: 84 - DNA, 36 bp, synthetic construct
  source 1..36: mol_type = other DNA
ggaaggttaaggcgtgtaggagttactcaatggagc

SEQ ID NO: 85 - DNA, 36 bp, synthetic construct
  source 1..36: mol_type = other DNA
agagttggagtgtgccgattcgacgtgtacaaggag

SEQ ID NO: 86 - DNA, 36 bp, synthetic construct
  source 1..36: mol_type = other DNA
tataggcgaacgggttggattagctacggacactcg

SEQ ID NO: 87 - DNA, 36 bp, synthetic construct
  source 1..36: mol_type = other DNA
gtctggtacgatggtcattacgttgttagtgctccg

SEQ ID NO: 88 - DNA, 36 bp, synthetic construct
  source 1..36: mol_type = other DNA
cctaacgctatccggaaccgactgatcacctacaac

SEQ ID NO: 89 - DNA, 36 bp, synthetic construct
  source 1..36: mol_type = other DNA
gctctgataggtgtgtcgtaacgtagatggtagtgg

SEQ ID NO: 90 - DNA, 36 bp, synthetic construct
  source 1..36: mol_type = other DNA
ccgaggatgataggacccgacaagttacccgaccaa

SEQ ID NO: 91 - DNA, 36 bp, synthetic construct
  source 1..36: mol_type = other DNA
cccgaggcctatacataacctactcacgcacagcaa

SEQ ID NO: 92 - DNA, 36 bp, synthetic construct
  source 1..36: mol_type = other DNA
ccaagagaccgctacactgaaacgatcccaacgagt

SEQ ID NO: 93 - DNA, 36 bp, synthetic construct
  source 1..36: mol_type = other DNA
tgttgacggtcggtaagtgtatagtcccatcgagtc

SEQ ID NO: 94 - DNA, 36 bp, synthetic construct
  source 1..36: mol_type = other DNA
gtacggtcgtcactatcttaacgctatgccactgac

SEQ ID NO: 95 - DNA, 36 bp, synthetic construct
  source 1..36: mol_type = other DNA
atgcgtgttggttcgtgttctagttgggacgttctg

SEQ ID NO: 96 - DNA, 36 bp, synthetic construct
  source 1..36: mol_type = other DNA
agcaagataggaccggagaactcgttaagctgtcac

SEQ ID NO: 97 - DNA, 36 bp, synthetic construct
  source 1..36: mol_type = other DNA
ggtcagtgacggcttgagatatgaggttcggcttgt

SEQ ID NO: 98 - DNA, 36 bp, synthetic construct
  source 1..36: mol_type = other DNA
cggtgggtgggtagttacatggttgcctgatatcga

SEQ ID NO: 99 - DNA, 36 bp, synthetic construct
  source 1..36: mol_type = other DNA
ccgcaagctatccatgtagtccgtcgaattctgtga

SEQ ID NO: 100 - DNA, 36 bp, synthetic construct
  source 1..36: mol_type = other DNA
caccagcaaccttacccgtcctacagaccatgatgc

SEQ ID NO: 101 - DNA, 36 bp, synthetic construct
  source 1..36: mol_type = other DNA
aactctagctccacggttaggccaacgaactgatcc

SEQ ID NO: 102 - DNA, 36 bp, synthetic construct
  source 1..36: mol_type = other DNA
ggctaactctactcccataacctagcgtgagactgc

SEQ ID NO: 103 - DNA, 36 bp, synthetic construct
  source 1..36: mol_type = other DNA
gctctgatcctaacgacggtggactcgaatgttact

SEQ ID NO: 104 - DNA, 36 bp, synthetic construct
  source 1..36: mol_type = other DNA
gacactcccatcgaacaaccaatcgtccatcctttg

SEQ ID NO: 105 - DNA, 36 bp, synthetic construct
  source 1..36: mol_type = other DNA
cttgagacagcgtgcatgctactaactccctaaccg

SEQ ID NO: 106 - DNA, 36 bp, synthetic construct
  source 1..36: mol_type = other DNA
ggataaggtctggcgaagatagggtccgaactaagt

SEQ ID NO: 107 - DNA, 36 bp, synthetic construct
  source 1..36: mol_type = other DNA
gaccgatcctagatgacgacgcgaaacaaccttcca

SEQ ID NO: 108 - DNA, 36 bp, synthetic construct
  source 1..36: mol_type = other DNA
ggtggtctatggcgactgtatgaggagcaaggtagg

SEQ ID NO: 109 - DNA, 36 bp, synthetic construct
  source 1..36: mol_type = other DNA
gcaggatcgaaactcagtatgggaggtctatgcgta

SEQ ID NO: 110 - DNA, 36 bp, synthetic construct
  source 1..36: mol_type = other DNA
gaggtcgctgtggtatagttagacggaataggtcag

SEQ ID NO: 111 - DNA, 36 bp, synthetic construct
  source 1..36: mol_type = other DNA
aggtctaatggaggaggacgtaatcacatcagtcgc

SEQ ID NO: 112 - DNA, 36 bp, synthetic construct
  source 1..36: mol_type = other DNA
gaacaaggcctaccgttagagagttgacagtccaag

SEQ ID NO: 113 - DNA, 36 bp, synthetic construct
  source 1..36: mol_type = other DNA
cgtaggtgcgagaattgagcgtgtatgtttgcctga

SEQ ID NO: 114 - DNA, 36 bp, synthetic construct
  source 1..36: mol_type = other DNA
atggtatgctgttcgggatgtaatgggaggacctga

SEQ ID NO: 115 - DNA, 36 bp, synthetic construct
  source 1..36: mol_type = other DNA
ccaccttccacgagtgagttgaccatagctagaacg

SEQ ID NO: 116 - DNA, 36 bp, synthetic construct
  source 1..36: mol_type = other DNA
gccaagacaagacacgaagccatcccaccgtactta

SEQ ID NO: 117 - DNA, 36 bp, synthetic construct
  source 1..36: mol_type = other DNA
agccctagttctgtcgaaagtatggatgggagcgtt

SEQ ID NO: 118 - DNA, 36 bp, synthetic construct
  source 1..36: mol_type = other DNA
gggagagtgacttaagccgaagtgagatctaggagg

SEQ ID NO: 119 - DNA, 36 bp, synthetic construct
  source 1..36: mol_type = other DNA
tatcggctgagtggagagtacctagattcgtcggtt

SEQ ID NO: 120 - DNA, 36 bp, synthetic construct
  source 1..36: mol_type = other DNA
gtagctgatcctgcgatacggtgagatagacattcc

SEQ ID NO: 121 - DNA, 36 bp, synthetic construct
  source 1..36: mol_type = other DNA
tagagtcccgatgagcaagtcagagcgagattgtgt

SEQ ID NO: 122 - DNA, 36 bp, synthetic construct
  source 1..36: mol_type = other DNA
ccgcaaagtaggtccaacatagcagtcgtgatcaac

SEQ ID NO: 123 - DNA, 36 bp, synthetic construct
  source 1..36: mol_type = other DNA
gaagtgtggaggttcgtctagtgaccggttgagttg

SEQ ID NO: 124 - DNA, 36 bp, synthetic construct
  source 1..36: mol_type = other DNA
gtcggatgtattggacgtcgatgatatggagtctgg

SEQ ID NO: 125 - DNA, 36 bp, synthetic construct
  source 1..36: mol_type = other DNA
ctaccacagacctcgagcaccgttcaacactaccag

SEQ ID NO: 126 - DNA, 36 bp, synthetic construct
  source 1..36: mol_type = other DNA
tttcccgtcccattaccgtaatcctcgctgacaact

SEQ ID NO: 127 - DNA, 36 bp, synthetic construct
  source 1..36: mol_type = other DNA
gcactccttaacaccgatctggccacgcatactaga

SEQ ID NO: 128 - DNA, 36 bp, synthetic construct
  source 1..36: mol_type = other DNA
ggcgtgtttggtccctatgtcctagccgaaatcttg

SEQ ID NO: 129 - DNA, 36 bp, synthetic construct
  source 1..36: mol_type = other DNA
tctagttaggtggccagtatactcatgcgacggttc

SEQ ID NO: 130 - DNA, 36 bp, synthetic construct
  source 1..36: mol_type = other DNA
ccaaacgaagcggtactcaggactcacaagcaatct

SEQ ID NO: 131 - DNA, 36 bp, synthetic construct
  source 1..36: mol_type = other DNA
ctagagggaggcgtgaaatgtagagcgaacggatca

SEQ ID NO: 132 - DNA, 36 bp, synthetic construct
  source 1..36: mol_type = other DNA
atcgtggttgctaggtcttaggcgtggctagtttca

SEQ ID NO: 133 - DNA, 18 bp, synthetic construct
  source 1..18: mol_type = other DNA
tagatagggcttcgagac

SEQ ID NO: 134 - DNA, 18 bp, synthetic construct
  source 1..18: mol_type = other DNA
agtctggttgagtggagc

SEQ ID NO: 135 - DNA, 18 bp, synthetic construct
  source 1..18: mol_type = other DNA
ttaggattgcagcggact

SEQ ID NO: 136 - DNA, 18 bp, synthetic construct
  source 1..18: mol_type = other DNA
gtgtgggatcggcaggta

SEQ ID NO: 137 - DNA, 18 bp, synthetic construct
  source 1..18: mol_type = other DNA
tatccgacctgaacttcc

SEQ ID NO: 138 - DNA, 18 bp, synthetic construct
  source 1..18: mol_type = other DNA
gggtacacggcactttac

SEQ ID NO: 139 - DNA, 18 bp, synthetic construct
  source 1..18: mol_type = other DNA
cggttaaggctcgaggaa

SEQ ID NO: 140 - DNA, 18 bp, synthetic construct
  source 1..18: mol_type = other DNA
ggttagcgtaagactgcc

SEQ ID NO: 141 - DNA, 18 bp, synthetic construct
  source 1..18: mol_type = other DNA
gttgcggaccgagcatta

SEQ ID NO: 142 - DNA, 18 bp, synthetic construct
  source 1..18: mol_type = other DNA
gagcggttaagtgggcag

SEQ ID NO: 143 - DNA, 18 bp, synthetic construct
  source 1..18: mol_type = other DNA
ttggttgggctcttactc

SEQ ID NO: 144 - DNA, 18 bp, synthetic construct
  source 1..18: mol_type = other DNA
ggtatggtatgtcggagg

SEQ ID NO: 145 - DNA, 18 bp, synthetic construct
  source 1..18: mol_type = other DNA
attagatgagagcggtgg

SEQ ID NO: 146 - DNA, 18 bp, synthetic construct
  source 1..18: mol_type = other DNA
taactggtcgtgtgatgg

SEQ ID NO: 147 - DNA, 18 bp, synthetic construct
  source 1..18: mol_type = other DNA
tttgatcggtggctacag

SEQ ID NO: 148 - DNA, 18 bp, synthetic construct
  source 1..18: mol_type = other DNA
atcctaccgacctccctt

SEQ ID NO: 149 - DNA, 18 bp, synthetic construct
  source 1..18: mol_type = other DNA
gtgtgactagcgtgggat

SEQ ID NO: 150 - DNA, 18 bp, synthetic construct
  source 1..18: mol_type = other DNA
cgtacttgcactatcccg

SEQ ID NO: 151 - DNA, 18 bp, synthetic construct
  source 1..18: mol_type = other DNA
tgaaccgaacagcccttt

SEQ ID NO: 152 - DNA, 18 bp, synthetic construct
  source 1..18: mol_type = other DNA
ctgacgcaccatactccc

SEQ ID NO: 153 - DNA, 18 bp, synthetic construct
  source 1..18: mol_type = other DNA
ttaggcagctagtcggat

SEQ ID NO: 154 - DNA, 18 bp, synthetic construct
  source 1..18: mol_type = other DNA
tggagctcgttgattctg

SEQ ID NO: 155 - DNA, 18 bp, synthetic construct
  source 1..18: mol_type = other DNA
taggatcggagtggatag

SEQ ID NO: 156 - DNA, 18 bp, synthetic construct
  source 1..18: mol_type = other DNA
tatgtagaccacccacac

SEQ ID NO: 157 - DNA, 18 bp, synthetic construct
  source 1..18: mol_type = other DNA
cgtcgaacaggagtggag

SEQ ID NO: 158 - DNA, 18 bp, synthetic construct
  source 1..18: mol_type = other DNA
tggtgttgcgaagaggta

SEQ ID NO: 159 - DNA, 18 bp, synthetic construct
  source 1..18: mol_type = other DNA
cgcgacacccaatacaac

SEQ ID NO: 160 - DNA, 18 bp, synthetic construct
  source 1..18: mol_type = other DNA
gttctaccgcatctcacc

SEQ ID NO: 161 - DNA, 18 bp, synthetic construct
  source 1..18: mol_type = other DNA
cgggaccaagagagtgtt

SEQ ID NO: 162 - DNA, 18 bp, synthetic construct
  source 1..18: mol_type = other DNA
tcgactatctcctcccta

SEQ ID NO: 163 - DNA, 18 bp, synthetic construct
  source 1..18: mol_type = other DNA
atatgcgaggaagggatg

SEQ ID NO: 164 - DNA, 18 bp, synthetic construct
  source 1..18: mol_type = other DNA
cgttcggtggatcttcgg

SEQ ID NO: 165 - DNA, 18 bp, synthetic construct
  source 1..18: mol_type = other DNA
acgagctcaaccctaatc

SEQ ID NO: 166 - DNA, 18 bp, synthetic construct
  source 1..18: mol_type = other DNA
gtggcgtgggatatggct

SEQ ID NO: 167 - DNA, 18 bp, synthetic construct
  source 1..18: mol_type = other DNA
gatggaatgggcacgaga

SEQ ID NO: 168 - DNA, 18 bp, synthetic construct
  source 1..18: mol_type = other DNA
cgcatggatagtcgtagc

SEQ ID NO: 169 - DNA, 18 bp, synthetic construct
  source 1..18: mol_type = other DNA
ctggatggcacgatatgg

SEQ ID NO: 170 - DNA, 18 bp, synthetic construct
  source 1..18: mol_type = other DNA
tgatacaggtcggtggca

SEQ ID NO: 171 - DNA, 18 bp, synthetic construct
  source 1..18: mol_type = other DNA
ttcgctgacacctaccct

SEQ ID NO: 172 - DNA, 18 bp, synthetic construct
  source 1..18: mol_type = other DNA
acagaagcggtaagggtt

SEQ ID NO: 173 - DNA, 18 bp, synthetic construct
  source 1..18: mol_type = other DNA
ctatcgcgtgtgctagag

SEQ ID NO: 174 - DNA, 18 bp, synthetic construct
  source 1..18: mol_type = other DNA
tgccgaggtatagacagg

SEQ ID NO: 175 - DNA, 18 bp, synthetic construct
  source 1..18: mol_type = other DNA
tcaaggtacggcagcaag

SEQ ID NO: 176 - DNA, 18 bp, synthetic construct
  source 1..18: mol_type = other DNA
gggttggtattgcagctc

SEQ ID NO: 177 - DNA, 18 bp, synthetic construct
  source 1..18: mol_type = other DNA
cggactaggaggatatgc

SEQ ID NO: 178 - DNA, 18 bp, synthetic construct
  source 1..18: mol_type = other DNA
ttatcggtcgttgcaggg

SEQ ID NO: 179 - DNA, 18 bp, synthetic construct
  source 1..18: mol_type = other DNA
tgatagttggatctgcgc

SEQ ID NO: 180 - DNA, 18 bp, synthetic construct
  source 1..18: mol_type = other DNA
agcgtgctgttactgagg

SEQ ID NO: 181 - DNA, 18 bp, synthetic construct
  source 1..18: mol_type = other DNA
atacgtggcattgggttg

SEQ ID NO: 182 - DNA, 18 bp, synthetic construct
  source 1..18: mol_type = other DNA
agttcgggtcgtggttag

SEQ ID NO: 183 - DNA, 18 bp, synthetic construct
  source 1..18: mol_type = other DNA
cgaccttcctattcaccc

SEQ ID NO: 184 - DNA, 18 bp, synthetic construct
  source 1..18: mol_type = other DNA
ggtacgctcaagggcata

SEQ ID NO: 185 - DNA, 18 bp, synthetic construct
  source 1..18: mol_type = other DNA
cacccacgactgacatgt

SEQ ID NO: 186 - DNA, 18 bp, synthetic construct
  source 1..18: mol_type = other DNA
caatgtgatctggacggc

SEQ ID NO: 187 - DNA, 18 bp, synthetic construct
  source 1..18: mol_type = other DNA
gatacgtcgtatgagggc

SEQ ID NO: 188 - DNA, 18 bp, synthetic construct
  source 1..18: mol_type = other DNA
cgaccctcaatacccaca

SEQ ID NO: 189 - DNA, 18 bp, synthetic construct
  source 1..18: mol_type = other DNA
tccttacgctaggtgtac

SEQ ID NO: 190 - DNA, 18 bp, synthetic construct
  source 1..18: mol_type = other DNA
acatgatgagtggtgcct

SEQ ID NO: 191 - DNA, 18 bp, synthetic construct
  source 1..18: mol_type = other DNA
acggtgttctctttcggc

SEQ ID NO: 192 - DNA, 18 bp, synthetic construct
  source 1..18: mol_type = other DNA
atatcctcgcaccttcgc

SEQ ID NO: 193 - DNA, 18 bp, synthetic construct
  source 1..18: mol_type = other DNA
tacccacacgagctagtt

SEQ ID NO: 194 - DNA, 18 bp, synthetic construct
  source 1..18: mol_type = other DNA
ccgacgtccatacccact

SEQ ID NO: 195 - DNA, 18 bp, synthetic construct
  source 1..18: mol_type = other DNA
ccgaacgtactactcagc

SEQ ID NO: 196 - DNA, 18 bp, synthetic construct
  source 1..18: mol_type = other DNA
gactgaacggaagaggtg

SEQ ID NO: 197 - DNA, 18 bp, synthetic construct
  source 1..18: mol_type = other DNA
gtgcaaggctaggatggt

SEQ ID NO: 198 - DNA, 18 bp, synthetic construct
  source 1..18: mol_type = other DNA
tactgccaccgatgatac

SEQ ID NO: 199 - DNA, 18 bp, synthetic construct
  source 1..18: mol_type = other DNA
atgtcgtgatacctcgct

SEQ ID NO: 200 - DNA, 18 bp, synthetic construct
  source 1..18: mol_type = other DNA
agcctatctgacagccac

SEQ ID NO: 201 - DNA, 18 bp, synthetic construct
  source 1..18: mol_type = other DNA
acgataacctgtacgcac

SEQ ID NO: 202 - DNA, 18 bp, synthetic construct
  source 1..18: mol_type = other DNA
caaacgtccgacatctcc

SEQ ID NO: 203 - DNA, 18 bp, synthetic construct
  source 1..18: mol_type = other DNA
aggaatcatcaagtgggc

SEQ ID NO: 204 - DNA, 18 bp, synthetic construct
  source 1..18: mol_type = other DNA
tggatggacactagatgc

SEQ ID NO: 205 - DNA, 18 bp, synthetic construct
  source 1..18: mol_type = other DNA
taacagggcaggaaacgg

SEQ ID NO: 206 - DNA, 18 bp, synthetic construct
  source 1..18: mol_type = other DNA
atgtagagatcgtgcagc

SEQ ID NO: 207 - DNA, 18 bp, synthetic construct
  source 1..18: mol_type = other DNA
ccaaacttccgctccact

SEQ ID NO: 208 - DNA, 18 bp, synthetic construct
  source 1..18: mol_type = other DNA
cgtaccaagagcatgggt

SEQ ID NO: 209 - DNA, 18 bp, synthetic construct
  source 1..18: mol_type = other DNA
accgatacgtacatggac

SEQ ID NO: 210 - DNA, 18 bp, synthetic construct
  source 1..18: mol_type = other DNA
ctcgccttggttaacgtg

SEQ ID NO: 211 - DNA, 18 bp, synthetic construct
  source 1..18: mol_type = other DNA
ttgcgctcttggtttgtg

SEQ ID NO: 212 - DNA, 18 bp, synthetic construct
  source 1..18: mol_type = other DNA
ctagcgtccactcccttt

SEQ ID NO: 213 - DNA, 18 bp, synthetic construct
  source 1..18: mol_type = other DNA
gttggatgcttgcggttc

SEQ ID NO: 214 - DNA, 18 bp, synthetic construct
  source 1..18: mol_type = other DNA
agtcgagcgttatgagga

SEQ ID NO: 215 - DNA, 18 bp, synthetic construct
  source 1..18: mol_type = other DNA
gtggagtattgagtcggc

SEQ ID NO: 216 - DNA, 18 bp, synthetic construct
  source 1..18: mol_type = other DNA
tacacgccttaaccttcc

SEQ ID NO: 217 - DNA, 18 bp, synthetic construct
  source 1..18: mol_type = other DNA
tcggcacactccaactct

SEQ ID NO: 218 - DNA, 18 bp, synthetic construct
  source 1..18: mol_type = other DNA
ccaacccgttcgcctata

SEQ ID NO: 219 - DNA, 18 bp, synthetic construct
  source 1..18: mol_type = other DNA
atgaccatcgtaccagac

SEQ ID NO: 220 - DNA, 18 bp, synthetic construct
  source 1..18: mol_type = other DNA
gttccggatagcgttagg

SEQ ID NO: 221 - DNA, 18 bp, synthetic construct
  source 1..18: mol_type = other DNA
cgacacacctatcagagc

SEQ ID NO: 222 - DNA, 18 bp, synthetic construct
  source 1..18: mol_type = other DNA
gggtcctatcatcctcgg

SEQ ID NO: 223 - DNA, 18 bp, synthetic construct
  source 1..18: mol_type = other DNA
ttatgtataggcctcggg

SEQ ID NO: 224 - DNA, 18 bp, synthetic construct
  source 1..18: mol_type = other DNA
agtgtagcggtctcttgg

SEQ ID NO: 225 - DNA, 18 bp, synthetic construct
  source 1..18: mol_type = other DNA
acttaccgaccgtcaaca

SEQ ID NO: 226 - DNA, 18 bp, synthetic construct
  source 1..18: mol_type = other DNA
agatagtgacgaccgtac

SEQ ID NO: 227 - DNA, 18 bp, synthetic construct
  source 1..18: mol_type = other DNA
acacgaaccaacacgcat

SEQ ID NO: 228 - DNA, 18 bp, synthetic construct
  source 1..18: mol_type = other DNA
ctccggtcctatcttgct

SEQ ID NO: 229 - DNA, 18 bp, synthetic construct
  source 1..18: mol_type = other DNA
ctcaagccgtcactgacc

SEQ ID NO: 230 - DNA, 18 bp, synthetic construct
  source 1..18: mol_type = other DNA
gtaactacccacccaccg

SEQ ID NO: 231 - DNA, 18 bp, synthetic construct
  source 1..18: mol_type = other DNA
tacatggatagcttgcgg

SEQ ID NO: 232 - DNA, 18 bp, synthetic construct
  source 1..18: mol_type = other DNA
cgggtaaggttgctggtg

SEQ ID NO: 233 - DNA, 18 bp, synthetic construct
  source 1..18: mol_type = other DNA
aaccgtggagctagagtt

SEQ ID NO: 234 - DNA, 18 bp, synthetic construct
  source 1..18: mol_type = other DNA
atgggagtagagttagcc

SEQ ID NO: 235 - DNA, 18 bp, synthetic construct
  source 1..18: mol_type = other DNA
cgtcgttaggatcagagc

SEQ ID NO: 236 - DNA, 18 bp, synthetic construct
  source 1..18: mol_type = other DNA
ttgttcgatgggagtgtc

SEQ ID NO: 237 - DNA, 18 bp, synthetic construct
  source 1..18: mol_type = other DNA
catgcacgctgtctcaag

SEQ ID NO: 238 - DNA, 18 bp, synthetic construct
  source 1..18: mol_type = other DNA
cttcgccagaccttatcc

SEQ ID NO: 239 - DNA, 18 bp, synthetic construct
  source 1..18: mol_type = other DNA
cgtcatctaggatcggtc

SEQ ID NO: 240 - DNA, 18 bp, synthetic construct
  source 1..18: mol_type = other DNA
cagtcgccatagaccacc

SEQ ID NO: 241 - DNA, 18 bp, synthetic construct
  source 1..18: mol_type = other DNA
actgagtttcgatcctgc

SEQ ID NO: 242 - DNA, 18 bp, synthetic construct
  source 1..18: mol_type = other DNA
ctataccacagcgacctc

SEQ ID NO: 243 - DNA, 18 bp, synthetic construct
  source 1..18: mol_type = other DNA
tcctcctccattagacct

SEQ ID NO: 244 - DNA, 18 bp, synthetic construct
  source 1..18: mol_type = other DNA
taacggtaggccttgttc

SEQ ID NO: 245 - DNA, 18 bp, synthetic construct
  source 1..18: mol_type = other DNA
tcaattctcgcacctacg

SEQ ID NO: 246 - DNA, 18 bp, synthetic construct
  source 1..18: mol_type = other DNA
tcccgaacagcataccat

SEQ ID NO: 247 - DNA, 18 bp, synthetic construct
  source 1..18: mol_type = other DNA
ctcactcgtggaaggtgg

SEQ ID NO: 248 - DNA, 18 bp, synthetic construct
  source 1..18: mol_type = other DNA
ttcgtgtcttgtcttggc

SEQ ID NO: 249 - DNA, 18 bp, synthetic construct
  source 1..18: mol_type = other DNA
ttcgacagaactagggct

SEQ ID NO: 250 - DNA, 18 bp, synthetic construct
  source 1..18: mol_type = other DNA
ggcttaagtcactctccc

SEQ ID NO: 251 - DNA, 18 bp, synthetic construct
  source 1..18: mol_type = other DNA
ctctccactcagccgata

SEQ ID NO: 252 - DNA, 18 bp, synthetic construct
  source 1..18: mol_type = other DNA
tatcgcaggatcagctac

SEQ ID NO: 253 - DNA, 18 bp, synthetic construct
  source 1..18: mol_type = other DNA
ttgctcatcgggactcta

SEQ ID NO: 254 - DNA, 18 bp, synthetic construct
  source 1..18: mol_type = other DNA
gttggacctactttgcgg

SEQ ID NO: 255 - DNA, 18 bp, synthetic construct
  source 1..18: mol_type = other DNA
gacgaacctccacacttc

SEQ ID NO: 256 - DNA, 18 bp, synthetic construct
  source 1..18: mol_type = other DNA
acgtccaatacatccgac

SEQ ID NO: 257 - DNA, 18 bp, synthetic construct
  source 1..18: mol_type = other DNA
gctcgaggtctgtggtag

SEQ ID NO: 258 - DNA, 18 bp, synthetic construct
  source 1..18: mol_type = other DNA
cggtaatgggacgggaaa

SEQ ID NO: 259 - DNA, 18 bp, synthetic construct
  source 1..18: mol_type = other DNA
atcggtgttaaggagtgc

SEQ ID NO: 260 - DNA, 18 bp, synthetic construct
  source 1..18: mol_type = other DNA
atagggaccaaacacgcc

SEQ ID NO: 261 - DNA, 18 bp, synthetic construct
  source 1..18: mol_type = other DNA
actggccacctaactaga

SEQ ID NO: 262 - DNA, 18 bp, synthetic construct
  source 1..18: mol_type = other DNA
gagtaccgcttcgtttgg

SEQ ID NO: 263 - DNA, 18 bp, synthetic construct
  source 1..18: mol_type = other DNA
tttcacgcctccctctag

SEQ ID NO: 264 - DNA, 18 bp, synthetic construct
  source 1..18: mol_type = other DNA
agacctagcaaccacgat

SEQ ID NO: 265 - DNA, 18 bp, synthetic construct
  source 1..18: mol_type = other DNA
gagtttagatagggaggg

SEQ ID NO: 266 - DNA, 18 bp, synthetic construct
  source 1..18: mol_type = other DNA
gctgtagaatggtatggg

SEQ ID NO: 267 - DNA, 18 bp, synthetic construct
  source 1..18: mol_type = other DNA
cggtgttgtatgatcgga

SEQ ID NO: 268 - DNA, 18 bp, synthetic construct
  source 1..18: mol_type = other DNA
gagaatggtgataacggt

SEQ ID NO: 269 - DNA, 18 bp, synthetic construct
  source 1..18: mol_type = other DNA
gacactgaaccctaccca

SEQ ID NO: 270 - DNA, 18 bp, synthetic construct
  source 1..18: mol_type = other DNA
ataagggtcgaagggaga

SEQ ID NO: 271 - DNA, 18 bp, synthetic construct
  source 1..18: mol_type = other DNA
gcggtgtttgagataggt

SEQ ID NO: 272 - DNA, 18 bp, synthetic construct
  source 1..18: mol_type = other DNA
cgttgggtagattggttg

SEQ ID NO: 273 - DNA, 18 bp, synthetic construct
  source 1..18: mol_type = other DNA
gcgttggttgtatgtagt

SEQ ID NO: 274 - DNA, 18 bp, synthetic construct
  source 1..18: mol_type = other DNA
atggtgatgaacgacaga

SEQ ID NO: 275 - DNA, 18 bp, synthetic construct
  source 1..18: mol_type = other DNA
gactcgtttagggaatgc

SEQ ID NO: 276 - DNA, 18 bp, synthetic construct
  source 1..18: mol_type = other DNA
ggttcggttagtcaaatc

SEQ ID NO: 277 - DNA, 18 bp, synthetic construct
  source 1..18: mol_type = other DNA
tctggcgtcgtgtttctg

SEQ ID NO: 278 - DNA, 18 bp, synthetic construct
  source 1..18: mol_type = other DNA
cttgtggtgagattgccg

SEQ ID NO: 279 - DNA, 18 bp, synthetic construct
  source 1..18: mol_type = other DNA
ggattaggtggttgcgtc

SEQ ID NO: 280 - DNA, 18 bp, synthetic construct
  source 1..18: mol_type = other DNA
cctgaaccttgtctccga

SEQ ID NO: 281 - DNA, 18 bp, synthetic construct
  source 1..18: mol_type = other DNA
aacggcatagagaagggt

SEQ ID NO: 282 - DNA, 18 bp, synthetic construct
  source 1..18: mol_type = other DNA
gatcttaccctatctgtc

SEQ ID NO: 283 - DNA, 18 bp, synthetic construct
  source 1..18: mol_type = other DNA
ctgcagccgataagtaga

SEQ ID NO: 284 - DNA, 18 bp, synthetic construct
  source 1..18: mol_type = other DNA
taagacatcgatacggac

SEQ ID NO: 285 - DNA, 18 bp, synthetic construct
  source 1..18: mol_type = other DNA
gcatagtgggttcatgtg

SEQ ID NO: 286 - DNA, 18 bp, synthetic construct
  source 1..18: mol_type = other DNA
tcttggttgtgcgtgtcg

SEQ ID NO: 287 - DNA, 18 bp, synthetic construct
  source 1..18: mol_type = other DNA
attcgctcgcaggcagtt

SEQ ID NO: 288 - DNA, 18 bp, synthetic construct
  source 1..18: mol_type = other DNA
ctccaagactcgcaacca

SEQ ID NO: 289 - DNA, 18 bp, synthetic construct
  source 1..18: mol_type = other DNA
gcggttggattggtaagt

SEQ ID NO: 290 - DNA, 18 bp, synthetic construct
  source 1..18: mol_type = other DNA
agacttgttcgagggtag

SEQ ID NO: 291 - DNA, 18 bp, synthetic construct
  source 1..18: mol_type = other DNA
cgatctccactacctatt

SEQ ID NO: 292 - DNA, 18 bp, synthetic construct
  source 1..18: mol_type = other DNA
gcccaattccaacacgta

SEQ ID NO: 293 - DNA, 18 bp, synthetic construct
  source 1..18: mol_type = other DNA
cggtgaaatcggagtaaa

SEQ ID NO: 294 - DNA, 18 bp, synthetic construct
  source 1..18: mol_type = other DNA
tcatccagcctctcgtgt

SEQ ID NO: 295 - DNA, 18 bp, synthetic construct
  source 1..18: mol_type = other DNA
ctctggaagtgcgtgatc

SEQ ID NO: 296 - DNA, 18 bp, synthetic construct
  source 1..18: mol_type = other DNA
tggaagggtcttaggttg

SEQ ID NO: 297 - DNA, 18 bp, synthetic construct
  source 1..18: mol_type = other DNA
cacgaacctgatactgac

SEQ ID NO: 298 - DNA, 18 bp, synthetic construct
  source 1..18: mol_type = other DNA
gcaaatctaggttccgaa

SEQ ID NO: 299 - DNA, 18 bp, synthetic construct
  source 1..18: mol_type = other DNA
gtttcgagaagtatgcag

SEQ ID NO: 300 - DNA, 18 bp, synthetic construct
  source 1..18: mol_type = other DNA
tgcagaaggttggtgagt

SEQ ID NO: 301 - DNA, 18 bp, synthetic construct
  source 1..18: mol_type = other DNA
aagttgtggattcgcgta

SEQ ID NO: 302 - DNA, 18 bp, synthetic construct
  source 1..18: mol_type = other DNA
aattcgcgtcagctaggt

SEQ ID NO: 303 - DNA, 18 bp, synthetic construct
  source 1..18: mol_type = other DNA
tctgcacgcttctagacc

SEQ ID NO: 304 - DNA, 18 bp, synthetic construct
  source 1..18: mol_type = other DNA
caaagctaggacggcatg

SEQ ID NO: 305 - DNA, 18 bp, synthetic construct
  source 1..18: mol_type = other DNA
tggaaggaagtggtgtta

SEQ ID NO: 306 - DNA, 18 bp, synthetic construct
  source 1..18: mol_type = other DNA
cagatgcgacgagaagct

SEQ ID NO: 307 - DNA, 18 bp, synthetic construct
  source 1..18: mol_type = other DNA
cattggcacagagttcga

SEQ ID NO: 308 - DNA, 18 bp, synthetic construct
  source 1..18: mol_type = other DNA
tctttcgtgttgttcgtc

SEQ ID NO: 309 - DNA, 18 bp, synthetic construct
  source 1..18: mol_type = other DNA
ggtgatctaggatggaac

SEQ ID NO: 310 - DNA, 18 bp, synthetic construct
  source 1..18: mol_type = other DNA
ggtggttactagtaggcg

SEQ ID NO: 311 - DNA, 18 bp, synthetic construct
  source 1..18: mol_type = other DNA
gtgttgggagtttgtcgt

SEQ ID NO: 312 - DNA, 18 bp, synthetic construct
  source 1..18: mol_type = other DNA
gttagtcgtccgtggtct

SEQ ID NO: 313 - DNA, 18 bp, synthetic construct
  source 1..18: mol_type = other DNA
aggtgcggttctagtagc

SEQ ID NO: 314 - DNA, 18 bp, synthetic construct
  source 1..18: mol_type = other DNA
ggtggaatctagcggata

SEQ ID NO: 315 - DNA, 18 bp, synthetic construct
  source 1..18: mol_type = other DNA
tcccgtgtctcatcacat

SEQ ID NO: 316 - DNA, 18 bp, synthetic construct
  source 1..18: mol_type = other DNA
gtggttccggtttgtcta

SEQ ID NO: 317 - DNA, 18 bp, synthetic construct
  source 1..18: mol_type = other DNA
ctggcacactaaagatca

SEQ ID NO: 318 - DNA, 18 bp, synthetic construct
  source 1..18: mol_type = other DNA
ggagatttgtagtcgctt

SEQ ID NO: 319 - DNA, 18 bp, synthetic construct
  source 1..18: mol_type = other DNA
agtaaggtgtgtagttgc

SEQ ID NO: 320 - DNA, 18 bp, synthetic construct
  source 1..18: mol_type = other DNA
tacgatgcaaacaacacg

SEQ ID NO: 321 - DNA, 18 bp, synthetic construct
  source 1..18: mol_type = other DNA
gggttggagtttgattgg

SEQ ID NO: 322 - DNA, 18 bp, synthetic construct
  source 1..18: mol_type = other DNA
cttgaagtctgagcgatg

SEQ ID NO: 323 - DNA, 18 bp, synthetic construct
  source 1..18: mol_type = other DNA
aagagcttgttggtgggt

SEQ ID NO: 324 - DNA, 18 bp, synthetic construct
  source 1..18: mol_type = other DNA
accagacaaccgcaaacc

SEQ ID NO: 325 - DNA, 18 bp, synthetic construct
  source 1..18: mol_type = other DNA
tgcacctccacaaacgat

SEQ ID NO: 326 - DNA, 18 bp, synthetic construct
  source 1..18: mol_type = other DNA
ctaaacttcccgtgcatt

SEQ ID NO: 327 - DNA, 18 bp, synthetic construct
  source 1..18: mol_type = other DNA
tactcaatgcacccagaa

SEQ ID NO: 328 - DNA, 18 bp, synthetic construct
  source 1..18: mol_type = other DNA
agattccactgtgatcga

SEQ ID NO: 329 - DNA, 18 bp, synthetic construct
  source 1..18: mol_type = other DNA
tcagtccaacgataacag

SEQ ID NO: 330 - DNA, 18 bp, synthetic construct
  source 1..18: mol_type = other DNA
ctacaccgaatacacgac

SEQ ID NO: 331 - DNA, 18 bp, synthetic construct
  source 1..18: mol_type = other DNA
ggatccgcttatgtgtag

SEQ ID NO: 332 - DNA, 18 bp, synthetic construct
  source 1..18: mol_type = other DNA
cctcctacaactttaccg

SEQ ID NO: 333 - DNA, 18 bp, synthetic construct
  source 1..18: mol_type = other DNA
gtaatggacggctttgga

SEQ ID NO: 334 - DNA, 18 bp, synthetic construct
  source 1..18: mol_type = other DNA
cacaccgtctatacactg

SEQ ID NO: 335 - DNA, 18 bp, synthetic construct
  source 1..18: mol_type = other DNA
gatcagggcaacatcggt

SEQ ID NO: 336 - DNA, 18 bp, synthetic construct
  source 1..18: mol_type = other DNA
tcagggaggagcgtattc

SEQ ID NO: 337 - DNA, 18 bp, synthetic construct
  source 1..18: mol_type = other DNA
cttgtagcttggtagggt

SEQ ID NO: 338 - DNA, 18 bp, synthetic construct
  source 1..18: mol_type = other DNA
taactcagaaccgtcgtc

SEQ ID NO: 339 - DNA, 18 bp, synthetic construct
  source 1..18: mol_type = other DNA
tctgttatgcacgctatc

SEQ ID NO: 340 - DNA, 18 bp, synthetic construct
  source 1..18: mol_type = other DNA
gagagtcatgtatagagc

SEQ ID NO: 341 - DNA, 18 bp, synthetic construct
  source 1..18: mol_type = other DNA
tactcagacccgtgctag

SEQ ID NO: 342 - DNA, 18 bp, synthetic construct
  source 1..18: mol_type = other DNA
catactctccctcaatag

SEQ ID NO: 343 - DNA, 18 bp, synthetic construct
  source 1..18: mol_type = other DNA
aggtatgtggcgacgatg

SEQ ID NO: 344 - DNA, 18 bp, synthetic construct
  source 1..18: mol_type = other DNA
gctcatcgatttgacaca

SEQ ID NO: 345 - DNA, 18 bp, synthetic construct
  source 1..18: mol_type = other DNA
atgatgagtagttgtccg

SEQ ID NO: 346 - DNA, 18 bp, synthetic construct
  source 1..18: mol_type = other DNA
acccaagcgtcacatgta

SEQ ID NO: 347 - DNA, 18 bp, synthetic construct
  source 1..18: mol_type = other DNA
gtcatttgttcggctagc

SEQ ID NO: 348 - DNA, 18 bp, synthetic construct
  source 1..18: mol_type = other DNA
gctccattgagtaactcc

SEQ ID NO: 349 - DNA, 18 bp, synthetic construct
  source 1..18: mol_type = other DNA
ctccttgtacacgtcgaa

SEQ ID NO: 350 - DNA, 18 bp, synthetic construct
  source 1..18: mol_type = other DNA
cgagtgtccgtagctaat

SEQ ID NO: 351 - DNA, 18 bp, synthetic construct
  source 1..18: mol_type = other DNA
cggagcactaacaacgta

SEQ ID NO: 352 - DNA, 18 bp, synthetic construct
  source 1..18: mol_type = other DNA
gttgtaggtgatcagtcg

SEQ ID NO: 353 - DNA, 18 bp, synthetic construct
  source 1..18: mol_type = other DNA
ccactaccatctacgtta

SEQ ID NO: 354 - DNA, 18 bp, synthetic construct
  source 1..18: mol_type = other DNA
ttggtcgggtaacttgtc

SEQ ID NO: 355 - DNA, 18 bp, synthetic construct
  source 1..18: mol_type = other DNA
ttgctgtgcgtgagtagg

SEQ ID NO: 356 - DNA, 18 bp, synthetic construct
  source 1..18: mol_type = other DNA
actcgttgggatcgtttc

SEQ ID NO: 357 - DNA, 18 bp, synthetic construct
  source 1..18: mol_type = other DNA
gactcgatgggactatac

SEQ ID NO: 358 - DNA, 18 bp, synthetic construct
  source 1..18: mol_type = other DNA
gtcagtggcatagcgtta

SEQ ID NO: 359 - DNA, 18 bp, synthetic construct
  source 1..18: mol_type = other DNA
cagaacgtcccaactaga

SEQ ID NO: 360 - DNA, 18 bp, synthetic construct
  source 1..18: mol_type = other DNA
gtgacagcttaacgagtt

SEQ ID NO: 361 - DNA, 18 bp, synthetic construct
  source 1..18: mol_type = other DNA
acaagccgaacctcatat

SEQ ID NO: 362 - DNA, 18 bp, synthetic construct
  source 1..18: mol_type = other DNA
tcgatatcaggcaaccat

SEQ ID NO: 363 - DNA, 18 bp, synthetic construct
  source 1..18: mol_type = other DNA
tcacagaattcgacggac

SEQ ID NO: 364 - DNA, 18 bp, synthetic construct
  source 1..18: mol_type = other DNA
gcatcatggtctgtagga

SEQ ID NO: 365 - DNA, 18 bp, synthetic construct
  source 1..18: mol_type = other DNA
ggatcagttcgttggcct

SEQ ID NO: 366 - DNA, 18 bp, synthetic construct
  source 1..18: mol_type = other DNA
gcagtctcacgctaggtt

SEQ ID NO: 367 - DNA, 18 bp, synthetic construct
  source 1..18: mol_type = other DNA
agtaacattcgagtccac

SEQ ID NO: 368 - DNA, 18 bp, synthetic construct
  source 1..18: mol_type = other DNA
caaaggatggacgattgg

SEQ ID NO: 369 - DNA, 18 bp, synthetic construct
  source 1..18: mol_type = other DNA
cggttagggagttagtag

SEQ ID NO: 370 - DNA, 18 bp, synthetic construct
  source 1..18: mol_type = other DNA
acttagttcggaccctat

SEQ ID NO: 371 - DNA, 18 bp, synthetic construct
  source 1..18: mol_type = other DNA
tggaaggttgtttcgcgt

SEQ ID NO: 372 - DNA, 18 bp, synthetic construct
  source 1..18: mol_type = other DNA
cctaccttgctcctcata

SEQ ID NO: 373 - DNA, 18 bp, synthetic construct
  source 1..18: mol_type = other DNA
tacgcatagacctcccat

SEQ ID NO: 374 - DNA, 18 bp, synthetic construct
  source 1..18: mol_type = other DNA
ctgacctattccgtctaa

SEQ ID NO: 375 - DNA, 18 bp, synthetic construct
  source 1..18: mol_type = other DNA
gcgactgatgtgattacg

SEQ ID NO: 376 - DNA, 18 bp, synthetic construct
  source 1..18: mol_type = other DNA
cttggactgtcaactctc

SEQ ID NO: 377 - DNA, 18 bp, synthetic construct
  source 1..18: mol_type = other DNA
tcaggcaaacatacacgc

SEQ ID NO: 378 - DNA, 18 bp, synthetic construct
  source 1..18: mol_type = other DNA
tcaggtcctcccattaca

SEQ ID NO: 379 - DNA, 18 bp, synthetic construct
  source 1..18: mol_type = other DNA
cgttctagctatggtcaa

SEQ ID NO: 380 - DNA, 18 bp, synthetic construct
  source 1..18: mol_type = other DNA
taagtacggtgggatggc

SEQ ID NO: 381 - DNA, 18 bp, synthetic construct
  source 1..18: mol_type = other DNA
aacgctcccatccatact

SEQ ID NO: 382 - DNA, 18 bp, synthetic construct
  source 1..18: mol_type = other DNA
cctcctagatctcacttc

SEQ ID NO: 383 - DNA, 18 bp, synthetic construct
  source 1..18: mol_type = other DNA
aaccgacgaatctaggta

SEQ ID NO: 384 - DNA, 18 bp, synthetic construct
  source 1..18: mol_type = other DNA
ggaatgtctatctcaccg

SEQ ID NO: 385 - DNA, 18 bp, synthetic construct
  source 1..18: mol_type = other DNA
acacaatctcgctctgac

SEQ ID NO: 386 - DNA, 18 bp, synthetic construct
  source 1..18: mol_type = other DNA
gttgatcacgactgctat

SEQ ID NO: 387 - DNA, 18 bp, synthetic construct
  source 1..18: mol_type = other DNA
caactcaaccggtcacta

SEQ ID NO: 388 - DNA, 18 bp, synthetic construct
  source 1..18: mol_type = other DNA
ccagactccatatcatcg

SEQ ID NO: 389 - DNA, 18 bp, synthetic construct
  source 1..18: mol_type = other DNA
ctggtagtgttgaacggt

SEQ ID NO: 390 - DNA, 18 bp, synthetic construct
  source 1..18: mol_type = other DNA
agttgtcagcgaggatta

SEQ ID NO: 391 - DNA, 18 bp, synthetic construct
  source 1..18: mol_type = other DNA
tctagtatgcgtggccag

SEQ ID NO: 392 - DNA, 18 bp, synthetic construct
  source 1..18: mol_type = other DNA
caagatttcggctaggac

SEQ ID NO: 393 - DNA, 18 bp, synthetic construct
  source 1..18: mol_type = other DNA
gaaccgtcgcatgagtat

SEQ ID NO: 394 - DNA, 18 bp, synthetic construct
  source 1..18: mol_type = other DNA
agattgcttgtgagtcct

SEQ ID NO: 395 - DNA, 18 bp, synthetic construct
  source 1..18: mol_type = other DNA
tgatccgttcgctctaca

SEQ ID NO: 396 - DNA, 18 bp, synthetic construct
  source 1..18: mol_type = other DNA
tgaaactagccacgccta

SEQ ID NO: 397 - DNA, 36 bp, synthetic construct
  source 1..36: mol_type = other DNA
cgtctctacccgaacggacggcgcccgttagattgt

SEQ ID NO: 398 - DNA, 36 bp, synthetic construct
  source 1..36: mol_type = other DNA
gtctatcgccccgacggagctagtcgacgacatgcg

SEQ ID NO: 399 - DNA, 36 bp, synthetic construct
  source 1..36: mol_type = other DNA
atgatcgcgtatcgcggtagctcgtcgttacgggcg

SEQ ID NO: 400 - DNA, 36 bp, synthetic construct
  source 1..36: mol_type = other DNA
cgattgtttacggcgccatgctcgacgcgaaccgac

SEQ ID NO: 401 - DNA, 36 bp, synthetic construct
  source 1..36: mol_type = other DNA
acgtccgcgcatcggataagcgcgatatgcgcgtac

SEQ ID NO: 402 - DNA, 36 bp, synthetic construct
  source 1..36: mol_type = other DNA
agcgcgctaactcgatattcgaacggctggtcggac

SEQ ID NO: 403 - DNA, 36 bp, synthetic construct
  source 1..36: mol_type = other DNA
accaaccggcgttacggtagtcgggacgtgactacc

SEQ ID NO: 404 - DNA, 36 bp, synthetic construct
  source 1..36: mol_type = other DNA
tcgttctactacgcggtctcgttcgaccgtgcggtt

SEQ ID NO: 405 - DNA, 36 bp, synthetic construct
  source 1..36: mol_type = other DNA
cgcgcgtaacgaccatcgaagtgtcgacgatcgacc

SEQ ID NO: 406 - DNA, 36 bp, synthetic construct
  source 1..36: mol_type = other DNA
cgtaggagactatcccgctcgcgactcaaatcgcgg

SEQ ID NO: 407 - DNA, 36 bp, synthetic construct
  source 1..36: mol_type = other DNA
gatttacgccgccgcggtatgaggcgaattgcgccg

SEQ ID NO: 408 - DNA, 36 bp, synthetic construct
  source 1..36: mol_type = other DNA
acggagatcgcgttagctctcgaacgatgcgcggtg

SEQ ID NO: 409 - DNA, 36 bp, synthetic construct
  source 1..36: mol_type = other DNA
tctgtaacgcccgaaacggcgcgattcgagtctagg

SEQ ID NO: 410 - DNA, 36 bp, synthetic construct
  source 1..36: mol_type = other DNA
aagcgtgcggtagcgtaagttcaatgcgcgtagtcg

SEQ ID NO: 411 - DNA, 36 bp, synthetic construct
  source 1..36: mol_type = other DNA
cgggcttgcacgacgattaagtgtcgacgatcgacc

SEQ ID NO: 412 - DNA, 36 bp, synthetic construct
  source 1..36: mol_type = other DNA
gtgcgtcatcgtcgtaaagacccgatatcgcgtccg

SEQ ID NO: 413 - DNA, 36 bp, synthetic construct
  source 1..36: mol_type = other DNA
gacgcgcaacggatcgatgatcgacgagtcggagtc

SEQ ID NO: 414 - DNA, 36 bp, synthetic construct
  source 1..36: mol_type = other DNA
gctcgacacgcgcaatgacgaccaaacgttagcacg

SEQ ID NO: 415 - DNA, 36 bp, synthetic construct
  source 1..36: mol_type = other DNA
cgacccgcggagcaactaagtacggattcgaccgtc

SEQ ID NO: 416 - DNA, 36 bp, synthetic construct; identical to SEQ ID NO: 414
  source 1..36: mol_type = other DNA

SEQ ID NO: 417 - DNA, 36 bp, synthetic construct
  source 1..36: mol_type = other DNA
gtattcggacgtagcggtttgccgggcgatcggttg

SEQ ID NO: 418 - DNA, 36 bp, synthetic construct
  source 1..36: mol_type = other DNA
atgcctatcgggtcgcgaccacgagacgtctaacgg

SEQ ID NO: 419 - DNA, 36 bp, synthetic construct
  source 1..36: mol_type = other DNA
cgtacgtgcggttcagctgtcggtagtcgctctcgc

SEQ ID NO: 420 - DNA, 36 bp, synthetic construct
  source 1..36: mol_type = other DNA
cgatgctgtgcgggcgtagtcaattgcgccgtacgc

SEQ ID NO: 421 - DNA, 36 bp, synthetic construct
  source 1..36: mol_type = other DNA
tggagccgacgggcagacgccgttccgctactatat

SEQ ID NO: 422 - DNA, 36 bp, synthetic construct
  source 1..36: mol_type = other DNA
gtggctccagcgtgttctcgaagtgcgagtacgcga

SEQ ID NO: 423 - DNA, 36 bp, synthetic construct
  source 1..36: mol_type = other DNA
cagagtgcactcacgaggctgtatcgcatggtcgcg

SEQ ID NO: 424 - DNA, 36 bp, synthetic construct
  source 1..36: mol_type = other DNA
ccggaccgtacattgccgggtttctctcggcgagga

SEQ ID NO: 425 - DNA, 36 bp, synthetic construct
  source 1..36: mol_type = other DNA
acatctgcacgccggtcgcagacgaaacgcttctca

SEQ ID NO: 426 - DNA, 36 bp, synthetic construct
  source 1..36: mol_type = other DNA
atcgtcgcgcggattcgctacgttcgtcgtagtgga

SEQ ID NO: 427 - DNA, 36 bp, synthetic construct
  source 1..36: mol_type = other DNA
agaacactgtgtggcgccgcccgatccgggtaccgc

SEQ ID NO: 428 - DNA, 36 bp, synthetic construct
  source 1..36: mol_type = other DNA
ctgccgcgacctgttgcggaggtcgacggagcgccg

SEQ ID NO: 429 - DNA, 36 bp, synthetic construct
  source 1..36: mol_type = other DNA
gcgcttcaagtggcgtctggctccggcgagttagag

SEQ ID NO: 430 - DNA, 36 bp, synthetic construct
  source 1..36: mol_type = other DNA
ggccgccattcacgccttgcgccgttaatcttggac

SEQ ID NO: 431 - DNA, 36 bp, synthetic construct
  source 1..36: mol_type = other DNA
atgtggtccgtggactatgcggtcattcccgccgtc

SEQ ID NO: 432 - DNA, 36 bp, synthetic construct
  source 1..36: mol_type = other DNA
accagttcacgctgggtcgcgcgaacccttatctgt

SEQ ID NO: 433 - DNA, 36 bp, synthetic construct
  source 1..36: mol_type = other DNA
ctccggcgggctttaatggcccggaccgccaatcat

SEQ ID NO: 434 - DNA, 36 bp, synthetic construct
  source 1..36: mol_type = other DNA
acctggccaagcgggaacgtctatctcgctgctgaa

SEQ ID NO: 435 - DNA, 36 bp, synthetic construct
  source 1..36: mol_type = other DNA
cgagttgggcgtcaatgtcggccgtcatgatgcacc

SEQ ID NO: 436 - DNA, 36 bp, synthetic construct
  source 1..36: mol_type = other DNA
gccgtcgttggtacctcgatcggtcccggaagcggc

SEQ ID NO: 437 - DNA, 36 bp, synthetic construct
  source 1..36: mol_type = other DNA
gcggatcggcccgcgggagtcggtgacagattcttg

SEQ ID NO: 438 - DNA, 36 bp, synthetic construct
  source 1..36: mol_type = other DNA
gaagcctggcggattggtaggccgctttccggactc

SEQ ID NO: 439 - DNA, 36 bp, synthetic construct
  source 1..36: mol_type = other DNA
ccaggccgtcaaacgtcggaggtccggctgcgacat

SEQ ID NO: 440 - DNA, 36 bp, synthetic construct
  source 1..36: mol_type = other DNA
caccgtcgagcaggccgtccttaaggctctaggctg

SEQ ID NO: 441 - DNA, 36 bp, synthetic construct
  source 1..36: mol_type = other DNA
ggccgcgcgctcgctagaggttcacgaaccttgtgc

SEQ ID NO: 442 - DNA, 36 bp, synthetic construct
  source 1..36: mol_type = other DNA
ggatgcgaaagccctcgcgccgaaatcgtggattct

SEQ ID NO: 443 - DNA, 36 bp, synthetic construct
  source 1..36: mol_type = other DNA
cgcccgttatcatacgagccctaggcgttggtccgg

SEQ ID NO: 444 - DNA, 36 bp, synthetic construct
  source 1..36: mol_type = other DNA
ggcgaaatcccgtgtgcgagagacgcgaaggcagat

SEQ ID NO: 445 - DNA, 36 bp, synthetic construct
  source 1..36: mol_type = other DNA
tgccgcagtgcgtaagctacgcgctcgttccatact

SEQ ID NO: 446 - DNA, 36 bp, synthetic construct
  source 1..36: mol_type = other DNA
tgccagggctcgaccagttggactcgcaggacatca

SEQ ID NO: 447 - DNA, 36 bp, synthetic construct
  source 1..36: mol_type = other DNA
ggcgggtccaagtatgccgactgcgatcgacaggat

SEQ ID NO: 448 - DNA, 36 bp, synthetic construct
  source 1..36: mol_type = other DNA
cctctcagcggtccgatcggctggaattcgccagct

SEQ ID NO: 449 - DNA, 36 bp, synthetic construct
  source 1..36: mol_type = other DNA
acatgcgtactgcgttccttccggtccgcgacgagt

SEQ ID NO: 450 - DNA, 36 bp, synthetic construct
  source 1..36: mol_type = other DNA
ctagcctcacgccctgcgaaccgaccgttgccatcg

SEQ ID NO: 451 - DNA, 36 bp, synthetic construct
  source 1..36: mol_type = other DNA
agatagtgggccatcggcactccgggctaagccggt

SEQ ID NO: 452 - DNA, 36 bp, synthetic construct
  source 1..36: mol_type = other DNA
ccacgtgcgccatacactcccgacgcctgtcctctc

SEQ ID NO: 453 - DNA, 36 bp, synthetic construct
  source 1..36: mol_type = other DNA
agctgccgaatctacaagggccgactagcagcctgc

SEQ ID NO: 454 - DNA, 36 bp, synthetic construct
  source 1..36: mol_type = other DNA
agcagcctaggcgcctttgtcggtttgactcacgtg

SEQ ID NO: 455 - DNA, 36 bp, synthetic construct
  source 1..36: mol_type = other DNA
atcgcgccttgtgtcgttccgtggatcagcaccgca

SEQ ID NO: 456 - DNA, 36 bp, synthetic construct
  source 1..36: mol_type = other DNA
gacctacgactcgtgtgccgcgcatgtcatggtgga

SEQ ID NO: 457 - DNA, 36 bp, synthetic construct
  source 1..36: mol_type = other DNA
aaccctaagggcgttgcgcgatgcacgcgcatggag

SEQ ID NO: 458 - DNA, 36 bp, synthetic construct
  source 1..36: mol_type = other DNA
cacggcctcggtggtcggccggagcatcagaccgta

SEQ ID NO: 459 - DNA, 36 bp, synthetic construct
  source 1..36: mol_type = other DNA
cgcgtatcccttcggcctttcggttcgtgtggtgtt

SEQ ID NO: 460 - DNA, 36 bp, synthetic construct
  source 1..36: mol_type = other DNA
tcagggtcccggttacgcgactataggaggagccga

SEQ ID NO: 461 - DNA, 36 bp, synthetic construct
  source 1..36: mol_type = other DNA
gagcccgcggttgttgataccgggactatgcgcgac

SEQ ID NO: 462 - DNA, 36 bp, synthetic construct
  source 1..36: mol_type = other DNA
agggagcgcgtggtagatccaattcgatacggccgt

SEQ ID NO: 463 - DNA, 36 bp, synthetic construct
  source 1..36: mol_type = other DNA
gcacgcagagcaatcaaccgataaggcacgccgagt

SEQ ID NO: 464 - DNA, 36 bp, synthetic construct
  source 1..36: mol_type = other DNA
gccacagggcgactcggagggactctgttcggatga

SEQ ID NO: 465 - DNA, 36 bp, synthetic construct
  source 1..36: mol_type = other DNA
agtgtggcggacaagcagtggctcgcgaggcagttc

SEQ ID NO: 466 - DNA, 36 bp, synthetic construct
  source 1..36: mol_type = other DNA
aggtcgaccggattcgttatccagcgcagcgtggtt

SEQ ID NO: 467 - DNA, 36 bp, synthetic construct
  source 1..36: mol_type = other DNA
tcaaatagcgctcccgtgcacaaggccgctcctagg

SEQ ID NO: 468 - DNA, 36 bp, synthetic construct
  source 1..36: mol_type = other DNA
acctggccgttccgcgacacaggcacgggctactct

SEQ ID NO: 469 - DNA, 36 bp, synthetic construct
  source 1..36: mol_type = other DNA
gcgggctagctgaacgtctttcccggccactgccta

SEQ ID NO: 470 - DNA, 36 bp, synthetic construct
  source 1..36: mol_type = other DNA
aggcaagtgggtacgcgttaacagtgagcgcggctc

SEQ ID NO: 471 - DNA, 36 bp, synthetic construct
  source 1..36: mol_type = other DNA
tgctcctaggtggatacatcgccgctcggtccacct

SEQ ID NO: 472 - DNA, 36 bp, synthetic construct
  source 1..36: mol_type = other DNA
ggcgatagcgcgtttactttggtccgcacgacacgt

SEQ ID NO: 473 - DNA, 36 bp, synthetic construct
  source 1..36: mol_type = other DNA
acgtcgaatgggtaccggcgcgcctacttcgctact

SEQ ID NO: 474 - DNA, 36 bp, synthetic construct
  source 1..36: mol_type = other DNA
aagtgcgatatgcgagcgctgttccgcatgccgcac

SEQ ID NO: 475 - DNA, 36 bp, synthetic construct
  source 1..36: mol_type = other DNA
ggcaggctgatcgggaccggcccgtaggttcgcttc

SEQ ID NO: 476 - DNA, 36 bp, synthetic construct
  source 1..36: mol_type = other DNA
catgagcgtgttgcgtcggacaaggagggagtcgct

SEQ ID NO: 477 - DNA, 36 bp, synthetic construct
  source 1..36: mol_type = other DNA
gctagggtgaatcgggcacgaggcgtcgcatgtcat

SEQ ID NO: 478 - DNA, 36 bp, synthetic construct
  source 1..36: mol_type = other DNA
agagggcctactcgttgacagcgcgacgccacaagc

SEQ ID NO: 479 - DNA, 36 bp, synthetic construct
  source 1..36: mol_type = other DNA
cggcgtcggcacgtcggtcgaacactatgcgaacac

SEQ ID NO: 480 - DNA, 36 bp, synthetic construct
  source 1..36: mol_type = other DNA
actggtgctgtagttcggcccggctcccagcaagaa

SEQ ID NO: 481 - DNA, 36 bp, synthetic construct
  source 1..36: mol_type = other DNA
agacggtcgaataaggtagcggcggccgactccgtc

SEQ ID NO: 482 - DNA, 36 bp, synthetic construct
  source 1..36: mol_type = other DNA
tatcacatggcggacgggtgcatacgccctgcgttc

SEQ ID NO: 483 - DNA, 36 bp, synthetic construct
  source 1..36: mol_type = other DNA
aggctcgcgatgaacggatgcgctaccgacatggct

SEQ ID NO: 484 - DNA, 36 bp, synthetic construct
  source 1..36: mol_type = other DNA
gcgcagtagctaagacgcgcgttaccatgccgccag

SEQ ID NO: 485 - DNA, 36 bp, synthetic construct
  source 1..36: mol_type = other DNA
agttggatcgcgcacgaccgaccgaggatcgcagac

SEQ ID NO: 486 - DNA, 36 bp, synthetic construct
  source 1..36: mol_type = other DNA
cgcggagcacggcgtccacttaatcgcacgcagcaa

SEQ ID NO: 487 - DNA, 36 bp, synthetic construct
  source 1..36: mol_type = other DNA
tcccgttgtcgatcctacccgccggtgcggattatg

SEQ ID NO: 488 - DNA, 36 bp, synthetic construct
  source 1..36: mol_type = other DNA
ggttcgctgttatcgggtgggaacgaccagcggtgc

SEQ ID NO: 489 - DNA, 36 bp, synthetic construct
  source 1..36: mol_type = other DNA
accaccaacactcggatagttctcgccgttccgcct

SEQ ID NO: 490 - DNA, 36 bp, synthetic construct
  source 1..36: mol_type = other DNA
ggtagggagcgcgaggcatgataggagcgatgtggt

SEQ ID NO: 491 - DNA, 36 bp, synthetic construct
  source 1..36: mol_type = other DNA
ggcagctgatgcattggcgtgcgcgccattacctcc

SEQ ID NO: 492 - DNA, 36 bp, synthetic construct
  source 1..36: mol_type = other DNA
caaggcacgccgcgttcggcacgcgacctaccagca

SEQ ID NO: 493 - DNA, 36 bp, synthetic construct
  source 1..36: mol_type = other DNA
ttgaggtcgccggaactacgcgagtccgcgaagagt

SEQ ID NO: 494 - DNA, 36 bp, synthetic construct
  source 1..36: mol_type = other DNA
gccctgaaacgtgtaagctcctgccgcgaaccgtaa

SEQ ID NO: 495 - DNA, 36 bp, synthetic construct
  source 1..36: mol_type = other DNA
tattcccgcccgagactcacgaggtcggttgccgtg

SEQ ID NO: 496 - DNA, 36 bp, synthetic construct
  source 1..36: mol_type = other DNA
tcagccagcggaatgggcggaccgttacggtcagta

SEQ ID NO: 497 - DNA, 36 bp, synthetic construct
  source 1..36: mol_type = other DNA
cggcggagcgttcgagtacaacctgtcttgagcgcg

SEQ ID NO: 498 - DNA, 36 bp, synthetic construct
  source 1..36: mol_type = other DNA
ctcatagtgacagcggatccgctcacggctccgtga

SEQ ID NO: 499 - DNA, 36 bp, synthetic construct
  source 1..36: mol_type = other DNA
gtagtctgcgtacgagtccgggcggtgcatgttcgt

SEQ ID NO: 500 - DNA, 36 bp, synthetic construct
  source 1..36: mol_type = other DNA
tgaccatgagcacgtcacccacgcgcatgcagtatc

SEQ ID NO: 501 - DNA, 36 bp, synthetic construct
  source 1..36: mol_type = other DNA
tgtttcggcctgtgagctgccggccctgtatgctag

SEQ ID NO: 502 - DNA, 36 bp, synthetic construct
  source 1..36: mol_type = other DNA
ctgcggacgttgaggcggctaatgggaagacgacct

SEQ ID NO: 503 - DNA, 36 bp, synthetic construct
  source 1..36: mol_type = other DNA
gacaaattaagagtcgcggcggaccgcgccttgact

SEQ ID NO: 504 - DNA, 36 bp, synthetic construct
  source 1..36: mol_type = other DNA
cagcagggttaatgcgcgcgctggcaacgttagctc

SEQ ID NO: 505 - DNA, 36 bp, synthetic construct
  source 1..36: mol_type = other DNA
ccacgagccctcaatgaggctggccgtccgaagatc

SEQ ID NO: 506 - DNA, 36 bp, synthetic construct
  source 1..36: mol_type = other DNA
atcgacgcattcggtgtggcccgtctggtggtagct

SEQ ID NO: 507 - DNA, 36 bp, synthetic construct
  source 1..36: mol_type = other DNA
cctgatggcgaggcttgcgacggcattgctcaggct

SEQ ID NO: 508 - DNA, 36 bp, synthetic construct
  source 1..36: mol_type = other DNA
cgcctagcgcgtggagctgcgcaaagtgtcacgaag

SEQ ID NO: 509 - DNA, 36 bp, synthetic construct
  source 1..36: mol_type = other DNA
cgggcccgaccacggcatgttccatcgtcggtcact

SEQ ID NO: 510 - DNA, 36 bp, synthetic construct
  source 1..36: mol_type = other DNA
tacatcgctggtgttcaccacgcagcgagtacggcg

SEQ ID NO: 511 - DNA, 36 bp, synthetic construct
  source 1..36: mol_type = other DNA
tcccgagtctcttcaagggcgtgggcgcttccagtt

SEQ ID NO: 512 - DNA, 36 bp, synthetic construct
  source 1..36: mol_type = other DNA
cgaagtcagcgctgaatgagcgggaccgttaccggt

SEQ ID NO: 513 - DNA, 36 bp, synthetic construct
  source 1..36: mol_type = other DNA
cgagtgctgccagcctggctggcctagcgcttccag

SEQ ID NO: 514 - DNA, 36 bp, synthetic construct
  source 1..36: mol_type = other DNA
acgtcagaatgctgggcagcctcaaggctcggagcc

SEQ ID NO: 515 - DNA, 36 bp, synthetic construct
  source 1..36: mol_type = other DNA
taccgttcacaaccacagggcgcgagcgcaaccgcg

SEQ ID NO: 516 - DNA, 36 bp, synthetic construct
  source 1..36: mol_type = other DNA
tcgacaccgagtttgccctcctatttgctggcggcg

SEQ ID NO: 517 - DNA, 36 bp, synthetic construct
  source 1..36: mol_type = other DNA
gtccgttagattggagttctgtcagcgccctcgcgc

SEQ ID NO: 518 - DNA, 36 bp, synthetic construct
  source 1..36: mol_type = other DNA
gtcgccgaacaccctgacttccaccgtgtgttccga

SEQ ID NO: 519 - DNA, 36 bp, synthetic construct
  source 1..36: mol_type = other DNA
ccgggatagctccacctgagtagccgcgccatctac

SEQ ID NO: 520 - DNA, 36 bp, synthetic construct
  source 1..36: mol_type = other DNA
tgcatgtttggtgtggatgccacagctcagcgcgag

SEQ ID NO: 521 - DNA, 36 bp, synthetic construct
  source 1..36: mol_type = other DNA
ggtcagggagctcgtgccaagacccacagaccgtcc

SEQ ID NO: 522 - DNA, 36 bp, synthetic construct
  source 1..36: mol_type = other DNA
tacggtcaagatcaccaggccggctgcgtcttgtca

SEQ ID NO: 523 - DNA, 36 bp, synthetic construct
  source 1..36: mol_type = other DNA
aaagactattgcccagcgggcgggctgactgaaggc

SEQ ID NO: 524 - DNA, 36 bp, synthetic construct
  source 1..36: mol_type = other DNA
tggctgccacgcattgaagcaaagacgcgaagacgg

SEQ ID NO: 525 - DNA, 36 bp, synthetic construct
  source 1..36: mol_type = other DNA
ttggctggatcccaagtctggcccgagggacggtac

SEQ ID NO: 526 - DNA, 36 bp, synthetic construct
  source 1..36: mol_type = other DNA
accggcctgcatttcagctctcggagcctgaagctg

SEQ ID NO: 527 - DNA, 36 bp, synthetic construct
  source 1..36: mol_type = other DNA
gttgaacgcacccgggcttcctctgggaatcgcaga

SEQ ID NO: 528 - DNA, 36 bp, synthetic construct
  source 1..36: mol_type = other DNA
atcacggacacccaagtggcgcgcctcttaccacgc

SEQ ID NO: 529 - DNA, 36 bp, synthetic construct
  source 1..36: mol_type = other DNA
ccgagacagtgcacgcctcacgtggacagctgccat

SEQ ID NO: 530 - DNA, 29 bp, synthetic construct
  source 1..29: mol_type = other DNA
acaaaggcagccacgcactccttccctgt

SEQ ID NO: 531 - DNA, 32 bp, synthetic construct
  source 1..32: mol_type = other DNA
acactctttccctacacgacgctcttccgatc

SEQ ID NO: 532 - DNA, 29 bp, synthetic construct
  source 1..29: mol_type = other DNA
ctccagcgagatgaccctcaccaaccact

SEQ ID NO: 533 - DNA, 33 bp, synthetic construct
  source 1..33: mol_type = other DNA
gtgactggagttcagacgtgtgctcttccgatc

SEQ ID NO: 534 - DNA, 19 bp, synthetic construct
  source 1..19: mol_type = other DNA
aatgatacggcgaccaccg

SEQ ID NO: 535 - DNA, 24 bp, synthetic construct
  source 1..24: mol_type = other DNA
caagcagaagacggcatacgagat

SEQ ID NO: 536 - DNA, 32 bp, synthetic construct
  source 1..32: mol_type = other DNA
aacgccaaacctacggctttacttcctgtggc

SEQ ID NO: 537 - DNA, 32 bp, synthetic construct
  source 1..32: mol_type = other DNA
tcttgagtcattcgcagggcatgtgccagacc

SEQ ID NO: 538 - DNA, 32 bp, synthetic construct
  source 1..32: mol_type = other DNA
tcggcgttgtctgctatcgttcttggcactcc

SEQ ID NO: 539 - DNA, 32 bp, synthetic construct
  source 1..32: mol_type = other DNA
ggagcaataaccataaggccgttgacaagccc

SEQ ID NO: 540 - DNA, 32 bp, synthetic construct
  source 1..32: mol_type = other DNA
ggcgtattgccttggttctggcagcctcattg

SEQ ID NO: 541 - DNA, 32 bp, synthetic construct
  source 1..32: mol_type = other DNA
cagcagagggaacgatttcaacttcctgtggc

SEQ ID NO: 542 - DNA, 32 bp, synthetic construct
  source 1..32: mol_type = other DNA
ctactgcaagggtgtctagaatgtgccagacc

SEQ ID NO: 543 - DNA, 32 bp, synthetic construct
  source 1..32: mol_type = other DNA
gaccgactcgtgaaacgtaatcttggcactcc

SEQ ID NO: 544 - DNA, 32 bp, synthetic construct
  source 1..32: mol_type = other DNA
acacattctttgcgcccagagttgacaagccc

SEQ ID NO: 545 - DNA, 32 bp, synthetic construct
  source 1..32: mol_type = other DNA
atttcattcgacacccggtcgcagcctcattg

SEQ ID NO: 546 - DNA, 32 bp, synthetic construct
  source 1..32: mol_type = other DNA
agccacaggaagtaaagccgtaggtttggcgt

SEQ ID NO: 547 - DNA, 33 bp, synthetic construct
  source 1..33: mol_type = other DNA
aggtctggcacatgccctgcgaatgactcaaga

SEQ ID NO: 548 - DNA, 33 bp, synthetic construct
  source 1..33: mol_type = other DNA
aggagtgccaagaacgatagcagacaacgccga

SEQ ID NO: 549 - DNA, 33 bp, synthetic construct
  source 1..33: mol_type = other DNA
agggcttgtcaacggccttatggttattgctcc

SEQ ID NO: 550 - DNA, 33 bp, synthetic construct
  source 1..33: mol_type = other DNA
acaatgaggctgccagaaccaaggcaatacgcc

SEQ ID NO: 551 - DNA, 33 bp, synthetic construct
  source 1..33: mol_type = other DNA
agccacaggaagttgaaatcgttccctctgctg

SEQ ID NO: 552 - DNA, 33 bp, synthetic construct
  source 1..33: mol_type = other DNA
aggtctggcacattctagacacccttgcagtag

SEQ ID NO: 553 - DNA, 33 bp, synthetic construct
  source 1..33: mol_type = other DNA
aggagtgccaagattacgtttcacgagtcggtc

SEQ ID NO: 554 - DNA, 33 bp, synthetic construct
  source 1..33: mol_type = other DNA
agggcttgtcaactctgggcgcaaagaatgtgt

SEQ ID NO: 555 - DNA, 33 bp, synthetic construct
  source 1..33: mol_type = other DNA
acaatgaggctgcgaccgggtgtcgaatgaaat

SEQ ID NO: 556 - DNA, 32 bp, synthetic construct
  source 1..32: mol_type = other DNA
tgttgcatctccacccggattgagccttcagc

SEQ ID NO: 557 - DNA, 32 bp, synthetic construct
  source 1..32: mol_type = other DNA
cacaacgggagctgtggaattggttcacctgg

SEQ ID NO: 558 - DNA, 32 bp, synthetic construct
  source 1..32: mol_type = other DNA
tggactaagactcgtcctccagcggacctaag

SEQ ID NO: 559 - DNA, 32 bp, synthetic construct
  source 1..32: mol_type = other DNA
gtatgatggtgttgcggcttctcgcttaacgc

SEQ ID NO: 560 - DNA, 32 bp, synthetic construct
  source 1..32: mol_type = other DNA
tctgagtgccagtgacttcacgcattcgcttg

SEQ ID NO: 561 - DNA, 32 bp, synthetic construct
  source 1..32: mol_type = other DNA
tacgacacactcgggctctatgggcttcatgg

SEQ ID NO: 562 - DNA, 32 bp, synthetic construct
  source 1..32: mol_type = other DNA
gtttgagtgaaggcggtccaacccttagtgcg

SEQ ID NO: 563 - DNA, 32 bp, synthetic construct
  source 1..32: mol_type = other DNA
ctataagtttgtcgtgcccgtgagccttcagc

SEQ ID NO: 564 - DNA, 32 bp, synthetic construct
  source 1..32: mol_type = other DNA
ggagtgacactgactacgtttggttcacctgg

SEQ ID NO: 565 - DNA, 32 bp, synthetic construct
  source 1..32: mol_type = other DNA
gtcaacgccctagcagacatagcggacctaag

SEQ ID NO: 566 - DNA, 32 bp, synthetic construct
  source 1..32: mol_type = other DNA
ccagaacctattgagcctgactcgcttaacgc

SEQ ID NO: 567 - DNA, 32 bp, synthetic construct
  source 1..32: mol_type = other DNA
aggtgttcgtacaatgaggccgcattcgcttg

SEQ ID NO: 568 - DNA, 32 bp, synthetic construct
  source 1..32: mol_type = other DNA
tggtcaagggcaactaatcctgggcttcatgg

SEQ ID NO: 569 - DNA, 32 bp, synthetic construct
  source 1..32: mol_type = other DNA
acaattacccgtttaccggcacccttagtgcg

SEQ ID NO: 570 - DNA, 33 bp, synthetic construct
  source 1..33: mol_type = other DNA
agctgaaggctcaatccgggtggagatgcaaca

SEQ ID NO: 571 - DNA, 33 bp, synthetic construct
  source 1..33: mol_type = other DNA
accaggtgaaccaattccacagctcccgttgtg

SEQ ID NO: 572 - DNA, 33 bp, synthetic construct
  source 1..33: mol_type = other DNA
acttaggtccgctggaggacgagtcttagtcca

SEQ ID NO: 573 - DNA, 33 bp, synthetic construct
  source 1..33: mol_type = other DNA
agcgttaagcgagaagccgcaacaccatcatac

SEQ ID NO: 574 - DNA, 33 bp, synthetic construct
  source 1..33: mol_type = other DNA
acaagcgaatgcgtgaagtcactggcactcaga

SEQ ID NO: 575 - DNA, 33 bp, synthetic construct
  source 1..33: mol_type = other DNA
accatgaagcccatagagcccgagtgtgtcgta

SEQ ID NO: 576 - DNA, 33 bp, synthetic construct
  source 1..33: mol_type = other DNA
acgcactaagggttggaccgccttcactcaaac

SEQ ID NO: 577 - DNA, 33 bp, synthetic construct
  source 1..33: mol_type = other DNA
agctgaaggctcacgggcacgacaaacttatag

SEQ ID NO: 578 - DNA, 33 bp, synthetic construct
  source 1..33: mol_type = other DNA
accaggtgaaccaaacgtagtcagtgtcactcc

SEQ ID NO: 579 - DNA, 33 bp, synthetic construct
  source 1..33: mol_type = other DNA
acttaggtccgctatgtctgctagggcgttgac

SEQ ID NO: 580 - DNA, 33 bp, synthetic construct
  source 1..33: mol_type = other DNA
agcgttaagcgagtcaggctcaataggttctgg

SEQ ID NO: 581 - DNA, 33 bp, synthetic construct
  source 1..33: mol_type = other DNA
acaagcgaatgcggcctcattgtacgaacacct

SEQ ID NO: 582 - DNA, 33 bp, synthetic construct
  source 1..33: mol_type = other DNA
accatgaagcccaggattagttgcccttgacca

SEQ ID NO: 583 - DNA, 33 bp, synthetic construct
  source 1..33: mol_type = other DNA
acgcactaagggtgccggtaaacgggtaattgt

SEQ ID NO: 584 - DNA, 36 bp, synthetic construct; identical to SEQ ID NO: 446
  source 1..36: mol_type = other DNA

SEQ ID NO: 585 - DNA, 36 bp, synthetic construct; identical to SEQ ID NO: 508
  source 1..36: mol_type = other DNA

SEQ ID NO: 586 - DNA, 36 bp, synthetic construct; identical to SEQ ID NO: 435
  source 1..36: mol_type = other DNA

SEQ ID NO: 587 - DNA, 36 bp, synthetic construct; identical to SEQ ID NO: 500
  source 1..36: mol_type = other DNA

SEQ ID NO: 588 - DNA, 18 bp, synthetic construct
  source 1..18: mol_type = other DNA
actggtcgagccctggca

SEQ ID NO: 589 - DNA, 18 bp, synthetic construct
  source 1..18: mol_type = other DNA
agctccacgcgctaggcg

SEQ ID NO: 590 - DNA, 18 bp, synthetic construct
  source 1..18: mol_type = other DNA
acattgacgcccaactcg

SEQ ID NO: 591 - DNA, 18 bp, synthetic construct
  source 1..18: mol_type = other DNA
gtgacgtgctcatggtca

SEQ ID NO: 592 - DNA, 18 bp, synthetic construct
  source 1..18: mol_type = other DNA
tgatgtcctgcgagtcca

SEQ ID NO: 593 - DNA, 18 bp, synthetic construct
  source 1..18: mol_type = other DNA
cttcgtgacactttgcgc

SEQ ID NO: 594 - DNA, 18 bp, synthetic construct
  source 1..18: mol_type = other DNA
ggtgcatcatgacggccg

SEQ ID NO: 595 - DNA, 18 bp, synthetic construct
  source 1..18: mol_type = other DNA
gatactgcatgcgcgtgg

(19) United States
(12) Patent Application Publication
(10) Pub. No.: US 2025/0243532 A1
(43) Pub. Date: Jul. 31, 2025

(54) OLIGONUCLEOTIDE CONJUGATES USEFUL FOR IN SITU TARGET DETECTION

(71) Applicant: Singular Genomics Systems, Inc., San Diego, CA (US)

(72) Inventor: Andrew Pawlowski, San Diego, CA (US)

(21) Appl. No.: 19/038,486

(22) Filed: Jan. 27, 2025

Related U.S. Application Data
(60) Provisional application No. 63/625,977, filed on Jan. 28, 2024.
(60) Provisional application No. 63/671,405, filed on Jul. 15, 2024.

(51) Int. Cl. C12Q 1/6841; G01N 21/64

(52) U.S. Cl. CPC C12Q 1/6841; G01N 21/6428; G01N 2021/6439

(57) ABSTRACT

Disclosed herein, inter alia, are compositions and methods useful for interrogating a cell and/or tissue.

### CROSS-REFERENCES TO RELATED APPLICATIONS

This application claims the benefit of U.S. Provisional Application No. 63/625,977, filed Jan. 28, 2024, and U.S. Provisional Application No. 63/671,405, filed Jul. 15, 2024, each of which are incorporated herein by reference in their entirety and for all purposes.

### SEQUENCE LISTING

The instant application contains a Sequence Listing which has been submitted electronically in XML format and is hereby incorporated by reference in its entirety. Said XML copy, created on Jan. 17, 2025, is named 00613001US.xml, and is 529,496 bytes in size.

### BACKGROUND

The field of cellular biology has recently been engaged in the intricate task of identifying and quantifying proteins within their native cellular environments. This endeavor, known as in situ protein detection, is pivotal for understanding the complex interplay of biomolecules within cells, thus offering insights into cellular functions, signaling pathways, and the underlying mechanisms of various diseases. Traditional methods of protein detection, such as immunohistochemistry and fluorescence microscopy, have provided substantial information. However, these techniques often face limitations in sensitivity, specificity, and the ability to simultaneously detect multiple proteins within the complex milieu of the cell. The recent development of molecular techniques has revolutionized the landscape of in situ protein detection. Integrating nucleic acid-based methods (e.g., sequencing) with traditional protein detection strategies (e.g., antibody-oligo conjugates) opens new avenues for enhancing sensitivity, specificity, and multiplexing. These advancements are crucial for high-throughput multiplexed studies, enabling the simultaneous detection of multiple proteins, and is particularly important in the study of cellular heterogeneity and in diseases like cancer, where the expression levels of numerous proteins can provide vital diagnostic and prognostic information. The ability to accurately and efficiently detect a wide array of proteins in situ is, therefore, a cornerstone in both basic biological research and clinical diagnostics.

Despite these advancements, challenges persist in optimizing molecular techniques for in situ protein detection. The need for high specificity and sensitivity necessitates the development of methods that can reliably distinguish between numerous protein targets in a complex cellular environment without unintended interactions with cellular components. This requires careful consideration of probe design, target accessibility, and signal amplification strategies. Furthermore, the integration of these molecular tools into existing microscopy and imaging platforms is essential for the visualization and quantification of proteins at the cellular and subcellular levels. Addressing these challenges is critical for advancing our understanding of cellular processes and for the development of novel diagnostic and therapeutic approaches. Disclosed herein, inter alia, are solutions to these and other problems in the art.

### BRIEF SUMMARY

In an aspect is provided a composition. In embodiments, the composition includes a specific binding agent covalently attached to an oligonucleotide, wherein the oligonucleotide includes a sequence at least 80% identical to a sequence selected from SEQ ID NO:1 to SEQ ID NO: 132. In embodiments, one or more polynucleotides are bound to the oligonucleotide. In embodiments, the oligonucleotide is hybridized to a first blocking oligonucleotide and a second blocking oligonucleotide. In embodiments, the oligonucleotide is 30 to 40 nucleotides. In embodiments, the oligonucleotide does not include five consecutive weak bases. In embodiments, the oligonucleotide does not include five consecutive strong bases. In embodiments, the oligonucleotide does not comprise secondary structure. In embodiments, the composition is in, on, or within a cell or tissue.

In an aspect is provided a method of detecting a target molecule (e.g., a protein, carbohydrate, or nucleic acid molecule). In embodiments, the method includes detecting the target molecule in or on a cell or tissue. In embodiments, the method includes binding a specific binding agent including an oligonucleotide to the target molecule in or on a cell or tissue, wherein the oligonucleotide includes a first blocking oligonucleotide hybridized to a first sequence of the oligonucleotide, and a second blocking oligonucleotide hybridized to a second sequence of the oligonucleotide. In embodiments, the method includes removing the blocking oligonucleotides. In embodiments, the method includes binding a polynucleotide probe including a first binding sequence and a second binding sequence to an oligonucleotide, wherein the oligonucleotide is covalently bound to a specific binding agent and includes a sequence selected from SEQ ID NO:1 to SEQ ID NO:132; binding a polynucleotide to the sequence and detecting the polynucleotide, thereby detecting the target molecule. In embodiments, detecting includes serially contacting the oligonucleotides with labeled probes (e.g., labeled oligonucleotides or labeled nucleotides).

### BRIEF DESCRIPTION OF THE DRAWINGS

FIG. 1 provides an illustration of a probe conjugate described herein (e.g., a specific binding agent described herein). The embodiment depicted in FIG. 1 includes an antibody covalently attached to two oligonucleotides via independent bioconjugate linkers. The oligonucleotide provides a sequence that is associated with the antibody, and so when the sequence of the oligonucleotide is inferred or detected the identity of the antibody and thus the target protein of interest is identified.

FIG. 2. Following binding of the antibody-oligo (Ab-O) conjugate to the cell or tissue, the oligonucleotides covalently attached to the Ab-O are detected with a circular, or circularizable, probe (e.g., a polynucleotide probe described herein). A type of circularizable probe is a padlock probe (PLP) which is a linear polynucleotide that is rendered into a circular polynucleotide following hybridization to the oligonucleotide and ligation of the 5′ and 3′ ends. Shown in the inset of FIG. 2 is the sequence (SEQ ID NO:1) provided by the oligonucleotide attached to the antibody and a polynucleotide probe bound thereto. The left side (LS) sequence is SEQ ID NO:133 and the right side (RS) sequence is provided as SEQ ID NO:265. The polynucleotide probe may further include a primer binding sequence, for example, a sequencing primer binding sequence (labelled as SP binding site) and an ID. The ID may be one or more nucleotides conferring the identity of the PLP. For example, the ID may be a unique molecular identifying (UMI) sequence, barcode nucleotide, or a barcode sequence. The ID may be a single nucleotide or an identifying sequence of 2, 3, 4, 5, 6, 7, 8, 9, 10, 11 or 12 nucleotides. While sequencing the LS and/or RS may provide identification information, it may be useful to sequence the ID nucleotide(s) as a means for error checking.

FIG. 3 provides a workflow for designing effective oligonucleotide sequences. A pool of initial oligonucleotides was generated using random sequences with a length of 36 base pairs. Following this initial generation, the pool underwent several filtering steps, though except for the first step, the order in which these steps were performed could have been interchanged. The oligonucleotides were first selected based on their GC content, ensuring that it fell within the range of 40-60% over an 18 base pair stretch, which represents the binding regions for LS and RS. Any oligonucleotides that had homopolymer sequences greater than or equal to 4 were then excluded. Oligonucleotides containing the sequences SSSSS and/or WWWWW were subsequently removed from the pool, wherein S denotes strong bases (G or C) and W denotes weak bases (A or T) according to IUPAC naming convention for DNA beyond just the standard nucleotides. Furthermore, oligonucleotides that demonstrated a strong tendency to form secondary structures were eliminated. The pool was further refined by eliminating any oligonucleotides that overlapped with known transcriptome sequences. Oligonucleotides that had overlaps with each other were also removed. Overlaps were determined using BLAST with a word size of 10 for both transcriptome and primer orthogonality, ensuring there cannot be any shared 10-mer sequences (complement or reverse complement). Oligonucleotides that might have overlapped with commonly used primer sequences such as sequencing primers or amplification primers were excluded from the pool. As a final filtering step, oligonucleotides that might have bound due to strong secondary structures were excluded. After these comprehensive filtering steps, the refined pool of oligonucleotides was experimentally verified to ensure the accuracy and suitability of the sequences for their intended application.

FIGS. 4A-4B. The antibody-oligo (Ab-O) conjugates (e.g., a specific binding agent described herein) may include a blocking strand that is substantially the same length as the oligonucleotide as illustrated in FIG. 4A. For example, if the oligonucleotide provided by the Ab-O conjugate includes SEQ ID NO:1, the single blocking strand includes both the LS (SEQ ID NO: 133) and RS (SEQ ID NO:265) sequences. Alternatively, as illustrated in FIG. 4B, the blocking oligonucleotide may be provided in two (or more) parts. In embodiments, a first blocking oligonucleotide includes the LS sequence (SEQ ID NO:133) and a second blocking oligonucleotide includes the RS sequence (SEQ ID NO:265).

FIG. 5 provides fluorescent images of proteins detected using the Ab-O conjugates described herein. The proteins include PD-1, PD-L1, CD56, CD8, HLA-DR, CD4, CD3, Ki-67, CD20, ATPase, CD45RA, and PanCk shown in individual channels and the composite image (left) for a tonsil tissue section. The scale bar is shown as 1000 μm.

FIG. 6 provides an illustration of the sequential collection of information to inform on the structure of a cell and/or tissue. Spectrally distinct dyes are used in the first set, and optionally reused in subsequent sets. For example, the first set includes Alexa Fluor® 532 (emission: 532 nm), Alexa Fluor® 594 (emission: 594 nm), Alexa Fluor® 647 (emission: 647 nm), and Alexa Fluor® 680 (emission: 680 nm) to illuminate the Golgi Apparatus, endoplasmic reticulum, actin, lysosomes, and specific cell surface receptors of a cell. Following cleavage and removal of the fluorophores, the second set of targeting molecules are incubated with the sample cell. The second set can then illuminate the nucleus, nucleoli, mitochondria, nuclear envelop, cell surface receptors, and plasma membrane. The sequential addition of cell paints can continue for N cycles providing additional information about the cell. The resulting images may be computationally processed and overlaid to provide a composite image of the cell and/or tissue.

### DETAILED DESCRIPTION

The aspects and embodiments described herein relate to effective probes and sequences that enable efficient target detection and minimize non-specific binding in samples.

### I. Definitions

All patents, patent applications, articles and publications mentioned herein, both supra and infra, are hereby expressly incorporated herein by reference in their entireties. The practice of the technology described herein will employ, unless indicated specifically to the contrary, conventional methods of chemistry, biochemistry, organic chemistry, molecular biology, bioinformatics, microbiology, recombinant DNA techniques, genetics, immunology, and cell biology that are within the skill of the art, many of which are described below for the purpose of illustration. Examples of such techniques are available in the literature. See, e.g., Singleton et al., DICTIONARY OF MICROBIOLOGY AND MOLECULAR BIOLOGY 2nd ed., J. Wiley & Sons (New York, NY 1994); and Sambrook and Green, Molecular Cloning: A Laboratory Manual, 4th Edition (2012). Methods, devices and materials similar or equivalent to those described herein can be used in the practice of this invention.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. Itis to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

As used herein, the singular terms “a”, “an”, and “the” include the plural reference unless the context clearly indicates otherwise. Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, the term “control” or “control experiment” is used in accordance with its plain and ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

As used herein, the term “complement” is used in accordance with its plain and ordinary meaning and refers to a nucleotide (e.g., RNA nucleotide or DNA nucleotide) or a sequence of nucleotides capable of base pairing with a complementary nucleotide or sequence of nucleotides (e.g., Watson-Crick base pairing). As described herein and commonly known in the art the complementary (matching) nucleotide of adenosine is thymidine and the complementary (matching) nucleotide of guanosine is cytosine. Thus, a complement may include a sequence of nucleotides that base paired with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. Another example of complementary sequences are a template sequence and an amplicon sequence polymerized by a polymerase along the template sequence. “Duplex” means at least two oligonucleotides and/or polynucleotides that are fully or partially complementary undergo Watson-Crick type base pairing among all or most of their nucleotides so that a stable complex is formed. Complementary single stranded nucleic acids and/or substantially complementary single stranded nucleic acids can hybridize to each other under hybridization conditions, thereby forming a nucleic acid that is partially or fully double stranded. When referring to a double-stranded polynucleotide including a first strand hybridized to a second strand, it is understood that each of the first strand and the second strand are independently single-stranded polynucleotides. All or a portion of a nucleic acid sequence may be substantially complementary to another nucleic acid sequence, in some embodiments. As referred to herein, “substantially complementary” refers to nucleotide sequences that can hybridize with each other under suitable hybridization conditions. Hybridization conditions can be altered to tolerate varying amounts of sequence mismatch within complementary nucleic acids that are substantially complementary. Substantially complementary portions of nucleic acids that can hybridize to each other can be 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other. In some embodiments substantially complementary portions of nucleic acids that can hybridize to each other are 100% complementary. Nucleic acids, or portions thereof, that are configured to hybridize to each other often include nucleic acid sequences that are substantially complementary to each other.

As used herein, the term “weak base” refers to refers to adenine (A) and thymine (T) bases as these nitrogenous bases form two hydrogen bond pairs and thus weaker bonds compared to the guanine (G) or cytosine (C) bases pairs.

As used herein, the term “strong base” refers to guanine (G) or cytosine (C) bases as these nitrogenous bases form three hydrogen bond pairs and thus stronger bonds compared to the adenine (A) and thymine (T) base pairs.

As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that complement one another (e.g., about 60%, preferably 65%, 70%, 75%, 80%, 85%, 90%, 91%, 92%, 93%, 94%, 95%, 96%, 97%, 98%, 99%, or higher complementarity over a specified region). In embodiments, two sequences are complementary when they are completely complementary, having 100% complementarity. In embodiments, sequences in a pair of complementary sequences form portions of a single polynucleotide with non-base-pairing nucleotides (e.g., as in a hairpin or loop structure, with or without an overhang) or portions of separate polynucleotides. In embodiments, one or both sequences in a pair of complementary sequences form portions of longer polynucleotides, which may or may not include additional regions of complementarity.

As used herein, the term “contacting” is used in accordance with its plain ordinary meaning and refers to the process of allowing at least two distinct species (e.g., chemical compounds including biomolecules, particles, solid supports, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a protein or enzyme.

As may be used herein, the terms “nucleic acid,” “nucleic acid molecule,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may include natural nucleic acid sequences and variants thereof, artificial nucleic acid sequences, or a combination of such sequences. As may be used herein, the terms “nucleic acid oligomer” and “oligonucleotide” are used interchangeably and are intended to include, but are not limited to, nucleic acids having a length of 200 nucleotides or less. In some embodiments, an oligonucleotide is a nucleic acid having a length of 2 to 200 nucleotides, 2 to 150 nucleotides, 5 to 150 nucleotides or 5 to 100 nucleotides. The terms “polynucleotide,” “oligonucleotide,” “oligo” or the like refer, in the usual and customary sense, to a linear sequence of nucleotides. Oligonucleotides are typically from about 5, 6, 7, 8, 9, 10, 12, 15, 25, 30, 40, 50 or more nucleotides in length, up to about 100 nucleotides in length. In some embodiments, an oligonucleotide is a primer configured for extension by a polymerase when the primer is annealed completely or partially to a complementary nucleic acid template. A primer is often a single stranded nucleic acid. In certain embodiments, a primer, or portion thereof, is substantially complementary to a portion of an adapter. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. In some embodiments, an oligonucleotide may be immobilized to a solid support.

As used herein, the terms “polynucleotide primer” and “primer” refers to any polynucleotide molecule that may hybridize to a polynucleotide template, be bound by a polymerase, and be extended in a template-directed process for nucleic acid synthesis (e.g., amplification and/or sequencing). The primer may be a separate polynucleotide from the polynucleotide template, or both may be portions of the same polynucleotide (e.g., as in a hairpin structure having a 3′ end that is extended along another portion of the polynucleotide to extend a double-stranded portion of the hairpin). Primers (e.g., forward or reverse primers) may be attached to a solid support. A primer can be of any length depending on the particular technique it will be used for. For example, PCR primers are generally between 10 and 40 nucleotides in length. The length and complexity of the nucleic acid fixed onto the nucleic acid template may vary. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. In certain embodiments, a primer has a length of 10 to 150 nucleotides, 15 to 150 nucleotides, 5 to 100 nucleotides, 5 to 50 nucleotides or 10 to 50 nucleotides. A primer typically has a length of 10 to 50 nucleotides. For example, a primer may have a length of 10 to 40, 10 to 30, 10 to 20, 25 to 50, 15 to 40, 15 to 30, 20 to 50, 20 to 40, or 20 to 30 nucleotides. In some embodiments, a primer has a length of 18 to 24 nucleotides. One of skill can adjust these factors to provide optimum hybridization and signal production for a given hybridization procedure. The primer permits the addition of a nucleotide residue thereto, or oligonucleotide or polynucleotide synthesis therefrom, under suitable conditions. In an embodiment the primer is a DNA primer, i.e., a primer consisting of, or largely consisting of, deoxyribonucleotide residues. The primers are designed to have a sequence that is the complement of a region of template/target DNA to which the primer hybridizes. The addition of a nucleotide residue to the 3′ end of a primer by formation of a phosphodiester bond results in a DNA extension product. The addition of a nucleotide residue to the 3′ end of the DNA extension product by formation of a phosphodiester bond results in a further DNA extension product. In another embodiment the primer is an RNA primer. In embodiments, a primer is hybridized to a target polynucleotide. A “primer” is complementary to a polynucleotide template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

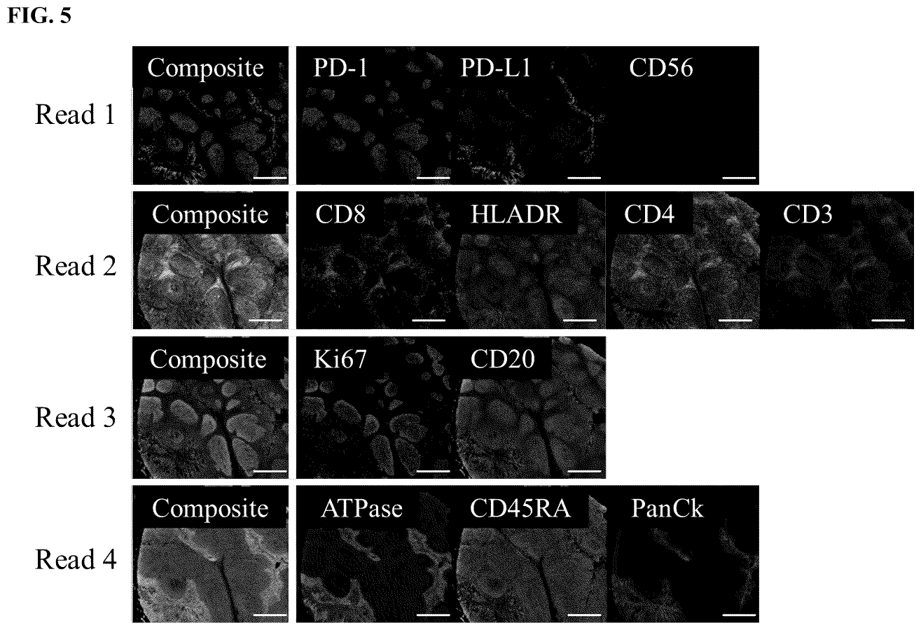

As used herein, the term “blocking oligonucleotide” refers to an oligonucleotide hybridized to the oligonucleotide described herein or a portion of the oligonucleotide described herein prior to the hybridization of the polynucleotide probe to the oligonucleotide. The blocking oligonucleotide serves to mitigate the nonspecific binding by the oligonucleotide attached to the specific binding agent described herein. The methods and compositions described herein are directed to in situ target detection and may employ one, two, or a plurality of blocking oligonucleotides prior to the hybridization of a polynucleotide probe to the oligonucleotide attached to the specific binding agent described herein. Examples of blocking oligonucleotides are provided in FIGS. 4A and 4B.

As used herein, the term “polynucleotide probe” refers to a polynucleotide including a first binding sequence and a second binding sequence to the oligonucleotide described herein (i.e., the oligonucleotide attached to the specific binding agent).

As used herein, the term “secondary structure” refers to the spatial conformation formed within or between segments of nucleic acids. These conformations are produced by intermolecular interactions and/or intramolecular interactions between nucleobases in segments of nucleic acids. Examples of secondary structure includes but is not limited to stems, inner loops, bulges, hairpins, G-quadruplexes, pseudoknots, and multiple-way junctions (see, e.g., Binet T. et al. BMC bioinformatics. 2023 Nov. 8; 24 (1): 422).

As used herein, the term “primer binding sequence” refers to a polynucleotide sequence that is complementary to at least a portion of a primer (e.g., a sequencing primer or an amplification primer). Primer binding sequences can be of any suitable length. In embodiments, a primer binding sequence is about or at least about 10, 15, 20, 25, 30, or more nucleotides in length. In embodiments, a primer binding sequence is 10-50, 15-30, or 20-25 nucleotides in length. The primer binding sequence may be selected such that the primer (e.g., sequencing primer) has the preferred characteristics to minimize secondary structure formation or minimize non-specific amplification, for example having a length of about 20-30 nucleotides; approximately 50% GC content, and a Tm of about 55° C. to about 65° C.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amio acid on a protein or polypeptide through a covalent, non-covalent or other interaction.

The order of elements within a nucleic acid molecule is typically described herein from 5′ to 3′, unless otherwise specified. In the case of a double-stranded molecule, the “top” strand is typically shown from 5′ to 3′, according to convention, and the order of elements is described herein with reference to the top strand.

The term “messenger RNA” or “mRNA” refers to an RNA that is without introns and is capable of being translated into a polypeptide. The term “RNA” refers to any ribonucleic acid, including but not limited to mRNA, tRNA (transfer RNA), rRNA (ribosomal RNA), and/or noncoding RNA (such as lncRNA (long noncoding RNA)). The term “cDNA” refers to a DNA that is complementary or identical to an RNA, in either single stranded or double stranded form.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

As used herein, the term “associated” or “associated with” can mean that two or more species are identifiable as being co-located at a point in time. An association can mean that two or more species are or were within a similar container. An association can be an informatics association, where for example digital information regarding two or more species is stored and can be used to determine that one or more of the species were co-located at a point in time. An association can also be a physical association. In some instances two or more associated species are “tethered”, “coated”, “attached”, or “immobilized” to one another or to a common solid or semisolid support (e.g. a receiving substrate). An association may refer to a relationship, or connection, between two entities. For example, a barcode sequence or barcode nucleotide may be associated with a particular target by binding a probe including the barcode sequence to the target. In embodiments, detecting the associated barcode provides detection of the target. Associated may refer to the relationship between a sample and the DNA molecules, RNA molecules, or polynucleotides originating from or derived from that sample. These relationships may be encoded in oligonucleotide barcodes, as described herein. A polynucleotide is associated with a sample if it is an endogenous polynucleotide, i.e., it occurs in the sample at the time the sample is obtained, or is derived from an endogenous polynucleotide. For example, the RNAs endogenous to a cell are associated with that cell. cDNAs resulting from reverse transcription of these RNAs, and DNA amplicons resulting from PCR amplification of the cDNAs, contain the sequences of the RNAs and are also associated with the cell. The polynucleotides associated with a sample need not be located or synthesized in the sample, and are considered associated with the sample even after the sample has been destroyed (for example, after a cell has been lysed). Barcoding can be used to determine which polynucleotides in a mixture are associated with a particular sample. In embodiments, a proximity probe is associated with a particular barcode, such that identifying the barcode identifies the probe with which it is associated. Because the proximity probe specifically binds to a target, identifying the barcode thus identifies the target.

As used herein, the terms “analogue” and “analog”, in reference to a chemical compound, refers to compound having a structure similar to that of another one, but differing from it in respect of one or more different atoms, functional groups, or substructures that are replaced with one or more other atoms, functional groups, or substructures. In the context of a nucleotide, a nucleotide analog refers to a compound that, like the nucleotide of which it is an analog, can be incorporated into a nucleic acid molecule (e.g., an extension product) by a suitable polymerase, for example, a DNA polymerase in the context of a nucleotide analogue. The terms also encompass nucleic acids containing known nucleotide analogs or modified backbone residues or linkages, which are synthetic, naturally occurring, or non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see, e.g., see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

As used herein, a “native” nucleotide is used in accordance with its plain and ordinary meaning and refers to a naturally occurring nucleotide that does not include an exogenous label (e.g., a fluorescent dye, or other label) or chemical modification such as may characterize a nucleotide analog. Examples of native nucleotides useful for carrying out procedures described herein include: dATP (2′-deoxyadenosine-5′-triphosphate); dGTP (2′-deoxyguanosine-5′-triphosphate); dCTP (2′-deoxycytidine-5′-triphosphate); dTTP (2′-deoxythymidine-5′-triphosphate); and dUTP (2′-deoxyuridine-5′-triphosphate).

As used herein, the term “modified nucleotide” refers to nucleotide modified in some manner. Typically, a nucleotide contains a single 5-carbon sugar moiety, a single nitrogenous base moiety and 1 to three phosphate moieties. In embodiments, a nucleotide can include a blocking moiety and/or a label moiety. A blocking moiety on a nucleotide prevents formation of a covalent bond between the 3′ hydroxyl moiety of the nucleotide and the 5′ phosphate of another nucleotide. A blocking moiety on a nucleotide can be reversible, whereby the blocking moiety can be removed or modified to allow the 3′ hydroxyl to form a covalent bond with the 5′ phosphate of another nucleotide. A blocking moiety can be effectively irreversible under particular conditions used in a method set forth herein. In embodiments, the blocking moiety is attached to the 3′ oxygen of the nucleotide and is independently —NH₂, —CN, —CH₃, C₂-C₆ allyl (e.g., —CH₂—CH═CH₂), methoxyalkyl (e.g., —CH₂—O—CH₃), or —CH₂N₃. In embodiments, the blocking moiety is attached to the 3′ oxygen of the nucleotide and is independently

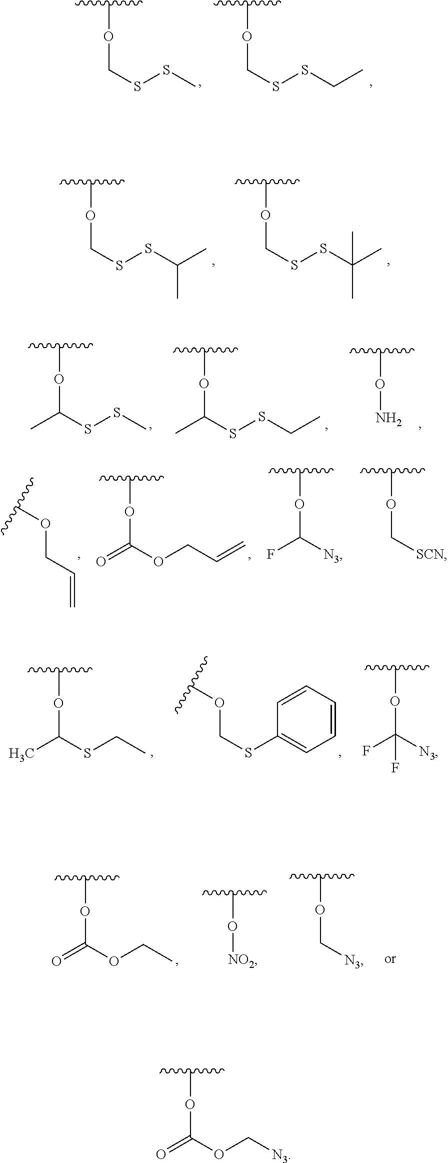

A label moiety of a modified nucleotide can be any moiety that allows the nucleotide to be detected, for example, using a spectroscopic method. Exemplary label moieties are fluorescent labels, mass labels, chemiluminescent labels, electrochemical labels, detectable labels and the like. One or more of the above moieties can be absent from a nucleotide used in the methods and compositions set forth herein. For example, a nucleotide can lack a label moiety or a blocking moiety or both. Examples of nucleotide analogues include, without limitation, 7-deaza-adenine, 7-deaza-guanine, the analogues of deoxynucleotides shown herein, analogues in which a label is attached through a cleavable linker to the 5-position of cytosine or thymine or to the 7-position of deaza-adenine or deaza-guanine, and analogues in which a small chemical moiety is used to cap the OH group at the 3′-position of deoxyribose. Nucleotide analogues and DNA polymerase-based DNA sequencing are also described in U.S. U.S. Pat. No. 6,664,079, which is incorporated herein by reference in its entirety for all purposes. Non-limiting examples of detectable labels include labels including fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the dye is a fluorescent dye. Non-limiting examples of dyes, some of which are commercially available, include CF® dyes (Biotium, Inc.), Alexa Fluor® dyes (Thermo Fisher), DyLight™ dyes (Thermo Fisher), Cy® dyes (GE Healthscience), IRDye® dyes (Li-Cor Biosciences, Inc.), and HiLyte™ dyes (Anaspec, Inc.). In embodiments, the label is a fluorophore.

In some embodiments, a nucleic acid includes a label. As used herein, the term “label” or “labels” is used in accordance with their plain and ordinary meanings and refer to molecules that can directly or indirectly produce or result in a detectable signal either by themselves or upon interaction with another molecule. Non-limiting examples of detectable labels include fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the label is a dye. In embodiments, the dye is a fluorescent dye. Non-limiting examples of dyes, some of which are commercially available, include CF® dyes (Biotium, Inc.), Alexa Fluor® dyes (Thermo Fisher), DyLight™ dyes (Thermo Fisher), Cy® dyes (GE Healthscience), IRDye® dyes (Li-Cor Biosciences, Inc.), and HiLyte™ dyes (Anaspec, Inc.). In embodiments, a particular nucleotide type is associated with a particular label, such that identifying the label identifies the nucleotide with which it is associated. In embodiments, the label is luciferin that reacts with luciferase to produce a detectable signal in response to one or more bases being incorporated into an elongated complementary strand, such as in pyrosequencing. In embodiment, a nucleotide includes a label (such as a dye). In embodiments, the label is not associated with any particular nucleotide, but detection of the label identifies whether one or more nucleotides having a known identity were added during an extension step (such as in the case of pyrosequencing). Examples of detectable agents (i.e., labels) include imaging agents, including fluorescent and luminescent substances, molecules, or compositions, including, but not limited to, a variety of organic or inorganic small molecules commonly referred to as “dyes,” “labels,” or “indicators.” Examples include fluorescein, rhodamine, acridine dyes, Alexa Fluor® dyes, and cyanine dyes. In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent molecule (e.g., acridine dye, cyanine, dye, fluorine dye, oxazine dye, phenanthridine dye, or rhodamine dye). The term “cyanine” or “cyanine moiety” as described herein refers to a detectable moiety containing two nitrogen groups separated by a polymethine chain. In embodiments, the cyanine moiety has 3 methine structures (i.e., cyanine 3 or Cy®3). In embodiments, the cyanine moiety has 5 methine structures (i.e., cyanine 5 or Cy®5). In embodiments, the cyanine moiety has 7 methine structures (i.e., cyanine 7 or Cy®7).

The term “nucleoside” refers, in the usual and customary sense, to a glycosylamine including a nucleobase and a five-carbon sugar (ribose or deoxyribose). Non-limiting examples of nucleosides include cytidine, uridine, adenosine, guanosine, thymidine and inosine. Nucleosides may be modified at the base and/or the sugar. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g., polynucleotides contemplated herein include any types of RNA, e.g., mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site www.ncbi.nlm.nih.gov/BLAST/or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

As used herein, the term “removable” group, e.g., a label or a blocking group or protecting group, is used in accordance with its plain and ordinary meaning and refers to a chemical group that can be removed from a nucleotide analogue such that a DNA polymerase can extend the nucleic acid (e.g., a primer or extension product) by the incorporation of at least one additional nucleotide. Removal may be by any suitable method, including enzymatic, chemical, or photolytic cleavage. Removal of a removable group, e.g., a blocking group, does not require that the entire removable group be removed, only that a sufficient portion of it be removed such that a DNA polymerase can extend a nucleic acid by incorporation of at least one additional nucleotide using a nucleotide or nucleotide analogue. In general, the conditions under which a removable group is removed are compatible with a process employing the removable group (e.g., an amplification process or sequencing process).

As used herein, the terms “reversible blocking groups” and “reversible terminators” are used in accordance with their plain and ordinary meanings and refer to a blocking moiety located, for example, at the 3′ position of a modified nucleotide and may be a chemically cleavable moiety such as an allyl group, an azidomethyl group or a methoxymethyl group, or may be an enzymatically cleavable group such as a phosphate ester. Non-limiting examples of nucleotide blocking moieties are described in applications WO 2004/018497, WO 96/07669, U.S. Pat. Nos. 7,057,026, 7,541,444, 5,763,594, 5,808,045, 5,872,244 and 6,232,465 the contents of which are incorporated herein by reference in their entirety. The nucleotides may be labelled or unlabeled. They may be modified with reversible terminators useful in methods provided herein and may be 3′-O-blocked reversible or 3′-unblocked reversible terminators. In nucleotides with 3′-O-blocked reversible terminators, the blocking group-OR [reversible terminating (capping) group] is linked to the oxygen atom of the 3′-OH of the pentose, while the label is linked to the base, which acts as a reporter and can be cleaved. The 3′-O-blocked reversible terminators are known in the art, and may be, for instance, a 3′-ONH₂ reversible terminator, a 3′-O-allyl reversible terminator, or a 3′-O-azidomethyl reversible terminator. In embodiments, the reversible terminator moiety is attached to the 3′-oxygen of the nucleotide, having the formula:

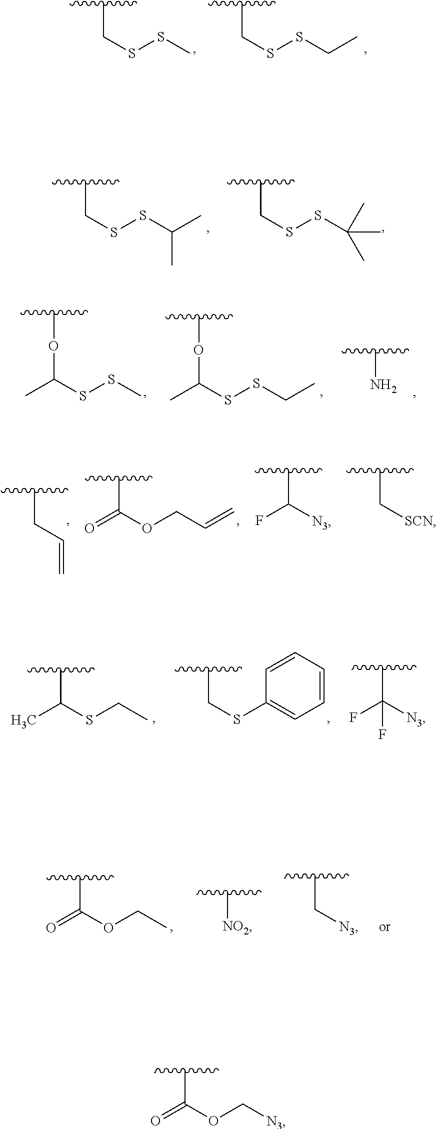

wherein the 3′ oxygen of the nucleotide is not shown in the formulae above. The term “allyl” as described herein refers to an unsubstituted methylene attached to a vinyl group (i.e., —CH—CH₂). In embodiments, the reversible terminator moiety is

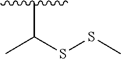 [SMILES: *C(C)SSC]

as described in U.S. Pat. No. 10,738,072, which is incorporated herein by reference for all purposes. For example, a nucleotide including a reversible terminator moiety may be represented by the formula:

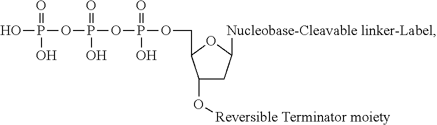

where the nucleobase is adenine or adenine analogue, thymine or thymine analogue, guanine or guanine analogue, or cytosine or cytosine analogue.

In some embodiments, a nucleic acid (e.g., a probe or a primer) includes a molecular identifier or a molecular barcode. As used herein, the term “molecular barcode” (which may be referred to as a “tag”, a “barcode”, a “molecular identifier”, an “identifier sequence” or a “unique molecular identifier” (UMI)) refers to any material (e.g., a nucleotide sequence, a nucleic acid molecule feature) that is capable of distinguishing an individual molecule in a large heterogeneous population of molecules. A barcode nucleotide serves a similar function, however refers to a single nucleotide. In embodiments, a barcode is unique in a pool of barcodes that differ from one another in sequence, or is uniquely associated with a particular sample polynucleotide in a pool of sample polynucleotides. In embodiments, every barcode in a pool of adapters is unique, such that sequencing reads including the barcode can be identified as originating from a single sample polynucleotide molecule on the basis of the barcode alone. In other embodiments, individual barcode sequences may be used more than once, but adapters including the duplicate barcodes are associated with different sequences and/or in different combinations of barcoded adaptors, such that sequence reads may still be uniquely distinguished as originating from a single sample polynucleotide molecule on the basis of a barcode and adjacent sequence information (e.g., sample polynucleotide sequence, and/or one or more adjacent barcodes). In embodiments, barcodes are about or at least about 5, 6, 7, 8, 9, 10, 15, 20, 25, 30, 40, 50, 75 or more nucleotides in length. In embodiments, barcodes are shorter than 20, 15, 10, 9, 8, 7, 6, or 5 nucleotides in length. In embodiments, barcodes are about 10 to about 50 nucleotides in length, such as about 15 to about 40 or about 20 to about 30 nucleotides in length. In a pool of different barcodes, barcodes may have the same or different lengths. In general, barcodes are of sufficient length and include sequences that are sufficiently different to allow the identification of sequencing reads that originate from the same sample polynucleotide molecule. In embodiments, each barcode in a plurality of barcodes differs from every other barcode in the plurality by at least three nucleotide positions, such as at least 3, 4, 5, 6, 7, 8, 9, 10, or more nucleotide positions. In some embodiments, substantially degenerate barcodes may be known as random. In some embodiments, a barcode may include a nucleic acid sequence from within a pool of known sequences. In some embodiments, the barcodes may be pre-defined. In embodiments, the barcodes are selected to form a known set of barcodes, e.g., the set of barcodes may be distinguished by a particular Hamming distance. In embodiments, each barcode sequence is unique within the known set of barcodes. In embodiments, each barcode sequence is associated with a particular oligonucleotide probe.

In embodiments, a nucleic acid (e.g., an adapter or primer) includes a sample barcode. In general, a “sample barcode” is a nucleotide sequence that is sufficiently different from other sample barcode to allow the identification of the sample source based on sample barcode sequence(s) with which they are associated. In embodiments, a plurality of nucleotides (e.g., all nucleotides from a particular sample source, or sub-sample thereof) are joined to a first sample barcode, while a different plurality of nucleotides (e.g., all nucleotides from a different sample source, or different subsample) are joined to a second sample barcode, thereby associating each plurality of polynucleotides with a different sample barcode indicative of sample source. In embodiments, each sample barcode in a plurality of sample barcodes differs from every other sample barcode in the plurality by at least three nucleotide positions, such as at least 3, 4, 5, 6, 7, 8, 9, 10, or more nucleotide positions. In some embodiments, substantially degenerate sample barcodes may be known as random. In some embodiments, a sample barcode may include a nucleic acid sequence from within a pool of known sequences. In some embodiments, the sample barcodes may be pre-defined. In embodiments, the sample barcode includes about 1 to about 10 nucleotides. In embodiments, the sample barcode includes about 3, 4, 5, 6, 7, 8, 9, or about 10 nucleotides. In embodiments, the sample barcode includes about 3 nucleotides. In embodiments, the sample barcode includes about 5 nucleotides. In embodiments, the sample barcode includes about 7 nucleotides. In embodiments, the sample barcode includes about 10 nucleotides. In embodiments, the sample barcode includes about 6 to about 10 nucleotides.

As used herein, the term “DNA polymerase” and “nucleic acid polymerase” are used in accordance with their plain ordinary meanings and refer to enzymes capable of synthesizing nucleic acid molecules from nucleotides (e.g., deoxyribonucleotides). Exemplary types of polymerases that may be used in the compositions and methods of the present disclosure include the nucleic acid polymerases such as DNA polymerase, DNA- or RNA-dependent RNA polymerase, and reverse transcriptase. In some cases, the DNA polymerase is 9°N polymerase or a variant thereof, E. coli DNA polymerase I, Bacteriophage T4 DNA polymerase, Sequenase, Taq DNA polymerase, DNA polymerase from Bacillus stearothermophilus, Bst 2.0 DNA polymerase, 9°N polymerase (exo−) A485L/Y409V, Phi29 DNA Polymerase (φ29 DNA Polymerase), T7 DNA polymerase, DNA polymerase II, DNA polymerase III holoenzyme, DNA polymerase IV, DNA polymerase V, VentR DNA polymerase, Therminator™ II DNA Polymerase, Therminator™ III DNA Polymerase, or Therminator™ IX DNA Polymerase. In embodiments, the polymerase is a protein polymerase. Typically, a DNA polymerase adds nucleotides to the 3′-end of a DNA strand, one nucleotide at a time. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ι DNA polymerase, Pol κ DNA polymerase, Pol ζ DNA polymerase, Pol γ DNA polymerase, Pol θ DNA polymerase, Pol υ DNA polymerase, or a thermophilic nucleic acid polymerase (e.g. Therminator γ, 9° N polymerase (exo−), Therminator II, Therminator III, or Therminator IX). In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044). In embodiments, the polymerase is an enzyme described in US 2021/0139884. For example, a polymerase catalyzes the addition of a next correct nucleotide to the 3′-OH group of the primer via a phosphodiester bond, thereby chemically incorporating the nucleotide into the primer. Optionally, the polymerase used in the provided methods is a processive polymerase. Optionally, the polymerase used in the provided methods is a distributive polymerase.

As used herein, the term “exonuclease activity” is used in accordance with its ordinary meaning in the art, and refers to the removal of a nucleotide from a nucleic acid by an enzyme (e.g. DNA polymerase, a lambda exonuclease, Exo I, Exo III, T5, Exo V, Exo VII or the like). For example, during polymerization, nucleotides are added to the 3′ end of the primer strand. Occasionally a DNA polymerase incorporates an incorrect nucleotide to the 3′-OH terminus of the primer strand, wherein the incorrect nucleotide cannot form a hydrogen bond to the corresponding base in the template strand. Such a nucleotide, added in error, is removed from the primer as a result of the 3′ to 5′ exonuclease activity of the DNA polymerase. In embodiments, exonuclease activity may be referred to as “proofreading.” When referring to 3′-5′ exonuclease activity, it is understood that the DNA polymerase facilitates a hydrolyzing reaction that breaks phosphodiester bonds at the 3′ end of a polynucleotide chain to excise the nucleotide. In embodiments, 3′-5′ exonuclease activity refers to the successive removal of nucleotides in single-stranded DNA in a 3′->5′ direction, releasing deoxyribonucleoside 5′-monophosphates one after another. Methods for quantifying exonuclease activity are known in the art, see for example Southworth et al, PNAS Vol 93, 8281-8285 (1996). In embodiments, 5′-3′ exonuclease activity refers to the successive removal of nucleotides in double-stranded DNA in a 5′->3′ direction. In embodiments, the 5′-3′ exonuclease is lambda exonuclease. For example, lambda exonuclease catalyzes the removal of 5′ mononucleotides from duplex DNA, with a preference for 5′ phosphorylated double-stranded DNA. In other embodiments, the 5′-3′ exonuclease is E. coli DNA Polymerase I.

As used herein, the term “incorporating” or “chemically incorporating,” when used in reference to a primer and cognate nucleotide, refers to the process of joining the cognate nucleotide to the primer or extension product thereof by formation of a phosphodiester bond.

As used herein, the term “selective” or “selectivity” or the like of a compound refers to the compound's ability to discriminate between molecular targets. For example, a chemical reagent may selectively modify one nucleotide type in that it reacts with one nucleotide type (e.g., cytosines) and not other nucleotide types (e.g., adenine, thymine, or guanine). When used in the context of sequencing, such as in “selectively sequencing,” this term refers to sequencing one or more target polynucleotides from an original starting population of polynucleotides, and not sequencing non-target polynucleotides from the starting population. Typically, selectively sequencing one or more target polynucleotides involves differentially manipulating the target polynucleotides based on known sequence. For example, target polynucleotides may be hybridized to a probe oligonucleotide that may be labeled (such as with a member of a binding pair) or bound to a surface. In embodiments, hybridizing a target polynucleotide to a probe oligonucleotide includes the step of displacing one strand of a double-stranded nucleic acid. Probe-hybridized target polynucleotides may then be separated from non-hybridized polynucleotides, such as by removing probe-bound polynucleotides from the starting population or by washing away polynucleotides that are not bound to a probe. The result is a selected subset of the starting population of polynucleotides, which is then subjected to sequencing, thereby selectively sequencing the one or more target polynucleotides.

As used herein, the term “template polynucleotide” refers to any polynucleotide molecule that may be bound by a polymerase and utilized as a template for nucleic acid synthesis. A template polynucleotide may be a target polynucleotide. In general, the term “target polynucleotide” refers to a nucleic acid molecule or polynucleotide in a starting population of nucleic acid molecules having a target sequence whose presence, amount, and/or nucleotide sequence, or changes in one or more of these, are desired to be determined. The target sequence may be a portion of a gene, a regulatory sequence, genomic DNA, cDNA, RNA including mRNA, miRNA, rRNA, or others. The target sequence may be a target sequence from a sample or a secondary target such as a product of an amplification reaction. A target polynucleotide is not necessarily any single molecule or sequence. For example, a target polynucleotide may be any one of a plurality of target polynucleotides in a reaction, or all polynucleotides in a given reaction, depending on the reaction conditions. For example, in a nucleic acid amplification reaction with random primers, all polynucleotides in a reaction may be amplified. As a further example, a collection of targets may be simultaneously assayed using polynucleotide primers directed to a plurality of targets in a single reaction. As yet another example, all or a subset of polynucleotides in a sample may be modified by the addition of a primer-binding sequence (such as by the ligation of adapters containing the primer binding sequence), rendering each modified polynucleotide a target polynucleotide in a reaction with the corresponding primer polynucleotide(s). In embodiments, the template polynucleotide includes a target nucleic acid sequence and one or more barcode sequences. In embodiments, the template polynucleotide is a barcode sequence.

As used herein, the terms “specific”, “specifically”, “specificity”, or the like of a compound refers to the compound's ability to cause a particular action, such as binding, to a particular molecular target with minimal or no action to other proteins in the cell.

The terms “attached,” “bind,” and “bound” as used herein are used in accordance with their plain and ordinary meanings and refer to an association between atoms or molecules. The association can be direct or indirect. For example, attached molecules may be directly bound to one another, e.g., by a covalent bond or non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). As a further example, two molecules may be bound indirectly to one another by way of direct binding to one or more intermediate molecules, thereby forming a complex.

“Specific binding” is where the binding is selective between two molecules. A particular example of specific binding is that which occurs between an antibody and an antigen. Typically, specific binding can be distinguished from non-specific when the dissociation constant (KD) is less than about 1×10⁻⁵ M or less than about 1×10⁻⁶ M or 1×10⁻⁷M. Specific binding can be detected, for example, by ELISA, immunoprecipitation, coprecipitation, with or without chemical crosslinking, two-hybrid assays and the like. In embodiments, the KD (equilibrium dissociation constant) between two specific binding molecules is less than 10⁻⁶ M, less than 10⁻⁷M, less than 10⁻⁸ M, less than 10⁻⁹ M, less than 10⁻¹⁰ M, less than 10⁻¹¹ M, or less than about 10⁻¹² M or less.

As used herein, the term “specific binding agent” refers to an agent that binds specifically to a particular biomolecule (e.g., carbohydrate, cell surface receptor, protein, nucleic acid, or lipid molecule). Examples of a specific binding reagent include, but are not limited to, an antibody or target-specific oligonucleotide.

As used herein, the terms “sequencing”, “sequence determination”, “determining a nucleotide sequence”, and the like include determination of a partial or complete sequence information (e.g., a sequence) of a polynucleotide being sequenced, and particularly physical processes for generating such sequence information. That is, the term includes sequence comparisons, consensus sequence determination, contig assembly, fingerprinting, and like levels of information about a target polynucleotide, as well as the express identification and ordering of nucleotides in a target polynucleotide. The term also includes the determination of the identification, ordering, and locations of one, two, or three of the four types of nucleotides within a target polynucleotide. In some embodiments, a sequencing process described herein includes contacting a template and an annealed primer with a suitable polymerase under conditions suitable for polymerase extension and/or sequencing.

As used herein, the term “polymer” refers to macromolecules having one or more structurally unique repeating units. The repeating units are referred to as “monomers,” which are polymerized for the polymer. Typically, a polymer is formed by monomers linked in a chain-like structure. A polymer formed entirely from a single type of monomer is referred to as a “homopolymer.” A polymer formed from two or more unique repeating structural units may be referred to as a “copolymer.” A polymer may be linear or branched, and may be random, block, polymer brush, hyperbranched polymer, bottlebrush polymer, dendritic polymer, or polymer micelles. The term “polymer” includes homopolymers, copolymers, tripolymers, tetra polymers and other polymeric molecules made from monomeric subunits. Copolymers include alternating copolymers, periodic copolymers, statistical copolymers, random copolymers, block copolymers, linear copolymers and branched copolymers. The term “polymerizable monomer” is used in accordance with its meaning in the art of polymer chemistry and refers to a compound that may covalently bind chemically to other monomer molecules (such as other polymerizable monomers that are the same or different) to form a polymer.

Polymers can be hydrophilic, hydrophobic or amphiphilic, as known in the art. Thus, “hydrophilic polymers” are substantially miscible with water and include, but are not limited to, polyethylene glycol and the like. “Hydrophobic polymers” are substantially immiscible with water and include, but are not limited to, polyethylene, polypropylene, polybutadiene, polystyrene, polymers disclosed herein, and the like. “Amphiphilic polymers” have both hydrophilic and hydrophobic properties and are typically copolymers having hydrophilic segment(s) and hydrophobic segment(s). Polymers include homopolymers, random copolymers, and block copolymers, as known in the art. The term “homopolymer” refers, in the usual and customary sense, to a polymer having a single monomeric unit. The term “copolymer” refers to a polymer derived from two or more monomeric species. The term “random copolymer” refers to a polymer derived from two or more monomeric species with no preferred ordering of the monomeric species. The term “block copolymer” refers to polymers having two or homopolymer subunits linked by covalent bond. Thus, the term “hydrophobic homopolymer” refers to a homopolymer which is hydrophobic. The term “hydrophobic block copolymer” refers to two or more homopolymer subunits linked by covalent bonds and which is hydrophobic.

As used herein, the term “hydrogel” refers to a three-dimensional polymeric structure that is substantially insoluble in water, but which is capable of absorbing and retaining large quantities of water to form a substantially stable, often soft and pliable, structure. In embodiments, water can penetrate in between polymer chains of a polymer network, subsequently causing swelling and the formation of a hydrogel. In embodiments, hydrogels are super-absorbent (e.g., containing more than about 90% water) and can be comprised of natural or synthetic polymers.

As used herein, the term “substrate” refers to a solid support material. The substrate can be non-porous or porous. The substrate can be rigid or flexible. As used herein, the terms “solid support” and “solid surface” refers to discrete solid or semi-solid surface. A solid support may encompass any type of solid, porous, or hollow sphere, ball, cylinder, or other similar configuration composed of plastic, ceramic, metal, or polymeric material (e.g., hydrogel) onto which a nucleic acid may be immobilized (e.g., covalently or non-covalently). A nonporous substrate generally provides a seal against bulk flow of liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefin copolymers, polyimides etc.), nylon, ceramics, resins, Zeonor®, silica or silica-based materials including silicon and modified silicon, carbon, metals, inorganic glasses, optical fiber bundles, photopatternable dry film resists, UV-cured adhesives and polymers. Particularly useful solid supports for some embodiments have at least one surface located within a flow cell. Solid surfaces can also be varied in their shape depending on the application in a method described herein. For example, a solid surface useful herein can be planar, or contain regions which are concave or convex. In embodiments, the geometry of the concave or convex regions (e.g., wells) of the solid surface conform to the size and shape of the particle to maximize the contact between as substantially circular particle. In embodiments, the wells of an array are randomly located such that nearest neighbor features have random spacing between each other. Alternatively, in embodiments the spacing between the wells can be ordered, for example, forming a regular pattern. The term solid substrate is encompassing of a substrate (e.g., a flow cell) having a surface including a polymer coating covalently attached thereto. In embodiments, the solid substrate is a flow cell. The term “flow cell” as used herein refers to a chamber including a solid surface across which one or more fluid reagents can be flowed. Examples of flow cells and related fluidic systems and detection platforms that can be readily used in the methods of the present disclosure are described, for example, in Bentley et al., Nature 456:53-59 (2008). In certain embodiments a substrate includes a surface (e.g., a surface of a flow cell, a surface of a tube, a surface of a chip), for example a metal surface (e.g., steel, gold, silver, aluminum, silicon and copper). In embodiments a substrate (e.g., a substrate surface) is coated and/or includes functional groups and/or inert materials. In certain embodiments a substrate includes a bead, a chip, a capillary, a plate, a membrane, a wafer (e.g., silicon wafers), a comb, or a pin for example. In some embodiments a substrate includes a bead and/or a nanoparticle. A substrate can be made of a suitable material, non-limiting examples of which include a plastic or a suitable polymer (e.g., polycarbonate, poly(vinyl alcohol), poly(divinylbenzene), polystyrene, polyamide, polyester, polyvinylidene difluoride (PVDF), polyethylene, polyurethane, polypropylene, and the like), borosilicate, glass, nylon, Wang resin, Merrifield resin, metal (e.g., iron, a metal alloy, sepharose, agarose, polyacrylamide, dextran, cellulose and the like or combinations thereof. In embodiments a substrate includes a magnetic material (e.g., iron, nickel, cobalt, platinum, aluminum, and the like). In embodiments a substrate includes a magnetic bead (e.g., DYNABEADS®, hematite, AMPure XP). Magnets can be used to purify and/or capture nucleic acids bound to certain substrates (e.g., substrates including a metal or magnetic material). The flow cell is typically a glass slide containing small fluidic channels (e.g., a glass slide 75 mm×25 mm×1 mm having one or more channels), through which sequencing solutions (e.g., polymerases, nucleotides, and buffers) may traverse. Though typically glass, suitable flow cell materials may include polymeric materials, plastics, silicon, quartz (fused silica), Borofloat® glass, silica, silica-based materials, carbon, metals, an optical fiber or optical fiber bundles, sapphire, or plastic materials such as COCs and epoxies. The particular material can be selected based on properties desired for a particular use. For example, materials that are transparent to a desired wavelength of radiation are useful for analytical techniques that will utilize radiation of the desired wavelength. Conversely, it may be desirable to select a material that does not pass radiation of a certain wavelength (e.g., being opaque, absorptive, or reflective). In embodiments, the material of the flow cell is selected due to the ability to conduct thermal energy. In embodiments, a flow cell includes inlet and outlet ports and a flow channel extending there between.

The term “surface” is intended to mean an external part or external layer of a substrate. The surface can be in contact with another material such as a gas, liquid, gel, polymer, organic polymer, second surface of a similar or different material, metal, or coat. The surface, or regions thereof, can be substantially flat. The substrate and/or the surface can have surface features such as wells, pits, channels, ridges, raised regions, pegs, posts or the like.

The term “microplate”, or “multiwell container” as used herein, refers to a substrate including a surface, the surface including a plurality of reaction chambers separated from each other by interstitial regions on the surface. In embodiments, the microplate has dimensions as provided and described by American National Standards Institute (ANSI) and Society for Laboratory Automation And Screening (SLAS); for example the tolerances and dimensions set forth in ANSI SLAS 1-2004 (R2012); ANSI SLAS 2-2004 (R2012); ANSI SLAS 3-2004 (R2012); ANSI SLAS 4-2004 (R2012); and ANSI SLAS 6-2012, which are incorporated herein by reference. The dimensions of the microplate as described herein and the arrangement of the reaction chambers may be compatible with an established format for automated laboratory equipment. In embodiments, the device described herein provides methods for high-throughput screening. High-throughput screening (HTS) refers to a process that uses a combination of modern robotics, data processing and control software, liquid handling devices, and/or sensitive detectors, to efficiently process a large amount of (e.g., thousands, hundreds of thousands, or millions) samples in biochemical, genetic, or pharmacological experiments, either in parallel or in sequence, within a reasonably short period of time (e.g., days). Preferably, the process is amenable to automation, such as robotic simultaneous handling of 96 samples, 384 samples, 1536 samples or more. A typical HTS robot tests up to 100,000 to a few hundred thousand compounds per day. The samples are often in small volumes, such as no more than 1 mL, 500 μL, 200 μL, 100 μL, 50 μL or less. Through this process, one can rapidly identify active compounds, small molecules, antibodies, proteins or polynucleotides in a cell.

The reaction chambers may be provided as wells of a multiwell container (alternatively referred to as reaction chambers), for example a microplate may contain 2, 4, 6, 12, 24, 48, 96, 384, or 1536 sample wells. In embodiments, the 96 and 384 wells are arranged in a 2:3 rectangular matrix. In embodiments, the 24 wells are arranged in a 3:8 rectangular matrix. In embodiments, the 48 wells are arranged in a 3:4 rectangular matrix. In embodiments, the reaction chamber is a microscope slide (e.g., a glass slide about 75 mm by about 25 mm). In embodiments the slide is a concavity slide (e.g., the slide includes a depression). In embodiments, the slide includes a coating for enhanced cell adhesion (e.g., poly-L-lysine, silanes, carbon nanotubes, polymers, epoxy resins, or gold). In embodiments, the microplate is about 5 inches by about 3.33 inches, and includes a plurality of 5 mm diameter wells. In embodiments, the microplate is about 5 inches by about 3.33 inches, and includes a plurality of 6 mm diameter wells. In embodiments, the microplate is about 5 inches by about 3.33 inches, and includes a plurality of 7 mm diameter wells. In embodiments, the microplate is about 5 inches by about 3.33 inches, and includes a plurality of 7.5 mm diameter wells. In embodiments, the microplate is 5 inches by 3.33 inches, and includes a plurality of 7.5 mm diameter wells. In embodiments, the microplate is about 5 inches by about 3.33 inches, and includes a plurality of 8 mm diameter wells. In embodiments, the microplate is a flat glass or plastic tray in which an array of wells are formed, wherein each well can hold between from a few microliters to hundreds of microliters of fluid reagents and samples. In embodiments, the microplate has a rectangular shape that measures 127.7 mm±0.5 mm in length by 85.4 mm±0.5 mm in width, and includes 6, 12, 24, 48, or 96 wells, wherein each well has an average diameter of about 5-7 mm. In embodiments, the microplate has a rectangular shape that measures 127.7 mm±0.5 mm in length by 85.4 mm±0.5 mm in width, and includes 6, 12, 24, 48, or 96 wells, wherein each well has an average diameter of about 6 mm.

The term “well” refers to a discrete concave feature in a substrate having a surface opening that is completely surrounded by interstitial region(s) of the surface. Wells can have any of a variety of shapes at their opening in a surface including but not limited to round, elliptical, square, polygonal, or star shaped (i.e., star shaped with any number of vertices). The cross section of a well taken orthogonally with the surface may be curved, square, polygonal, hyperbolic, conical, or angular. The wells of a microplate are available in different shapes, for example F-Bottom: flat bottom; C-Bottom: bottom with minimal rounded edges; V-Bottom: V-shaped bottom; or U-Bottom: U-shaped bottom. In embodiments, the well is substantially square. In embodiments, the well is square. In embodiments, the well is F-bottom. In embodiments, the microplate includes 24 substantially round flat bottom wells. In embodiments, the microplate includes 48 substantially round flat bottom wells. In embodiments, the microplate includes 96 substantially round flat bottom wells. In embodiments, the microplate includes 384 substantially square flat bottom wells.

The discrete regions (i.e., features, wells) of the microplate may have defined locations in a regular array, which may correspond to a rectilinear pattern, circular pattern, hexagonal pattern, or the like. In embodiments, the pattern of wells includes concentric circles of regions, spiral patterns, rectilinear patterns, hexagonal patterns, and the like. In embodiments, the pattern of wells is arranged in a rectilinear or hexagonal pattern A regular array of such regions is advantageous for detection and data analysis of signals collected from the arrays during an analysis. These discrete regions are separated by interstitial regions. As used herein, the term “interstitial region” refers to an area in a substrate or on a surface that separates other areas of the substrate or surface. For example, an interstitial region can separate one concave feature of an array from another concave feature of the array. The two regions that are separated from each other can be discrete, lacking contact with each other. In another example, an interstitial region can separate a first portion of a feature from a second portion of a feature. In embodiments the interstitial region is continuous whereas the features are discrete, for example, as is the case for an array of wells in an otherwise continuous surface. The separation provided by an interstitial region can be partial or full separation. In embodiments, interstitial regions have a surface material that differs from the surface material of the wells (e.g., the interstitial region contains a photoresist and the surface of the well is glass). In embodiments, interstitial regions have a surface material that is the same as the surface material of the wells (e.g., both the surface of the interstitial region and the surface of well contain a polymer or copolymer).

As used herein, the term “sequencing reaction mixture” is used in accordance with its plain and ordinary meaning and refers to an aqueous mixture that contains the reagents necessary to allow dNTP or dNTP analogue (e.g., a modified nucleotide) to add a nucleotide to a DNA strand by a DNA polymerase. In embodiments, the sequencing reaction mixture includes a buffer. In embodiments, the buffer includes an acetate buffer, 3-(N-morpholino) propanesulfonic acid (MOPS) buffer, N-(2-Acetamido)-2-aminoethanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer. In embodiments, the sequencing reaction mixture includes nucleotides, wherein the nucleotides include a reversible terminating moiety and a label covalently linked to the nucleotide via a cleavablelinker. In embodiments, the sequencing reaction mixture includes a buffer, DNA polymerase, detergent (e.g., Triton™ X), a chelator (e.g., EDTA), and/or salts (e.g., ammonium sulfate, magnesium chloride, sodium chloride, or potassium chloride).

As used herein, the term “sequencing cycle” is used in accordance with its plain and ordinary meaning and refers to incorporating one or more nucleotides (e.g., nucleotide analogues) to the 3′ end of a polynucleotide with a polymerase, and detecting one or more labels that identify the one or more nucleotides incorporated. In embodiments, one nucleotide (e.g., a modified nucleotide) is incorporated per sequencing cycle. The sequencing may be accomplished by, for example, sequencing by synthesis, pyrosequencing, and the like. In embodiments, a sequencing cycle includes extending a complementary polynucleotide by incorporating a first nucleotide using a polymerase, wherein the polynucleotide is hybridized to a template nucleic acid, detecting the first nucleotide, and identifying the first nucleotide. In embodiments, to begin a sequencing cycle, one or more differently labeled nucleotides and a DNA polymerase can be introduced. Following nucleotide addition, signals produced (e.g., via excitation and emission of a detectable label) can be detected to determine the identity of the incorporated nucleotide (based on the labels on the nucleotides). Reagents can then be added to remove the 3′ reversible terminator and to remove labels from each incorporated base. Reagents, enzymes, and other substances can be removed between steps by washing. Cycles may include repeating these steps, and the sequence of each cluster is read over the multiple repetitions. In embodiments, a sequencing cycle incorporates one modified nucleotide into a primer hybridized to a template.

As used herein, the term “extension” or “elongation” is used in accordance with their plain and ordinary meanings and refer to synthesis by a polymerase of a new polynucleotide strand complementary to a template strand by adding free nucleotides (e.g., dNTPs) from a reaction mixture that are complementary to the template in the 5′-to-3′ direction. Extension includes condensing the 5′-phosphate group of the dNTPs with the 3′-hydroxy group at the end of the nascent (elongating) DNA strand.

As used herein, the term “sequencing read” is used in accordance with its plain and ordinary meaning and refers to an inferred sequence of nucleotide bases (or nucleotide base probabilities) corresponding to all or part of a single polynucleotide fragment. A sequencing read may include 1, 2, 5, 10, 20, 30, 40, 50, 60, 70, 80, 90, 100, 150, 200, 250, or more nucleotide bases. In embodiments, a sequencing read includes reading a barcode sequence and a template nucleotide sequence. In embodiments, a sequencing read includes reading a template nucleotide sequence. In embodiments, a sequencing read includes reading a barcode and not a template nucleotide sequence. Reads of length 20-40 base pairs (bp) are referred to as ultra-short. Typical sequencers produce read lengths in the range of 100-500 bp. Read length is a factor which can affect the results of biological studies. For example, longer read lengths improve the resolution of de novo genome assembly and detection of structural variants. In embodiments, a sequencing read includes reading a barcode and a template nucleotide sequence. In embodiments, a sequencing read includes reading a template nucleotide sequence. In embodiments, a sequencing read includes reading a barcode and not a template nucleotide sequence. In embodiments, a sequencing read includes a computationally derived string corresponding to the detected label. In some embodiments, a sequencing read may include 300, 400, 500, 600, 700, 800, 900, 1,000, 1,100, 1,200, 1,300, 1,400, 1,500, or more nucleotide bases.

As used herein, the term “code,” means a system of rules to convert information, such as signals obtained from a detection apparatus, into another form or representation, such as a base call or nucleic acid sequence. For example, signals that are produced by one or more incorporated nucleotides can be encoded by a digit. The digit can have several potential values, each value encoding a different signal state. For example, a binary digit will have a first value for a first signal state and a second value for a second signal state. A digit can have a higher radix including, for example, a ternary digit having three potential values, a quaternary digit having four potential values, etc. A series of digits can form a codeword. The length of the codeword is the same as the number of sequencing steps performed. Exemplary codes include, but are not limited to, a Hamming code. A Hamming code is used in accordance with its ordinary meaning in computer science, mathematics, telecommunication sciences and refers to a code that can be used to detect and correct the errors that can occur when the data is moved or stored. The Hamming distance refers to the difference in integer number between two codewords of equal length, and may be determined using known techniques in the art such as the Hamming distance test or the Hamming distance algorithm. For example, for two codewords (i.e., two sequenced barcodes that have been converted to a string of integers), a difference of 0 indicates that the codewords (i.e., the sequences) are identical. A difference of 1 in integer value indicates a Hamming distance of 1, thus 1 base difference between the oligos. Hamming distance is the number of positions for which the corresponding bit values in the two strings are different. In other words, the test measures the minimum number of substitutions that would be necessary to change one bit string into the other.

The term “multiplexing” as used herein refers to an analytical method in which the presence and/or amount of multiple targets, e.g., multiple nucleic acid target sequences, can be assayed simultaneously by using the methods and devices as described herein, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic. As used herein, the term “multiplex” is used to refer to an assay in which multiple (i.e. at least two) different biomolecules are assayed at the same time, and more particularly in the same aliquot of the sample, or in the same reaction mixture. In embodiments, more than two different biomolecules are assayed at the same time. In embodiments, at least 2, 4, 6, 8, 10, 20, 50, 100, 200, 300, 400, 500, 600, 700, 800, 900, 1000, 1100, 1200, 1300, 1400 or 1500 or more biomolecules are detected according to the present method.

Complementary single stranded nucleic acids and/or substantially complementary single stranded nucleic acids can hybridize to each other under hybridization conditions, thereby forming a nucleic acid that is partially or fully double stranded. All or a portion of a nucleic acid sequence may be substantially complementary to another nucleic acid sequence, in some embodiments. As referred to herein, “substantially complementary” refers to nucleotide sequences that can hybridize with each other under suitable hybridization conditions. Hybridization conditions can be altered to tolerate varying amounts of sequence mismatch within complementary nucleic acids that are substantially complementary. Substantially complementary portions of nucleic acids that can hybridize to each other can be 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other. In some embodiments substantially complementary portions of nucleic acids that can hybridize to each other are 100% complementary. Nucleic acids, or portions thereof, that are configured to hybridize to each other often include nucleic acid sequences that are substantially complementary to each other.

“Hybridize” shall mean the annealing of a nucleic acid sequence to another nucleic acid sequence (e.g., one single-stranded nucleic acid (such as a primer) to another nucleic acid) based on the well-understood principle of sequence complementarity. In an embodiment the other nucleic acid is a single-stranded nucleic acid. In some embodiments, one portion of a nucleic acid hybridizes to itself, such as in the formation of a hairpin structure. The propensity for hybridization between nucleic acids depends on the temperature and ionic strength of their milieu, the length of the nucleic acids and the degree of complementarity. The effect of these parameters on hybridization is described in, for example, Sambrook J., Fritsch E. F., Maniatis T., Molecular cloning: a laboratory manual, Cold Spring Harbor Laboratory Press, New York (1989). As used herein, hybridization of a primer, or of a DNA extension product, respectively, is extendable by creation of a phosphodiester bond with an available nucleotide or nucleotide analogue capable of forming a phosphodiester bond, therewith. For example, hybridization can be performed at a temperature ranging from 15° C. to 95° C. In some embodiments, the hybridization is performed at a temperature of about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C. In other embodiments, the stringency of the hybridization can be further altered by the addition or removal of components of the buffered solution.

As used herein, “specifically hybridizes” refers to preferential hybridization under hybridization conditions where two nucleic acids, or portions thereof, that are substantially complementary, hybridize to each other and not to other nucleic acids that are not substantially complementary to either of the two nucleic acids. For example, specific hybridization includes the hybridization of a primer or capture nucleic acid to a portion of a target nucleic acid (e.g., a template, or adapter portion of a template) that is substantially complementary to the primer or capture nucleic acid. In some embodiments nucleic acids, or portions thereof, that are configured to specifically hybridize are often about 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more or 100% complementary to each other over a contiguous portion of nucleic acid sequence. A specific hybridization discriminates over non-specific hybridization interactions (e.g., two nucleic acids that a not configured to specifically hybridize, e.g., two nucleic acids that are 80% or less, 70% or less, 60% or less or 50% or less complementary) by about 2-fold or more, often about 10-fold or more, and sometimes about 100-fold or more, 1000-fold or more, 10,000-fold or more, 100,000-fold or more, or 1,000,000-fold or more. Two nucleic acid strands that are hybridized to each other can form a duplex which includes a double stranded portion of nucleic acid.

As used herein, the term “adjacent,” refers to two nucleotide sequences in a nucleic acid, can refer to nucleotide sequences separated by 0 to about 20 nucleotides, more specifically, in a range of about 1 to about 10 nucleotides, or to sequences that directly abut one another. As those of skill in the art appreciate, two nucleotide sequences that that are to ligated together will generally directly abut one another.

A nucleic acid can be amplified by a suitable method. The term “amplification,” “amplified” or “amplifying” as used herein refers to subjecting a target nucleic acid in a sample to a process that linearly or exponentially generates amplicon nucleic acids having the same or substantially the same (e.g., substantially identical) nucleotide sequence as the target nucleic acid, or segment thereof, and/or a complement thereof (which may be referred to herein as an “amplification product” or “amplification products”). In some embodiments an amplification reaction comprises a suitable thermal stable polymerase. Thermal stable polymerases are known and are stable for prolonged periods of time, at temperature greater than 80° C. when compared to common polymerases found in most mammals. In certain embodiments the term “amplification,” “amplified” or “amplifying” refers to a method that includes a polymerase chain reaction (PCR). Conditions conducive to amplification (i.e., amplification conditions) are known and often include at least a suitable polymerase, a suitable template, a suitable primer or set of primers, suitable nucleotides (e.g., dNTPs), a suitable buffer, and application of suitable annealing, hybridization and/or extension times and temperatures. In certain embodiments an amplified product (e.g., an amplicon) can contain one or more additional and/or different nucleotides than the template sequence, or portion thereof, from which the amplicon was generated (e.g., a primer can contain “extra” nucleotides (such as a 5′ portion that does not hybridize to the template), or one or more mismatched bases within a hybridizing portion of the primer).

As used herein, the term “rolling circle amplification (RCA)” refers to a nucleic acid amplification reaction that amplifies a circular nucleic acid template (e.g., single-stranded DNA circles) via a rolling circle mechanism. Rolling circle amplification reaction is initiated by the hybridization of a primer to a circular, often single-stranded, nucleic acid template. The nucleic acid polymerase then extends the primer that is hybridized to the circular nucleic acid template by continuously progressing around the circular nucleic acid template to replicate the sequence of the nucleic acid template over and over again (rolling circle mechanism). The rolling circle amplification typically produces concatemers including tandem repeat units of the circular nucleic acid template sequence. The rolling circle amplification may be a linear RCA (LRCA), exhibiting linear amplification kinetics (e.g., RCA using a single specific primer), or may be an exponential RCA (ERCA) exhibiting exponential amplification kinetics. Rolling circle amplification may also be performed using multiple primers (multiply primed rolling circle amplification or MPRCA) leading to hyper-branched concatemers. For example, in a double-primed RCA, one primer may be complementary, as in the linear RCA, to the circular nucleic acid template, whereas the other may be complementary to the tandem repeat unit nucleic acid sequences of the RCA product. Consequently, the double-primed RCA may proceed as a chain reaction with exponential (geometric) amplification kinetics featuring a ramifying cascade of multiple-hybridization, primer-extension, and strand-displacement events involving both the primers. This often generates a discrete set of concatemeric, double-stranded nucleic acid amplification products. The rolling circle amplification may be performed in-vitro under isothermal conditions using a suitable nucleic acid polymerase such as Phi29 DNA polymerase. RCA may be performed by using any of the DNA polymerases that are known in the art (e.g., a Phi29 DNA polymerase, a Bst DNA polymerase, or SD polymerase).

A nucleic acid can be amplified by a thermocycling method or by an isothermal amplification method. In some embodiments a rolling circle amplification method is used. In some embodiments amplification takes place on a solid support (e.g., within a flow cell) where a nucleic acid, nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support.

In some embodiments solid phase amplification includes a nucleic acid amplification reaction including only one species of oligonucleotide primer immobilized to a surface or substrate. In certain embodiments solid phase amplification includes a plurality of different immobilized oligonucleotide primer species. In some embodiments solid phase amplification may include a nucleic acid amplification reaction including one species of oligonucleotide primer immobilized on a solid surface and a second different oligonucleotide primer species in solution. Multiple different species of immobilized or solution-based primers can be used. Non-limiting examples of solid phase nucleic acid amplification reactions include interfacial amplification, bridge PCR amplification, emulsion PCR, WildFire amplification (e.g., US patent publication US20130012399), the like or combinations thereof.

As used herein, the terms “cluster” and “colony” are used interchangeably to refer to a discrete site on a solid support that includes a plurality of immobilized polynucleotides and a plurality of immobilized complementary polynucleotides. The term “clustered array” refers to an array formed from such clusters or colonies. In this context the term “array” is not to be understood as requiring an ordered arrangement of clusters. The term “array” is used in accordance with its ordinary meaning in the art, and refers to a population of different molecules that are attached to one or more solid-phase substrates such that the different molecules can be differentiated from each other according to their relative location. An array can include different molecules that are each located at different addressable features on a solid-phase substrate. The molecules of the array can be nucleic acid primers, nucleic acid probes, nucleic acid templates or nucleic acid enzymes such as polymerases or ligases. Arrays useful in the invention can have densities that ranges from about 2 different features to many millions, billions or higher. The density of an array can be from 2 to as many as a billion or more different features per square cm. For example an array can have at least about 100 features/cm², at least about 1,000 features/cm², at least about 10,000 features/cm², at least about 100,000 features/cm², at least about 10,000,000 features/cm², at least about 100,000,000 features/cm², at least about 1,000,000,000 features/cm², at least about 2,000,000,000 features/cm² or higher. In embodiments, the arrays have features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

Provided herein are methods, systems, and compositions for analyzing a sample (e.g., sequencing nucleic acids within a sample) in situ. The term “in situ” is used in accordance with its ordinary meaning in the art and refers to a sample surrounded by at least a portion of its native environment, such as may preserve the relative position of two or more elements. For example, an extracted human cell obtained is considered in situ when the cell is retained in its local microenvironment so as to avoid extracting the target (e.g., nucleic acid molecules or proteins) away from their native environment. An in situ sample (e.g., a cell) can be obtained from a suitable subject. An in situ cell sample may refer to a cell and its surrounding milieu, or a tissue. A sample can be isolated or obtained directly from a subject or part thereof. In embodiments, the methods described herein (e.g., sequencing a plurality of target nucleic acids of a cell in situ) are applied to an isolated cell (i.e., a cell not surrounded by least a portion of its native environment). For the avoidance of any doubt, when the method is performed within a cell (e.g., an isolated cell) the method may be considered in situ. In some embodiments, a sample is obtained indirectly from an individual or medical professional. A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, ear, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, ear, nails, the like, parts thereof or combinations thereof. A sample may include cells or tissues that are normal, healthy, diseased (e.g., infected), and/or cancerous (e.g., cancer cells). A sample obtained from a subject may include cells or cellular material (e.g., nucleic acids) of multiple organisms (e.g., virus nucleic acid, fetal nucleic acid, bacterial nucleic acid, parasite nucleic acid). A sample may include a cell and RNA transcripts. A sample can include nucleic acids obtained from one or more subjects. In some embodiments a sample includes nucleic acid obtained from a single subject. A subject can be any living or non-living organism, including but not limited to a human, non-human animal, plant, bacterium, fungus, virus, or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a plant. In some embodiments, a subject is a human subject. A subject can be a patient (e.g., a human patient). In some embodiments a subject is suspected of having a genetic variation or a disease or condition associated with a genetic variation.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer. A protein may refer to a protein expressed in or on a cell.

A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

As used herein, a “single cell” refers to one cell. Single cells useful in the methods described herein can be obtained from a tissue of interest, or from a biopsy, blood sample, or cell culture. Additionally, cells from specific organs, tissues, tumors, neoplasms, or the like can be obtained and used in the methods described herein. In general, cells from any population can be used in the methods, such as a population of prokaryotic or eukaryotic organisms, including bacteria or yeast.

The term “cellular component” is used in accordance with its ordinary meaning in the art and refers to any organelle, nucleic acid, protein, or analyte that is found in a prokaryotic, eukaryotic, archaeal, or other organismic cell type. Examples of cellular components (e.g., a component of a cell) include RNA transcripts, proteins, membranes, lipids, and other analytes.

A “gene” refers to a polynucleotide that is capable of conferring biological function after being transcribed and/or translated.

As used herein, the terms “biomolecule” or “analyte” refer to an agent (e.g., a compound, macromolecule, or small molecule), and the like derived from a biological system (e.g., an organism, a cell, or a tissue). The biomolecule may contain multiple individual components that collectively construct the biomolecule, for example, in embodiments, the biomolecule is a polynucleotide wherein the polynucleotide is composed of nucleotide monomers. The biomolecule may be or may include DNA, RNA, organelles, carbohydrates, lipids, proteins, or any combination thereof. These components may be extracellular. In some examples, the biomolecule may be referred to as a clump or aggregate of combinations of components. In some instances, the biomolecule may include one or more constituents of a cell but may not include other constituents of the cell. In embodiments, a biomolecule is a molecule produced by a biological system (e.g., an organism). The biomolecule may be any substance (e.g. molecule) or entity that is desired to be detected by the method of the invention. The biomolecule is the “target” of the assay method of the invention. The biomolecule may accordingly be any compound that may be desired to be detected, for example a peptide or protein, or nucleic acid molecule or a small molecule, including organic and inorganic molecules. The biomolecule may be a cell or a microorganism, including a virus, or a fragment or product thereof. Biomolecules of particular interest may thus include proteinaceous molecules such as peptides, polypeptides, proteins or prions or any molecule which includes a protein or polypeptide component, etc., or fragments thereof. The biomolecule may be a single molecule or a complex that contains two or more molecular subunits, which may or may not be covalently bound to one another, and which may be the same or different. Thus, in addition to cells or microorganisms, such a complex biomolecule may also be a protein complex. Such a complex may thus be a homo- or hetero-multimer. Aggregates of molecules e.g., proteins may also be target analytes, for example aggregates of the same protein or different proteins. The biomolecule may also be a complex between proteins or peptides and nucleic acid molecules such as DNA or RNA. Of particular interest may be the interactions between proteins and nucleic acids, e.g., regulatory factors, such as transcription factors, and interactions between DNA or RNA molecules.

As used herein, “biomaterial” refers to any biological material produced by an organism. In some embodiments, biomaterial includes secretions, extracellular matrix, proteins, lipids, organelles, membranes, cells, portions thereof, and combinations thereof. In some embodiments, cellular material includes secretions, extracellular matrix, proteins, lipids, organelles, membranes, cells, portions thereof, and combinations thereof. In some embodiments, biomaterial includes viruses. In some embodiments, the biomaterial is a replicating virus and thus includes virus infected cells. In embodiments, a biological sample includes biomaterials.

The term “organelle” as used herein refers to an entity of cell associated with a particular function. In embodiments, an organelle refers to a specialized subunit within a cell that has a specific function, and is usually separately enclosed within its own lipid bilayer. Examples of organelles include the nucleus, mitochondria, endoplasmic reticulum, Golgi apparatus, lysosomes, and chloroplasts (in plant cells). Although most organelles are functional units within cells, some organelles function extend outside of cells, such as cilia, flagellum, archaellum, and the trichocyst. In embodiments, the organelle is a membrane bound organelle. In embodiments, the organelle is a non-membrane bound organelle. Non-membrane bounded organelles, also called biomolecular complexes, are assemblies of macromolecules such as the ribosome, the spliceosome, the proteasome, the nucleosome, and the centriole. Commonly detected organelles includes the nucleus, which is often visualized using dyes such as DAPI, Hoechst, and SYTO Green, mitochondria are with MitoTracker™ dyes and Rhodamine 123, endoplasmic reticulum (ER) utilizing dyes like ER-Tracker® Green/Red or DiOC6, the Golgi apparatus is stained with BODIPY™ FL C5-Ceramide and NBD C6-Ceramide, lysosomes are typically stained using LysoTracker™ dyes and Acridine Orange, and peroxisomes may be stained with Peroxisome-Tracker® Red and Peroxy Green dyes. Although not membrane-bound, ribosomes may detected using antibodies such as anti-RPL10 or anti-RPS6. Additionally, the cytoskeleton, specifically actin filaments, is frequently stained to study cell shape with Phalloidin conjugates and Alexa Fluor® Phalloidin being widely used. In embodiments, the organelle is a biomolecular complex including a plurality of subunits. In embodiments, the organelle is a macromolecule. In embodiments, the organelle is a eukaryotic organelle. In embodiments, the organelle is the cell membrane, the endoplasmic reticulum, a flagellum, a Golgi apparatus, a mitochondria, the nucleus, a vacuole. In embodiments, the organelle is a lysosome. In embodiments, the organelle is the nucleolus.

In some embodiments, a sample includes one or more nucleic acids, or fragments thereof. A sample can include nucleic acids obtained from one or more subjects. In some embodiments a sample includes nucleic acid obtained from a single subject. In some embodiments, a sample includes a mixture of nucleic acids. A mixture of nucleic acids can include two or more nucleic acid species having different nucleotide sequences, different fragment lengths, different origins (e.g., genomic origins, cell or tissue origins, subject origins, the like or combinations thereof), or combinations thereof. A sample may include synthetic nucleic acid.

A subject can be any living or non-living organism, including but not limited to a human, non-human animal, plant, bacterium, fungus, virus or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a human subject. A subject can be a patient (e.g., a human patient). In some embodiments a subject is suspected of having a genetic variation or a disease or condition associated with a genetic variation.

The methods and kits of the present disclosure may be applied, mutatis mutandis, to the sequencing of RNA, or to determining the identity of a ribonucleotide.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., packaging, buffers, written instructions for performing a method, etc.) from one location to another. For example, kits include one or more enclosures (e.g., boxes) containing the relevant reaction reagents and/or supporting materials. As used herein, the term “fragmented kit” refers to a delivery system including two or more separate containers that each contain a subportion of the total kit components. The containers may be delivered to the intended recipient together or separately. For example, a first container may contain an enzyme for use in an assay, while a second container contains oligonucleotides. In contrast, a “combined kit” refers to a delivery system containing all of the components of a reaction assay in a single container (e.g., in a single box housing each of the desired components). The term “kit” includes both fragmented and combined kits.

As used herein the term “determine” can be used to refer to the act of ascertaining, establishing or estimating. A determination can be probabilistic. For example, a determination can have an apparent likelihood of at least 50%, 75%, 90%, 95%, 98%, 99%, 99.9% or higher. In some cases, a determination can have an apparent likelihood of 100%. An exemplary determination is a maximum likelihood analysis or report. As used herein, the term “identify,” when used in reference to a thing, can be used to refer to recognition of the thing, distinction of the thing from at least one other thing or categorization of the thing with at least one other thing. The recognition, distinction or categorization can be probabilistic. For example, a thing can be identified with an apparent likelihood of at least 50%, 75%, 90%, 95%, 98%, 99%, 99.9% or higher. A thing can be identified based on a result of a maximum likelihood analysis. In some cases, a thing can be identified with an apparent likelihood of 100%.

The terms “bioconjugate group,” “bioconjugate reactive moiety,” and “bioconjugate reactive group” refer to a chemical moiety which participates in a reaction to form a bioconjugate linker (e.g., covalent linker). Non-limiting examples of bioconjugate reactive groups and the resulting bioconjugate reactive linkers may be found in the Bioconjugate Table below:

| Bioconjugate reactive group 1 | Bioconjugate reactive group 2 | |
|---|---|---|
| (e.g., electrophilic | (e.g., nucleophilic bioconjugate | Resulting Bioconjugate |
| bioconjugate reactive moiety) | reactive moiety) | reactive linker |
| activated esters | amines/anilines | carboxamides |
| acrylamides | thiols | thioethers |
| acyl azides | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides |
| aryl halides | thiols | thiophenols |
| aryl halides | amines | aryl amines |
| aziridines | thiols | thioethers |
| boronates | glycols | boronate esters |
| carbodiimides | carboxylic acids | N-acylureas or anhydrides |
| diazoalkanes | carboxylic acids | esters |
| epoxides | thiols | thioethers |
| haloacetamides | thiols | thioethers |
| haloplatinate | amino | platinum complex |
| haloplatinate | heterocycle | platinum complex |
| haloplatinate | thiol | platinum complex |
| halotriazines | amines/anilines | aminotriazines |
| halotriazines | alcohols/phenols | triazinyl ethers |
| halotriazines | thiols | triazinyl thioethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonate esters | thiols | thioethers |
| sulfonate esters | carboxylic acids | esters |
| sulfonate esters | alcohols | ethers |
| sulfonyl halides | amines/anilines | sulfonamides |
| sulfonyl halides | phenols/alcohols | sulfonate esters |

As used herein, the term “bioconjugate reactive moiety” and “bioconjugate reactive group” refers to a moiety or group capable of forming a bioconjugate (e.g., covalent linker) as a result of the association between atoms or molecules of bioconjugate reactive groups. The association can be direct or indirect. For example, a conjugate between a first bioconjugate reactive group (e.g., —NH₂, —COOH, —N-hydroxy succinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxy succinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive groups used for bioconjugate chemistries herein include, for example: (a) carboxyl groups and various derivatives thereof including, but not limited to, N-hydroxy succinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (1) metal silicon oxide bonding; (m) metal bonding to reactive phosphorus groups (e.g., phosphines) to form, for example, phosphate diester bonds.; (n) azides coupled to alkynes using copper catalyzed cycloaddition click chemistry; (o) biotin conjugate can react with avidin or strepavidin to form a avidin-biotin complex or streptavidin-biotin complex.

An “antibody” (Ab) is a protein that binds specifically to a particular substance, known as an “antigen” (Ag). An “antibody” or “antigen-binding fragment” is an immunoglobulin that binds a specific “epitope.” The term encompasses polyclonal, monoclonal, and chimeric antibodies. In nature, antibodies are generally produced by lymphocytes in response to immune challenge, such as by infection or immunization. An “antigen” (Ag) is any substance that reacts specifically with antibodies or T lymphocytes (T cells). An antibody may include the entire antibody as well as any antibody fragments capable of binding the antigen or antigenic fragment of interest. Examples include complete antibody molecules, antibody fragments, such as Fab, F(ab′)2, CDRs, VL, VH, and any other portion of an antibody which is capable of specifically binding to an antigen. Antibodies used herein are immunospecific for, and therefore specifically and selectively bind to, for example, proteins either detected (e.g., biological targets of interest) or used for detection (e.g., probes containing oligonucleotide barcodes) in the methods and devices as described herein.

The term “covalent linker” is used in accordance with its ordinary meaning and refers to a divalent moiety which connects at least two moieties to form a molecule.

The term “non-covalent linker” is used in accordance with its ordinary meaning and refers to a divalent moiety which includes at least two molecules that are not covalently linked to each other but are capable of interacting with each other via a non-covalent bond (e.g., electrostatic interactions (e.g., ionic bond, hydrogen bond, halogen bond) or van der Waals interactions (e.g., dipole-dipole, dipole-induced dipole, London dispersion). In embodiments, the non-covalent linker is the result of two molecules that are not covalently linked to each other that interact with each other via a non-covalent bond.

As used herein a “genetically modifying agent” is a substance that alters the genetic sequence of a cell following exposure to the cell, resulting in an agent-mediated nucleic acid sequence. In embodiments, the genetically modifying agent is a small molecule, protein, pathogen (e.g., virus or bacterium), toxin, oligonucleotide, or antigen. In embodiments, the genetically modifying agent is a virus (e.g., influenza) and the agent-mediated nucleic acid sequence is the nucleic acid sequence that develops within a T-cell upon cellular exposure and contact with the virus. In embodiments, the genetically modifying agent modulates the expression of a nucleic acid sequence in a cell relative to a control (e.g., the absence of the genetically modifying agent).

Where a range of values is provided, it is understood that each intervening value, to the tenth of the unit of the lower limit unless the context clearly indicates otherwise, between the upper and lower limit of that range, and any other stated or unstated intervening value in, or smaller range of values within, that stated range is encompassed within the invention. The upper and lower limits of any such smaller range (within a more broadly recited range) may independently be included in the smaller ranges, or as particular values themselves, and are also encompassed within the invention, subject to any specifically excluded limit in the stated range. Where the stated range includes one or both of the limits, ranges excluding either or both of those included limits are also included in the invention.

As used herein, the term “upstream” refers to a region in the nucleic acid sequence that is towards the 5′ end of a particular reference point, and the term “downstream” refers to a region in the nucleic acid sequence that is toward the 3′ end of the reference point.

As used herein, the terms “incubate,” and “incubation refer collectively to altering the temperature of an object in a controlled manner such that conditions are sufficient for conducting the desired reaction. Thus, it is envisioned that the terms encompass heating a receptacle (e.g., a microplate) to a desired temperature and maintaining such temperature for a fixed time interval. Also included in the terms is the act of subjecting a receptacle to one or more heating and cooling cycles (i.e., “temperature cycling” or “thermal cycling”). While temperature cycling typically occurs at relatively high rates of change in temperature, the term is not limited thereto, and may encompass any rate of change in temperature.

As used herein, “biological activity” may include the in vivo activities of a compound or physiological responses that result upon in vivo administration of a compound, composition or other mixture. Biological activity, thus, may encompass therapeutic effects and pharmaceutical activity of such compounds, compositions and mixtures. Biological activities may be observed in vitro systems designed to test or use such activities.

The term “isolated” means altered or removed from the natural state. For example, a nucleic acid or a polypeptide naturally present in a living animal is not isolated, but the same nucleic acid or polypeptide partially or completely separated from the coexisting materials of its natural state is isolated. An isolated nucleic acid or protein can exist in substantially purified form, or can exist in a non-native environment such as, for example, a host cell. In embodiments, “isolated” refers to a nucleic acid, polynucleotide, polypeptide, protein, or other component that is partially or completely separated from components with which it is normally associated (other proteins, nucleic acids, cells, etc.).

The term “synthetic target” as used herein refers to a modified protein or nucleic acid such as those constructed by synthetic methods. In embodiments, a synthetic target is artificial or engineered, or derived from or contains an artificial or engineered protein or nucleic acid (e.g., non-natural or not wild type). For example, a polynucleotide that is inserted or removed such that it is not associated with nucleotide sequences that normally flank the polynucleotide as it is found in nature is a synthetic target polynucleotide.

The term “nucleic acid sequencing device” and the like means an integrated system of one or more chambers, ports, and channels that are interconnected and in fluid communication and designed for carrying out an analytical reaction or process, either alone or in cooperation with an appliance or instrument that provides support functions, such as sample introduction, fluid and/or reagent driving means, temperature control, detection systems, data collection and/or integration systems, for the purpose of determining the nucleic acid sequence of a template polynucleotide. Nucleic acid sequencing devices may further include valves, pumps, and specialized functional coatings on interior walls. Nucleic acid sequencing devices may include a receiving unit, or platen, that orients the flow cell such that a maximal surface area of the flow cell is available to be exposed to an optical lens. Other nucleic acid sequencing devices include those provided by Singular Genomics™ (e.g., the G4™ system), Illumina™ (e.g., HiSeq™, MiSeq™, NextSeq™, or NovaSeq™ systems), Life Technologies™ (e.g., ABI PRISM™, or SOLiD™ systems), Pacific Biosciences (e.g., systems using SMRT™ Technology such as the Sequel™ or RS II™ systems), or Qiagen (e.g., Genereader™ system). Nucleic acid sequencing devices may further include fluidic reservoirs (e.g., bottles), valves, pressure sources, pumps, sensors, control systems, valves, pumps, and specialized functional coatings on interior walls. In embodiments, the device includes a plurality of a sequencing reagent reservoirs and a plurality of clustering reagent reservoirs. In embodiments, the clustering reagent reservoir includes amplification reagents (e.g., an aqueous buffer containing enzymes, salts, and nucleotides, denaturants, crowding agents, etc.) In embodiments, the reservoirs include sequencing reagents (such as an aqueous buffer containing enzymes, salts, and nucleotides); a wash solution (an aqueous buffer); a cleave solution (an aqueous buffer containing a cleaving agent, such as a reducing agent); or a cleaning solution (a dilute bleach solution, dilute NaOH solution, dilute HCl solution, dilute antibacterial solution, or water). The fluid of each of the reservoirs can vary. The fluid can be, for example, an aqueous solution which may contain buffers (e.g., saline-sodium citrate (SSC), ascorbic acid, tris(hydroxymethyl)aminomethane or “Tris”), aqueous salts (e.g., KCl or (NH₄)₂SO₄)), nucleotides, polymerases, cleaving agent (e.g., tri-n-butyl-phosphine, triphenyl phosphine and its sulfonated versions (i.e., tris(3-sulfophenyl)-phosphine, TPPTS), and tri(carboxyethyl)phosphine (TCEP) and its salts, cleaving agent scavenger compounds (e.g., 2′-Dithiobisethanamine or 11-Azido-3,6,9-trioxaundecane-1-amine), chelating agents (e.g., EDTA), detergents, surfactants, crowding agents, or stabilizers (e.g., PEG, Tween, BSA). Non-limited examples of reservoirs include cartridges, pouches, vials, containers, and eppendorf tubes. In embodiments, the device is configured to perform fluorescent imaging. In embodiments, the device includes one or more light sources (e.g., one or more lasers). In embodiments, the illuminator or light source is a radiation source (i.e., an origin or generator of propagated electromagnetic energy) providing incident light to the sample. A radiation source can include an illumination source producing electromagnetic radiation in the ultraviolet (UV) range (about 200 to 390 nm), visible (VIS) range (about 390 to 770 nm), or infrared (IR) range (about 0.77 to 25 microns), or other range of the electromagnetic spectrum. In embodiments, the illuminator or light source is a lamp such as an arc lamp or quartz halogen lamp. In embodiments, the illuminator or light source is a coherent light source. In embodiments, the light source is a laser, LED (light emitting diode), a mercury or tungsten lamp, or a super-continuous diode. In embodiments, the light source provides excitation beams having a wavelength between 200 nm to 1500 nm. In embodiments, the laser provides excitation beams having a wavelength of 405 nm, 470 nm, 488 nm, 514 nm, 520 nm, 532 nm, 561 nm, 633 nm, 639 nm, 640 nm, 800 nm, 808 nm, 912 nm, 1024 nm, or 1500 nm. In embodiments, the illuminator or light source is a light-emitting diode (LED). The LED can be, for example, an Organic Light Emitting Diode (OLED), a Thin Film Electroluminescent Device (TFELD), or a Quantum dot based inorganic organic LED. The LED can include a phosphorescent OLED (PHOLED). In embodiments, the nucleic acid sequencing device includes an imaging system (e.g., an imaging system as described herein). The imaging system capable of exciting one or more of the identifiable labels (e.g., a fluorescent label) linked to a nucleotide and thereafter obtain image data for the identifiable labels. The image data (e.g., detection data) may be analyzed by another component within the device. The imaging system may include a system described herein and may include a fluorescence spectrophotometer including an objective lens and/or a solid-state imaging device. The solid-state imaging device may include a charge coupled device (CCD) and/or a complementary metal oxide semiconductor (CMOS). The system may also include circuitry and processors, including systems using microcontrollers, reduced instruction set computers (RISC), application specific integrated circuits (ASICs), field programmable gate array (FPGAs), logic circuits, and any other circuit or processor capable of executing functions described herein. The set of instructions may be in the form of a software program. As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. In embodiments, the device includes a thermal control assembly useful to control the temperature of the reagents.

The term “image” is used according to its ordinary meaning and refers to a representation of all or part of an object. The representation may be an optically detected reproduction. For example, an image can be obtained from fluorescent, luminescent, scatter, or absorption signals. The part of the object that is present in an image can be the surface or other xy plane of the object. Typically, an image is a 2 dimensional representation of a 3 dimensional object. An image may include signals at differing intensities (i.e., signal levels). An image can be provided in a computer readable format or medium. An image is derived from the collection of focus points of light rays coming from an object (e.g., the sample), which may be detected by any image sensor.

As used herein, the term “signal” is intended to include, for example, fluorescent, luminescent, scatter, or absorption impulse or electromagnetic wave transmitted or received. Signals can be detected in the ultraviolet (UV) range (about 200 to 390 nm), visible (VIS) range (about 391 to 770 nm), infrared (IR) range (about 0.771 to 25 microns), or other range of the electromagnetic spectrum. The term “signal level” refers to an amount or quantity of detected energy or coded information. For example, a signal may be quantified by its intensity, wavelength, energy, frequency, power, luminance, or a combination thereof. Other signals can be quantified according to characteristics such as voltage, current, electric field strength, magnetic field strength, frequency, power, temperature, etc. Absence of signal is understood to be a signal level of zero or a signal level that is not meaningfully distinguished from noise.

The term “xy coordinates” refers to information that specifies location, size, shape, and/or orientation in an xy plane. The information can be, for example, numerical coordinates in a Cartesian system. The coordinates can be provided relative to one or both of the x and y axes or can be provided relative to another location in the xy plane (e.g., a fiducial). The term “xy plane” refers to a 2 dimensional area defined by straight line axes x and y. When used in reference to a detecting apparatus and an object observed by the detector, the xy plane may be specified as being orthogonal to the direction of observation between the detector and object being detected.

As used herein, the term “tissue section” refers to a piece of tissue that has been obtained from a subject, optionally fixed and attached to a surface, e.g., a microscope slide or solid support as described herein.

“PD-L1” or “Programmed death-ligand 1”, also known as cluster of differentiation 274 (CD274), or B7 homolog 1 (B7-H1) is used according to its common, ordinary meaning and refers to proteins of the same or similar names and functional fragments and homologs thereof. Programmed death-ligand 1 (PD-L1) is a 40 kDa type 1 transmembrane protein, and is capable of binding to its receptor, PD-1, found on activated T cells, B cells, and myeloid cells. PD-L1 also has an appreciable affinity for the costimulatory molecule CD80 (B7-1). PD-L1 is typically expressed on macrophages. The term includes any recombinant or naturally occurring form of PD-L1 (e.g., “Programmed death-ligand 1”; Entrez Gene 29126, OMIM 605402, UniProtKB Q9NZQ7, and/or RefSeq (protein) NP_054862.1). The term includes PD-L1 and variants thereof that maintain PD-L1 activity (e.g., within at least 30%, 40%, 50%, 60%, 70%, 80%, 90%, 95%, or 100% activity compared to PD-L1). In embodiments, PD-L1 is an immune checkpoint. In embodiments, PD-L1 is human PD-L1.

“CD8” or “cluster of differentiation 8” refers to a transmembrane glycoprotein and is used according to its common, ordinary meaning and refers to proteins of the same or similar names and functional fragments and homologs thereof. The term includes any recombinant or naturally occurring form of CD8 (e.g. “Cluster of Differentiation 8”; Entrez Gene 925, OMIM 186910 (CD8A), OMIM 186730 (CD8B), UniProtKB P01732 (CD8A), UniProtKB P10966 (CD8B), and/or RefSeq (protein) NP_001759.3). The term includes CD8 and variants thereof that maintain CD8 activity (e.g. within at least 30%, 40%, 50%, 60%, 70%, 80%, 90%, 95%, or 100% activity compared to CD8). Along with the T-cell receptor (TCR), the CD8 co-receptor plays a role in T cell signaling and aiding with cytotoxic T cell-antigen interactions. CD8 typically forms a dimer, consisting of a pair of CD8 chains: a CD8-α (CD8A) and CD8-β (CD8B) chain. The CD8 protein is predominantly expressed on the surface of cytotoxic T cells, but can also be found on natural killer cells, cortical thymocytes, and dendritic cells. In embodiments, CD8 is human CD8. In embodiments, is associated with cytotoxic T cell infiltration.

“CD3” or “cluster of differentiation 3” refers to a protein complex and is used according to its common, ordinary meaning and refers to proteins of the same or similar names and functional fragments and homologs thereof. CD3 is composed of four distinct protein chains, a CD3γ chain, a CD3δ chain, and two CD3ε chains. The term includes any recombinant or naturally occurring form of CD3 (e.g., “CD3 molecule”; Entrez Gene 915 (CD3D), Entrez Gene 916 (CD3E), Entrez Gene 917 (CD3G), OMIM 186790 (CD3D), OMIM 186830 (CD3E), OMIM 186740 (CD3G), UniProtKB P04234, UniProtKB P07766 (CD3E), UniProtKB P09693

(CD3G)). The term includes CD3 and variants thereof that maintain CD3 activity (e.g. within at least 30%, 40%, 50%, 60%, 70%, 80%, 90%, 95%, or 100% activity compared to CD3). The CD3γ, CD3δ, and CD3ε chains are related cell-surface proteins of the immunoglobulin superfamily containing an extracellular immunoglobulin domain. In embodiments, CD3 is a universal T cell marker. In embodiments, CD3 is human CD3.

“CD3e” is used according to its common, ordinary meaning and refers to proteins of the same or similar names and functional fragments and homologs thereof. The term includes any recombinant or naturally occurring form of CD3e (e.g. “CD3 epsilon molecule”; Entrez Gene 916, UniProtKB P07766). The term includes CD3e and variants thereof that maintain CD3e activity (e.g. within at least 30%, 40%, 50%, 60%, 70%, 80%, 90%, 95%, or 100% activity compared to CD3e). The CD3e protein, which together with CD3-gamma, -delta and -zeta, and the T-cell receptor alpha/beta and gamma/delta heterodimers, forms the T cell receptor-CD3 complex. The detection of the CD3e marker is useful as a T cell marker. In embodiments, CD3e is a universal T cell marker. In embodiments, CD3e is human CD3e.

“PD1” or “PD-1” or “programmed cell death protein 1”, also known as CD279, is a cell surface receptor on T cells and B cells and is used according to its common, ordinary meaning and refers to proteins of the same or similar names and functional fragments and homologs thereof. The term includes any recombinant or naturally occurring form of PD1 (e.g., “Programmed cell death protein 1”; Entrez Gene 5133, OMIM 600244, UniProtKB Q15116, and/or RefSeq (protein) NP_005009.2). The term includes PD1 and variants thereof that maintain PD1 activity (e.g., within at least 30%, 40%, 50%, 60%, 70%, 80%, 90%, 95%, or 100% activity compared to PD1). In embodiments, PD-1 is bound to another protein called PD-L1. In embodiments, PD1 is an immune checkpoint receptor. In embodiments, PD1 is human PD1.

“CD45” is used according to its common, ordinary meaning and refers to proteins of the same or similar names and functional fragments and homologs thereof. The term includes any recombinant or naturally occurring form of CD45 (e.g., “Protein tyrosine phosphatase, receptor type C”; Entrez Gene 5788, OMIM 151460, UniProtKB P08575, and/or RefSeq (protein) NP_002829.2). The term includes CD45 and variants thereof that maintain CD45 activity (e.g., within at least 30%, 40%, 50%, 60%, 70%, 80%, 90%, 95%, or 100% activity compared to CD45). Six different human isoforms of CD45 mRNAs have been isolated, which contain all three exons (ABC isoform), two of the three exons (AB and BC isoform), only one exon (A isoform and B isoform), or no exons (O isoform). All of the isoforms have the same eight amino acids at their amino-terminus, which are followed by the various combinations of A, B, and C peptides (66, 47, and 48 amino acids long, respectively). The remaining regions (the 383-amino-acid extracellular region, the 22-aminoacid transmembrane peptide, and the 707 amino acid-cytoplasmic region) have the identical sequences in all isoforms. As a result of the variability of the N-terminal region of CD45, antibodies raised against the CD45 protein recognize either all of the CD45 isoforms or only a subset of them (CD45R). The suffix RA, RB, or RO indicates the requirement of the amino acid residues corresponding to exon A (RA), exon B (RB), or a lack of amino acid residues corresponding to exon A, B and C (RO) for the CD45 epitope expression, respectively. Thus, “CD45RO” refers to an isoform of CD45 corresponding to UniProtKB P08575-4 and “CD45RA” refers to an isoform of CD45 corresponding to UniProtKB P0875-8. CD45RO contains exon 3, 7 and 8 and lacks the RA, RB and RC exons of the CD45 gene. The CD45RO isoform is expressed on activated and memory T cells, some B cell subsets, activated monocytes/macrophages, and granulocytes. CD45RA contains exon 4 but lacks exon 5 and 6. CD45RA is typically expressed on naïve T cells. In embodiments, CD45RA is human CD45RA.

“CD4” or “cluster of differentiation 4” refers to a glycoprotein that serves as a co-receptor for the T-cell receptor (TCR) and is used according to its common, ordinary meaning and refers to proteins of the same or similar names and functional fragments and homologs thereof. The term includes any recombinant or naturally occurring form of CD4 (e.g., “Cluster of Differentiation 4”; Entrez Gene 920, OMIM 186940, UniProtKB P01730, and/or RefSeq (protein) NP_000607.1). The term includes CD4 and variants thereof that maintain CD4 activity (e.g., within at least 30%, 40%, 50%, 60%, 70%, 80%, 90%, 95%, or 100% activity compared to CD4). CD4 is typically found on the surface of immune cells such as helper T cells, monocytes, macrophages, and dendritic cells. In embodiments, CD4 is human CD4.

“CD68” or “cluster of differentiation 68” refers to a transmembrane glycoprotein and is used according to its common, ordinary meaning and refers to proteins of the same or similar names and functional fragments and homologs thereof. The term includes any recombinant or naturally occurring form of CD68 (e.g. “CD68 molecule”; Entrez Gene 968, OMIM 153634, UniProtKB P34810, and/or RefSeq (protein) NP_001035149.1). The term includes CD68 and variants thereof that maintain CD68 activity (e.g. within at least 30%, 40%, 50%, 60%, 70%, 80%, 90%, 95%, or 100% activity compared to CD68). The human CD68 protein is encoded by the CD68 gene which maps to chromosome 17. Typically, in humans, CD68 is a glycosylated glycoprotein that is highly expressed in macrophages and other mononuclear phagocytes. In embodiments, CD68 is human CD68.

“CD11c” or “Integrin, alpha X (complement component 3 receptor 4 subunit) (ITGAX)” refers to an integrin alpha X chain protein and is used according to its common, ordinary meaning and refers to proteins of the same or similar names and functional fragments and homologs thereof. The term includes any recombinant or naturally occurring form of CD11c (e.g., “Integrin alpha-X”; Entrez Gene 3687, OMIM 151510, UniProtKB P20702, and/or RefSeq (protein) NP_000878). The term includes CD11c and variants thereof that maintain CD11c activity (e.g., within at least 30%, 40%, 50%, 60%, 70%, 80%, 90%, 95%, or 100% activity compared to CD11c). CD11c is a type I transmembrane protein found at high levels on most human dendritic cells, but also on monocytes, macrophages, neutrophils, and some B cells. In embodiments, CD11c is human CD11c.

“FoxP3” or “forkhead box P3”, also known as scurfin, is a protein involved in immune system responses and is used according to its common, ordinary meaning and refers to proteins of the same or similar names and functional fragments and homologs thereof. The term includes any recombinant or naturally occurring form of FoxP3 (e.g., “Forkhead Box P3”; Entrez Gene 50943, OMIM 300292, UniProtKB Q9BZS1, and/or RefSeq (protein) NP_001135417). The term includes FoxP3 and variants thereof that maintain FoxP3 activity (e.g., within at least 30%, 40%, 50%, 60%, 70%, 80%, 90%, 95%, or 100% activity compared to FoxP3). FoxP3 is a marker of natural T regulatory cells and adaptive/induced T regulatory cells. In embodiments, FoxP3 is human FoxP3.

“α-SMA” or “ACTA2” or “actin alpha 2” is an actin protein and is used according to its common, ordinary meaning and refers to proteins of the same or similar names and functional fragments and homologs thereof. The term includes any recombinant or naturally occurring form of α-SMA (e.g. “Alpha-smooth muscle actin”; Entrez Gene 59, OMIM 102620, UniProtKB P62736, and/or RefSeq (protein) NP_001091.1). The term includes α-SMA and variants thereof that maintain α-SMA activity (e.g. within at least 30%, 40%, 50%, 60%, 70%, 80%, 90%, 95%, or 100% activity compared to α-SMA). In embodiments, α-SMA is human α-SMA.

“CD20” refers to a B lymphocyte cell surface protein and is used according to its common, ordinary meaning and refers to proteins of the same or similar names and functional fragments and homologs thereof. The term includes any recombinant or naturally occurring form of CD20 (e.g. “B-lymphocyte antigen CD20”; Entrez Gene 931, OMIM 112210, UniProtKB P11836, and/or RefSeq (protein) NP_068769.1). The term includes CD20 and variants thereof that maintain CD20 activity (e.g. within at least 30%, 40%, 50%, 60%, 70%, 80%, 90%, 95%, or 100% activity compared to CD20). In embodiments, CD20 is human CD20.

“Ki67” or “Antigen Kiel 67” is a protein encoded by the MKI67 gene and is used according to its common, ordinary meaning and refers to proteins of the same or similar names and functional fragments and homologs thereof. The term includes any recombinant or naturally occurring form of Ki67 (e.g., “Marker of proliferation Ki-67”; Entrez Gene 4288, OMIM 176741, UniProtKB P46013, and/or RefSeq (protein) NP_002408.1). The term includes Ki67 and variants thereof that maintain Ki67 activity (e.g., within at least 30%, 40%, 50%, 60%, 70%, 80%, 90%, 95%, or 100% activity compared to Ki67). In embodiments, Ki67 is human Ki-67.

“CD56” or “Neural cell adhesion molecule (NCAM)” is a glycoprotein and is used according to its common, ordinary meaning and refers to proteins of the same or similar names and functional fragments and homologs thereof. The term includes any recombinant or naturally occurring form of CD56 (e.g., “Neural Cell Adhesion Molecule 1”; Entrez Gene 4684, OMIM 116920, UniProtKB P13591, and/or RefSeq (protein) NP_000606). The term includes CD56 and variants thereof that maintain CD56 activity (e.g., within at least 30%, 40%, 50%, 60%, 70%, 80%, 90%, 95%, or 100% activity compared to CD56). The expression of CD56 is associated with natural killer cells. In embodiments, CD56 is human CD56.

“CD31” or “Platelet endothelial cell adhesion molecule (PECAM-1)” refers to a protein encoded by the PECAM1 gene and is used according to its common, ordinary meaning and refers to proteins of the same or similar names and functional fragments and homologs thereof. The term includes any recombinant or naturally occurring form of CD31 (e.g. “Platelet Endothelial Cell Adhesion Molecule”; Entrez Gene 5175, OMIM 173445, UniProtKB P16284, and/or RefSeq (protein) NP_000433.3). The term includes CD31 and variants thereof that maintain CD31 activity (e.g. within at least 30%, 40%, 50%, 60%, 70%, 80%, 90%, 95%, or 100% activity compared to CD31). In embodiments, CD31 is human CD31.

“CTLA-4” or “Cytotoxic T-lymphocyte associated protein 4”, also referred to as CD152, refers to a protein receptor encoded by the CDLA4 gene in humans and is used according to its common, ordinary meaning and refers to proteins of the same or similar names and functional fragments and homologs thereof. The term includes any recombinant or naturally occurring form of CTLA-4 (e.g., “Cytotoxic T-Lymphocyte Associated Protein 4”; Entrez Gene 1493, OMIM 123890, UniProtKB P16410, and/or RefSeq (protein) NP_005205.1). The term includes CTLA-4 and variants thereof that maintain CTLA-4 activity (e.g., within at least 30%, 40%, 50%, 60%, 70%, 80%, 90%, 95%, or 100% activity compared to CTLA-4). In embodiments, CDLA-4 is human CDLA-4.

“PanCK” is used according to its common, ordinary meaning and refers to proteins of the same or similar names and functional fragments and homologs thereof. The term includes any recombinant or naturally occurring form of PanCK (e.g., “Pan Cytokeratin”; UniProtKB P12035). The term includes PanCK and variants thereof that maintain PanCK activity (e.g., within at least 30%, 40%, 50%, 60%, 70%, 80%, 90%, 95%, or 100% activity compared to PanCK). Cytokeratins are proteins of keratin-containing intermediate filaments found in the intracytoplasmic cytoskeleton of epithelial tissue. In embodiments, PanCK is human PanCK.

“HLA-DR” or “HLA-DRA” refers to HLA class II histocompatibility antigen, DR alpha chain encoded by the HLA-DRA gene and is used according to its common, ordinary meaning and refers to proteins of the same or similar names and functional fragments and homologs thereof. The term includes any recombinant or naturally occurring form of HLA-DR (e.g., “Human Leukocyte Antigen-DR”; Entrez Gene 3122, OMIM 142860, UniProtKB P01903, and/or RefSeq (protein) NP_061984). The term includes HLA-DR and variants thereof that maintain HLA-DR activity (e.g., within at least 30%, 40%, 50%, 60%, 70%, 80%, 90%, 95%, or 100% activity compared to HLA-DR). In embodiments, HLA-DR is human HLA-DR.

“Vimentin” refers to a protein encoded by the VIM gene and is used according to its common, ordinary meaning and refers to proteins of the same or similar names and functional fragments and homologs thereof. The term includes any recombinant or naturally occurring form of Vimentin (e.g., “Vimentin”; Entrez Gene 7431, OMIM 193060, UniProtKB P08670, and/or RefSeq (protein) NP_003371.1). The term includes Vimentin and variants thereof that maintain Vimentin activity (e.g., within at least 30%, 40%, 50%, 60%, 70%, 80%, 90%, 95%, or 100% activity compared to Vimentin). In embodiments, Vimentin is human Vimentin.

“γH2AX” is used according to its common, ordinary meaning and refers to proteins of the same or similar names and functional fragments and homologs thereof. The term includes any recombinant or naturally occurring form of γH2AX (e.g., “phosphorylated H2A histone family member X”; UniProtKB P16104). The term includes γH2AX and variants thereof that maintain γH2AX activity (e.g., within at least 30%, 40%, 50%, 60%, 70%, 80%, 90%, 95%, or 100% activity compared to γH2AX). In embodiments, γH2AX is human γH2AX.

The term “conjugate” is used in its accordance with its plain and ordinary meaning and refers to a composition containing at least two components linked together. The individual components may be linked directly through one or more covalent bonds, or one or more ionic bonds, or by chelation, or mixtures thereof. The linkage, or conjugation, may include one or more spacer groups between the one or more linkages joining the one or more individual components, or may be between the individual component and the linkage. The individual components that may be linked together may include biologically derived biopolymers, modified biopolymers, biologically derived biomolecules, and synthetically derived molecules. For example, the conjugate may comprise a first component, such as a protein, that may be linked, i.e., conjugated, directly through one or more covalent bonds to a second component, such as an oligonucleotide, to form a conjugate. The conjugate and/or the linkage of the conjugate may be stable to thermolysis, stable to hydrolysis, may be biocompatible, or combinations thereof.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### II. Compositions & Kits

In an aspect is provided a composition. In embodiments, the composition includes a specific binding reagent (alternatively referred to herein as a specific binding agent) covalently attached to an oligonucleotide. In embodiments, the oligonucleotide includes a sequence at least 80% identical to a sequence selected from SEQ ID NO:1 to SEQ ID NO:132. In embodiments, the oligonucleotide includes a sequence selected from SEQ ID NO:1 to SEQ ID NO: 132. In embodiments, the oligonucleotide is hybridized to a first blocking oligonucleotide and a second blocking oligonucleotide. In embodiments, the oligonucleotide is 30 to 40 nucleotides. In embodiments, the oligonucleotide does not include five consecutive weak bases. In embodiments, the oligonucleotide does not include five consecutive strong bases. In embodiments, the oligonucleotide does not include secondary structure.

In embodiments, the specific binding agent is a monoclonal antibody or a polyclonal antibody. In embodiments, the specific binding agent is capable of binding (e.g., capable of specifically binding) to an actin filament of a cell, a plasma membrane of a cell, a mitochondria of a cell, the endoplasmic reticulum of a cell, a tubule of the endoplasmic reticulum, a cisternae of the endoplasmic reticulum, sheets and tubules of the endoplasmic reticulum, a nuclear envelope of the endoplasmic reticulum, a Golgi apparatus of a cell, cisternae of the Golgi apparatus, a lysosome of a cell, phosphatidylserine, a cell surface carbohydrate, or a transferrin receptor. In embodiments, the specific binding agent is capable of binding a carbohydrate on a cell surface. In embodiments, the specific binding agent is capable of binding a glycolipid, a glycoprotein, an α-glucopyranosyl residue on a cell membrane, an N-acetylglucosaminyl residue on a cell membrane, an N-acetylneuraminic acid (sialic acid) on a cell membrane, peroxisome, a nucleus, an endosome, or a cytoskeletal protein. In embodiments, the cytoskeletal protein includes talin. In embodiments, the cytoskeletal protein includes tubulin. In embodiments, the specific binding agent is a monovalent phalloidin molecule, monovalent wheat germ agglutinin molecule, monovalent concanavalin A molecule, an annexin molecule, transferrin molecule, lectin molecule, or Hoescht 33342. In embodiments, the specific binding agent is a cell paint (see, e.g., Gustafsdottir S. M. et al. PLOS One. 2013 Dec. 2; 8 (12):e80999).

In embodiments, the specific binding agent is an antibody, single-chain Fv fragment (scFv), antibody fragment-antigen binding (Fab), affimer, or an aptamer. In embodiments, the specific binding reagent is an antibody. In embodiments, the specific binding reagent is a single-chain Fv fragment (scFv). In embodiments, the specific binding reagent is an antibody fragment-antigen binding (Fab). In embodiments, the specific binding reagent is an affimer. In embodiments, the specific binding reagent is an aptamer. In embodiments, the specific binding agent is specific for a target molecule described herein. In embodiments, the specific binding agent include on average between 1.0 and 5, or between 1 and 2.5 oligonucleotides conjugated to the specific binding agent. In embodiments, the specific binding agent includes, on average, one oligonucleotide conjugated to the specific binding agent. In embodiments, the specific binding agent includes, on average, two oligonucleotides conjugated to the specific binding agent. In embodiments, the specific binding agent includes, on average, three oligonucleotides conjugated to the specific binding agent. In embodiments, the specific binding agent includes, on average, four oligonucleotides conjugated to the specific binding agent. In embodiments, the specific binding agent includes, on average, five oligonucleotides conjugated to the specific binding agent.

The stoichiometry of the conjugation reaction to form the antibody-oligonucleotide conjugates, for example, the antibody-oligonucleotide conjugates, may include one equivalent of antibody (e.g., a modified antibody to include a bioconjugate reactive moiety) and at least 0.5 equivalents of modified oligonucleotide (e.g., modified to include a bioconjugate reactive moiety, such that upon reacting with the bioconjugate reactive moiety on the antibody a bioconjugate linkage is formed). Other examples are at least 1.0 equivalent, at least 1.5 equivalents, at least 2.0 equivalents, at least 2.5 equivalents, at least 3.0 equivalents, at least 3.5 equivalents, or at least 4.0 equivalents of oligonucleotide. The stoichiometry of the conjugation reaction to form the conjugates, may include one equivalent of antibody (e.g., a modified antibody to include a bioconjugate reactive moiety) and between about 0.5 and about 2.0 of modified oligonucleotide, for example, between about 1.5 and about 2.5 equivalents, between about 2.0 and about 2.5 equivalents, between about 2.0 and about 3.0 equivalents, between about 2.5 and about 3.5 equivalents, between about 3.0 and about 3.5 equivalents, between about 3.0 and about 4.0 equivalents, or between about 3.5 and about 4.5 equivalents modified oligonucleotide. In embodiments, the stoichiometry of the conjugation reaction may be adjusted to form antibody-oligonucleotide conjugates that retain sufficient immunoreactivity of the antibody. A suitable modified oligonucleotide may be prepared by incorporating amino groups either 3′,5′ or internally using other methods and reagents. For example, the modified oligonucleotide may be prepared by reacting with a moiety that is a bifunctional molecular reagent, such as an aromatic aldehyde or ketone, aromatic hydrazino or oxyamino modification reagent, to incorporate a hydrazino or oxyamino function respectively. For example, the modified oligonucleotide may be prepared by reacting with a bifunctional molecular reagent containing a first reactive component that forms a covalent bond with the oligonucleotide, and a second reactive component that may form a linkage with a complementary reactive component on a modified antibody (e.g., an antibody containing a bioconjugate reactive moiety). In embodiments, the second reactive component may be protected such that it will not react until removed following incorporation onto the oligonucleotide. In embodiments, the bioconjugate reactive moiety is HyNic (6-HydrazinoNicotinamide). In embodiments, the modified antibody includes a HyNic-modified biomolecule (i.e., covalently modified to display a hydrazinonicotinate reactive moiety). The modified oligonucleotide may also include a 4-FB-modified oligonucleotide (i.e., covalently modified to display a 4-formylbenzamide moiety).

In embodiments, specific binding entails a binding affinity, expressed as a K_{D} (such as a K_{D} measured by surface plasmon resonance at an appropriate temperature, such as 37° C.). In embodiments, the K_{D} of a specific binding interaction is less than about 100 nM, 50 nM, 10 nM, 1 nM, 0.05 nM, or lower. In embodiments, the K_{D} of a specific binding interaction is about 0.01-100 nM, 0.1-50 nM, or 1-10 nM. In embodiments, the K_{D} of a specific binding interaction is less than 10 nM. The binding affinity of an antibody can be readily determined by one of ordinary skill in the art (for example, by Scatchard analysis). A variety of immunoassay formats can be used to select antibodies specifically immunoreactive with a particular antigen. For example, solid-phase ELISA immunoassays are routinely used to select monoclonal antibodies specifically immunoreactive with an analyte. See Harlow and Lane, ANTIBODIES: A LABORATORY MANUAL, Cold Springs Harbor Publications, New York, (1988) for a description of immunoassay formats and conditions that can be used to determine specific immunoreactivity. Typically, a specific or selective reaction will be at least twice background signal to noise and more typically more than 10 to 100 times greater than background.

Specific binding agents such as the Ab-O conjugates, as described herein, individually consist of a biomolecule-binding domain with specificity to the target analyte or biomolecule, and a nucleic acid domain linked, coupled, or conjugated thereto. For example, the biomolecule-binding domain can be for example a nucleic acid “aptamer” (Fredriksson et al (2002) Nat Biotech 20:473-477) or can be proteinaceous, such as a monoclonal or polyclonal antibody (Gullberg et al (2004) PNAS USA 101:8420-8424).

In embodiments, the antibody is selected from the antibody table provided supra. A suitable antibody or immunoglobulin may include, for example, natural antibodies, artificial antibodies, genetically engineered antibodies, monovalent antibodies, polyvalent antibodies, monoclonal antibodies, polyclonal antibodies, camelids, monobodies, scFvs and/or fragments or derivatives thereof. In embodiments, the antibody or immunoglobulin molecules may be monoclonal, polyclonal, monospecific, polyspecific, humanized, single-chain, chimeric, camelid single domain, shark single domain, synthetic, recombinant, hybrid, mutated, CDR-grafted antibodies, and/or fragments or derivatives thereof. In embodiments, antibodies may be derived from mammal species, for example, rat, mouse, goat, guinea pig, donkey, rabbit, horse, lama, camel, or avian species, such as chicken or duck. Naturally-occurring antibodies, referred to as immunoglobulins, belong to one of the immunoglobulin classes IgG, IgM, IgA, IgD, and IgE. Antibodies can also be produced synthetically. For example, recombinant antibodies, which are monoclonal antibodies, can be synthesized using synthetic genes by recovering the antibody genes from source cells, amplifying into an appropriate vector, and introducing the vector into a host to cause the host to express the recombinant antibody. In general, recombinant antibodies can be cloned from any species of antibody-producing animal using suitable oligonucleotide primers and/or hybridization probes. Recombinant techniques can be used to generate antibodies and antibody fragments, including non-endogenous species. Affimer proteins, which are affinity reagents that typically have a molecular weight of about 12-14 kDa. Affimer proteins generally bind to a target (e.g., a target protein) with both high affinity and specificity. Examples of such targets include, but are not limited to, ubiquitin chains, immunoglobulins, and C-reactive protein. In some embodiments, affimer proteins are derived from cysteine protease inhibitors, and include peptide loops and a variable N-terminal sequence that provides the binding site. The antibody may be chemically modified to include a hapten, for example a small molecule or a peptide. The hapten may be a nitrophenyl group, a dinitrophenyl group, a digoxygenin, a biotin, a Myc tag, a FLAG tag, an HA tag, an S tag, a Streptag, a His tag, a V5 tag, a ReAsh tag, a F1Ash tag, a biotinylation tag, or Sfp tag.

Antibody Table.

| | Catalog | |
|---|---|---|
| Antibody | Number | Company |
| Anti-ALDH1/2 | sc-166362 | Santa Cruz |
| Anti-ALDH1A1 | ab215996 | Abcam |
| Anti-ALDH1A1 | BCN.3.1.2A7 | CDI |
| Anti-CB1 | 93815 | CST |
| Receptor | | |
| Anti-CD11c | — | Abcam |
| Anti-CD20 | — | eBiosciences |
| Anti-CD31 | — | Abcam |
| Anti-CD3e | — | Abcam |
| Anti-CD4 | — | Abcam |
| Anti-CD45RA | — | Biolegend |
| Anti-CD56 | — | Sigma- |
| | | Aldrich |
| Anti-CD68 | — | |
| Anti-CD8 | — | Abcam |
| Anti-CTIP2 | ab18465 | Abcam |
| Anti-CTLA-4 | — | CST |
| Anti-FoxP3 | — | CST |
| Anti-Histone H3 | ab32388 | Abcam |
| (phosphor S28) | | |
| Anti-HLA-DR | — | Abcam |
| Anti-Ki67 | ab15580 | Abcam |
| Anti-Ki-67 | — | Abcam |
| Anti-MAP2 | 8707 | CST |
| Anti-Meis2 | sc-81986 | Santa Cruz |
| Anti-nNOS | ab1376 | Abcam |
| Anti-Olig2 | ab220796 | Abcam |
| Anti-Orexin A | sc-80263 | Santa Cruz |
| Anti-PanCK | — | Biolegend |
| Anti-PCDH20 | LS-C139337 | LSBio |
| Anti-PCP4 | BCN16.2.1A10 | CDI |
| Anti-PD-1 | — | Abcam |
| Anti-PDGFRA | 3174 | CST |
| Anti-PD-L1 | — | Abcam |
| Anti-Reelin | 20689-1-AP | ProteinTech |
| Anti-S100B | HX552.1.1C1 | CDI |
| Anti-S100B | HX552.1.1D12 | CDI |
| Anti-SATB2 | ab51502 | Abcam |
| Anti- | MA5-27599 | Invitrogen |
| Synaptotagmin 6 | | |

In embodiments, the specific binding agent is an enzyme, enzyme mutant, peptide, Molecular Imprinted Polymer (MIP), DARPin (Designed Ankyrin Repeat Protein), peptoid, lectin, siRNA, or miRNA molecule. In embodiments, the specific binding agent is an enzyme. In embodiments, the specific binding agent is an enzyme mutant. In embodiments, the specific binding agent is a peptide. In embodiments, the specific binding agent is a Molecular Imprinted Polymer (MIP). In embodiments, the specific binding agent is a DARPin (Designed Ankyrin Repeat Protein). In embodiments, the specific binding agent is a peptoid. In embodiments, the specific binding agent is a lectin. In embodiments, the specific binding agent is an siRNA molecule. In embodiments, the specific binding agent is a miRNA molecule.

In embodiments, the oligonucleotide is about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides. In embodiments, the oligonucleotide is 20 to 30 nucleotides. In embodiments, the oligonucleotide is 30 to 40 nucleotides. In embodiments, the oligonucleotide is 40 to 50 nucleotides.

In embodiments, the oligonucleotide does not include 2, 3, 4, 5, 6, 7, 8, 9, or 10 consecutive weak bases. In embodiments, the oligonucleotide does not include 2, 3, 4, 5, 6, 7, 8, 9, or 10 consecutive strong bases. In embodiments, the oligonucleotide does not include 2, 3, 4, 5, 6, 7, 8, 9, or 10 consecutive weak bases or 2, 3, 4, 5, 6, 7, 8, 9, or 10 consecutive strong bases. In embodiments, the oligonucleotide does not include 2, 3, 4, 5, 6, 7, 8, 9, or 10 consecutive weak bases and does not include 2, 3, 4, 5, 6, 7, 8, 9, or 10 consecutive strong bases.

In embodiments, the oligonucleotide does not include 3 consecutive weak bases. In embodiments, the oligonucleotide does not include 3 consecutive strong bases. In embodiments, the oligonucleotide does not include 3 consecutive weak bases or 3 consecutive strong bases. In embodiments, the oligonucleotide does not include 3 consecutive weak bases and does not include 3 consecutive strong bases. In embodiments, the oligonucleotide does not include 5 consecutive weak bases. In embodiments, the oligonucleotide does not include 5 consecutive strong bases. In embodiments, the oligonucleotide does not include 5 consecutive weak bases or 5 consecutive strong bases. In embodiments, the oligonucleotide does not include 5 consecutive weak bases and does not include 5 consecutive strong bases. In embodiments, the oligonucleotide does not include 7 consecutive weak bases. In embodiments, the oligonucleotide does not include 7 consecutive strong bases. In embodiments, the oligonucleotide does not include 7 consecutive weak bases or 7 consecutive strong bases. In embodiments, the oligonucleotide does not include 7 consecutive weak bases and does not include 7 consecutive strong bases. In embodiments, the oligonucleotide does not include secondary structure.

In embodiments, the oligonucleotide includes a first sequence capable of hybridizing with a first blocking oligonucleotide. In embodiments, the oligonucleotide includes a second sequence capable of hybridizing with a second blocking oligonucleotide. In embodiments, the oligonucleotide includes a first sequence capable of hybridizing with a first blocking oligonucleotide and a second sequence capable of hybridizing with a second blocking oligonucleotide.

In embodiments, the first blocking oligonucleotide includes a sequence at least 80%, 85%, 90%, 95%, or 98% identical to SEQ ID NO:133, SEQ ID NO:134, SEQ ID NO:135, SEQ ID NO: 136, SEQ ID NO: 137, SEQ ID NO: 138, SEQ ID NO:139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO:148, SEQ ID NO: 149, SEQ ID NO: 150, SEQ ID NO: 151, SEQ ID NO: 152, SEQ ID NO:153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO:162, SEQ ID NO: 163, SEQ ID NO: 164, SEQ ID NO: 165, SEQ ID NO: 166, SEQ ID NO:167, SEQ ID NO:168, SEQ ID NO: 169, SEQ ID NO: 170, SEQ ID NO: 171, SEQ ID NO:172, SEQ ID NO:173, SEQ ID NO: 174, SEQ ID NO: 175, SEQ ID NO: 176, SEQ ID NO:177, SEQ ID NO:178, SEQ ID NO: 179, SEQ ID NO:180, SEQ ID NO: 181, SEQ ID NO:182, SEQ ID NO: 183, SEQ ID NO: 184, SEQ ID NO: 185, SEQ ID NO: 186, SEQ ID NO:187, SEQ ID NO:188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO:200, SEQ ID NO: 201, SEQ ID NO:202, SEQ ID NO:203, SEQ ID NO:204, SEQ ID NO:205, SEQ ID NO: 206, SEQ ID NO:207, SEQ ID NO:208, SEQ ID NO:209, SEQ ID NO:210, SEQ ID NO: 211, SEQ ID NO:212, SEQ ID NO:213, SEQ ID NO:214, SEQ ID NO:215, SEQ ID NO: 216, SEQ ID NO:217, SEQ ID NO:218, SEQ ID NO:219, SEQ ID NO:220, SEQ ID NO: 221, SEQ ID NO:222, SEQ ID NO:223, SEQ ID NO:224, SEQ ID NO:225, SEQ ID NO: 226, SEQ ID NO:227, SEQ ID NO:228, SEQ ID NO:229, SEQ ID NO:230, SEQ ID NO: 231, SEQ ID NO:232, SEQ ID NO:233, SEQ ID NO:234, SEQ ID NO:235, SEQ ID NO: 236, SEQ ID NO:237, SEQ ID NO:238, SEQ ID NO:239, SEQ ID NO:240, SEQ ID NO: 241, SEQ ID NO:242, SEQ ID NO:243, SEQ ID NO:244, SEQ ID NO:245, SEQ ID NO: 246, SEQ ID NO:247, SEQ ID NO:248, SEQ ID NO:249, SEQ ID NO:250, SEQ ID NO: 251, SEQ ID NO:252, SEQ ID NO:253, SEQ ID NO:254, SEQ ID NO:255, SEQ ID NO: 256, SEQ ID NO:257, SEQ ID NO:258, SEQ ID NO:259, SEQ ID NO:260, SEQ ID NO: 261, SEQ ID NO:262, SEQ ID NO:263, SEQ ID NO:264, or SEQ ID NO:265.

In embodiments, the second blocking oligonucleotide includes a sequence at least 80%, 85%, 90%, 95%, or 98% identical to SEQ ID NO:133, SEQ ID NO: 134, SEQ ID NO: 135, SEQ ID NO: 136, SEQ ID NO: 137, SEQ ID NO: 138, SEQ ID NO: 139, SEQ ID NO:140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO:152, SEQ ID NO:153, SEQ ID NO: 154, SEQ ID NO:155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO:163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO:175, SEQ ID NO: 176, SEQ ID NO: 177, SEQ ID NO:178, SEQ ID NO: 179, SEQ ID NO: 180, SEQ ID NO: 181, SEQ ID NO:182, SEQ ID NO:183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO:192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO:200, SEQ ID NO: 201, SEQ ID NO:202, SEQ ID NO:203, SEQ ID NO:204, SEQ ID NO:205, SEQ ID NO: 206, SEQ ID NO:207, SEQ ID NO:208, SEQ ID NO:209, SEQ ID NO:210, SEQ ID NO: 211, SEQ ID NO:212, SEQ ID NO:213, SEQ ID NO:214, SEQ ID NO:215, SEQ ID NO: 216, SEQ ID NO:217, SEQ ID NO:218, SEQ ID NO:219, SEQ ID NO:220, SEQ ID NO: 221, SEQ ID NO:222, SEQ ID NO:223, SEQ ID NO:224, SEQ ID NO:225, SEQ ID NO: 226, SEQ ID NO:227, SEQ ID NO:228, SEQ ID NO:229, SEQ ID NO:230, SEQ ID NO: 231, SEQ ID NO:232, SEQ ID NO:233, SEQ ID NO:234, SEQ ID NO:235, SEQ ID NO: 236, SEQ ID NO:237, SEQ ID NO:238, SEQ ID NO:239, SEQ ID NO:240, SEQ ID NO: 241, SEQ ID NO:242, SEQ ID NO:243, SEQ ID NO:244, SEQ ID NO:245, SEQ ID NO: 246, SEQ ID NO:247, SEQ ID NO:248, SEQ ID NO:249, SEQ ID NO:250, SEQ ID NO: 251, SEQ ID NO:252, SEQ ID NO:253, SEQ ID NO:254, SEQ ID NO:255, SEQ ID NO: 256, SEQ ID NO:257, SEQ ID NO:258, SEQ ID NO:259, SEQ ID NO:260, SEQ ID NO: 261, SEQ ID NO:262, SEQ ID NO:263, SEQ ID NO:264, or SEQ ID NO:265.

In embodiments, the oligonucleotide is covalently attached to a specific binding agent, wherein the specific binding agent is an antibody, single-chain Fv fragment (scFv), affimer, aptamer, single-domain antibody (sdAb), or antibody fragment-antigen binding (Fab). In embodiments, the oligonucleotide is covalently attached to an antibody or single-domain antibody (sdAb). In embodiments, the oligonucleotide is covalently attached to an antibody. In embodiments, the oligonucleotide is covalently attached to a single-chain Fv fragment (scFv). In embodiments, the oligonucleotide is covalently attached to an affimer. In embodiments, the oligonucleotide is covalently attached to an aptamer. In embodiments, the oligonucleotide is covalently attached to a single-domain antibody (sdAb). In embodiments, the oligonucleotide is covalently attached to an antibody fragment-antigen binding (Fab). In embodiments, the oligonucleotide is covalently attached to an antibody or single-domain antibody (sdAb). In embodiments, the oligonucleotide is covalently attached to an enzyme. In embodiments, the oligonucleotide is covalently attached to a peptide. In embodiments, the oligonucleotide is covalently attached to a Molecular Imprinted Polymer (MIP). In embodiments, the oligonucleotide is covalently attached to a DARPin (Designed Ankyrin Repeat Protein). In embodiments, the oligonucleotide is covalently attached to a peptoid. In embodiments, the oligonucleotide is covalently attached to a lectin. The design and preparation of protein-specific binding agent oligonucleotide conjugates is known, for example various different binding moieties which may be used, the design of probe oligonucleotides, and the coupling of such oligonucleotides to the binding moieties to form the conjugates. The details and principles may be applied to the design of the probes for use in the methods described herein. For example, reference may be made to WO 2007/107743, U.S. Pat. Nos. 7,306,904 and 6,878,515 which are incorporated herein by reference. To minimize interference with the antibody binding affinity, the conjugation of the oligonucleotide to the antibody may target the intramolecular disulfide bonds present at the junction of the Fc and Fab regions of the antibodies. For example, an antibody may be modified using a bis-alkylating reagent, bis-sulfone methyltetrazine, for a trans-cyclooctene-methyltetrazine (TCO-metet) ligation reaction. The oligonucleotide may include a 5′ amine, which is further functionalized with a TCO moiety, by mixing TCO-PEG4-NHS Ester (Click Chemistry Tools, Cat. No. A137) in 0.1 M sodium bicarbonate buffer with 40% (v/v) formamide (Sigma Aldrich) at room temperature for 12 h.

In embodiments, the oligonucleotide is attached to a specific binding agent (e.g., an antibody) via a linker (e.g., a bioconjugate linker). In embodiments, the oligonucleotide is attached to the protein-specific binding agent via a linker formed by reacting a first bioconjugate reactive moiety (e.g., the bioconjugate reactive moiety includes an amine moiety, aldehyde moiety, alkyne moiety, azide moiety, carboxylic acid moiety, dibenzocyclooctyne (DBCO) moiety, tetrazine moiety, epoxy moiety, isocyanate moiety, furan moiety, maleimide moiety, thiol moiety, or transcyclooctene (TCO) moiety) with a second bioconjugate reactive moiety. In embodiments, the oligonucleotide includes a barcode, wherein the barcode is a known sequence associated with the specific binding agent. In embodiments, the barcode is at least 5, 6, 7, 8, 9, 10, 11, 12, 13, 14, or 15 nucleotides in length. In embodiments, the barcode is 5, 6, 7, 8, 9, 10, 11, 12, 13, 14, or 15 nucleotides in length.

Specific antibodies tagged with known oligonucleotide sequences can be synthesized by using bifunctional crosslinkers reactive towards thiol (via maleimide) and amine (via NHS) moieties. For example, a 5′-thiol-modified oligonucleotide could be conjugated to a crosslinker via maleimide chemistry and purified. The oligos with a 5′-NHS-ester would then be added to a solution of antibodies and reacted with amine residues on the antibodies surface to generate tagged antibodies capable of binding analytes with target epitopes. These tagged antibodies include oligonucleotide sequence(s). The one or more oligonucleotide sequences may include a barcode, binding sequences (e.g., primer binding sequence or sequences complementary to hybridization pads), and/or unique molecular identifier (UMI) sequences.

In embodiments, the composition further includes one or more polynucleotide(s) hybridized to the oligonucleotide. In embodiments, the composition includes a first polynucleotide and a second polynucleotide hybridized to the oligonucleotide. In embodiments, the first and second polynucleotides bind to adjacent sequence. Alternatively, in embodiments, the first and second polynucleotides bind to the oligonucleotide wherein a gap sequence is formed between the first and second polynucleotides. In embodiments, the polynucleotide described herein is a polynucleotide probe. In embodiments, the composition includes a polynucleotide probe hybridized to the oligonucleotide, wherein the polynucleotide probe includes a first binding sequence and a second binding sequence to the oligonucleotide.

In embodiments, the polynucleotide includes a fluorophore. One embodiment may utilize cyanine-based fluorophores, such as Cy®3 or Cy®5, known for their strong absorption and fluorescence properties, making them suitable for high-sensitivity applications. Another embodiment might incorporate fluorescein-based fluorophores, like FITC, which are characterized by their high quantum yield and are commonly used in molecular biology. Additionally, rhodamine derivatives could be used, offering robust photostability and a broad range of excitation and emission spectra. In a further embodiment, the polynucleotide may be conjugated with BODIPY fluorophores, notable for their small size, high fluorescence quantum yield, and stability under various chemical conditions. In embodiments, the oligonucleotide is attached to the antibody at the 5′ end and the fluorophore is attached to the oligonucleotide at the 3′ end. In embodiments, the oligonucleotide is attached to the antibody at the 3′ end and the fluorophore is attached to the oligonucleotide at the 5′ end.

In embodiments, the attachment of fluorophore(s) to polynucleotides can be achieved through several methods, each tailored to the specific requirements of the application. One common approach involves the covalent attachment of the fluorophore to a nucleotide at the 5′ end of the polynucleotide. This is typically done during the synthesis of the oligonucleotide using phosphoramidite chemistry, allowing for precise incorporation. Alternatively, the fluorophore can be covalently bonded at the 3′ end of the polynucleotide. This method often involves post-synthesis techniques, such as enzymatic ligation or chemical coupling using activated esters of the fluorophore, targeting the 3′ hydroxyl group. Furthermore, internal labeling of the polynucleotide is a viable approach, where the fluorophore is attached at a specific internal nucleotide. For example, one method involves the intercalation of the fluorophore between base pairs of the polynucleotide. Intercalating fluorophores, such as ethidium bromide, insert themselves between the stacked bases of the DNA double helix without forming a covalent bond, offering a non-covalent mode of attachment. In embodiments, the fluorophore can be covalently linked to the polynucleotide via a linker molecule. This linker, which provides spatial separation between the fluorophore and the nucleic acid backbone, can be instrumental in reducing quenching and enhancing the fluorescence signals.

In embodiments, the polynucleotide includes a primer binding sequence. In embodiments, the polynucleotide is referred to as a padlock probe or a circularizable oligonucleotide. In embodiments, the padlock probe includes a primer binding sequence from a known set of primer binding sequences. In embodiments, the polynucleotide includes only one primer binding sequence, wherein the primer binding sequence serves as the amplification primer binding sequence and sequencing primer binding sequence. In embodiments, the polynucleotide includes at least two primer binding sequences from a known set of primer binding sequences. In embodiments, the polynucleotide includes two or more primer binding sequences from a known set of primer binding sequences. In embodiments, a plurality of polynucleotides may include up to 20 different primer binding sequences from a known set of primer binding sequences. In embodiments, the plurality of polynucleotides includes up to 10 different primer binding sequences from a known set of primer binding sequences. In embodiments, the plurality of polynucleotides includes up to 5 different primer binding sequences from a known set of primer binding sequences. In embodiments, the polynucleotide includes two or more sequencing primer binding sequences from a known set of sequencing primer binding sequences. In embodiments, the polynucleotide includes 1, 2, 3, 4, 5, 6, 7, 8, 9, or 10 primer binding sequences from a known set of primer binding sequences. In embodiments, the polynucleotide includes two or more different primer binding sequences from a known set of primer binding sequences. In embodiments, the polynucleotide includes 1, 2, 3, 4, 5, 6, 7, 8, 9, or 10 different primer binding sequences from a known set of primer binding sequences. In embodiments, the polynucleotide includes 2 to 5 primer binding sequences from a known set of primer binding sequences. In embodiments, the polynucleotide includes 2 to 5 different primer binding sequences from a known set of primer binding sequences. In embodiments, the polynucleotide includes 2 to 5 sequencing primer binding sequences from a known set of sequencing primer binding sequences. In embodiments, the polynucleotide includes 2 to 5 different sequencing primer binding sequences from a known set of sequencing primer binding sequences.

In embodiments, the polynucleotide includes about 50 to about 150 nucleotides. In embodiments, the polynucleotide includes about 70 to about 130 nucleotides. In embodiments, the polynucleotide includes about 50 to about 300 nucleotides. In embodiments, the polynucleotide includes about 50 to about 500 nucleotides. In embodiments, the polynucleotide includes about or more than about 50, 75, 100, 125, 150, 175, 200, 250, 300, 350, 400, or 500 nucleotides. In embodiments, the polynucleotide includes less than about 50, 75, 100, 125, 150, 175, 200, 250, 300, 350, 400, or 500 nucleotides. In embodiments, the polynucleotide (i.e., polynucleotide probe) includes about 70, 75, 80, 85, 90, 95, 100, 105, 110, 115, 120, 125, 130, 135, 140, 145, or 150 nucleotides.

In embodiments, the polynucleotide includes at least one amplification primer binding sequence or at least one sequencing primer binding sequence. The amplification primer binding sequence refers to a nucleotide sequence that is complementary to a primer useful in initiating amplification (i.e., an amplification primer). Likewise, a sequencing primer binding sequence is a nucleotide sequence that is complementary to a primer useful in initiating sequencing (i.e., a sequencing primer). Primer binding sequences usually have a length in the range of between 3 to 36 nucleotides, also 5 to 24 nucleotides, also from 14 to 36 nucleotides. In embodiments, an amplification primer and a sequencing primer are complementary to the same primer binding sequence, or overlapping primer binding sequences. In embodiments, an amplification primer and a sequencing primer are complementary to different primer binding sequences.

In embodiments, the polynucleotide includes a barcode sequence. In embodiments, the polynucleotide includes a barcode nucleotide. In embodiments, the barcode sequence is at least 1, 2, 3, 4, 5, 6, 7, 8, 9, 10, 11, 12, 13, 14, or 15 nucleotides in length. In embodiments, the barcode (i.e., the barcode sequence) is at least 5, 6, 7, 8, 9, 10, 11, 12, 13, 14, or 15 nucleotides in length. In embodiments, the barcode is 5, 6, 7, 8, 9, 10, 11, 12, 13, 14, or 15 nucleotides in length. In embodiments, the barcode is 10 to 15 nucleotides in length. In embodiments, the barcode is at least about 1, 2, 3, 4, 5, 6, 7, 8, 9, 10, 15, 20, 25, 30, 35, 40, 45, 50 or more nucleotides in length. In embodiments, the barcode can be at most about 300, 200, 100, 90, 80, 70, 60, 50, 40, 30, 20, 15, 12, 10, 9, 8, 7, 6, 5, 4 or fewer or more nucleotides in length. In embodiments, the barcode includes between about 5 to about 8, about 5 to about 10, about 5 to about 15, about 5 to about 20, about 10 to about 150 nucleotides. In embodiments, the barcode includes between 5 to 8, 5 to 10, 5 to 15, 5 to 20, 10 to 150 nucleotides. In embodiments, the barcode is 10 nucleotides. In embodiments, the barcode may include a unique sequence (e.g., a barcode sequence) that gives the barcode its identifying functionality. The unique sequence may be random or non-random. Attachment of the barcode sequence (via binding of a polynucleotide described herein or polynucleotide probe described herein that is conjugated to the barcode sequence) to a protein or nucleic acid of interest (i.e., the target) may associate the barcode sequence with the protein or nucleic acid of interest. The barcode may then be used to identify the protein or nucleic acid of interest during sequencing, even when other proteins or nucleic acids of interest (e.g., including different oligonucleotide barcodes) are present. In embodiments, the barcode consists only of a unique barcode sequence. In embodiments, the 5′ end of a barcoded oligonucleotide is phosphorylated. In embodiments, the barcode is known (i.e., the nucleic sequence is known before sequencing) and is sorted into a basis-set according to their Hamming distance. Oligonucleotide barcodes (e.g., barcode sequences included in an oligonucleotide probe) can be associated with a target of interest by knowing, a priori, the target of interest, such as a gene or protein. In embodiments, the barcodes further include one or more sequences capable of specifically binding a gene or nucleic acid sequence of interest.

In embodiments, the barcode sequence is selected from a known set of barcode sequences. In embodiments, each barcode sequence is unique within the known set of barcodes. In embodiments, the barcodes are selected to form a known set of barcodes, e.g., the set of barcodes may be distinguished by a particular Hamming distance.

In embodiments, the barcodes in the known set of barcodes have a specified Hamming distance. In embodiments, the Hamming distance is 4 to 15. In embodiments, the Hamming distance is 8 to 12. In embodiments, the Hamming distance is 10. In embodiments, the Hamming distance is 0 to 100. In embodiments, the Hamming distance is 0 to 15. In embodiments, the Hamming distance is 0 to 10. In embodiments, the Hamming distance is 1 to 10. In embodiments, the Hamming distance is 5 to 10. In embodiments, the Hamming distance is 1 to 100. In embodiments, the Hamming distance between any two barcode sequences of the set is at least 2, 3, 4, or 5. In embodiments, the Hamming distance between any two barcode sequences of the set is at least 3. In embodiments, the Hamming distance between any two barcode sequences of the set is at least 4.

In embodiments, the polynucleotide includes a barcode nucleotide. A barcode nucleotide refers to a single nucleotide which may serve as a differentiating feature among targets. Detecting four different targets using a single nucleotide as a barcode may involve the use of a common primer and the incorporation of differently colored labeled nucleotides into the primer, rendering simultaneous detection of multiple targets. For example, one may bind a common primer to each of the four separate targets (e.g., amplification products arising from four separate target molecules). This common primer is designed to hybridize to a specific region shared among the targets, serving as a starting point for the subsequent incorporation of nucleotides. With a polymerase, differently colored labeled nucleotides are incorporated into the newly synthesized DNA strand opposite the barcode nucleotide. Each of the four types of nucleotides (adenine, thymine, cytosine, and guanine) is tagged with a unique fluorescent dye, with each dye emitting a distinct color upon excitation. For instance, adenine might be tagged with a green dye, thymine with blue, cytosine with red, and guanine with yellow. As the primer is extended, a colored nucleotide is incorporated to a position complementary to the barcode nucleotide. Detection is then based on the color emitted upon fluorescence excitation. For example, if the barcode nucleotide is adenine, then the complementary thymine, labeled with a blue fluorophore, is incorporated into the extending strand. The presence of the target adenine is then identified by the emission of a blue fluorescence signal. This color-coded system allows for the distinct identification of each of the four targets based on the specific fluorescence emitted by the incorporated nucleotides.

In embodiments, the polynucleotide includes locked nucleic acids (LNAs), Bis-locked nucleic acids (bisLNAs), twisted intercalating nucleic acids (TINAs), bridged nucleic acids (BNAs), 2′-O-methyl RNA:DNA chimeric nucleic acids, minor groove binder (MGB) nucleic acids, morpholino nucleic acids, C5-modified pyrimidine nucleic acids, peptide nucleic acids (PNAs), or combinations thereof. In embodiments, the polynucleotide includes one or more LNA nucleotides. In embodiments, the sequence complementary to the first hybridization sequence and/or the second sequence complementary to the second hybridization sequence of the polynucleotide includes one or more LNA nucleotides.

In embodiments, the oligonucleotide includes a sequence at least 80%, 85%, 90%, 95%, or 98% identical to a sequence selected from SEQ ID NO: 1 to SEQ ID NO:132. In embodiments, the oligonucleotide includes a sequence at least 85% identical to a sequence selected from SEQ ID NO: 1 to SEQ ID NO:132. In embodiments, the oligonucleotide includes a sequence at least 90% identical to a sequence selected from SEQ ID NO:1 to SEQ ID NO:132. In embodiments, the oligonucleotide includes a sequence at least 95% identical to a sequence selected from SEQ ID NO: 1 to SEQ ID NO:132. In embodiments, the oligonucleotide includes a sequence at least 98% identical to a sequence selected from SEQ ID NO:1 to SEQ ID NO:132. In embodiments, the oligonucleotide includes a sequence selected from SEQ ID NO: 1 to SEQ ID NO:132.

In embodiments, the oligonucleotide includes a sequence 80%, 85%, 90%, 95%, or 98% identical to a sequence selected from SEQ ID NO:1 to SEQ ID NO:132. In embodiments, the oligonucleotide includes a sequence 85% identical to a sequence selected from SEQ ID NO:1 to SEQ ID NO:132. In embodiments, the oligonucleotide includes a sequence 90% identical to a sequence selected from SEQ ID NO:1 to SEQ ID NO:132. In embodiments, the oligonucleotide includes a sequence 95% identical to a sequence selected from SEQ ID NO:1 to SEQ ID NO: 132. In embodiments, the oligonucleotide includes a sequence 98% identical to a sequence selected from SEQ ID NO:1 to SEQ ID NO:132.

In embodiments, the oligonucleotide includes a sequence at least 80%, 85%, 90%, 95%, or 98% identical to SEQ ID NO:1. In embodiments, the oligonucleotide includes SEQ ID NO:1. In embodiments, the oligonucleotide includes a sequence at least 80%, 85%, 90%, 95%, or 98% identical to SEQ ID NO:2. In embodiments, the oligonucleotide includes SEQ ID NO:2. In embodiments, the oligonucleotide includes a sequence at least 80%, 85%, 90%, 95%, or 98% identical to SEQ ID NO:3. In embodiments, the oligonucleotide includes SEQ ID NO:3. In embodiments, the oligonucleotide includes a sequence at least 80%, 85%, 90%, 95%, or 98% identical to SEQ ID NO:4. In embodiments, the oligonucleotide includes SEQ ID NO:4. In embodiments, the oligonucleotide includes a sequence at least 80%, 85%, 90%, 95%, or 98% identical to SEQ ID NO:5. In embodiments, the oligonucleotide includes SEQ ID NO:5. In embodiments, the oligonucleotide includes a sequence at least 80%, 85%, 90%, 95%, or 98% identical to SEQ ID NO:6. In embodiments, the oligonucleotide includes SEQ ID NO:6. In embodiments, the oligonucleotide includes a sequence at least 80%, 85%, 90%, 95%, or 98% identical to SEQ ID NO:7. In embodiments, the oligonucleotide includes SEQ ID NO:7. In embodiments, the oligonucleotide includes a sequence at least 80%, 85%, 90%, 95%, or 98% identical to SEQ ID NO:8. In embodiments, the oligonucleotide includes SEQ ID NO:8. In embodiments, the oligonucleotide includes a sequence at least 80%, 85%, 90%, 95%, or 98% identical to SEQ ID NO:9. In embodiments, the oligonucleotide includes SEQ ID NO:9. In embodiments, the oligonucleotide includes a sequence at least 80%, 85%, 90%, 95%, or 98% identical to SEQ ID NO:10. In embodiments, the oligonucleotide includes SEQ ID NO:10.

In embodiments, the oligonucleotide includes a sequence at least 80%, 85%, 90%, 95%, or 98% identical to SEQ ID NO:11. In embodiments, the oligonucleotide includes SEQ ID NO: 11. In embodiments, the oligonucleotide includes a sequence at least 80%, 85%, 90%, 95%, or 98% identical to SEQ ID NO:12. In embodiments, the oligonucleotide includes SEQ ID NO: 12. In embodiments, the oligonucleotide includes a sequence at least 80%, 85%, 90%, 95%, or 98% identical to SEQ ID NO:13. In embodiments, the oligonucleotide includes SEQ ID NO: 13. In embodiments, the oligonucleotide includes a sequence at least 80%, 85%, 90%, 95%, or 98% identical to SEQ ID NO:14. In embodiments, the oligonucleotide includes SEQ ID NO: 14. In embodiments, the oligonucleotide includes a sequence at least 80%, 85%, 90%, 95%, or 98% identical to SEQ ID NO:15. In embodiments, the oligonucleotide includes SEQ ID NO: 15. In embodiments, the oligonucleotide includes a sequence at least 80%, 85%, 90%, 95%, or 98% identical to SEQ ID NO:16. In embodiments, the oligonucleotide includes SEQ ID NO: 16. In embodiments, the oligonucleotide includes a sequence at least 80%, 85%, 90%, 95%, or 98% identical to SEQ ID NO:17. In embodiments, the oligonucleotide includes SEQ ID NO: 17. In embodiments, the oligonucleotide includes a sequence at least 80%, 85%, 90%, 95%, or 98% identical to SEQ ID NO:18. In embodiments, the oligonucleotide includes SEQ ID NO: 18. In embodiments, the oligonucleotide includes a sequence at least 80%, 85%, 90%, 95%, or 98% identical to SEQ ID NO:19. In embodiments, the oligonucleotide includes SEQ ID NO: 19. In embodiments, the oligonucleotide includes a sequence at least 80%, 85%, 90%, 95%, or 98% identical to SEQ ID NO:20. In embodiments, the oligonucleotide includes SEQ ID NO: 20.

In embodiments, the oligonucleotide includes a sequence at least 80%, 85%, 90%, 95%, or 98% identical to SEQ ID NO:21. In embodiments, the oligonucleotide includes SEQ ID NO: 21. In embodiments, the oligonucleotide includes a sequence at least 80%, 85%, 90%, 95%, or 98% identical to SEQ ID NO:22. In embodiments, the oligonucleotide includes SEQ ID NO: 22. In embodiments, the oligonucleotide includes a sequence at least 80%, 85%, 90%, 95%, or 98% identical to SEQ ID NO:23. In embodiments, the oligonucleotide includes SEQ ID NO: 23. In embodiments, the oligonucleotide includes a sequence at least 80%, 85%, 90%, 95%, or 98% identical to SEQ ID NO:24. In embodiments, the oligonucleotide includes SEQ ID NO: 24. In embodiments, the oligonucleotide includes a sequence at least 80%, 85%, 90%, 95%, or 98% identical to SEQ ID NO:25. In embodiments, the oligonucleotide includes SEQ ID NO: 25. In embodiments, the oligonucleotide includes a sequence at least 80%, 85%, 90%, 95%, or 98% identical to SEQ ID NO:26. In embodiments, the oligonucleotide includes SEQ ID NO: 26. In embodiments, the oligonucleotide includes a sequence at least 80%, 85%, 90%, 95%, or 98% identical to SEQ ID NO:27. In embodiments, the oligonucleotide includes SEQ ID NO: 27. In embodiments, the oligonucleotide includes a sequence at least 80%, 85%, 90%, 95%, or 98% identical to SEQ ID NO:28. In embodiments, the oligonucleotide includes SEQ ID NO: 28. In embodiments, the oligonucleotide includes a sequence at least 80%, 85%, 90%, 95%, or 98% identical to SEQ ID NO:29. In embodiments, the oligonucleotide includes SEQ ID NO: 29. In embodiments, the oligonucleotide includes a sequence at least 80%, 85%, 90%, 95%, or 98% identical to SEQ ID NO:30. In embodiments, the oligonucleotide includes SEQ ID NO: 30.

In embodiments, the oligonucleotide includes a sequence at least 80%, 85%, 90%, 95%, or 98% identical to SEQ ID NO:31. In embodiments, the oligonucleotide includes SEQ ID NO: 31. In embodiments, the oligonucleotide includes a sequence at least 80%, 85%, 90%, 95%, or 98% identical to SEQ ID NO:32. In embodiments, the oligonucleotide includes SEQ ID NO: 32. In embodiments, the oligonucleotide includes a sequence at least 80%, 85%, 90%, 95%, or 98% identical to SEQ ID NO:33. In embodiments, the oligonucleotide includes SEQ ID NO: 33. In embodiments, the oligonucleotide includes a sequence at least 80%, 85%, 90%, 95%, or 98% identical to SEQ ID NO:34. In embodiments, the oligonucleotide includes SEQ ID NO: 34. In embodiments, the oligonucleotide includes a sequence at least 80%, 85%, 90%, 95%, or 98% identical to SEQ ID NO:35. In embodiments, the oligonucleotide includes SEQ ID NO: 35. In embodiments, the oligonucleotide includes a sequence at least 80%, 85%, 90%, 95%, or 98% identical to SEQ ID NO:36. In embodiments, the oligonucleotide includes SEQ ID NO: 36. In embodiments, the oligonucleotide includes a sequence at least 80%, 85%, 90%, 95%, or 98% identical to SEQ ID NO:37. In embodiments, the oligonucleotide includes SEQ ID NO: 37. In embodiments, the oligonucleotide includes a sequence at least 80%, 85%, 90%, 95%, or 98% identical to SEQ ID NO:38. In embodiments, the oligonucleotide includes SEQ ID NO: 38. In embodiments, the oligonucleotide includes a sequence at least 80%, 85%, 90%, 95%, or 98% identical to SEQ ID NO:39. In embodiments, the oligonucleotide includes SEQ ID NO: 39. In embodiments, the oligonucleotide includes a sequence at least 80%, 85%, 90%, 95%, or 98% identical to SEQ ID NO:40. In embodiments, the oligonucleotide includes SEQ ID NO: 40.

In embodiments, the oligonucleotide includes a sequence at least 80%, 85%, 90%, 95%, or 98% identical to SEQ ID NO:41. In embodiments, the oligonucleotide includes SEQ ID NO: 41. In embodiments, the oligonucleotide includes a sequence at least 80%, 85%, 90%, 95%, or 98% identical to SEQ ID NO:42. In embodiments, the oligonucleotide includes SEQ ID NO: 42. In embodiments, the oligonucleotide includes a sequence at least 80%, 85%, 90%, 95%, or 98% identical to SEQ ID NO:43. In embodiments, the oligonucleotide includes SEQ ID NO: 43. In embodiments, the oligonucleotide includes a sequence at least 80%, 85%, 90%, 95%, or 98% identical to SEQ ID NO:44. In embodiments, the oligonucleotide includes SEQ ID NO: 44. In embodiments, the oligonucleotide includes a sequence at least 80%, 85%, 90%, 95%, or 98% identical to SEQ ID NO:45. In embodiments, the oligonucleotide includes SEQ ID NO: 45. In embodiments, the oligonucleotide includes a sequence at least 80%, 85%, 90%, 95%, or 98% identical to SEQ ID NO:46. In embodiments, the oligonucleotide includes SEQ ID NO: 46. In embodiments, the oligonucleotide includes a sequence at least 80%, 85%, 90%, 95%, or 98% identical to SEQ ID NO:47. In embodiments, the oligonucleotide includes SEQ ID NO: 47. In embodiments, the oligonucleotide includes a sequence at least 80%, 85%, 90%, 95%, or 98% identical to SEQ ID NO:48. In embodiments, the oligonucleotide includes SEQ ID NO: 48. In embodiments, the oligonucleotide includes a sequence at least 80%, 85%, 90%, 95%, or 98% identical to SEQ ID NO:49. In embodiments, the oligonucleotide includes SEQ ID NO: 49. In embodiments, the oligonucleotide includes a sequence at least 80%, 85%, 90%, 95%, or 98% identical to SEQ ID NO:50. In embodiments, the oligonucleotide includes SEQ ID NO: 50.

In embodiments, the oligonucleotide includes a sequence at least 80%, 85%, 90%, 95%, 98% identical to SEQ ID NO:51. In embodiments, the oligonucleotide includes SEQ ID NO:51. In embodiments, the oligonucleotide includes a sequence at least 80%, 85%, 90%, 95%, 98% identical to SEQ ID NO:52. In embodiments, the oligonucleotide includes SEQ ID NO:52. In embodiments, the oligonucleotide includes a sequence at least 80%, 85%, 90%, 95%, 98% identical to SEQ ID NO:53. In embodiments, the oligonucleotide includes SEQ ID NO:53. In embodiments, the oligonucleotide includes a sequence at least 80%, 85%, 90%, 95%, 98% identical to SEQ ID NO:54. In embodiments, the oligonucleotide includes SEQ ID NO:54. In embodiments, the oligonucleotide includes a sequence at least 80%, 85%, 90%, 95%, 98% identical to SEQ ID NO:55. In embodiments, the oligonucleotide includes SEQ ID NO:55. In embodiments, the oligonucleotide includes a sequence at least 80%, 85%, 90%, 95%, 98% identical to SEQ ID NO:56. In embodiments, the oligonucleotide includes SEQ ID NO:56. In embodiments, the oligonucleotide includes a sequence at least 80%, 85%, 90%, 95%, 98% identical to SEQ ID NO:57. In embodiments, the oligonucleotide includes SEQ ID NO:57. In embodiments, the oligonucleotide includes a sequence at least 80%, 85%, 90%, 95%, 98% identical to SEQ ID NO:58. In embodiments, the oligonucleotide includes SEQ ID NO:58. In embodiments, the oligonucleotide includes a sequence at least 80%, 85%, 90%, 95%, 98% identical to SEQ ID NO:59. In embodiments, the oligonucleotide includes SEQ ID NO:59. In embodiments, the oligonucleotide includes a sequence at least 80%, 85%, 90%, 95%, 98% identical to SEQ ID NO:60. In embodiments, the oligonucleotide includes SEQ ID NO:60.

In embodiments, the oligonucleotide includes a sequence at least 80%, 85%, 90%, 95%, 98% identical to SEQ ID NO:61. In embodiments, the oligonucleotide includes SEQ ID NO:61. In embodiments, the oligonucleotide includes a sequence at least 80%, 85%, 90%, 95%, 98% identical to SEQ ID NO:62. In embodiments, the oligonucleotide includes SEQ ID NO:62. In embodiments, the oligonucleotide includes a sequence at least 80%, 85%, 90%, 95%, 98% identical to SEQ ID NO:63. In embodiments, the oligonucleotide includes SEQ ID NO:63. In embodiments, the oligonucleotide includes a sequence at least 80%, 85%, 90%, 95%, 98% identical to SEQ ID NO:64. In embodiments, the oligonucleotide includes SEQ ID NO:64. In embodiments, the oligonucleotide includes a sequence at least 80%, 85%, 90%, 95%, 98% identical to SEQ ID NO:65. In embodiments, the oligonucleotide includes SEQ ID NO:65. In embodiments, the oligonucleotide includes a sequence at least 80%, 85%, 90%, 95%, 98% identical to SEQ ID NO:66. In embodiments, the oligonucleotide includes SEQ ID NO:66. In embodiments, the oligonucleotide includes a sequence at least 80%, 85%, 90%, 95%, 98% identical to SEQ ID NO:67. In embodiments, the oligonucleotide includes SEQ ID NO:67. In embodiments, the oligonucleotide includes a sequence at least 80%, 85%, 90%, 95%, 98% identical to SEQ ID NO:68. In embodiments, the oligonucleotide includes SEQ ID NO:68. In embodiments, the oligonucleotide includes a sequence at least 80%, 85%, 90%, 95%, 98% identical to SEQ ID NO:69. In embodiments, the oligonucleotide includes SEQ ID NO:69.

In embodiments, the oligonucleotide includes a sequence at least 80%, 85%, 90%, 95%, 98% identical to SEQ ID NO:70. In embodiments, the oligonucleotide includes SEQ ID NO:70. In embodiments, the oligonucleotide includes a sequence at least 80%, 85%, 90%, 95%, 98% identical to SEQ ID NO:71. In embodiments, the oligonucleotide includes SEQ ID NO:71. In embodiments, the oligonucleotide includes a sequence at least 80%, 85%, 90%, 95%, 98% identical to SEQ ID NO:72. In embodiments, the oligonucleotide includes SEQ ID NO:72. In embodiments, the oligonucleotide includes a sequence at least 80%, 85%, 90%, 95%, 98% identical to SEQ ID NO:73. In embodiments, the oligonucleotide includes SEQ ID NO:73. In embodiments, the oligonucleotide includes a sequence at least 80%, 85%, 90%, 95%, 98% identical to SEQ ID NO:74. In embodiments, the oligonucleotide includes SEQ ID NO:74. In embodiments, the oligonucleotide includes a sequence at least 80%, 85%, 90%, 95%, 98% identical to SEQ ID NO:75. In embodiments, the oligonucleotide includes SEQ ID NO:75. In embodiments, the oligonucleotide includes a sequence at least 80%, 85%, 90%, 95%, 98% identical to SEQ ID NO:76. In embodiments, the oligonucleotide includes SEQ ID NO:76. In embodiments, the oligonucleotide includes a sequence at least 80%, 85%, 90%, 95%, 98% identical to SEQ ID NO:77. In embodiments, the oligonucleotide includes SEQ ID NO:77. In embodiments, the oligonucleotide includes a sequence at least 80%, 85%, 90%, 95%, 98% identical to SEQ ID NO:78. In embodiments, the oligonucleotide includes SEQ ID NO:78. In embodiments, the oligonucleotide includes a sequence at least 80%, 85%, 90%, 95%, 98% identical to SEQ ID NO:79. In embodiments, the oligonucleotide includes SEQ ID NO:79.

In embodiments, the oligonucleotide includes a sequence at least 80%, 85%, 90%, 95%, 98% identical to SEQ ID NO:80. In embodiments, the oligonucleotide includes SEQ ID NO:80. In embodiments, the oligonucleotide includes a sequence at least 80%, 85%, 90%, 95%, 98% identical to SEQ ID NO:81. In embodiments, the oligonucleotide includes SEQ ID NO:81. In embodiments, the oligonucleotide includes a sequence at least 80%, 85%, 90%, 95%, 98% identical to SEQ ID NO:82. In embodiments, the oligonucleotide includes SEQ ID NO:82. In embodiments, the oligonucleotide includes a sequence at least 80%, 85%, 90%, 95%, 98% identical to SEQ ID NO:83. In embodiments, the oligonucleotide includes SEQ ID NO:83. In embodiments, the oligonucleotide includes a sequence at least 80%, 85%, 90%, 95%, 98% identical to SEQ ID NO:84. In embodiments, the oligonucleotide includes SEQ ID NO:84. In embodiments, the oligonucleotide includes a sequence at least 80%, 85%, 90%, 95%, 98% identical to SEQ ID NO:85. In embodiments, the oligonucleotide includes SEQ ID NO:85. In embodiments, the oligonucleotide includes a sequence at least 80%, 85%, 90%, 95%, 98% identical to SEQ ID NO:86. In embodiments, the oligonucleotide includes SEQ ID NO:86. In embodiments, the oligonucleotide includes a sequence at least 80%, 85%, 90%, 95%, 98% identical to SEQ ID NO:87. In embodiments, the oligonucleotide includes SEQ ID NO:87. In embodiments, the oligonucleotide includes a sequence at least 80%, 85%, 90%, 95%, 98% identical to SEQ ID NO:88. In embodiments, the oligonucleotide includes SEQ ID NO:88. In embodiments, the oligonucleotide includes a sequence at least 80%, 85%, 90%, 95%, 98% identical to SEQ ID NO:89. In embodiments, the oligonucleotide includes SEQ ID NO:89.

In embodiments, the oligonucleotide includes a sequence at least 80%, 85%, 90%, 95%, 98% identical to SEQ ID NO:90. In embodiments, the oligonucleotide includes SEQ ID NO:90. In embodiments, the oligonucleotide includes a sequence at least 80%, 85%, 90%, 95%, 98% identical to SEQ ID NO:91. In embodiments, the oligonucleotide includes SEQ ID NO:91. In embodiments, the oligonucleotide includes a sequence at least 80%, 85%, 90%, 95%, 98% identical to SEQ ID NO:92. In embodiments, the oligonucleotide includes SEQ ID NO:92. In embodiments, the oligonucleotide includes a sequence at least 80%, 85%, 90%, 95%, 98% identical to SEQ ID NO:93. In embodiments, the oligonucleotide includes SEQ ID NO:93. In embodiments, the oligonucleotide includes a sequence at least 80%, 85%, 90%, 95%, 98% identical to SEQ ID NO:94. In embodiments, the oligonucleotide includes SEQ ID NO:94. In embodiments, the oligonucleotide includes a sequence at least 80%, 85%, 90%, 95%, 98% identical to SEQ ID NO:95. In embodiments, the oligonucleotide includes SEQ ID NO:95. In embodiments, the oligonucleotide includes a sequence at least 80%, 85%, 90%, 95%, 98% identical to SEQ ID NO:96. In embodiments, the oligonucleotide includes SEQ ID NO:96. In embodiments, the oligonucleotide includes a sequence at least 80%, 85%, 90%, 95%, 98% identical to SEQ ID NO:97. In embodiments, the oligonucleotide includes SEQ ID NO:97. In embodiments, the oligonucleotide includes a sequence at least 80%, 85%, 90%, 95%, 98% identical to SEQ ID NO:98. In embodiments, the oligonucleotide includes SEQ ID NO:98. In embodiments, the oligonucleotide includes a sequence at least 80%, 85%, 90%, 95%, 98% identical to SEQ ID NO:99. In embodiments, the oligonucleotide includes SEQ ID NO:99.

In embodiments, the oligonucleotide includes a sequence at least 80%, 85%, 90%, 95%, 98% identical to SEQ ID NO:100. In embodiments, the oligonucleotide includes SEQ ID NO: 100. In embodiments, the oligonucleotide includes a sequence at least 80%, 85%, 90%, 95%, 98% identical to SEQ ID NO:101. In embodiments, the oligonucleotide includes SEQ ID NO: 101. In embodiments, the oligonucleotide includes a sequence at least 80%, 85%, 90%, 95%, 98% identical to SEQ ID NO:102. In embodiments, the oligonucleotide includes SEQ ID NO: 102. In embodiments, the oligonucleotide includes a sequence at least 80%, 85%, 90%, 95%, 98% identical to SEQ ID NO:103. In embodiments, the oligonucleotide includes SEQ ID NO: 103. In embodiments, the oligonucleotide includes a sequence at least 80%, 85%, 90%, 95%, 98% identical to SEQ ID NO:104. In embodiments, the oligonucleotide includes SEQ ID NO: 104. In embodiments, the oligonucleotide includes a sequence at least 80%, 85%, 90%, 95%, 98% identical to SEQ ID NO:105. In embodiments, the oligonucleotide includes SEQ ID NO: 105. In embodiments, the oligonucleotide includes a sequence at least 80%, 85%, 90%, 95%, 98% identical to SEQ ID NO:106. In embodiments, the oligonucleotide includes SEQ ID NO: 106. In embodiments, the oligonucleotide includes a sequence at least 80%, 85%, 90%, 95%, 98% identical to SEQ ID NO:107. In embodiments, the oligonucleotide includes SEQ ID NO: 107. In embodiments, the oligonucleotide includes a sequence at least 80%, 85%, 90%, 95%, 98% identical to SEQ ID NO:108. In embodiments, the oligonucleotide includes SEQ ID NO: 108. In embodiments, the oligonucleotide includes a sequence at least 80%, 85%, 90%, 95%, 98% identical to SEQ ID NO:109. In embodiments, the oligonucleotide includes SEQ ID NO: 109.

In embodiments, the oligonucleotide includes a sequence at least 80%, 85%, 90%, 95%, 98% identical to SEQ ID NO:110. In embodiments, the oligonucleotide includes SEQ ID NO: 110. In embodiments, the oligonucleotide includes a sequence at least 80%, 85%, 90%, 95%, 98% identical to SEQ ID NO:111. In embodiments, the oligonucleotide includes SEQ ID NO: 111. In embodiments, the oligonucleotide includes a sequence at least 80%, 85%, 90%, 95%, 98% identical to SEQ ID NO:112. In embodiments, the oligonucleotide includes SEQ ID NO: 112. In embodiments, the oligonucleotide includes a sequence at least 80%, 85%, 90%, 95%, 98% identical to SEQ ID NO:113. In embodiments, the oligonucleotide includes SEQ ID NO: 113. In embodiments, the oligonucleotide includes a sequence at least 80%, 85%, 90%, 95%, 98% identical to SEQ ID NO:114. In embodiments, the oligonucleotide includes SEQ ID NO: 114. In embodiments, the oligonucleotide includes a sequence at least 80%, 85%, 90%, 95%, 98% identical to SEQ ID NO:115. In embodiments, the oligonucleotide includes SEQ ID NO: 115. In embodiments, the oligonucleotide includes a sequence at least 80%, 85%, 90%, 95%, 98% identical to SEQ ID NO:116. In embodiments, the oligonucleotide includes SEQ ID NO: 116. In embodiments, the oligonucleotide includes a sequence at least 80%, 85%, 90%, 95%, 98% identical to SEQ ID NO:117. In embodiments, the oligonucleotide includes SEQ ID NO: 117. In embodiments, the oligonucleotide includes a sequence at least 80%, 85%, 90%, 95%, 98% identical to SEQ ID NO:118. In embodiments, the oligonucleotide includes SEQ ID NO: 118. In embodiments, the oligonucleotide includes a sequence at least 80%, 85%, 90%, 95%, 98% identical to SEQ ID NO:119. In embodiments, the oligonucleotide includes SEQ ID NO: 119.

In embodiments, the oligonucleotide includes a sequence at least 80%, 85%, 90%, 95%, 98% identical to SEQ ID NO:120. In embodiments, the oligonucleotide includes SEQ ID NO: 120. In embodiments, the oligonucleotide includes a sequence at least 80%, 85%, 90%, 95%, 98% identical to SEQ ID NO:121. In embodiments, the oligonucleotide includes SEQ ID NO: 121. In embodiments, the oligonucleotide includes a sequence at least 80%, 85%, 90%, 95%, 98% identical to SEQ ID NO:122. In embodiments, the oligonucleotide includes SEQ ID NO: 122. In embodiments, the oligonucleotide includes a sequence at least 80%, 85%, 90%, 95%, 98% identical to SEQ ID NO:123. In embodiments, the oligonucleotide includes SEQ ID NO: 123. In embodiments, the oligonucleotide includes a sequence at least 80%, 85%, 90%, 95%, 98% identical to SEQ ID NO:124. In embodiments, the oligonucleotide includes SEQ ID NO: 124. In embodiments, the oligonucleotide includes a sequence at least 80%, 85%, 90%, 95%, 98% identical to SEQ ID NO:125. In embodiments, the oligonucleotide includes SEQ ID NO: 125. In embodiments, the oligonucleotide includes a sequence at least 80%, 85%, 90%, 95%, 98% identical to SEQ ID NO:126. In embodiments, the oligonucleotide includes SEQ ID NO: 126. In embodiments, the oligonucleotide includes a sequence at least 80%, 85%, 90%, 95%, 98% identical to SEQ ID NO:127. In embodiments, the oligonucleotide includes SEQ ID NO: 127. In embodiments, the oligonucleotide includes a sequence at least 80%, 85%, 90%, 95%, 98% identical to SEQ ID NO:128. In embodiments, the oligonucleotide includes SEQ ID NO: 128. In embodiments, the oligonucleotide includes a sequence at least 80%, 85%, 90%, 95%, 98% identical to SEQ ID NO:129. In embodiments, the oligonucleotide includes SEQ ID NO: 129.

In embodiments, the oligonucleotide includes a sequence at least 80%, 85%, 90%, 95%, 98% identical to SEQ ID NO:130. In embodiments, the oligonucleotide includes SEQ ID NO: 130. In embodiments, the oligonucleotide includes a sequence at least 80%, 85%, 90%, 95%, 98% identical to SEQ ID NO:131. In embodiments, the oligonucleotide includes SEQ ID NO: 131. In embodiments, the oligonucleotide includes a sequence at least 80%, 85%, 90%, 95%, 98% identical to SEQ ID NO:132. In embodiments, the oligonucleotide includes SEQ ID NO: 132.

In embodiments, the polynucleotide described herein or polynucleotide probe described herein includes a sequence at least 80% identical to a sequence selected from SEQ ID NO:133 to SEQ ID NO:264. In embodiments, the polynucleotide described herein or polynucleotide probe described herein includes a sequence at least 85% identical to a sequence selected from SEQ ID NO: 133 to SEQ ID NO:264. In embodiments, the polynucleotide described herein or polynucleotide probe described herein includes a sequence at least 90% identical to a sequence selected from SEQ ID NO:133 to SEQ ID NO:264. In embodiments, the polynucleotide described herein or polynucleotide probe described herein includes a sequence at least 95% identical to a sequence selected from SEQ ID NO: 133 to SEQ ID NO:264. In embodiments, the polynucleotide described herein or polynucleotide probe described herein includes a sequence at least 98% identical to a sequence selected from SEQ ID NO:133 to SEQ ID NO:264. In embodiments, the polynucleotide described herein or polynucleotide probe described herein includes a sequence selected from SEQ ID NO:133 to SEQ ID NO:264.

In embodiments, the polynucleotide described herein or polynucleotide probe described herein includes a sequence at least 80% identical to a sequence selected from SEQ ID NO:265 to SEQ ID NO:396. In embodiments, the polynucleotide described herein or polynucleotide probe described herein includes a sequence at least 85% identical to a sequence selected from SEQ ID NO: 265 to SEQ ID NO:396. In embodiments, the polynucleotide described herein or polynucleotide probe described herein includes a sequence at least 90% identical to a sequence selected from SEQ ID NO:265 to SEQ ID NO:396. In embodiments, the polynucleotide described herein or polynucleotide probe described herein includes a sequence at least 95% identical to a sequence selected from SEQ ID NO:265 to SEQ ID NO:396. In embodiments, the polynucleotide described herein or polynucleotide probe described herein includes a sequence at least 98% identical to a sequence selected from SEQ ID NO:265 to SEQ ID NO:396. In embodiments, the polynucleotide described herein or polynucleotide probe described herein includes a sequence selected from SEQ ID NO:265 to SEQ ID NO:396.

In embodiments, the first polynucleotide includes a sequence at least 80% identical to a sequence selected from SEQ ID NO:133 to SEQ ID NO:264 and the second polynucleotide includes a sequence at least 80% identical to a sequence selected from SEQ ID NO:265 to SEQ ID NO: 396.

In embodiments, the polynucleotide described herein or polynucleotide probe described herein includes two hybridization sequences. For example, in embodiments the polynucleotide described herein or polynucleotide probe described herein includes a first sequence at least 80% identical to a sequence selected from SEQ ID NO:133 to SEQ ID NO:264 and a second sequence at least 80% identical to a sequence selected from SEQ ID NO:265 to SEQ ID NO:396.

In embodiments, the oligonucleotide includes a sequence at least 80% identical to a sequence selected from SEQ ID NO:1 to SEQ ID NO:132 and the polynucleotide described herein or polynucleotide probe described herein includes a sequence at least 80% identical to a sequence selected from SEQ ID NO:133 to SEQ ID NO:264. In embodiments, the oligonucleotide includes a sequence at least 80% identical to SEQ ID NO:1 and the polynucleotide includes a sequence at least 80% identical to SEQ ID NO:133. In embodiments, the oligonucleotide includes a sequence at least 80% identical to SEQ ID NO:2 and the polynucleotide includes a sequence at least 80% identical to SEQ ID NO:134. In embodiments, the oligonucleotide includes a sequence at least 80% identical to SEQ ID NO:3 and the polynucleotide includes a sequence at least 80% identical to SEQ ID NO:135. In embodiments, the oligonucleotide includes a sequence at least 80% identical to SEQ ID NO:4 and the polynucleotide includes a sequence at least 80% identical to SEQ ID NO:136. In embodiments, the oligonucleotide includes a sequence at least 80% identical to SEQ ID NO:5 and the polynucleotide includes a sequence at least 80% identical to SEQ ID NO:137. In embodiments, the oligonucleotide includes a sequence at least 80% identical to SEQ ID NO:6 and the polynucleotide includes a sequence at least 80% identical to SEQ ID NO:138. In embodiments, the oligonucleotide includes a sequence at least 80% identical to SEQ ID NO:7 and the polynucleotide includes a sequence at least 80% identical to SEQ ID NO:139. In embodiments, the oligonucleotide includes a sequence at least 80% identical to SEQ ID NO:8 and the polynucleotide includes a sequence at least 80% identical to SEQ ID NO:140. In embodiments, the oligonucleotide includes a sequence at least 80% identical to SEQ ID NO:9 and the polynucleotide includes a sequence at least 80% identical to SEQ ID NO:141. In embodiments, the oligonucleotide includes a sequence at least 80% identical to SEQ ID NO: 10 and the polynucleotide includes a sequence at least 80% identical to SEQ ID NO:142. In embodiments, the oligonucleotide includes a sequence at least 80% identical to SEQ ID NO:11 and the polynucleotide includes a sequence at least 80% identical to SEQ ID NO:143. In embodiments, the oligonucleotide includes a sequence at least 80% identical to SEQ ID NO: 12 and the polynucleotide includes a sequence at least 80% identical to SEQ ID NO:144. In embodiments, the oligonucleotide includes a sequence at least 80% identical to SEQ ID NO:13 and the polynucleotide includes a sequence at least 80% identical to SEQ ID NO:145. In embodiments, the oligonucleotide includes a sequence at least 80% identical to SEQ ID NO: 14 and the polynucleotide includes a sequence at least 80% identical to SEQ ID NO:146. In embodiments, the oligonucleotide includes a sequence at least 80% identical to SEQ ID NO:15 and the polynucleotide includes a sequence at least 80% identical to SEQ ID NO:147. In embodiments, the oligonucleotide includes a sequence at least 80% identical to SEQ ID NO:16 and the polynucleotide includes a sequence at least 80% identical to SEQ ID NO:148. In embodiments, the oligonucleotide includes a sequence at least 80% identical to SEQ ID NO:17 and the polynucleotide includes a sequence at least 80% identical to SEQ ID NO:149. In embodiments, the oligonucleotide includes a sequence at least 80% identical to SEQ ID NO:18 and the polynucleotide includes a sequence at least 80% identical to SEQ ID NO: 150. In embodiments, the oligonucleotide includes a sequence at least 80% identical to SEQ ID NO:19 and the polynucleotide includes a sequence at least 80% identical to SEQ ID NO:151. In embodiments, the oligonucleotide includes a sequence at least 80% identical to SEQ ID NO:20 and the polynucleotide includes a sequence at least 80% identical to SEQ ID NO:152. In embodiments, the oligonucleotide includes a sequence at least 80% identical to SEQ ID NO:21 and the polynucleotide includes a sequence at least 80% identical to SEQ ID NO:153. In embodiments, the oligonucleotide includes a sequence at least 80% identical to SEQ ID NO:22 and the polynucleotide includes a sequence at least 80% identical to SEQ ID NO:154. In embodiments, the oligonucleotide includes a sequence at least 80% identical to SEQ ID NO:23 and the polynucleotide includes a sequence at least 80% identical to SEQ ID NO:155. In embodiments, the oligonucleotide includes a sequence at least 80% identical to SEQ ID NO:24 and the polynucleotide includes a sequence at least 80% identical to SEQ ID NO:156. In embodiments, the oligonucleotide includes a sequence at least 80% identical to SEQ ID NO:25 and the polynucleotide includes a sequence at least 80% identical to SEQ ID NO:157. In embodiments, the oligonucleotide includes a sequence at least 80% identical to SEQ ID NO:26 and the polynucleotide includes a sequence at least 80% identical to SEQ ID NO:158. In embodiments, the oligonucleotide includes a sequence at least 80% identical to SEQ ID NO:27 and the polynucleotide includes a sequence at least 80% identical to SEQ ID NO:159. In embodiments, the oligonucleotide includes a sequence at least 80% identical to SEQ ID NO:28 and the polynucleotide includes a sequence at least 80% identical to SEQ ID NO:160. In embodiments, the oligonucleotide includes a sequence at least 80% identical to SEQ ID NO:29 and the polynucleotide includes a sequence at least 80% identical to SEQ ID NO: 161. In embodiments, the oligonucleotide includes a sequence at least 80% identical to SEQ ID NO:30 and the polynucleotide includes a sequence at least 80% identical to SEQ ID NO:162. In embodiments, the oligonucleotide includes a sequence at least 80% identical to SEQ ID NO:31 and the polynucleotide includes a sequence at least 80% identical to SEQ ID NO:163. In embodiments, the oligonucleotide includes a sequence at least 80% identical to SEQ ID NO:32 and the polynucleotide includes a sequence at least 80% identical to SEQ ID NO:164. In embodiments, the oligonucleotide includes a sequence at least 80% identical to SEQ ID NO:33 and the polynucleotide includes a sequence at least 80% identical to SEQ ID NO:165. In embodiments, the oligonucleotide includes a sequence at least 80% identical to SEQ ID NO:34 and the polynucleotide includes a sequence at least 80% identical to SEQ ID NO:166. In embodiments, the oligonucleotide includes a sequence at least 80% identical to SEQ ID NO:35 and the polynucleotide includes a sequence at least 80% identical to SEQ ID NO:167. In embodiments, the oligonucleotide includes a sequence at least 80% identical to SEQ ID NO:36 and the polynucleotide includes a sequence at least 80% identical to SEQ ID NO:168. In embodiments, the oligonucleotide includes a sequence at least 80% identical to SEQ ID NO:37 and the polynucleotide includes a sequence at least 80% identical to SEQ ID NO:169. In embodiments, the oligonucleotide includes a sequence at least 80% identical to SEQ ID NO:38 and the polynucleotide includes a sequence at least 80% identical to SEQ ID NO:170. In embodiments, the oligonucleotide includes a sequence at least 80% identical to SEQ ID NO:39 and the polynucleotide includes a sequence at least 80% identical to SEQ ID NO:171. In embodiments, the oligonucleotide includes a sequence at least 80% identical to SEQ ID NO:40 and the polynucleotide includes a sequence at least 80% identical to SEQ ID NO:172. In embodiments, the oligonucleotide includes a sequence at least 80% identical to SEQ ID NO:41 and the polynucleotide includes a sequence at least 80% identical to SEQ ID NO:173. In embodiments, the oligonucleotide includes a sequence at least 80% identical to SEQ ID NO:42 and the polynucleotide includes a sequence at least 80% identical to SEQ ID NO:174. In embodiments, the oligonucleotide includes a sequence at least 80% identical to SEQ ID NO:43 and the polynucleotide includes a sequence at least 80% identical to SEQ ID NO:175. In embodiments, the oligonucleotide includes a sequence at least 80% identical to SEQ ID NO:44 and the polynucleotide includes a sequence at least 80% identical to SEQ ID NO:176. In embodiments, the oligonucleotide includes a sequence at least 80% identical to SEQ ID NO:45 and the polynucleotide includes a sequence at least 80% identical to SEQ ID NO:177. In embodiments, the oligonucleotide includes a sequence at least 80% identical to SEQ ID NO:46 and the polynucleotide includes a sequence at least 80% identical to SEQ ID NO:178. In embodiments, the oligonucleotide includes a sequence at least 80% identical to SEQ ID NO:47 and the polynucleotide includes a sequence at least 80% identical to SEQ ID NO:179. In embodiments, the oligonucleotide includes a sequence at least 80% identical to SEQ ID NO:48 and the polynucleotide includes a sequence at least 80% identical to SEQ ID NO: 180. In embodiments, the oligonucleotide includes a sequence at least 80% identical to SEQ ID NO:49 and the polynucleotide includes a sequence at least 80% identical to SEQ ID NO:181. In embodiments, the oligonucleotide includes a sequence at least 80% identical to SEQ ID NO:50 and the polynucleotide includes a sequence at least 80% identical to SEQ ID NO:182. In embodiments, the oligonucleotide includes a sequence at least 80% identical to SEQ ID NO:51 and the polynucleotide includes a sequence at least 80% identical to SEQ ID NO:183. In embodiments, the oligonucleotide includes a sequence at least 80% identical to SEQ ID NO:52 and the polynucleotide includes a sequence at least 80% identical to SEQ ID NO:184. In embodiments, the oligonucleotide includes a sequence at least 80% identical to SEQ ID NO:53 and the polynucleotide includes a sequence at least 80% identical to SEQ ID NO:185. In embodiments, the oligonucleotide includes a sequence at least 80% identical to SEQ ID NO:54 and the polynucleotide includes a sequence at least 80% identical to SEQ ID NO:186. In embodiments, the oligonucleotide includes a sequence at least 80% identical to SEQ ID NO:55 and the polynucleotide includes a sequence at least 80% identical to SEQ ID NO:187. In embodiments, the oligonucleotide includes a sequence at least 80% identical to SEQ ID NO:56 and the polynucleotide includes a sequence at least 80% identical to SEQ ID NO:188. In embodiments, the oligonucleotide includes a sequence at least 80% identical to SEQ ID NO:57 and the polynucleotide includes a sequence at least 80% identical to SEQ ID NO:189. In embodiments, the oligonucleotide includes a sequence at least 80% identical to SEQ ID NO:58 and the polynucleotide includes a sequence at least 80% identical to SEQ ID NO: 190. In embodiments, the oligonucleotide includes a sequence at least 80% identical to SEQ ID NO:59 and the polynucleotide includes a sequence at least 80% identical to SEQ ID NO:191. In embodiments, the oligonucleotide includes a sequence at least 80% identical to SEQ ID NO:60 and the polynucleotide includes a sequence at least 80% identical to SEQ ID NO:192. In embodiments, the oligonucleotide includes a sequence at least 80% identical to SEQ ID NO:61 and the polynucleotide includes a sequence at least 80% identical to SEQ ID NO: 193. In embodiments, the oligonucleotide includes a sequence at least 80% identical to SEQ ID NO:62 and the polynucleotide includes a sequence at least 80% identical to SEQ ID NO:194. In embodiments, the oligonucleotide includes a sequence at least 80% identical to SEQ ID NO:63 and the polynucleotide includes a sequence at least 80% identical to SEQ ID NO: 195. In embodiments, the oligonucleotide includes a sequence at least 80% identical to SEQ ID NO:64 and the polynucleotide includes a sequence at least 80% identical to SEQ ID NO:196. In embodiments, the oligonucleotide includes a sequence at least 80% identical to SEQ ID NO:65 and the polynucleotide includes a sequence at least 80% identical to SEQ ID NO:197. In embodiments, the oligonucleotide includes a sequence at least 80% identical to SEQ ID NO:66 and the polynucleotide includes a sequence at least 80% identical to SEQ ID NO:198. In embodiments, the oligonucleotide includes a sequence at least 80% identical to SEQ ID NO:67 and the polynucleotide includes a sequence at least 80% identical to SEQ ID NO:199. In embodiments, the oligonucleotide includes a sequence at least 80% identical to SEQ ID NO:68 and the polynucleotide includes a sequence at least 80% identical to SEQ ID NO:200. In embodiments, the oligonucleotide includes a sequence at least 80% identical to SEQ ID NO:69 and the polynucleotide includes a sequence at least 80% identical to SEQ ID NO:201. In embodiments, the oligonucleotide includes a sequence at least 80% identical to SEQ ID NO:70 and the polynucleotide includes a sequence at least 80% identical to SEQ ID NO:202. In embodiments, the oligonucleotide includes a sequence at least 80% identical to SEQ ID NO:71 and the polynucleotide includes a sequence at least 80% identical to SEQ ID NO:203. In embodiments, the oligonucleotide includes a sequence at least 80% identical to SEQ ID NO:72 and the polynucleotide includes a sequence at least 80% identical to SEQ ID NO:204. In embodiments, the oligonucleotide includes a sequence at least 80% identical to SEQ ID NO:73 and the polynucleotide includes a sequence at least 80% identical to SEQ ID NO:205. In embodiments, the oligonucleotide includes a sequence at least 80% identical to SEQ ID NO:74 and the polynucleotide includes a sequence at least 80% identical to SEQ ID NO:206. In embodiments, the oligonucleotide includes a sequence at least 80% identical to SEQ ID NO:75 and the polynucleotide includes a sequence at least 80% identical to SEQ ID NO:207. In embodiments, the oligonucleotide includes a sequence at least 80% identical to SEQ ID NO:76 and the polynucleotide includes a sequence at least 80% identical to SEQ ID NO:208. In embodiments, the oligonucleotide includes a sequence at least 80% identical to SEQ ID NO:77 and the polynucleotide includes a sequence at least 80% identical to SEQ ID NO:209. In embodiments, the oligonucleotide includes a sequence at least 80% identical to SEQ ID NO:78 and the polynucleotide includes a sequence at least 80% identical to SEQ ID NO:210. In embodiments, the oligonucleotide includes a sequence at least 80% identical to SEQ ID NO:79 and the polynucleotide includes a sequence at least 80% identical to SEQ ID NO:211. In embodiments, the oligonucleotide includes a sequence at least 80% identical to SEQ ID NO:80 and the polynucleotide includes a sequence at least 80% identical to SEQ ID NO:212. In embodiments, the oligonucleotide includes a sequence at least 80% identical to SEQ ID NO:81 and the polynucleotide includes a sequence at least 80% identical to SEQ ID NO:213. In embodiments, the oligonucleotide includes a sequence at least 80% identical to SEQ ID NO:82 and the polynucleotide includes a sequence at least 80% identical to SEQ ID NO:214. In embodiments, the oligonucleotide includes a sequence at least 80% identical to SEQ ID NO:83 and the polynucleotide includes a sequence at least 80% identical to SEQ ID NO:215. In embodiments, the oligonucleotide includes a sequence at least 80% identical to SEQ ID NO:84 and the polynucleotide includes a sequence at least 80% identical to SEQ ID NO:216. In embodiments, the oligonucleotide includes a sequence at least 80% identical to SEQ ID NO:85 and the polynucleotide includes a sequence at least 80% identical to SEQ ID NO:217. In embodiments, the oligonucleotide includes a sequence at least 80% identical to SEQ ID NO:86 and the polynucleotide includes a sequence at least 80% identical to SEQ ID NO:218. In embodiments, the oligonucleotide includes a sequence at least 80% identical to SEQ ID NO:87 and the polynucleotide includes a sequence at least 80% identical to SEQ ID NO:219. In embodiments, the oligonucleotide includes a sequence at least 80% identical to SEQ ID NO:88 and the polynucleotide includes a sequence at least 80% identical to SEQ ID NO:220. In embodiments, the oligonucleotide includes a sequence at least 80% identical to SEQ ID NO:89 and the polynucleotide includes a sequence at least 80% identical to SEQ ID NO:221. In embodiments, the oligonucleotide includes a sequence at least 80% identical to SEQ ID NO:90 and the polynucleotide includes a sequence at least 80% identical to SEQ ID NO:222. In embodiments, the oligonucleotide includes a sequence at least 80% identical to SEQ ID NO:91 and the polynucleotide includes a sequence at least 80% identical to SEQ ID NO:223. In embodiments, the oligonucleotide includes a sequence at least 80% identical to SEQ ID NO:92 and the polynucleotide includes a sequence at least 80% identical to SEQ ID NO:224. In embodiments, the oligonucleotide includes a sequence at least 80% identical to SEQ ID NO:93 and the polynucleotide includes a sequence at least 80% identical to SEQ ID NO:225. In embodiments, the oligonucleotide includes a sequence at least 80% identical to SEQ ID NO:94 and the polynucleotide includes a sequence at least 80% identical to SEQ ID NO:226. In embodiments, the oligonucleotide includes a sequence at least 80% identical to SEQ ID NO:95 and the polynucleotide includes a sequence at least 80% identical to SEQ ID NO:227. In embodiments, the oligonucleotide includes a sequence at least 80% identical to SEQ ID NO:96 and the polynucleotide includes a sequence at least 80% identical to SEQ ID NO:228. In embodiments, the oligonucleotide includes a sequence at least 80% identical to SEQ ID NO:97 and the polynucleotide includes a sequence at least 80% identical to SEQ ID NO:229. In embodiments, the oligonucleotide includes a sequence at least 80% identical to SEQ ID NO:98 and the polynucleotide includes a sequence at least 80% identical to SEQ ID NO:230. In embodiments, the oligonucleotide includes a sequence at least 80% identical to SEQ ID NO:99 and the polynucleotide includes a sequence at least 80% identical to SEQ ID NO:231. In embodiments, the oligonucleotide includes a sequence at least 80% identical to SEQ ID NO: 100 and the polynucleotide includes a sequence at least 80% identical to SEQ ID NO:232. In embodiments, the oligonucleotide includes a sequence at least 80% identical to SEQ ID NO: 101 and the polynucleotide includes a sequence at least 80% identical to SEQ ID NO:233. In embodiments, the oligonucleotide includes a sequence at least 80% identical to SEQ ID NO:102 and the polynucleotide includes a sequence at least 80% identical to SEQ ID NO:234. In embodiments, the oligonucleotide includes a sequence at least 80% identical to SEQ ID NO:103 and the polynucleotide includes a sequence at least 80% identical to SEQ ID NO:235. In embodiments, the oligonucleotide includes a sequence at least 80% identical to SEQ ID NO: 104 and the polynucleotide includes a sequence at least 80% identical to SEQ ID NO:236. In embodiments, the oligonucleotide includes a sequence at least 80% identical to SEQ ID NO:105 and the polynucleotide includes a sequence at least 80% identical to SEQ ID NO:237. In embodiments, the oligonucleotide includes a sequence at least 80% identical to SEQ ID NO:106 and the polynucleotide includes a sequence at least 80% identical to SEQ ID NO:238. In embodiments, the oligonucleotide includes a sequence at least 80% identical to SEQ ID NO:107 and the polynucleotide includes a sequence at least 80% identical to SEQ ID NO:239. In embodiments, the oligonucleotide includes a sequence at least 80% identical to SEQ ID NO:108 and the polynucleotide includes a sequence at least 80% identical to SEQ ID NO:240. In embodiments, the oligonucleotide includes a sequence at least 80% identical to SEQ ID NO:109 and the polynucleotide includes a sequence at least 80% identical to SEQ ID NO:241. In embodiments, the oligonucleotide includes a sequence at least 80% identical to SEQ ID NO:110 and the polynucleotide includes a sequence at least 80% identical to SEQ ID NO:242. In embodiments, the oligonucleotide includes a sequence at least 80% identical to SEQ ID NO:111 and the polynucleotide includes a sequence at least 80% identical to SEQ ID NO:243. In embodiments, the oligonucleotide includes a sequence at least 80% identical to SEQ ID NO:112 and the polynucleotide includes a sequence at least 80% identical to SEQ ID NO:244. In embodiments, the oligonucleotide includes a sequence at least 80% identical to SEQ ID NO:113 and the polynucleotide includes a sequence at least 80% identical to SEQ ID NO:245. In embodiments, the oligonucleotide includes a sequence at least 80% identical to SEQ ID NO:114 and the polynucleotide includes a sequence at least 80% identical to SEQ ID NO:246. In embodiments, the oligonucleotide includes a sequence at least 80% identical to SEQ ID NO:115 and the polynucleotide includes a sequence at least 80% identical to SEQ ID NO:247. In embodiments, the oligonucleotide includes a sequence at least 80% identical to SEQ ID NO: 116 and the polynucleotide includes a sequence at least 80% identical to SEQ ID NO:248. In embodiments, the oligonucleotide includes a sequence at least 80% identical to SEQ ID NO:117 and the polynucleotide includes a sequence at least 80% identical to SEQ ID NO:249. In embodiments, the oligonucleotide includes a sequence at least 80% identical to SEQ ID NO: 118 and the polynucleotide includes a sequence at least 80% identical to SEQ ID NO:250. In embodiments, the oligonucleotide includes a sequence at least 80% identical to SEQ ID NO: 119 and the polynucleotide includes a sequence at least 80% identical to SEQ ID NO:251. In embodiments, the oligonucleotide includes a sequence at least 80% identical to SEQ ID NO: 120 and the polynucleotide includes a sequence at least 80% identical to SEQ ID NO:252. In embodiments, the oligonucleotide includes a sequence at least 80% identical to SEQ ID NO: 121 and the polynucleotide includes a sequence at least 80% identical to SEQ ID NO:253. In embodiments, the oligonucleotide includes a sequence at least 80% identical to SEQ ID NO:122 and the polynucleotide includes a sequence at least 80% identical to SEQ ID NO:254. In embodiments, the oligonucleotide includes a sequence at least 80% identical to SEQ ID NO:123 and the polynucleotide includes a sequence at least 80% identical to SEQ ID NO:255. In embodiments, the oligonucleotide includes a sequence at least 80% identical to SEQ ID NO:124 and the polynucleotide includes a sequence at least 80% identical to SEQ ID NO:256. In embodiments, the oligonucleotide includes a sequence at least 80% identical to SEQ ID NO: 125 and the polynucleotide includes a sequence at least 80% identical to SEQ ID NO:257. In embodiments, the oligonucleotide includes a sequence at least 80% identical to SEQ ID NO:126 and the polynucleotide includes a sequence at least 80% identical to SEQ ID NO:258. In embodiments, the oligonucleotide includes a sequence at least 80% identical to SEQ ID NO:127 and the polynucleotide includes a sequence at least 80% identical to SEQ ID NO:259. In embodiments, the oligonucleotide includes a sequence at least 80% identical to SEQ ID NO:128 and the polynucleotide includes a sequence at least 80% identical to SEQ ID NO:260. In embodiments, the oligonucleotide includes a sequence at least 80% identical to SEQ ID NO:129 and the polynucleotide includes a sequence at least 80% identical to SEQ ID NO:261. In embodiments, the oligonucleotide includes a sequence at least 80% identical to SEQ ID NO:130 and the polynucleotide includes a sequence at least 80% identical to SEQ ID NO:262. In embodiments, the oligonucleotide includes a sequence at least 80% identical to SEQ ID NO: 131 and the polynucleotide includes a sequence at least 80% identical to SEQ ID NO:263. In embodiments, the oligonucleotide includes a sequence at least 80% identical to SEQ ID NO: 132 and the polynucleotide includes a sequence at least 80% identical to SEQ ID NO:264.

In embodiments, the oligonucleotide includes a sequence from SEQ ID NO:1 to SEQ ID NO: 132 and the polynucleotide described herein or polynucleotide probe described herein includes a sequence selected from SEQ ID NO:133 to SEQ ID NO:264. In embodiments, the oligonucleotide includes SEQ ID NO:1 and the polynucleotide includes SEQ ID NO: 133. In embodiments, the oligonucleotide includes SEQ ID NO:2 and the polynucleotide includes SEQ ID NO: 134. In embodiments, the oligonucleotide includes SEQ ID NO:3 and the polynucleotide includes SEQ ID NO: 135. In embodiments, the oligonucleotide includes SEQ ID NO:4 and the polynucleotide includes SEQ ID NO:136. In embodiments, the oligonucleotide includes SEQ ID NO: 5 and the polynucleotide includes SEQ ID NO:137. In embodiments, the oligonucleotide includes SEQ ID NO:6 and the polynucleotide includes SEQ ID NO:138. In embodiments, the oligonucleotide includes SEQ ID NO:7 and the polynucleotide includes SEQ ID NO: 139. In embodiments, the oligonucleotide includes SEQ ID NO:8 and the polynucleotide includes SEQ ID NO: 140. In embodiments, the oligonucleotide includes SEQ ID NO:9 and the polynucleotide includes SEQ ID NO: 141. In embodiments, the oligonucleotide includes SEQ ID NO: 10 and the polynucleotide includes SEQ ID NO:142. In embodiments, the oligonucleotide includes SEQ ID NO: 11 and the polynucleotide includes SEQ ID NO:143. In embodiments, the oligonucleotide includes SEQ ID NO:12 and the polynucleotide includes SEQ ID NO:144. In embodiments, the oligonucleotide includes SEQ ID NO: 13 and the polynucleotide includes SEQ ID NO:145. In embodiments, the oligonucleotide includes SEQ ID NO:14 and the polynucleotide includes SEQ ID NO: 146. In embodiments, the oligonucleotide includes SEQ ID NO:15 and the polynucleotide includes SEQ ID NO:147. In embodiments, the oligonucleotide includes SEQ ID NO: 16 and the polynucleotide includes SEQ ID NO:148. In embodiments, the oligonucleotide includes SEQ ID NO:17 and the polynucleotide includes SEQ ID NO:149. In embodiments, the oligonucleotide includes SEQ ID NO:18 and the polynucleotide includes SEQ ID NO:150. In embodiments, the oligonucleotide includes SEQ ID NO:19 and the polynucleotide includes SEQ ID NO: 151. In embodiments, the oligonucleotide includes SEQ ID NO:20 and the polynucleotide includes SEQ ID NO:152. In embodiments, the oligonucleotide includes SEQ ID NO: 21 and the polynucleotide includes SEQ ID NO:153. In embodiments, the oligonucleotide includes SEQ ID NO:22 and the polynucleotide includes SEQ ID NO: 154. In embodiments, the oligonucleotide includes SEQ ID NO:23 and the polynucleotide includes SEQ ID NO:155. In embodiments, the oligonucleotide includes SEQ ID NO:24 and the polynucleotide includes SEQ ID NO: 156. In embodiments, the oligonucleotide includes SEQ ID NO:25 and the polynucleotide includes SEQ ID NO:157. In embodiments, the oligonucleotide includes SEQ ID NO: 26 and the polynucleotide includes SEQ ID NO:158. In embodiments, the oligonucleotide includes SEQ ID NO:27 and the polynucleotide includes SEQ ID NO:159. In embodiments, the oligonucleotide includes SEQ ID NO:28 and the polynucleotide includes SEQ ID NO:160. In embodiments, the oligonucleotide includes SEQ ID NO:29 and the polynucleotide includes SEQ ID NO: 161. In embodiments, the oligonucleotide includes SEQ ID NO:30 and the polynucleotide includes SEQ ID NO:162. In embodiments, the oligonucleotide includes SEQ ID NO: 31 and the polynucleotide includes SEQ ID NO:163. In embodiments, the oligonucleotide includes SEQ ID NO:32 and the polynucleotide includes SEQ ID NO:164. In embodiments, the oligonucleotide includes SEQ ID NO:33 and the polynucleotide includes SEQ ID NO:165. In embodiments, the oligonucleotide includes SEQ ID NO:34 and the polynucleotide includes SEQ ID NO: 166. In embodiments, the oligonucleotide includes SEQ ID NO:35 and the polynucleotide includes SEQ ID NO:167. In embodiments, the oligonucleotide includes SEQ ID NO: 36 and the polynucleotide includes SEQ ID NO:168. In embodiments, the oligonucleotide includes SEQ ID NO:37 and the polynucleotide includes SEQ ID NO:169. In embodiments, the oligonucleotide includes SEQ ID NO:38 and the polynucleotide includes SEQ ID NO:170. In embodiments, the oligonucleotide includes SEQ ID NO:39 and the polynucleotide includes SEQ ID NO: 171. In embodiments, the oligonucleotide includes SEQ ID NO:40 and the polynucleotide includes SEQ ID NO:172. In embodiments, the oligonucleotide includes SEQ ID NO: 41 and the polynucleotide includes SEQ ID NO:173. In embodiments, the oligonucleotide includes SEQ ID NO:42 and the polynucleotide includes SEQ ID NO:174. In embodiments, the oligonucleotide includes SEQ ID NO:43 and the polynucleotide includes SEQ ID NO: 175. In embodiments, the oligonucleotide includes SEQ ID NO:44 and the polynucleotide includes SEQ ID NO: 176. In embodiments, the oligonucleotide includes SEQ ID NO:45 and the polynucleotide includes SEQ ID NO:177. In embodiments, the oligonucleotide includes SEQ ID NO: 46 and the polynucleotide includes SEQ ID NO:178. In embodiments, the oligonucleotide includes SEQ ID NO:47 and the polynucleotide includes SEQ ID NO:179. In embodiments, the oligonucleotide includes SEQ ID NO:48 and the polynucleotide includes SEQ ID NO:180. In embodiments, the oligonucleotide includes SEQ ID NO:49 and the polynucleotide includes SEQ ID NO: 181. In embodiments, the oligonucleotide includes SEQ ID NO:50 and the polynucleotide includes SEQ ID NO:182. In embodiments, the oligonucleotide includes SEQ ID NO: 51 and the polynucleotide includes SEQ ID NO: 183. In embodiments, the oligonucleotide includes SEQ ID NO:52 and the polynucleotide includes SEQ ID NO: 184. In embodiments, the oligonucleotide includes SEQ ID NO:53 and the polynucleotide includes SEQ ID NO:185. In embodiments, the oligonucleotide includes SEQ ID NO:54 and the polynucleotide includes SEQ ID NO: 186. In embodiments, the oligonucleotide includes SEQ ID NO:55 and the polynucleotide includes SEQ ID NO:187. In embodiments, the oligonucleotide includes SEQ ID NO: 56 and the polynucleotide includes SEQ ID NO:188. In embodiments, the oligonucleotide includes SEQ ID NO:57 and the polynucleotide includes SEQ ID NO: 189. In embodiments, the oligonucleotide includes SEQ ID NO:58 and the polynucleotide includes SEQ ID NO:190. In embodiments, the oligonucleotide includes SEQ ID NO:59 and the polynucleotide includes SEQ ID NO: 191. In embodiments, the oligonucleotide includes SEQ ID NO:60 and the polynucleotide includes SEQ ID NO:192. In embodiments, the oligonucleotide includes SEQ ID NO: 61 and the polynucleotide includes SEQ ID NO:193. In embodiments, the oligonucleotide includes SEQ ID NO:62 and the polynucleotide includes SEQ ID NO:194. In embodiments, the oligonucleotide includes SEQ ID NO:63 and the polynucleotide includes SEQ ID NO:195. In embodiments, the oligonucleotide includes SEQ ID NO:64 and the polynucleotide includes SEQ ID NO: 196. In embodiments, the oligonucleotide includes SEQ ID NO:65 and the polynucleotide includes SEQ ID NO:197. In embodiments, the oligonucleotide includes SEQ ID NO: 66 and the polynucleotide includes SEQ ID NO:198. In embodiments, the oligonucleotide includes SEQ ID NO:67 and the polynucleotide includes SEQ ID NO: 199. In embodiments, the oligonucleotide includes SEQ ID NO:68 and the polynucleotide includes SEQ ID NO:200. In embodiments, the oligonucleotide includes SEQ ID NO:69 and the polynucleotide includes SEQ ID NO: 201. In embodiments, the oligonucleotide includes SEQ ID NO:70 and the polynucleotide includes SEQ ID NO:202. In embodiments, the oligonucleotide includes SEQ ID NO: 71 and the polynucleotide includes SEQ ID NO:203. In embodiments, the oligonucleotide includes SEQ ID NO:72 and the polynucleotide includes SEQ ID NO:204. In embodiments, the oligonucleotide includes SEQ ID NO:73 and the polynucleotide includes SEQ ID NO:205. In embodiments, the oligonucleotide includes SEQ ID NO:74 and the polynucleotide includes SEQ ID NO: 206. In embodiments, the oligonucleotide includes SEQ ID NO:75 and the polynucleotide includes SEQ ID NO:207. In embodiments, the oligonucleotide includes SEQ ID NO: 76 and the polynucleotide includes SEQ ID NO:208. In embodiments, the oligonucleotide includes SEQ ID NO:77 and the polynucleotide includes SEQ ID NO:209. In embodiments, the oligonucleotide includes SEQ ID NO:78 and the polynucleotide includes SEQ ID NO:210. In embodiments, the oligonucleotide includes SEQ ID NO:79 and the polynucleotide includes SEQ ID NO: 211. In embodiments, the oligonucleotide includes SEQ ID NO:80 and the polynucleotide includes SEQ ID NO:212. In embodiments, the oligonucleotide includes SEQ ID NO: 81 and the polynucleotide includes SEQ ID NO:213. In embodiments, the oligonucleotide includes SEQ ID NO:82 and the polynucleotide includes SEQ ID NO:214. In embodiments, the oligonucleotide includes SEQ ID NO:83 and the polynucleotide includes SEQ ID NO:215. In embodiments, the oligonucleotide includes SEQ ID NO:84 and the polynucleotide includes SEQ ID NO: 216. In embodiments, the oligonucleotide includes SEQ ID NO:85 and the polynucleotide includes SEQ ID NO:217. In embodiments, the oligonucleotide includes SEQ ID NO: 86 and the polynucleotide includes SEQ ID NO:218. In embodiments, the oligonucleotide includes SEQ ID NO:87 and the polynucleotide includes SEQ ID NO:219. In embodiments, the oligonucleotide includes SEQ ID NO:88 and the polynucleotide includes SEQ ID NO:220. In embodiments, the oligonucleotide includes SEQ ID NO:89 and the polynucleotide includes SEQ ID NO: 221. In embodiments, the oligonucleotide includes SEQ ID NO:90 and the polynucleotide includes SEQ ID NO:222. In embodiments, the oligonucleotide includes SEQ ID NO: 91 and the polynucleotide includes SEQ ID NO:223. In embodiments, the oligonucleotide includes SEQ ID NO:92 and the polynucleotide includes SEQ ID NO:224. In embodiments, the oligonucleotide includes SEQ ID NO:93 and the polynucleotide includes SEQ ID NO:225. In embodiments, the oligonucleotide includes SEQ ID NO:94 and the polynucleotide includes SEQ ID NO: 226. In embodiments, the oligonucleotide includes SEQ ID NO:95 and the polynucleotide includes SEQ ID NO:227. In embodiments, the oligonucleotide includes SEQ ID NO: 96 and the polynucleotide includes SEQ ID NO:228. In embodiments, the oligonucleotide includes SEQ ID NO:97 and the polynucleotide includes SEQ ID NO:229. In embodiments, the oligonucleotide includes SEQ ID NO:98 and the polynucleotide includes SEQ ID NO:230. In embodiments, the oligonucleotide includes SEQ ID NO:99 and the polynucleotide includes SEQ ID NO: 231. In embodiments, the oligonucleotide includes SEQ ID NO: 100 and the polynucleotide includes SEQ ID NO:232. In embodiments, the oligonucleotide includes SEQ ID NO: 101 and the polynucleotide includes SEQ ID NO:233. In embodiments, the oligonucleotide includes SEQ ID NO:102 and the polynucleotide includes SEQ ID NO:234. In embodiments, the oligonucleotide includes SEQ ID NO:103 and the polynucleotide includes SEQ ID NO:235. In embodiments, the oligonucleotide includes SEQ ID NO:104 and the polynucleotide includes SEQ ID NO:236. In embodiments, the oligonucleotide includes SEQ ID NO:105 and the polynucleotide includes SEQ ID NO:237. In embodiments, the oligonucleotide includes SEQ ID NO: 106 and the polynucleotide includes SEQ ID NO:238. In embodiments, the oligonucleotide includes SEQ ID NO: 107 and the polynucleotide includes SEQ ID NO:239. In embodiments, the oligonucleotide includes SEQ ID NO:108 and the polynucleotide includes SEQ ID NO:240. In embodiments, the oligonucleotide includes SEQ ID NO:109 and the polynucleotide includes SEQ ID NO:241. In embodiments, the oligonucleotide includes SEQ ID NO: 110 and the polynucleotide includes SEQ ID NO:242. In embodiments, the oligonucleotide includes SEQ ID NO: 111 and the polynucleotide includes SEQ ID NO:243. In embodiments, the oligonucleotide includes SEQ ID NO:112 and the polynucleotide includes SEQ ID NO:244. In embodiments, the oligonucleotide includes SEQ ID NO:113 and the polynucleotide includes SEQ ID NO:245. In embodiments, the oligonucleotide includes SEQ ID NO:114 and the polynucleotide includes SEQ ID NO:246. In embodiments, the oligonucleotide includes SEQ ID NO:115 and the polynucleotide includes SEQ ID NO:247. In embodiments, the oligonucleotide includes SEQ ID NO: 116 and the polynucleotide includes SEQ ID NO:248. In embodiments, the oligonucleotide includes SEQ ID NO:117 and the polynucleotide includes SEQ ID NO:249. In embodiments, the oligonucleotide includes SEQ ID NO:118 and the polynucleotide includes SEQ ID NO:250. In embodiments, the oligonucleotide includes SEQ ID NO:119 and the polynucleotide includes SEQ ID NO:251. In embodiments, the oligonucleotide includes SEQ ID NO: 120 and the polynucleotide includes SEQ ID NO:252. In embodiments, the oligonucleotide includes SEQ ID NO: 121 and the polynucleotide includes SEQ ID NO:253. In embodiments, the oligonucleotide includes SEQ ID NO:122 and the polynucleotide includes SEQ ID NO:254. In embodiments, the oligonucleotide includes SEQ ID NO:123 and the polynucleotide includes SEQ ID NO:255. In embodiments, the oligonucleotide includes SEQ ID NO:124 and the polynucleotide includes SEQ ID NO:256. In embodiments, the oligonucleotide includes SEQ ID NO:125 and the polynucleotide includes SEQ ID NO:257. In embodiments, the oligonucleotide includes SEQ ID NO: 126 and the polynucleotide includes SEQ ID NO:258. In embodiments, the oligonucleotide includes SEQ ID NO: 127 and the polynucleotide includes SEQ ID NO:259. In embodiments, the oligonucleotide includes SEQ ID NO:128 and the polynucleotide includes SEQ ID NO:260. In embodiments, the oligonucleotide includes SEQ ID NO:129 and the polynucleotide includes SEQ ID NO:261. In embodiments, the oligonucleotide includes SEQ ID NO:130 and the polynucleotide includes SEQ ID NO:262. In embodiments, the oligonucleotide includes SEQ ID NO: 131 and the polynucleotide includes SEQ ID NO:263. In embodiments, the oligonucleotide includes SEQ ID NO: 132 and the polynucleotide includes SEQ ID NO:264.

In embodiments, the oligonucleotide includes a sequence at least 80% identical to a sequence selected from SEQ ID NO:1 to SEQ ID NO:132 and the polynucleotide described herein or polynucleotide probe described herein includes a sequence at least 80% identical to a sequence selected from SEQ ID NO:265 to SEQ ID NO:396. In embodiments, the oligonucleotide includes a sequence at least 80% identical to SEQ ID NO:1 and the polynucleotide includes a sequence at least 80% identical to SEQ ID NO:265. In embodiments, the oligonucleotide includes a sequence at least 80% identical to SEQ ID NO:2 and the polynucleotide includes a sequence at least 80% identical to SEQ ID NO:266. In embodiments, the oligonucleotide includes a sequence at least 80% identical to SEQ ID NO:3 and the polynucleotide includes a sequence at least 80% identical to SEQ ID NO:267. In embodiments, the oligonucleotide includes a sequence at least 80% identical to SEQ ID NO:4 and the polynucleotide includes a sequence at least 80% identical to SEQ ID NO:268. In embodiments, the oligonucleotide includes a sequence at least 80% identical to SEQ ID NO:5 and the polynucleotide includes a sequence at least 80% identical to SEQ ID NO:269. In embodiments, the oligonucleotide includes a sequence at least 80% identical to SEQ ID NO:6 and the polynucleotide includes a sequence at least 80% identical to SEQ ID NO:270. In embodiments, the oligonucleotide includes a sequence at least 80% identical to SEQ ID NO:7 and the polynucleotide includes a sequence at least 80% identical to SEQ ID NO:271. In embodiments, the oligonucleotide includes a sequence at least 80% identical to SEQ ID NO:8 and the polynucleotide includes a sequence at least 80% identical to SEQ ID NO:272. In embodiments, the oligonucleotide includes a sequence at least 80% identical to SEQ ID NO:9 and the polynucleotide includes a sequence at least 80% identical to SEQ ID NO:273. In embodiments, the oligonucleotide includes a sequence at least 80% identical to SEQ ID NO: 10 and the polynucleotide includes a sequence at least 80% identical to SEQ ID NO:274. In embodiments, the oligonucleotide includes a sequence at least 80% identical to SEQ ID NO: 11 and the polynucleotide includes a sequence at least 80% identical to SEQ ID NO:275. In embodiments, the oligonucleotide includes a sequence at least 80% identical to SEQ ID NO: 12 and the polynucleotide includes a sequence at least 80% identical to SEQ ID NO:276. In embodiments, the oligonucleotide includes a sequence at least 80% identical to SEQ ID NO: 13 and the polynucleotide includes a sequence at least 80% identical to SEQ ID NO:277. In embodiments, the oligonucleotide includes a sequence at least 80% identical to SEQ ID NO:14 and the polynucleotide includes a sequence at least 80% identical to SEQ ID NO:278. In embodiments, the oligonucleotide includes a sequence at least 80% identical to SEQ ID NO:15 and the polynucleotide includes a sequence at least 80% identical to SEQ ID NO:279. In embodiments, the oligonucleotide includes a sequence at least 80% identical to SEQ ID NO: 16 and the polynucleotide includes a sequence at least 80% identical to SEQ ID NO:280. In embodiments, the oligonucleotide includes a sequence at least 80% identical to SEQ ID NO:17 and the polynucleotide includes a sequence at least 80% identical to SEQ ID NO:281. In embodiments, the oligonucleotide includes a sequence at least 80% identical to SEQ ID NO:18 and the polynucleotide includes a sequence at least 80% identical to SEQ ID NO:282. In embodiments, the oligonucleotide includes a sequence at least 80% identical to SEQ ID NO:19 and the polynucleotide includes a sequence at least 80% identical to SEQ ID NO:283. In embodiments, the oligonucleotide includes a sequence at least 80% identical to SEQ ID NO:20 and the polynucleotide includes a sequence at least 80% identical to SEQ ID NO:284. In embodiments, the oligonucleotide includes a sequence at least 80% identical to SEQ ID NO:21 and the polynucleotide includes a sequence at least 80% identical to SEQ ID NO:285. In embodiments, the oligonucleotide includes a sequence at least 80% identical to SEQ ID NO:22 and the polynucleotide includes a sequence at least 80% identical to SEQ ID NO:286. In embodiments, the oligonucleotide includes a sequence at least 80% identical to SEQ ID NO:23 and the polynucleotide includes a sequence at least 80% identical to SEQ ID NO:287. In embodiments, the oligonucleotide includes a sequence at least 80% identical to SEQ ID NO:24 and the polynucleotide includes a sequence at least 80% identical to SEQ ID NO:288. In embodiments, the oligonucleotide includes a sequence at least 80% identical to SEQ ID NO:25 and the polynucleotide includes a sequence at least 80% identical to SEQ ID NO:289. In embodiments, the oligonucleotide includes a sequence at least 80% identical to SEQ ID NO:26 and the polynucleotide includes a sequence at least 80% identical to SEQ ID NO:290. In embodiments, the oligonucleotide includes a sequence at least 80% identical to SEQ ID NO:27 and the polynucleotide includes a sequence at least 80% identical to SEQ ID NO:291. In embodiments, the oligonucleotide includes a sequence at least 80% identical to SEQ ID NO:28 and the polynucleotide includes a sequence at least 80% identical to SEQ ID NO:292. In embodiments, the oligonucleotide includes a sequence at least 80% identical to SEQ ID NO:29 and the polynucleotide includes a sequence at least 80% identical to SEQ ID NO:293. In embodiments, the oligonucleotide includes a sequence at least 80% identical to SEQ ID NO:30 and the polynucleotide includes a sequence at least 80% identical to SEQ ID NO:294. In embodiments, the oligonucleotide includes a sequence at least 80% identical to SEQ ID NO:31 and the polynucleotide includes a sequence at least 80% identical to SEQ ID NO:295. In embodiments, the oligonucleotide includes a sequence at least 80% identical to SEQ ID NO:32 and the polynucleotide includes a sequence at least 80% identical to SEQ ID NO:296. In embodiments, the oligonucleotide includes a sequence at least 80% identical to SEQ ID NO:33 and the polynucleotide includes a sequence at least 80% identical to SEQ ID NO:297. In embodiments, the oligonucleotide includes a sequence at least 80% identical to SEQ ID NO:34 and the polynucleotide includes a sequence at least 80% identical to SEQ ID NO:298. In embodiments, the oligonucleotide includes a sequence at least 80% identical to SEQ ID NO:35 and the polynucleotide includes a sequence at least 80% identical to SEQ ID NO:299. In embodiments, the oligonucleotide includes a sequence at least 80% identical to SEQ ID NO:36 and the polynucleotide includes a sequence at least 80% identical to SEQ ID NO:300. In embodiments, the oligonucleotide includes a sequence at least 80% identical to SEQ ID NO:37 and the polynucleotide includes a sequence at least 80% identical to SEQ ID NO:301. In embodiments, the oligonucleotide includes a sequence at least 80% identical to SEQ ID NO:38 and the polynucleotide includes a sequence at least 80% identical to SEQ ID NO:302. In embodiments, the oligonucleotide includes a sequence at least 80% identical to SEQ ID NO:39 and the polynucleotide includes a sequence at least 80% identical to SEQ ID NO:303. In embodiments, the oligonucleotide includes a sequence at least 80% identical to SEQ ID NO:40 and the polynucleotide includes a sequence at least 80% identical to SEQ ID NO:304. In embodiments, the oligonucleotide includes a sequence at least 80% identical to SEQ ID NO:41 and the polynucleotide includes a sequence at least 80% identical to SEQ ID NO:305. In embodiments, the oligonucleotide includes a sequence at least 80% identical to SEQ ID NO:42 and the polynucleotide includes a sequence at least 80% identical to SEQ ID NO:306. In embodiments, the oligonucleotide includes a sequence at least 80% identical to SEQ ID NO:43 and the polynucleotide includes a sequence at least 80% identical to SEQ ID NO:307. In embodiments, the oligonucleotide includes a sequence at least 80% identical to SEQ ID NO:44 and the polynucleotide includes a sequence at least 80% identical to SEQ ID NO:308. In embodiments, the oligonucleotide includes a sequence at least 80% identical to SEQ ID NO:45 and the polynucleotide includes a sequence at least 80% identical to SEQ ID NO:309. In embodiments, the oligonucleotide includes a sequence at least 80% identical to SEQ ID NO:46 and the polynucleotide includes a sequence at least 80% identical to SEQ ID NO:310. In embodiments, the oligonucleotide includes a sequence at least 80% identical to SEQ ID NO:47 and the polynucleotide includes a sequence at least 80% identical to SEQ ID NO:311. In embodiments, the oligonucleotide includes a sequence at least 80% identical to SEQ ID NO:48 and the polynucleotide includes a sequence at least 80% identical to SEQ ID NO:312. In embodiments, the oligonucleotide includes a sequence at least 80% identical to SEQ ID NO:49 and the polynucleotide includes a sequence at least 80% identical to SEQ ID NO:313. In embodiments, the oligonucleotide includes a sequence at least 80% identical to SEQ ID NO:50 and the polynucleotide includes a sequence at least 80% identical to SEQ ID NO:314. In embodiments, the oligonucleotide includes a sequence at least 80% identical to SEQ ID NO:51 and the polynucleotide includes a sequence at least 80% identical to SEQ ID NO:315. In embodiments, the oligonucleotide includes a sequence at least 80% identical to SEQ ID NO:52 and the polynucleotide includes a sequence at least 80% identical to SEQ ID NO:316. In embodiments, the oligonucleotide includes a sequence at least 80% identical to SEQ ID NO:53 and the polynucleotide includes a sequence at least 80% identical to SEQ ID NO:317. In embodiments, the oligonucleotide includes a sequence at least 80% identical to SEQ ID NO:54 and the polynucleotide includes a sequence at least 80% identical to SEQ ID NO:318. In embodiments, the oligonucleotide includes a sequence at least 80% identical to SEQ ID NO:55 and the polynucleotide includes a sequence at least 80% identical to SEQ ID NO:319. In embodiments, the oligonucleotide includes a sequence at least 80% identical to SEQ ID NO:56 and the polynucleotide includes a sequence at least 80% identical to SEQ ID NO:320. In embodiments, the oligonucleotide includes a sequence at least 80% identical to SEQ ID NO:57 and the polynucleotide includes a sequence at least 80% identical to SEQ ID NO:321. In embodiments, the oligonucleotide includes a sequence at least 80% identical to SEQ ID NO:58 and the polynucleotide includes a sequence at least 80% identical to SEQ ID NO:322. In embodiments, the oligonucleotide includes a sequence at least 80% identical to SEQ ID NO:59 and the polynucleotide includes a sequence at least 80% identical to SEQ ID NO:323. In embodiments, the oligonucleotide includes a sequence at least 80% identical to SEQ ID NO:60 and the polynucleotide includes a sequence at least 80% identical to SEQ ID NO:324. In embodiments, the oligonucleotide includes a sequence at least 80% identical to SEQ ID NO:61 and the polynucleotide includes a sequence at least 80% identical to SEQ ID NO:325. In embodiments, the oligonucleotide includes a sequence at least 80% identical to SEQ ID NO:62 and the polynucleotide includes a sequence at least 80% identical to SEQ ID NO:326. In embodiments, the oligonucleotide includes a sequence at least 80% identical to SEQ ID NO:63 and the polynucleotide includes a sequence at least 80% identical to SEQ ID NO:327. In embodiments, the oligonucleotide includes a sequence at least 80% identical to SEQ ID NO:64 and the polynucleotide includes a sequence at least 80% identical to SEQ ID NO:328. In embodiments, the oligonucleotide includes a sequence at least 80% identical to SEQ ID NO:65 and the polynucleotide includes a sequence at least 80% identical to SEQ ID NO:329. In embodiments, the oligonucleotide includes a sequence at least 80% identical to SEQ ID NO:66 and the polynucleotide includes a sequence at least 80% identical to SEQ ID NO:330. In embodiments, the oligonucleotide includes a sequence at least 80% identical to SEQ ID NO:67 and the polynucleotide includes a sequence at least 80% identical to SEQ ID NO:331. In embodiments, the oligonucleotide includes a sequence at least 80% identical to SEQ ID NO:68 and the polynucleotide includes a sequence at least 80% identical to SEQ ID NO:332. In embodiments, the oligonucleotide includes a sequence at least 80% identical to SEQ ID NO:69 and the polynucleotide includes a sequence at least 80% identical to SEQ ID NO:333. In embodiments, the oligonucleotide includes a sequence at least 80% identical to SEQ ID NO:70 and the polynucleotide includes a sequence at least 80% identical to SEQ ID NO:334. In embodiments, the oligonucleotide includes a sequence at least 80% identical to SEQ ID NO:71 and the polynucleotide includes a sequence at least 80% identical to SEQ ID NO:335. In embodiments, the oligonucleotide includes a sequence at least 80% identical to SEQ ID NO:72 and the polynucleotide includes a sequence at least 80% identical to SEQ ID NO:336. In embodiments, the oligonucleotide includes a sequence at least 80% identical to SEQ ID NO:73 and the polynucleotide includes a sequence at least 80% identical to SEQ ID NO:337. In embodiments, the oligonucleotide includes a sequence at least 80% identical to SEQ ID NO:74 and the polynucleotide includes a sequence at least 80% identical to SEQ ID NO:338. In embodiments, the oligonucleotide includes a sequence at least 80% identical to SEQ ID NO:75 and the polynucleotide includes a sequence at least 80% identical to SEQ ID NO:339. In embodiments, the oligonucleotide includes a sequence at least 80% identical to SEQ ID NO:76 and the polynucleotide includes a sequence at least 80% identical to SEQ ID NO:340. In embodiments, the oligonucleotide includes a sequence at least 80% identical to SEQ ID NO:77 and the polynucleotide includes a sequence at least 80% identical to SEQ ID NO:341. In embodiments, the oligonucleotide includes a sequence at least 80% identical to SEQ ID NO:78 and the polynucleotide includes a sequence at least 80% identical to SEQ ID NO:342. In embodiments, the oligonucleotide includes a sequence at least 80% identical to SEQ ID NO:79 and the polynucleotide includes a sequence at least 80% identical to SEQ ID NO:343. In embodiments, the oligonucleotide includes a sequence at least 80% identical to SEQ ID NO:80 and the polynucleotide includes a sequence at least 80% identical to SEQ ID NO:344. In embodiments, the oligonucleotide includes a sequence at least 80% identical to SEQ ID NO:81 and the polynucleotide includes a sequence at least 80% identical to SEQ ID NO:345. In embodiments, the oligonucleotide includes a sequence at least 80% identical to SEQ ID NO:82 and the polynucleotide includes a sequence at least 80% identical to SEQ ID NO:346. In embodiments, the oligonucleotide includes a sequence at least 80% identical to SEQ ID NO:83 and the polynucleotide includes a sequence at least 80% identical to SEQ ID NO:347. In embodiments, the oligonucleotide includes a sequence at least 80% identical to SEQ ID NO:84 and the polynucleotide includes a sequence at least 80% identical to SEQ ID NO:348. In embodiments, the oligonucleotide includes a sequence at least 80% identical to SEQ ID NO:85 and the polynucleotide includes a sequence at least 80% identical to SEQ ID NO:349. In embodiments, the oligonucleotide includes a sequence at least 80% identical to SEQ ID NO:86 and the polynucleotide includes a sequence at least 80% identical to SEQ ID NO:350. In embodiments, the oligonucleotide includes a sequence at least 80% identical to SEQ ID NO:87 and the polynucleotide includes a sequence at least 80% identical to SEQ ID NO:351. In embodiments, the oligonucleotide includes a sequence at least 80% identical to SEQ ID NO:88 and the polynucleotide includes a sequence at least 80% identical to SEQ ID NO:352. In embodiments, the oligonucleotide includes a sequence at least 80% identical to SEQ ID NO:89 and the polynucleotide includes a sequence at least 80% identical to SEQ ID NO:353. In embodiments, the oligonucleotide includes a sequence at least 80% identical to SEQ ID NO:90 and the polynucleotide includes a sequence at least 80% identical to SEQ ID NO:354. In embodiments, the oligonucleotide includes a sequence at least 80% identical to SEQ ID NO:91 and the polynucleotide includes a sequence at least 80% identical to SEQ ID NO:355. In embodiments, the oligonucleotide includes a sequence at least 80% identical to SEQ ID NO:92 and the polynucleotide includes a sequence at least 80% identical to SEQ ID NO:356. In embodiments, the oligonucleotide includes a sequence at least 80% identical to SEQ ID NO:93 and the polynucleotide includes a sequence at least 80% identical to SEQ ID NO:357. In embodiments, the oligonucleotide includes a sequence at least 80% identical to SEQ ID NO:94 and the polynucleotide includes a sequence at least 80% identical to SEQ ID NO:358. In embodiments, the oligonucleotide includes a sequence at least 80% identical to SEQ ID NO:95 and the polynucleotide includes a sequence at least 80% identical to SEQ ID NO:359. In embodiments, the oligonucleotide includes a sequence at least 80% identical to SEQ ID NO:96 and the polynucleotide includes a sequence at least 80% identical to SEQ ID NO:360. In embodiments, the oligonucleotide includes a sequence at least 80% identical to SEQ ID NO:97 and the polynucleotide includes a sequence at least 80% identical to SEQ ID NO:361. In embodiments, the oligonucleotide includes a sequence at least 80% identical to SEQ ID NO:98 and the polynucleotide includes a sequence at least 80% identical to SEQ ID NO:362. In embodiments, the oligonucleotide includes a sequence at least 80% identical to SEQ ID NO:99 and the polynucleotide includes a sequence at least 80% identical to SEQ ID NO:363. In embodiments, the oligonucleotide includes a sequence at least 80% identical to SEQ ID NO:100 and the polynucleotide includes a sequence at least 80% identical to SEQ ID NO:364. In embodiments, the oligonucleotide includes a sequence at least 80% identical to SEQ ID NO:101 and the polynucleotide includes a sequence at least 80% identical to SEQ ID NO:365. In embodiments, the oligonucleotide includes a sequence at least 80% identical to SEQ ID NO:102 and the polynucleotide includes a sequence at least 80% identical to SEQ ID NO:366. In embodiments, the oligonucleotide includes a sequence at least 80% identical to SEQ ID NO:103 and the polynucleotide includes a sequence at least 80% identical to SEQ ID NO:367. In embodiments, the oligonucleotide includes a sequence at least 80% identical to SEQ ID NO:104 and the polynucleotide includes a sequence at least 80% identical to SEQ ID NO:368. In embodiments, the oligonucleotide includes a sequence at least 80% identical to SEQ ID NO: 105 and the polynucleotide includes a sequence at least 80% identical to SEQ ID NO:369. In embodiments, the oligonucleotide includes a sequence at least 80% identical to SEQ ID NO: 106 and the polynucleotide includes a sequence at least 80% identical to SEQ ID NO:370. In embodiments, the oligonucleotide includes a sequence at least 80% identical to SEQ ID NO: 107 and the polynucleotide includes a sequence at least 80% identical to SEQ ID NO:371. In embodiments, the oligonucleotide includes a sequence at least 80% identical to SEQ ID NO: 108 and the polynucleotide includes a sequence at least 80% identical to SEQ ID NO:372. In embodiments, the oligonucleotide includes a sequence at least 80% identical to SEQ ID NO:109 and the polynucleotide includes a sequence at least 80% identical to SEQ ID NO:373. In embodiments, the oligonucleotide includes a sequence at least 80% identical to SEQ ID NO:110 and the polynucleotide includes a sequence at least 80% identical to SEQ ID NO:374. In embodiments, the oligonucleotide includes a sequence at least 80% identical to SEQ ID NO:111 and the polynucleotide includes a sequence at least 80% identical to SEQ ID NO:375. In embodiments, the oligonucleotide includes a sequence at least 80% identical to SEQ ID NO:112 and the polynucleotide includes a sequence at least 80% identical to SEQ ID NO:376. In embodiments, the oligonucleotide includes a sequence at least 80% identical to SEQ ID NO:113 and the polynucleotide includes a sequence at least 80% identical to SEQ ID NO:377. In embodiments, the oligonucleotide includes a sequence at least 80% identical to SEQ ID NO:114 and the polynucleotide includes a sequence at least 80% identical to SEQ ID NO:378. In embodiments, the oligonucleotide includes a sequence at least 80% identical to SEQ ID NO:115 and the polynucleotide includes a sequence at least 80% identical to SEQ ID NO:379. In embodiments, the oligonucleotide includes a sequence at least 80% identical to SEQ ID NO:116 and the polynucleotide includes a sequence at least 80% identical to SEQ ID NO:380. In embodiments, the oligonucleotide includes a sequence at least 80% identical to SEQ ID NO:117 and the polynucleotide includes a sequence at least 80% identical to SEQ ID NO:381. In embodiments, the oligonucleotide includes a sequence at least 80% identical to SEQ ID NO:118 and the polynucleotide includes a sequence at least 80% identical to SEQ ID NO:382. In embodiments, the oligonucleotide includes a sequence at least 80% identical to SEQ ID NO:119 and the polynucleotide includes a sequence at least 80% identical to SEQ ID NO:383. In embodiments, the oligonucleotide includes a sequence at least 80% identical to SEQ ID NO: 120 and the polynucleotide includes a sequence at least 80% identical to SEQ ID NO:384. In embodiments, the oligonucleotide includes a sequence at least 80% identical to SEQ ID NO:121 and the polynucleotide includes a sequence at least 80% identical to SEQ ID NO:385. In embodiments, the oligonucleotide includes a sequence at least 80% identical to SEQ ID NO: 122 and the polynucleotide includes a sequence at least 80% identical to SEQ ID NO:386. In embodiments, the oligonucleotide includes a sequence at least 80% identical to SEQ ID NO:123 and the polynucleotide includes a sequence at least 80% identical to SEQ ID NO:387. In embodiments, the oligonucleotide includes a sequence at least 80% identical to SEQ ID NO:124 and the polynucleotide includes a sequence at least 80% identical to SEQ ID NO:388. In embodiments, the oligonucleotide includes a sequence at least 80% identical to SEQ ID NO: 125 and the polynucleotide includes a sequence at least 80% identical to SEQ ID NO:389. In embodiments, the oligonucleotide includes a sequence at least 80% identical to SEQ ID NO: 126 and the polynucleotide includes a sequence at least 80% identical to SEQ ID NO:390. In embodiments, the oligonucleotide includes a sequence at least 80% identical to SEQ ID NO:127 and the polynucleotide includes a sequence at least 80% identical to SEQ ID NO:391. In embodiments, the oligonucleotide includes a sequence at least 80% identical to SEQ ID NO:128 and the polynucleotide includes a sequence at least 80% identical to SEQ ID NO:392. In embodiments, the oligonucleotide includes a sequence at least 80% identical to SEQ ID NO:129 and the polynucleotide includes a sequence at least 80% identical to SEQ ID NO:393. In embodiments, the oligonucleotide includes a sequence at least 80% identical to SEQ ID NO:130 and the polynucleotide includes a sequence at least 80% identical to SEQ ID NO:394. In embodiments, the oligonucleotide includes a sequence at least 80% identical to SEQ ID NO:131 and the polynucleotide includes a sequence at least 80% identical to SEQ ID NO:395. In embodiments, the oligonucleotide includes a sequence at least 80% identical to SEQ ID NO:132 and the polynucleotide includes a sequence at least 80% identical to SEQ ID NO:396.

In embodiments, the oligonucleotide includes a sequence a sequence selected from SEQ ID NO:1 to SEQ ID NO:132 and the polynucleotide described herein or polynucleotide probe described herein includes a sequence selected from SEQ ID NO: 133 to SEQ ID NO:396. In embodiments, the oligonucleotide includes SEQ ID NO:1 and the polynucleotide includes SEQ ID NO:265. In embodiments, the oligonucleotide includes SEQ ID NO:2 and the polynucleotide includes SEQ ID NO:266. In embodiments, the oligonucleotide includes SEQ ID NO: 3 and the polynucleotide includes SEQ ID NO:267. In embodiments, the oligonucleotide includes SEQ ID NO:4 and the polynucleotide includes SEQ ID NO:268. In embodiments, the oligonucleotide includes SEQ ID NO:5 and the polynucleotide includes SEQ ID NO:269. In embodiments, the oligonucleotide includes SEQ ID NO:6 and the polynucleotide includes SEQ ID NO: 270. In embodiments, the oligonucleotide includes SEQ ID NO:7 and the polynucleotide includes SEQ ID NO:271. In embodiments, the oligonucleotide includes SEQ ID NO:8 and the polynucleotide includes SEQ ID NO:272. In embodiments, the oligonucleotide includes SEQ ID NO: 9 and the polynucleotide includes SEQ ID NO:273. In embodiments, the oligonucleotide includes SEQ ID NO: 10 and the polynucleotide includes SEQ ID NO:274. In embodiments, the oligonucleotide includes SEQ ID NO:11 and the polynucleotide includes SEQ ID NO:275. In embodiments, the oligonucleotide includes SEQ ID NO:12 and the polynucleotide includes SEQ ID NO: 276. In embodiments, the oligonucleotide includes SEQ ID NO: 13 and the polynucleotide includes SEQ ID NO:277. In embodiments, the oligonucleotide includes SEQ ID NO: 14 and the polynucleotide includes SEQ ID NO:278. In embodiments, the oligonucleotide includes SEQ ID NO:15 and the polynucleotide includes SEQ ID NO:279. In embodiments, the oligonucleotide includes SEQ ID NO:16 and the polynucleotide includes SEQ ID NO:280. In embodiments, the oligonucleotide includes SEQ ID NO:17 and the polynucleotide includes SEQ ID NO: 281. In embodiments, the oligonucleotide includes SEQ ID NO:18 and the polynucleotide includes SEQ ID NO:282. In embodiments, the oligonucleotide includes SEQ ID NO: 19 and the polynucleotide includes SEQ ID NO:283. In embodiments, the oligonucleotide includes SEQ ID NO:20 and the polynucleotide includes SEQ ID NO:284. In embodiments, the oligonucleotide includes SEQ ID NO:21 and the polynucleotide includes SEQ ID NO:285. In embodiments, the oligonucleotide includes SEQ ID NO:22 and the polynucleotide includes SEQ ID NO: 286. In embodiments, the oligonucleotide includes SEQ ID NO:23 and the polynucleotide includes SEQ ID NO:287. In embodiments, the oligonucleotide includes SEQ ID NO: 24 and the polynucleotide includes SEQ ID NO:288. In embodiments, the oligonucleotide includes SEQ ID NO:25 and the polynucleotide includes SEQ ID NO:289. In embodiments, the oligonucleotide includes SEQ ID NO:26 and the polynucleotide includes SEQ ID NO:290. In embodiments, the oligonucleotide includes SEQ ID NO:27 and the polynucleotide includes SEQ ID NO: 291. In embodiments, the oligonucleotide includes SEQ ID NO:28 and the polynucleotide includes SEQ ID NO:292. In embodiments, the oligonucleotide includes SEQ ID NO: 29 and the polynucleotide includes SEQ ID NO:293. In embodiments, the oligonucleotide includes SEQ ID NO:30 and the polynucleotide includes SEQ ID NO:294. In embodiments, the oligonucleotide includes SEQ ID NO:31 and the polynucleotide includes SEQ ID NO:295. In embodiments, the oligonucleotide includes SEQ ID NO:32 and the polynucleotide includes SEQ ID NO: 296. In embodiments, the oligonucleotide includes SEQ ID NO:33 and the polynucleotide includes SEQ ID NO:297. In embodiments, the oligonucleotide includes SEQ ID NO: 34 and the polynucleotide includes SEQ ID NO:298. In embodiments, the oligonucleotide includes SEQ ID NO:35 and the polynucleotide includes SEQ ID NO:299. In embodiments, the oligonucleotide includes SEQ ID NO:36 and the polynucleotide includes SEQ ID NO:300. In embodiments, the oligonucleotide includes SEQ ID NO:37 and the polynucleotide includes SEQ ID NO: 301. In embodiments, the oligonucleotide includes SEQ ID NO:38 and the polynucleotide includes SEQ ID NO:302. In embodiments, the oligonucleotide includes SEQ ID NO: 39 and the polynucleotide includes SEQ ID NO:303. In embodiments, the oligonucleotide includes SEQ ID NO:40 and the polynucleotide includes SEQ ID NO:304. In embodiments, the oligonucleotide includes SEQ ID NO:41 and the polynucleotide includes SEQ ID NO:305. In embodiments, the oligonucleotide includes SEQ ID NO:42 and the polynucleotide includes SEQ ID NO: 306. In embodiments, the oligonucleotide includes SEQ ID NO:43 and the polynucleotide includes SEQ ID NO:307. In embodiments, the oligonucleotide includes SEQ ID NO: 44 and the polynucleotide includes SEQ ID NO:308. In embodiments, the oligonucleotide includes SEQ ID NO:45 and the polynucleotide includes SEQ ID NO:309. In embodiments, the oligonucleotide includes SEQ ID NO:46 and the polynucleotide includes SEQ ID NO:310. In embodiments, the oligonucleotide includes SEQ ID NO:47 and the polynucleotide includes SEQ ID NO: 311. In embodiments, the oligonucleotide includes SEQ ID NO:48 and the polynucleotide includes SEQ ID NO:312. In embodiments, the oligonucleotide includes SEQ ID NO: 49 and the polynucleotide includes SEQ ID NO:313. In embodiments, the oligonucleotide includes SEQ ID NO:50 and the polynucleotide includes SEQ ID NO:314. In embodiments, the oligonucleotide includes SEQ ID NO:51 and the polynucleotide includes SEQ ID NO:315. In embodiments, the oligonucleotide includes SEQ ID NO:52 and the polynucleotide includes SEQ ID NO: 316. In embodiments, the oligonucleotide includes SEQ ID NO:53 and the polynucleotide includes SEQ ID NO:317. In embodiments, the oligonucleotide includes SEQ ID NO: 54 and the polynucleotide includes SEQ ID NO:318. In embodiments, the oligonucleotide includes SEQ ID NO:55 and the polynucleotide includes SEQ ID NO:319. In embodiments, the oligonucleotide includes SEQ ID NO:56 and the polynucleotide includes SEQ ID NO:320. In embodiments, the oligonucleotide includes SEQ ID NO:57 and the polynucleotide includes SEQ ID NO: 321. In embodiments, the oligonucleotide includes SEQ ID NO:58 and the polynucleotide includes SEQ ID NO:322. In embodiments, the oligonucleotide includes SEQ ID NO: 59 and the polynucleotide includes SEQ ID NO:323. In embodiments, the oligonucleotide includes SEQ ID NO:60 and the polynucleotide includes SEQ ID NO:324. In embodiments, the oligonucleotide includes SEQ ID NO:61 and the polynucleotide includes SEQ ID NO:325. In embodiments, the oligonucleotide includes SEQ ID NO:62 and the polynucleotide includes SEQ ID NO: 326. In embodiments, the oligonucleotide includes SEQ ID NO:63 and the polynucleotide includes SEQ ID NO:327. In embodiments, the oligonucleotide includes SEQ ID NO: 64 and the polynucleotide includes SEQ ID NO:328. In embodiments, the oligonucleotide includes SEQ ID NO:65 and the polynucleotide includes SEQ ID NO:329. In embodiments, the oligonucleotide includes SEQ ID NO:66 and the polynucleotide includes SEQ ID NO:330. In embodiments, the oligonucleotide includes SEQ ID NO:67 and the polynucleotide includes SEQ ID NO: 331. In embodiments, the oligonucleotide includes SEQ ID NO:68 and the polynucleotide includes SEQ ID NO:332. In embodiments, the oligonucleotide includes SEQ ID NO: 69 and the polynucleotide includes SEQ ID NO:333. In embodiments, the oligonucleotide includes SEQ ID NO:70 and the polynucleotide includes SEQ ID NO:334. In embodiments, the oligonucleotide includes SEQ ID NO:71 and the polynucleotide includes SEQ ID NO:335. In embodiments, the oligonucleotide includes SEQ ID NO:72 and the polynucleotide includes SEQ ID NO: 336. In embodiments, the oligonucleotide includes SEQ ID NO:73 and the polynucleotide includes SEQ ID NO:337. In embodiments, the oligonucleotide includes SEQ ID NO: 74 and the polynucleotide includes SEQ ID NO:338. In embodiments, the oligonucleotide includes SEQ ID NO:75 and the polynucleotide includes SEQ ID NO:339. In embodiments, the oligonucleotide includes SEQ ID NO:76 and the polynucleotide includes SEQ ID NO:340. In embodiments, the oligonucleotide includes SEQ ID NO:77 and the polynucleotide includes SEQ ID NO: 341. In embodiments, the oligonucleotide includes SEQ ID NO:78 and the polynucleotide includes SEQ ID NO:342. In embodiments, the oligonucleotide includes SEQ ID NO: 79 and the polynucleotide includes SEQ ID NO:343. In embodiments, the oligonucleotide includes SEQ ID NO:80 and the polynucleotide includes SEQ ID NO:344. In embodiments, the oligonucleotide includes SEQ ID NO:81 and the polynucleotide includes SEQ ID NO:345. In embodiments, the oligonucleotide includes SEQ ID NO:82 and the polynucleotide includes SEQ ID NO: 346. In embodiments, the oligonucleotide includes SEQ ID NO:83 and the polynucleotide includes SEQ ID NO:347. In embodiments, the oligonucleotide includes SEQ ID NO: 84 and the polynucleotide includes SEQ ID NO:348. In embodiments, the oligonucleotide includes SEQ ID NO:85 and the polynucleotide includes SEQ ID NO:349. In embodiments, the oligonucleotide includes SEQ ID NO:86 and the polynucleotide includes SEQ ID NO:350. In embodiments, the oligonucleotide includes SEQ ID NO:87 and the polynucleotide includes SEQ ID NO: 351. In embodiments, the oligonucleotide includes SEQ ID NO:88 and the polynucleotide includes SEQ ID NO:352. In embodiments, the oligonucleotide includes SEQ ID NO: 89 and the polynucleotide includes SEQ ID NO:353. In embodiments, the oligonucleotide includes SEQ ID NO:90 and the polynucleotide includes SEQ ID NO:354. In embodiments, the oligonucleotide includes SEQ ID NO:91 and the polynucleotide includes SEQ ID NO:355. In embodiments, the oligonucleotide includes SEQ ID NO:92 and the polynucleotide includes SEQ ID NO: 356. In embodiments, the oligonucleotide includes SEQ ID NO:93 and the polynucleotide includes SEQ ID NO:357. In embodiments, the oligonucleotide includes SEQ ID NO: 94 and the polynucleotide includes SEQ ID NO:358. In embodiments, the oligonucleotide includes SEQ ID NO:95 and the polynucleotide includes SEQ ID NO:359. In embodiments, the oligonucleotide includes SEQ ID NO:96 and the polynucleotide includes SEQ ID NO:360. In embodiments, the oligonucleotide includes SEQ ID NO:97 and the polynucleotide includes SEQ ID NO: 361. In embodiments, the oligonucleotide includes SEQ ID NO:98 and the polynucleotide includes SEQ ID NO:362. In embodiments, the oligonucleotide includes SEQ ID NO: 99 and the polynucleotide includes SEQ ID NO:363. In embodiments, the oligonucleotide includes SEQ ID NO:100 and the polynucleotide includes SEQ ID NO:364. In embodiments, the oligonucleotide includes SEQ ID NO:101 and the polynucleotide includes SEQ ID NO:365. In embodiments, the oligonucleotide includes SEQ ID NO:102 and the polynucleotide includes SEQ ID NO:366. In embodiments, the oligonucleotide includes SEQ ID NO:103 and the polynucleotide includes SEQ ID NO:367. In embodiments, the oligonucleotide includes SEQ ID NO: 104 and the polynucleotide includes SEQ ID NO:368. In embodiments, the oligonucleotide includes SEQ ID NO:105 and the polynucleotide includes SEQ ID NO:369. In embodiments, the oligonucleotide includes SEQ ID NO:106 and the polynucleotide includes SEQ ID NO:370. In embodiments, the oligonucleotide includes SEQ ID NO:107 and the polynucleotide includes SEQ ID NO:371. In embodiments, the oligonucleotide includes SEQ ID NO:108 and the polynucleotide includes SEQ ID NO:372. In embodiments, the oligonucleotide includes SEQ ID NO: 109 and the polynucleotide includes SEQ ID NO:373. In embodiments, the oligonucleotide includes SEQ ID NO:110 and the polynucleotide includes SEQ ID NO:374. In embodiments, the oligonucleotide includes SEQ ID NO:111 and the polynucleotide includes SEQ ID NO:375. In embodiments, the oligonucleotide includes SEQ ID NO:112 and the polynucleotide includes SEQ ID NO:376. In embodiments, the oligonucleotide includes SEQ ID NO: 113 and the polynucleotide includes SEQ ID NO:377. In embodiments, the oligonucleotide includes SEQ ID NO: 114 and the polynucleotide includes SEQ ID NO:378. In embodiments, the oligonucleotide includes SEQ ID NO:115 and the polynucleotide includes SEQ ID NO:379. In embodiments, the oligonucleotide includes SEQ ID NO:116 and the polynucleotide includes SEQ ID NO:380. In embodiments, the oligonucleotide includes SEQ ID NO:117 and the polynucleotide includes

SEQ ID NO:381. In embodiments, the oligonucleotide includes SEQ ID NO: 118 and the polynucleotide includes SEQ ID NO:382. In embodiments, the oligonucleotide includes SEQ ID NO: 119 and the polynucleotide includes SEQ ID NO:383. In embodiments, the oligonucleotide includes SEQ ID NO:120 and the polynucleotide includes SEQ ID NO:384. In embodiments, the oligonucleotide includes SEQ ID NO: 121 and the polynucleotide includes SEQ ID NO:385. In embodiments, the oligonucleotide includes SEQ ID NO:122 and the polynucleotide includes SEQ ID NO:386. In embodiments, the oligonucleotide includes SEQ ID NO: 123 and the polynucleotide includes SEQ ID NO:387. In embodiments, the oligonucleotide includes SEQ ID NO: 124 and the polynucleotide includes SEQ ID NO:388. In embodiments, the oligonucleotide includes SEQ ID NO:125 and the polynucleotide includes SEQ ID NO:389. In embodiments, the oligonucleotide includes SEQ ID NO: 126 and the polynucleotide includes SEQ ID NO:390. In embodiments, the oligonucleotide includes SEQ ID NO:127 and the polynucleotide includes SEQ ID NO:391. In embodiments, the oligonucleotide includes SEQ ID NO:128 and the polynucleotide includes SEQ ID NO:392. In embodiments, the oligonucleotide includes SEQ ID NO: 129 and the polynucleotide includes SEQ ID NO:393. In embodiments, the oligonucleotide includes SEQ ID NO: 130 and the polynucleotide includes SEQ ID NO:394. In embodiments, the oligonucleotide includes SEQ ID NO:131 and the polynucleotide includes SEQ ID NO:395. In embodiments, the oligonucleotide includes SEQ ID NO:132 and the polynucleotide includes SEQ ID NO:396.

In an aspect is provided a cell, wherein the cell includes the composition as described herein. In another aspect is a tissue, wherein the tissue includes the composition as described herein. In another aspect is provided a target molecule, wherein the target molecule is bound to a composition as described herein. In embodiments, the target molecule is PD-L1, CD8, CD3, PD-1, CD45, CD4, CD68, CD11c, FoxP3, α-SMA, CD20, Ki67, CD56, CD31, CTLA-4/CD152, CTLA-4/CD153, p53, PanCK, CD45RO, CD45RA, and/or HLA-DR. In embodiments, the target molecule is selected from the group: PD-L1, CD8, CD3, PD-1, CD45, CD4, CD68, CD11c, FoxP3, α-SMA, CD20, Ki67, CD56, CD31, CTLA-4, PanCK, CD45RO, CD45RA, ATPase, Pan-Cadherin, Vimentin, Beta-2-microglobulin, and HLA-DR.

In an aspect is provided a cell or tissue including an oligonucleotide described herein, wherein the oligonucleotide is covalently attached to a specific binding agent that is an antibody, single-chain Fv fragment (scFv), antibody fragment-antigen binding (Fab), affimer, aptamer, enzyme, peptide, Molecular Imprinted Polymer (MIP), DARPin (Designed Ankyrin Repeat Protein), peptoid, or lectin. In embodiments, the cell or tissue further includes a first blocking oligonucleotide hybridized to a first sequence of the oligonucleotide described herein, and a second blocking oligonucleotide hybridized to a second sequence of the oligonucleotide described herein. In embodiments, the cell further includes a polynucleotide probe including a first binding sequence and a second binding sequence hybridized to the oligonucleotide described herein.

In embodiments, the target molecule is PD-L1. PD-L1 may be highly expressed in a variety of malignancies, for example lung cancer. Detecting PD-L1 may be useful in determining the effectiveness of gene therapy or systemic immunotherapy in blocking the PD-1 and PD-L1 checkpoints. In embodiments, detecting PD-L1 in TNBC cells or tissues is useful for evaluating patients for immunotherapy.

In embodiments, the target molecule is CD8 (e.g., CD8A and/or CD8B). In embodiments, detecting CD8 is useful for determining cytotoxic T cell infiltration into a cell or tissue.

In embodiments, the target molecule is CD3. In embodiments, the target molecule is CD3E (CD3 epsilon). CD3 is typically bound to the membranes of all mature T-cells.

In embodiments, the target molecule is PD1. PD1 often shows high and sustained expression levels during persistent antigen encounter, and is expressed by conventional T cells, regulatory T cells, B cells, and natural killer cells. In embodiments, detecting PD1 is useful as an immune checkpoint protein. In embodiments, Programmed cell death 1 receptor (PD-1) and its ligand programmed cell death ligand 1 (PD-L1) are immune checkpoint proteins found on the cell surface of T cells. Under physiological conditions their interaction results in T cell immune suppression, but diseases (e.g., cancer) disrupt normal function to escape immune detection.

In embodiments, the target molecule is CD4. CD4 is expressed in most neoplasms derived from T helper cells. For example, it is possible to use CD4 detection on tissue samples to identify most forms of peripheral T cell lymphoma and related malignant conditions. CD4 is also associated with a number of autoimmune diseases such as vitiligo and type I diabetes mellitus.

In embodiments, the target molecule is CD68. CD68 is typically found in the cytoplasmic granules of a range of different blood cells and myocytes, and is useful as a diagnostic marker for cells of the macrophage lineage, including monocytes, histiocytes, giant cells, Kupffer cells, and osteoclasts. Due to the presence of CD68 in macrophages, detecting CD68 is useful in diagnosing conditions related to proliferation or abnormality of these cells, such as malignant histiocytosis, histiocytic lymphoma, and Gaucher's disease. CD68 alone or in combination with other cell markers of tumor-associated macrophages is correlated with positive predictive value as a prognostic marker of survival in cancer patients.

In embodiments, the target molecule is CD11c. CD11c is a marker for dendritic cells, which are bone-marrow-derived cells found in the blood, epithelial and lymphoid tissues. CD11c is an approximately 150 kDa type I transmembrane glycoprotein protein and part of the complement receptor 4 (CR4). Together with CR3, named also Mac-1 or CD11b/CD18, CR4 belongs to the 2 integrin family of adhesion molecules. CD11c is not restricted to dendritic cells, but also expressed by other immune cells, such as macrophages in the alveoli and adipose tissues.

In embodiments, the target molecule is FoxP3. Alterations in numbers of regulatory T cells and in particular those that express Foxp3 are found in a number of disease states. For example, down-regulation of Foxp3 expression has been reported in tumor samples derived from breast, prostate, and ovarian cancer patients. In embodiments, detection of human FoxP3 is associated with regulatory T cell detection.

In embodiments, the target molecule is ACTA2 (commonly referred to as alpha-smooth muscle actin or α-SMA) is used as a marker of myofibroblast formation.

In embodiments, the target molecule is CD20. CD20 is expressed on all stages of B cell development from pre-B cells in the bone-marrow through immature, naive, mature and memory cells in lymphoid tissues and blood, though the expression is lost in terminally differentiated plasma cells. CD20 is a transmembrane protein consisting of four hydrophobic transmembrane domains, one intracellular domain and two extracellular loops. In embodiments, the detection of CD20 is associated with B cell malignancies, such as B-cell lymphomas, hairy cell leukemia, B-cell chronic lymphocytic leukemia, and melanoma cancer stem cells. Due to CD20 remains present on the cells of most B-cell neoplasms, and is absent on otherwise similar appearing T-cell neoplasms, detecting CD20 can be useful in diagnosing conditions such as B-cell lymphomas and leukemias.

In embodiments, the target molecule is Ki-67. Ki-67 is a nuclear protein that is associated with cellular proliferation and ribosomal RNA transcription.

In embodiments, the target molecule is CD56. Normal cells that stain positively for CD56 include natural killer cells, activated T cells, the brain and cerebellum, and neuroendocrine tissues. Additional tumor tissue sections that are CD56 positive include CD56-positive include tumor tissue section associated with myeloma, myeloid leukemia, neuroendocrine tumors, Wilms' tumor, neuroblastoma, extranodal NK/T-cell lymphoma, nasal type, pancreatic acinar cell carcinoma, pheochromocytoma, paraganglioma, and small cell lung carcinoma.

In embodiments, the target molecule is CD31. CD31 is used primarily to demonstrate the presence of endothelial cells in tissue sections. CD31 is typically found on the surface of platelets, monocytes, neutrophils, and some types of T-cells, and endothelial cell intercellular junctions. Expression of CD31 is correlated hemangioma, angiosarcoma, Kaposi's sarcoma, breast carcinoma, glioblastoma, colon carcinoma, and skin carcinoma.

In embodiments, the target molecule is CTLA-4. CTLA-4 is expressed in regulatory T cells and typically upregulated in conventional T cells after activation.

In embodiments, the target molecule is PanCK.

In embodiments, the target molecule is HLA-DR (alternatively referred to as HLA-DRA). HLA-DR is expressed on the surface of various antigen presenting cells such as B lymphocytes, dendritic cells, and monocytes/macrophages, and plays a role in the immune system and response by presenting peptides derived from extracellular proteins, in particular, pathogen-derived peptides to T cells.

In embodiments, the target molecule is vimentin. Vimentin is a type III intermediate filament (IF) protein and is a cytoskeletal component of mesenchymal cells. Vimentin is used as a marker of mesenchymally-derived cells or cells undergoing an epithelial-to-mesenchymal transition (EMT). Vimentin is useful at ascertaining the cell shape.

In embodiments, the target molecule is CD45. CD45 is an antigen expressed on immunological and hematological systems but is not expressed on red blood cells and platelets. Aberrant expression of CD45 is associated with leukemia and lymphoma. Expression of the six isoforms of CD45 contributes to lymphocyte survival, cytokine response, and T-cell receptor signaling.

In embodiments, the target molecule is CD45RO. CD45RO is an isoform of CD45 and is expressed by human memory T cells and activated T lymphocytes. In embodiments, the target molecule is CD45RA. CD45RA is an isoform of CD45 and is expressed in a subset of T cells, B cells, and monocytes.

In embodiments, the target molecule is ATPase. ATPase represents a superfamily involved in the synthesis of ATP from the phosphorylation of ADP, which is coupled by the transport of proton ions across membranes.

In embodiments, the target molecule is pan-cadherin. Pan-cadherin is a member of the cadherin protein family, and is a cell-cell adhesion glycoprotein that is critical for tissue architecture, cell differentiation, cell shape, cell polarity, growth and migration, particularly in epithelial tissues.

In embodiments, the target molecule is beta 2-microglobulin (B2M). The expression of this marker indicates the activation of the immune system. Aberrant expression of B2M is also observed in hematologic and renal disorders.

In embodiments, the cell forms part of a tissue in situ. In embodiments, the cell is an isolated single cell. In embodiments, the cell is a prokaryotic cell. In embodiments, the cell is a eukaryotic cell. In embodiments, the cell is a bacterial cell, a fungal cell, a plant cell, or a mammalian cell. In embodiments, the cell is a stem cell. In embodiments, the stem cell is an embryonic stem cell, a tissue-specific stem cell, a mesenchymal stem cell, or an induced pluripotent stem cell. In embodiments, the cell is an endothelial cell, muscle cell, myocardial, smooth muscle cell, skeletal muscle cell, mesenchymal cell, epithelial cell; hematopoietic cell, such as lymphocytes, including T cell, e.g., (Th1 T cell, Th2 T cell, ThO T cell, cytotoxic T cell); B cell, pre-B cell; monocytes; dendritic cell; neutrophils; or a macrophage. In embodiments, the cell is a stem cell, an immune cell, a cancer cell (e.g., a circulating tumor cell or cancer stem cell), a viral-host cell, or a cell that selectively binds to a desired target. In embodiments, the cell includes a T cell receptor gene sequence, a B cell receptor gene sequence, or an immunoglobulin gene sequence. In embodiments, the cell includes a Toll-like receptor (TLR) gene sequence. In embodiments, the cell includes a gene sequence corresponding to an immunoglobulin light chain polypeptide and a gene sequence corresponding to an immunoglobulin heavy chain polypeptide. In embodiments, the cell is a genetically modified cell. In embodiments, the cell is a circulating tumor cell or cancer stem cell.

In embodiments, the cell is an adherent cell (e.g., epithelial cell, endothelial cell, or neural cell). Adherent cells are usually derived from tissues of organs and attach to a substrate (e.g., epithelial cells adhere to an extracellular matrix coated substrate via transmembrane adhesion protein complexes). Adherent cells typically require a substrate, e.g., tissue culture plastic, which may be coated with extracellular matrix (e.g., collagen and laminin) components to increase adhesion properties and provide other signals needed for growth and differentiation. In embodiments, the cell is a leukocyte (i.e., a white-blood cell). In embodiments, leukocyte is a granulocyte (neutrophil, eosinophil, or basophil), monocyte, or lymphocyte (T cells and B cells). In embodiments, the cell is a lymphocyte. In embodiments, the cell is a neuronal cell, an endothelial cell, epithelial cell, germ cell, plasma cell, a muscle cell, peripheral blood mononuclear cell (PBMC), a myocardial cell, or a retina cell. In embodiments, the cell is a suspension cell (e.g., a cell free-floating in the culture medium, such a lymphoblast or hepatocyte). In embodiments, the cell is a glial cell (e.g., astrocyte, radial glia), pericyte, or stem cell (e.g., a neural stem cell). In embodiments, the cell is a neuronal cell. In embodiments, the cell is an endothelial cell. In embodiments, the cell is an epithelial cell. In embodiments, the cell is a germ cell. In embodiments, the cell is a plasma cell. In embodiments, the cell is a muscle cell. In embodiments, the cell is a peripheral blood mononuclear cell (PBMC). In embodiments, the cell is a myocardial cell. In embodiments, the cell is a retina cell. In embodiments, the cell is a lymphoblast. In embodiments, the cell is a hepatocyte. In embodiments, the cell is a glial cell. In embodiments, the cell is an astrocyte. In embodiments, the cell is a radial glia. In embodiments, the cell is a pericyte. In embodiments, the cell is a stem cell. In embodiments, the cell is a neural stem cell.

In embodiments, the cell is an immune cell. In embodiments, the immune cell is a granulocyte, a mast cell, a monocyte, a neutrophil, a dendritic cell, or a natural killer (NK) cell. In embodiments, the immune cell is an adaptive cell, such as a T cell, NK cell, or a B cell. In embodiments, the cell includes a T cell receptor gene sequence, a B cell receptor gene sequence, or an immunoglobulin gene sequence. In embodiments, the immune cell is a granulocyte. In embodiments, the immune cell is a mast cell. In embodiments, the immune cell is a monocyte. In embodiments, the immune cell is a neutrophil. In embodiments, the immune cell is a dendritic cell. In embodiments, the immune cell is a natural killer (NK) cell. In embodiments, the immune cell is a T cell. In embodiments, the immune cell is a B cell. In embodiments, the cell includes a T cell receptor gene sequence. In embodiments, the cell includes a B cell receptor gene sequence. In embodiments, the cell includes an immunoglobulin gene sequence. In embodiments, the cell is a cancer cell. In embodiments, the cancer cell includes a cancer-associated gene (e.g., an oncogene associated with kinases and genes involved in DNA repair) or a cancer-associated biomarker. A “biomarker” is a substance that is associated with a particular characteristic, such as a disease or condition. A change in the levels of a biomarker may correlate with the risk or progression of a disease or with the susceptibility of the disease to a given treatment. In embodiments, the cancer is Acute Myeloid Leukemia, Adrenocortical Carcinoma, Bladder Urothelial Carcinoma, Breast Ductal Carcinoma, Breast Lobular Carcinoma, Cervical Carcinoma, Cholangiocarcinoma, Colorectal Adenocarcinoma, Esophageal Carcinoma, Gastric Adenocarcinoma, Glioblastoma Multiforme, Head and Neck Squamous Cell Carcinoma, Hepatocellular Carcinoma, Kidney Chromophobe Carcinoma, Kidney Clear Cell Carcinoma, Kidney Papillary Cell Carcinoma, Lower Grade Glioma, Lung Adenocarcinoma, Lung Squamous Cell Carcinoma, Mesothelioma, Ovarian Serous Adenocarcinoma, Pancreatic Ductal Adenocarcinoma, Paraganglioma & Pheochromocytoma, Prostate Adenocarcinoma, Sarcoma, Skin Cutaneous Melanoma, Testicular Germ Cell Cancer, Thymoma, Thyroid Papillary Carcinoma, Uterine Carcinosarcoma, Uterine Corpus Endometrioid Carcinoma, or Uveal Melanoma.

In embodiments, the cell in situ is obtained from a subject (e.g., human or animal tissue). Once obtained, the cell is placed in an artificial environment in plastic or glass containers supported with specialized medium containing essential nutrients and growth factors to support proliferation. In embodiments, the cell is permeabilized and immobilized to a solid support surface. In embodiments, the cell is permeabilized and immobilized to an array (i.e., to discrete locations arranged in an array). In embodiments, the cell is immobilized to a solid support surface.

In embodiments, the methods are performed in situ on isolated cells or in tissue sections that have been prepared according to methodologies known in the art. Methods for permeabilization and fixation of cells and tissue samples are known in the art, as exemplified by Cremer et al., The Nucleus: Volume 1: Nuclei and Subnuclear Components, R. Hancock (ed.) 2008; and Larsson et al., Nat. Methods (2010) 7:395-397, the content of each of which is incorporated herein by reference in its entirety. In embodiments, the cell is cleared (e.g., digested) of proteins, lipids, or both proteins and lipids.

In embodiments, the cell is immobilized to a substrate. The cell may have been cultured on the surface, or the cell may have been initially cultured in suspension and then fixed to the surface. Substrates can be two- or three-dimensional and can include a planar surface (e.g., a glass slide). A substrate can include glass (e.g., controlled pore glass (CPG)), quartz, plastic (such as polystyrene (low cross-linked and high cross-linked polystyrene), polycarbonate, polypropylene and poly(methymethacrylate)), acrylic copolymer, polyamide, silicon, metal (e.g., alkanethiolate-derivatized gold), cellulose, nylon, latex, dextran, gel matrix (e.g., silica gel), polyacrolein, or composites. In embodiments, the substrate is a borosilicate glass substrate with a composition including SiO2, Al₂O₃, B₂O₃, Li₂O, Na₂O, K₂O, MgO, CaO, SrO, BaO, ZnO, TiO₂, ZrO₂, P₂O₅, or a combination thereof (see e.g., U.S. Pat. No. 10,974,990). In embodiments, the substrate is an alkaline earth boro-aluminosilicate glass substrate. In embodiments, the substrate includes a polymeric coating, optionally containing bioconjugate reactive moieties capable of affixing the sample. Suitable three-dimensional substrates include, for example, spheres, microparticles, beads, membranes, slides, plates, micromachined chips, tubes (e.g., capillary tubes), microwells, microfluidic devices, channels, filters, or any other structure suitable for anchoring a sample. In embodiments, the substrate is not a flow cell. In embodiments, the substrate includes a polymer matrix material (e.g., polyacrylamide, cellulose, alginate, polyamide, cross-linked agarose, cross-linked dextran or cross-linked polyethylene glycol), which may be referred to herein as a “matrix”, “synthetic matrix”, “exogenous polymer” or “exogenous hydrogel”. In embodiments, a matrix may refer to the various components and organelles of a cell, for example, the cytoskeleton (e.g., actin and tubulin), endoplasmic reticulum, Golgi apparatus, vesicles, etc. In embodiments, the matrix is endogenous to a cell. In embodiments, the matrix is exogenous to a cell. In embodiments, the matrix includes both the intracellular and extracellular components of a cell. In embodiments, polynucleotide primers may be immobilized on a matrix including the various components and organelles of a cell. Immobilization of polynucleotide primers on a matrix of cellular components and organelles of a cell is accomplished as described herein, for example, through the interaction/reaction of complementary bioconjugate reactive moieties. In embodiments, the exogenous polymer may be a matrix or a network of extracellular components that act as a point of attachment (e.g., act as an anchor) for the cell to a substrate.

In embodiments, the solid support or substrate described herein includes one or more channels. In embodiments, the solid support or substrate includes a channel bored into solid support or substrate. In embodiments, the solid support or substrate includes a plurality of channels solid support or substrate. In embodiments, the solid support or substrate includes 2, 3, or 4 channels bored into solid support or substrate. In embodiments, the width of the channel is from about 1 to 5 mm, 5 mm to 10 mm, or 10 mm to 15 mm. In embodiments, the channel is a reaction chamber on the solid support or substrate. In embodiments, the cell or tissue is immobilized in a channel bored onto the solid support or substrate.

In embodiments, the cell is exposed to paraformaldehyde (i.e., by contacting the cell with paraformaldehyde). Any suitable permeabilization and fixation technologies can be used for making the cell available for the detection methods provided herein. In embodiments the method includes affixing single cells or tissues to a transparent substrate. Exemplary tissues include those from skin tissue, muscle tissue, bone tissue, organ tissue and the like. In embodiments, the method includes immobilizing the cell in situ to a substrate and permeabilized for delivering probes, enzymes, nucleotides and other components required in the reactions. In embodiments, the cell includes many cells from a tissue section in which the original spatial relationships of the cells are retained. In embodiments, the cell in situ is within a Formalin-Fixed Paraffin-Embedded (FFPE) sample. In embodiments, the cell is subjected to paraffin removal methods, such as methods involving incubation with a hydrocarbon solvent, such as xylene or hexane, followed by two or more washes with decreasing concentrations of an alcohol, such as ethanol. The cell may be rehydrated in a buffer, such as PBS, TBS or MOPs. In embodiments, the FFPE sample is incubated with xylene and washed using ethanol to remove the embedding wax, followed by treatment with Proteinase K to permeabilized the tissue. In embodiments, the cell is fixed with a chemical fixing agent. In embodiments, the chemical fixing agent is formaldehyde or glutaraldehyde. In embodiments, the chemical fixing agent is glyoxal or dioxolane. In embodiments, the chemical fixing agent includes one or more of ethanol, methanol, 2-propanol, acetone, and glyoxal. In embodiments, the chemical fixing agent includes formalin, Greenfix®, Greenfix® Plus, UPM, CyMol®, HOPE®, CytoSkelFix™, F-Solv®, FineFIX®, RCL2/KINFix, UMFIX, Glyo-Fixx®, Histochoice®, or PAXgene® In embodiments, the cell is fixed within a synthetic three-dimensional matrix (e.g., polymeric material). In embodiments, the synthetic matrix includes polymeric-crosslinking material. In embodiments, the material includes polyacrylamide, poly-ethylene glycol (PEG), poly(acrylate-co-acrylic acid) (PAA), or Poly(N-isopropylacrylamide) (NIPAM).

The biological sample can be a biological tissue, cultured cells, or cells taken from an animal subject of interest. In embodiments, the biological sample includes material that is human origin or mouse origin. In embodiments, the biological sample is fresh, frozen, or fixed. In embodiments it can be a section or core obtained from a formalin-fixed paraffin-embedded (FFPE) tissue block. The sample can include material from a tissue section, tissue micro-array (TMA), cell pellet, core biopsy, needle biopsy, or cells obtained from a blood or serum sample. In embodiments, the biological sample is immobilized on a surface of a functionalized slide, a functionalized plate, a functionalized well, or a functionalized film. The biological sample can be contacted with two or more (e.g., 3 or more, 4 or more, 5 or more, 6 or more, 8 or more, 10 or more, 12 or more, 15 or more, 20 or more, 30 or more, 40 or more, 50 or more, 60 or more, 80 or more, or even more) antibodies, antibody fragments, or a combination thereof. The sample can be contacted with a cocktail of all the antibodies or antibody fragments, or combinations of multiple subsets of the total number of antibodies.

In embodiments, the composition includes a specific binding agent covalently attached to an oligonucleotide, wherein the oligonucleotide includes a sequence at least 80% identical to a sequence selected from SEQ ID NO:397 to SEQ ID NO:529.

In an aspect is provided an oligonucleotide including a sequence formed according to the computer-implemented method described herein. In embodiments, the oligonucleotide including a sequence formed according to the computer-implemented method described herein does not include a GC percentage greater than 60% over a portion of the oligonucleotide sequence. In embodiments, the oligonucleotide including a sequence formed according to the computer-implemented method described herein does not include five consecutive strong bases and or five consecutive weak bases. In embodiments, the oligonucleotide including a sequence formed according to the computer-implemented method described herein does not include secondary structure. In embodiments, the oligonucleotide including a sequence formed according to the computer-implemented method described herein does not include complementarity to the transcriptome. In embodiments, the oligonucleotide including a sequence formed according to the computer-implemented method described herein does not include complementarity to the genome. In embodiments, the oligonucleotide including a sequence formed according to the computer-implemented method described herein does not include a homopolymer sequence greater than 4 nucleotides.

In an aspect is provided a kit. In embodiments, the kit includes the composition as described herein. In embodiments, the kit includes labeled nucleotides including differently labeled nucleotides, enzymes, buffers, oligonucleotides, and related solvents and solutions. In embodiments, the kit includes a padlock probe (e.g., a polynucleotide as described herein or polynucleotide probe described herein). In embodiments, the kit includes a specific binding agent described herein, wherein the specific binding agent includes an oligonucleotide described herein. The kit may also include a template nucleic acid (DNA and/or RNA), one or more primer polynucleotides, nucleoside triphosphates (including, e.g., deoxyribonucleotides, dideoxynucleotides, ribonucleotides, labeled nucleotides, and/or modified nucleotides), buffers, salts, and/or labels (e.g., fluorophores). In embodiments, the kit includes components useful for circularizing template polynucleotides using chemical ligation techniques. In embodiments, the kit includes components useful for circularizing template polynucleotides using a ligation enzyme (e.g., CircLigase™ enzyme, Taq DNA Ligase, HiFi Taq DNA Ligase, T4 ligase, or Ampligase® DNA Ligase). In embodiments the ligation enzyme is an RNA-dependent DNA ligase (e.g., SplintR® ligase). For example, such a kit further includes the following components: (a) reaction buffer for controlling pH and providing an optimized salt composition for a ligation enzyme (e.g., CircLigase™ enzyme, Taq DNA Ligase, HiFi Taq DNA Ligase, T4 ligase, or Ampligase™ DNA Ligase), and (b) ligation enzyme cofactors. In embodiments, the kit further includes instructions for use thereof. In embodiments, kits described herein include a polymerase. In embodiments, the polymerase is a DNA polymerase. In embodiments, the DNA polymerase is a thermophilic nucleic acid polymerase. In embodiments, the DNA polymerase is a modified archaeal DNA polymerase. In embodiments, the kit includes a sequencing solution. In embodiments, the sequencing solution include labeled nucleotides including differently labeled nucleotides, wherein the label (or lack thereof) identifies the type of nucleotide. For example, each adenine nucleotide, or analog thereof; a thymine nucleotide; a cytosine nucleotide, or analog thereof; and a guanine nucleotide, or analog thereof may be labeled with a different fluorescent label.

In embodiments, the kit includes, without limitation, nucleic acid primers, probes, adapters, enzymes, and the like, and are each packaged in a container, such as, without limitation, a vial, tube or bottle, in a package suitable for commercial distribution, such as, without limitation, a box, a sealed pouch, a blister pack and a carton. The package typically contains a label or packaging insert indicating the uses of the packaged materials. As used herein, “packaging materials” includes any article used in the packaging for distribution of reagents in a kit, including without limitation containers, vials, tubes, bottles, pouches, blister packaging, labels, tags, instruction sheets and package inserts.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, digital storage medium, etc., on which the information has been recorded. Yet another means that may be present is a website address which may be used via the Internet to access the information at a removed site. Any convenient means may be present in the kits.

Adapters and/or primers may be supplied in the kits ready for use, as concentrates-requiring dilution before use, or in a lyophilized or dried form requiring reconstitution prior to use. If required, the kits may further include a supply of a suitable diluent for dilution or reconstitution of the primers and/or adapters. Optionally, the kits may further include supplies of reagents, buffers, enzymes, and dNTPs for use in carrying out nucleic acid amplification and/or sequencing. Further components which may optionally be supplied in the kit include sequencing primers suitable for sequencing templates prepared using the methods described herein.

In an aspect is provided a kit including a circularizable probe (e.g., a polynucleotide probe described herein), a ligase, and a specific binding agent attached to a target polynucleotide, wherein the circularizable probe includes a first hybridization sequence capable of hybridizing to a first sequence of a target polynucleotide, a second hybridization sequence capable of hybridizing to a second sequence of the target polynucleotide. In embodiments, the circularizable probe includes a barcode sequence. In embodiments, the target polynucleotide is an oligonucleotide described herein. In embodiments, the target polynucleotide has a sequence selected from SEQ ID NO:1 to SEQ ID NO:132. In embodiments, the target polynucleotide has a sequence selected from SEQ ID NO:397 to SEQ ID NO:529.

Padlock probes (e.g., circularizable oligonucleotides, also referred to as circularizable probes) are specialized ligation probes, examples of which are known in the art, see for example Nilsson M, et al. Science. 1994; 265 (5181): 2085-2088), and has been applied to detect transcribed RNA in cells, see for example Christian A T, et al. Proc Natl Acad. Sci USA. 2001; 98 (25): 14238-14243, both of which are incorporated herein by reference in their entireties. In embodiments, the padlock probe is approximately 50 to 200 nucleotides. In embodiments, a padlock probe has a first domain that is capable of hybridizing to a first target sequence domain, and a second ligation domain, capable of hybridizing to an adjacent second sequence domain. The configuration of the padlock probe is such that upon ligation of the first and second ligation domains of the padlock probe, the probe forms a circular polynucleotide, and forms a complex with the sequence (i.e., the sequence it hybridized to, the target sequence) wherein the target sequence is “inserted” into the loop of the circle. Padlock probes are useful for the methods provided herein and include, for example, padlock probes for genomic analyses, as exemplified by Gore, A. et al. Nature 471, 63-67 (2011); Porreca, G. J. et al. Nat Methods 4, 931-936 (2007); Li, J. B. et al. Genome Res 19, 1606-1615 (2009), Zhang, K. et al. Nat Methods 6, 613-618 (2009); Noggle, S. et al. Nature 478, 70-75 (2011); and Li, J. B. et al. Science 324, 1210-1213 (2009), the content of each of which are incorporated by reference in their entirety.

In embodiments, the polynucleotide probe described herein is a circularizable probe. In embodiments, the circularizable probe (e.g., the circularizable oligonucleotide) comprises a 5′ end and a 3′ end, wherein a first region at the 5′ end is complementary to a first sequence of a target polynucleotide, and wherein a second region at the 3′ end is complementary to a second sequence of the target polynucleotide. In embodiments, the first sequence and the second sequence of the target polynucleotide are adjacent to each other. In embodiments, the first sequence and the second sequence of the target polynucleotide are separated by 1 or more nucleotides. In embodiments, the first sequence and the second sequence of the target polynucleotide are separated by 1, 5, 10, 20, 30, 40, 50, 75, 100, or more nucleotides. In embodiments, the first sequence and the second sequence of the target polynucleotide flank a target sequence. In embodiments, the target sequence is a barcode sequence. In embodiments, the gap sequence is 5, 10, 15, 20, 25, 30, 40, 50, 60, 70, 80, 90, 100, 125, 150, 175, or 200 nucleotides. In embodiments, the gap sequence is 5 to 150 nucleotides. In embodiments, the gap sequence is 1, 2, 3, 4, or 5 nucleotides.

In embodiments, the kit can further include one or more biological stain(s) (e.g., any of the biological stains as described herein). For example, the kit can further include eosin and hematoxylin. In other examples, the kit can include a biological stain such as acridine orange, Bismarck brown, carmine, coomassie blue, crystal violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, safranin, or any combination thereof. In embodiments, the kit is designed for staining tissue samples for imaging and detecting target molecules (e.g., proteins) can be significantly expanded beyond the inclusion of fluorophores. For instance, the kit can include eosin and hematoxylin, which are classic histological stains. Eosin, a red dye, typically stains acidic components of the cell such as cytoplasmic proteins, while hematoxylin, a basic dye, binds to nucleic acids, coloring the cell nucleus blue. This combination is widely used in histopathology for detailed tissue structure visualization. Moreover, the kit can encompass stains such as acridine orange, a nucleic acid-selective fluorescent cationic dye, and Bismarck brown, which is often used for staining backgrounds in histological tissue sections. Carmine, another potential inclusion, is a natural red dye used for staining glycogen, while Coomassie blue is a popular choice for protein staining in gel electrophoresis. Crystal violet, a triarylmethane dye, can be included for staining cell walls and nuclei, and DAPI, a fluorescent stain that binds strongly to A-T rich regions in DNA, is useful in fluorescence microscopy. Ethidium bromide, a fluorescent intercalator, is also a valuable addition for its role in nucleic acid staining, especially in gel electrophoresis. Further, the kit can include acid fuchsine, used in Masson's trichrome stain; Hoechst stains, which are cell-permeable, DNA-specific blue fluorescent dyes; and iodine, commonly used in Gram staining and for staining starch in plant cells. Methyl green and methylene blue, both traditional histological stains, can be included for their affinity towards nucleic acids. Neutral red, a vital stain that accumulates in lysosomes, Nile blue and Nile red, both used for staining lipids, and osmium tetroxide, a heavy metal stain for lipid bilayers in electron microscopy, can be part of the kit. Propidium iodide, a popular red-fluorescent nuclear and chromosome counterstain, along with rhodamine, may be utilized. Safranin, commonly used in Gram staining, can be included for its ability to stain cell components like nuclei, cytoplasm, and cell walls in various colors, enhancing the contrast and detail in tissue imaging.

### III. Methods

In an aspect is provided a method of detecting a target molecule (e.g., a protein, carbohydrate, or nucleic acid molecule). In embodiments, the method includes detecting the target molecule in or on a cell or tissue. In embodiments, the method includes binding a specific binding agent including an oligonucleotide that includes a sequence (e.g., a sequence selected from SEQ ID NO:1 to SEQ ID NO:132) to the target molecule in or on a cell or tissue, wherein the oligonucleotide includes a first blocking oligonucleotide hybridized to a first sequence of the oligonucleotide, and a second blocking oligonucleotide hybridized to a second sequence of the oligonucleotide. In embodiments, the method includes removing the blocking oligonucleotides. In embodiments, the method includes binding a polynucleotide probe including a first binding sequence and a second binding sequence to an oligonucleotide, wherein the oligonucleotide is covalently bound to a specific binding agent and includes a sequence (e.g., a sequence selected from SEQ ID NO:1 to SEQ ID NO:132); binding a polynucleotide to the sequence and detecting the polynucleotide, thereby detecting the target molecule. In embodiments, detecting includes serially contacting the oligonucleotides with labeled probes (e.g., labeled oligonucleotides or labeled nucleotides). In embodiments, the oligonucleotide is double stranded.

In an aspect is provided a method of making an antibody-oligonucleotide conjugate composition. In embodiments, methods for purifying conjugates may involve metal chelation chromatography that utilizes the interaction of a metal ion, for example, Ni²⁺ ion, Zn²⁺ ion, Cu²⁺ ion, Fe²⁺ ion, or Co²⁺ ion and the antibody. For example, an aqueous mixture of antibody-oligonucleotide conjugate and free, or substantially free, oligonucleotide, may be contacted with a water insoluble stationary phase which has the metal ion chelated to the phase. In embodiments, the conjugate chelates with the metal ion whereas neither of the specified free oligonucleotide chelate. In certain embodiments, subsequent washing of the phase with a mild buffer may remove, or substantially remove, the unbound oligonucleotides. In certain embodiments, the antibody-oligonucleotide conjugate may then be eluted from the phase and recovered in a form free, sufficiently free, or substantially free, of unconjugated oligonucleotide.

In embodiments, detecting includes hybridizing a fluorescently labeled oligonucleotide to the extension product and detecting an emission light from the fluorescently labeled oligonucleotide. In embodiments, the method includes serially binding, detecting, and removing the fluorescently labeled oligonucleotide to detect a barcode sequence associated with the biomolecular interaction. For example, fluorescently labeled oligonucleotide can be removed completely after imaging by dislodging the oligonucleotides from the amplification product. In embodiments, the fluorescently labeled oligonucleotide can contain fluorophores which can be cleaved off enzymatically or chemically after imaging/detecting. In embodiments, the detection probe includes detecting a fluorescently labeled probe. The phrase “labeled probes” refers to mixture of nucleic acids that are detectably labeled, e.g., fluorescently labeled, such that the presence of the probe, as well as, any target sequence to which the probe is bound can be detected by assessing the presence of the label. In some embodiments, the probes are about 30-300 bases in length, 40-300 bases in length, or 70-300 bases in length. In some embodiments, the probes are relatively uniform in length (e.g., an average length+/−10 bases). The probes may be uniformly labeled based on position of label and/or number of labels within the probe. In some embodiments, the probes are single-stranded. In some embodiments, the probes are double-stranded. Additional detection probes and related properties may be found in, e.g., U.S. Pat. Pub. US 2011/0039735, which is incorporated herein by reference in its entirety.

In embodiments, the method includes immobilizing a cell or tissue onto a substrate described herein, wherein the cell or tissue includes the target molecule or target biomolecule to be detected. In embodiments, the method includes immobilizing a plurality of tissues onto a substrate described herein, wherein the tissue includes the target molecule or target biomolecule to be detected. In embodiments, the method includes immobilizing 24 tissue sections (10 mm×17 mm sections). In embodiments, the method includes immobilizing 40 tissue sections (10 mm×10 mm sections). In embodiments, the method includes immobilizing 128 tissue sections (4 m×4 m sections).

The cell or tissue may be manipulated prior to immobilizing the cell or tissue onto a solid support using known techniques in the art (see, e.g., PCT Publication WO2023076832A1). In embodiments, the method further includes cutting a sample portion from the biological sample (e.g., including cells or tissues) using a punch device such that the punch device contains the sample portion; mounting the punch device containing the sample portion onto a substrate or support as described herein (e.g., inverting the punch device); pushing the sample portion out of the punch device using a piston, so that all or a portion thereof of the sample portion is positioned on a substrate or support as described herein. In embodiments, the method further includes cutting a sample portion from the biological sample using two or more punch devices such that each punch device contains a different the sample portion; mounting each punch device containing the sample portion onto a substrate or support as described herein; pushing the sample portions out of the punch devices using one or more pistons so that the sample portions are positioned onto a substrate or support as described herein.

In embodiments, the method includes binding a specific binding agent including an oligonucleotide described herein to a target molecule. In embodiments, the method includes binding a specific binding agent including an oligonucleotide described herein to a target molecule, wherein the oligonucleotide includes a first blocking oligonucleotide hybridized to a first sequence of the oligonucleotide and a second blocking oligonucleotide hybridized to a second sequence of the oligonucleotide (see, e.g., FIGS. 4A and 4B). In embodiments, binding a specific binding agent described herein to a target molecule includes incubation in a buffer at 30° C. to 40° C. In embodiments, binding a specific binding agent described herein to a target molecule includes incubation in a buffer at 40° C. to 50° C. In embodiments, binding a specific binding agent described herein to a target molecule includes incubation in a buffer at 50° C. to 60° C. In embodiments, binding a specific binding agent described herein to a target molecule includes incubation in a buffer at about 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., or 65° C.

In embodiments, the method includes removing the blocking oligonucleotides (e.g., the first blocking oligonucleotide and the second blocking oligonucleotide hybridized to the first sequence of the oligonucleotide and the second sequence of the oligonucleotide, respectively). In embodiments, the method includes removing the blocking oligonucleotides prior to binding the polynucleotide probe described herein. In embodiments, removing the blocking oligonucleotides includes incubation in a buffer at about 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., or 65° C.

In embodiments, the method includes binding a polynucleotide probe comprising a first binding sequence and a second binding sequence to an oligonucleotide, wherein the oligonucleotide includes a sequence selected from SEQ ID NO:1 to SEQ ID NO:132 and is attached to the target molecule; amplifying the polynucleotide probe to form an amplification product comprising one or more copies of the first binding sequence and the second binding sequence; hybridizing a primer to the amplification product and incorporating a labeled nucleotide into the primer, thereby detecting the target molecule. In embodiments, prior to amplifying the polynucleotide probe the method includes ligating the ends of the polynucleotide probe together to form a circular polynucleotide.

In embodiments, the method includes binding a polynucleotide probe including a first binding sequence and a second binding sequence to an oligonucleotide, wherein the oligonucleotide is covalently bound to a specific binding agent and includes a sequence selected from SEQ ID NO:1 to SEQ ID NO:132; amplifying the polynucleotide probe to form an amplification product including one or more copies of the first binding sequence and the second binding sequence; hybridizing a primer to the amplification product and incorporating a labeled nucleotide into the primer, thereby detecting the target molecule. In embodiments, the method includes sequencing the amplification product. In embodiments, prior to amplifying the polynucleotide probe the method includes ligating the ends of the polynucleotide probe together to form a circular polynucleotide.

In embodiments, binding the polynucleotide probe including a first binding sequence and a second binding sequence to an oligonucleotide includes incubation in a buffer at 40° C. to 50° C. In embodiments, binding the polynucleotide probe including a first binding sequence and a second binding sequence to an oligonucleotide includes incubation in a buffer at 50° C. to 60° C. In embodiments, binding the polynucleotide probe to the oligonucleotide includes incubation in a buffer at about 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., or 65° C.

In embodiments, amplifying the circular polynucleotide includes hybridizing a primer to the circular polynucleotide and extending the primer with a strand-displacing polymerase. In embodiments, the method further includes amplifying the circular oligonucleotide by extending an amplification primer with a polymerase (e.g., a strand-displacing polymerase), wherein the primer extension generates an extension product including multiple complements of the circular oligonucleotide, referred to as an amplicon. An amplicon typically contains multiple, tandem copies of the circularized nucleic acid molecule of the corresponding sample nucleic acid. The number of copies can be varied by appropriate modification of the reaction conditions, such as varying the number of amplification cycles, using polymerases of varying processivity in the amplification reaction, or varying the length of time that the amplification reaction is run. In embodiments, the extension product includes three or more copies of the circular oligonucleotide. In embodiments, the circular oligonucleotide is copied about 3-50 times (i.e., the extension product includes about 3 to 50 complements of the circular oligonucleotide). In embodiments, the circular oligonucleotide is copied about 50-100 times (i.e., the extension product includes about 50 to 100 complements of the circular oligonucleotide). In embodiments, the circular oligonucleotide is copied about 100-300 times (i.e., the extension product includes about 100 to 300 complements of the circular oligonucleotide). In embodiments, the method includes hybridizing an amplification primer and oligonucleotide primer in the same reaction (e.g., simultaneously). In embodiments, the oligonucleotide is extended as an amplification primer after generating the circular oligonucleotide (e.g., the 3′ end of the oligonucleotide hybridized to the circular oligonucleotide is extended with a polymerase). In embodiments, the method includes contacting the target with an amplification primer and oligonucleotide primer in the same reaction (e.g., simultaneously). In embodiments, the method includes fixing the amplification products (e.g., contacting the amplification product with formalin).

In embodiments, the amplification method includes a standard dNTP mixture including dATP, dCTP, dGTP and dTTP (for DNA) or dATP, dCTP, dGTP and dUTP (for RNA). In embodiments, the amplification method includes a mixture of standard dNTPs and modified nucleotides that contain functional moieties (e.g., bioconjugate reactive groups) that serve as attachment points to the cell or the matrix in which the cell is embedded (e.g., a hydrogel). In embodiments, the amplification method includes a mixture of standard dNTPs and modified nucleotides that contain functional moieties (e.g., bioconjugate reactive groups) that participate in the formation of a bioconjugate linker. The modified nucleotides may react and link the amplification product to the surrounding cell scaffold and milieu. For example, amplifying may include an extension reaction wherein the polymerase incorporates a modified nucleotide into the amplification product, wherein the modified nucleotide includes a bioconjugate reactive moiety (e.g., an alkynyl moiety) attached to the nucleobase. The bioconjugate reactive moiety of the modified nucleotide participates in the formation of a bioconjugate linker by reacting with a complementary bioconjugate reactive moiety present in the cell (e.g., a crosslinking agent, such as NHS-PEG-azide, or an amine moiety) thereby attaching the amplification product to the internal scaffold of the cell. In embodiments, the functional moiety can be covalently cross-linked, copolymerize with or otherwise non-covalently bound to the matrix. In embodiments, the functional moiety can react with a cross-linker. In embodiments, the functional moiety can be part of a ligand-ligand binding pair. Suitable exemplary functional moieties include an amine, acrydite, alkyne, biotin, azide, and thiol. In embodiments of crosslinking, the functional moiety is cross-linked to modified dNTP or dUTP or both. In embodiments, suitable exemplary cross-linker reactive groups include imidoester (DMP), succinimide ester (NHS), maleimide (Sulfo-SMCC), carbodiimide (DCC, EDC) and phenyl azide. Cross-linkers within the scope of the present disclosure may include a spacer moiety. In embodiments, such spacer moieties may be functionalized. In embodiments, such spacer moieties may be chemically stable. In embodiments, such spacer moieties may be of sufficient length to allow amplification of the nucleic acid bound to the matrix. In embodiments, suitable exemplary spacer moieties include polyethylene glycol, carbon spacers, photo-cleavable spacers and other spacers known to those of skill in the art and the like. In embodiments, amplification reactions include standard dNTPs and a modified nucleotide (e.g., amino-allyl dUTP, 5-TCO-PEG4-dUTP, C8-Alkyne-dUTP, 5-Azidomethyl-dUTP, 5-Vinyl-dUTP, or 5-Ethynyl dLTTP). For example, during amplification a mixture of standard dNTPs and aminoallyl deoxyuridine 5′-triphosphate (dUTP) nucleotides may be incorporated into the amplicon and subsequently cross-linked to the cell protein matrix by using a cross-linking reagent (e.g., an amine-reactive crosslinking agent with PEG spacers, such as (PEGylated bis(sulfosuccinimidyl)suberate) (BS(PEG)₉)).

In embodiments, amplifying includes incubating the circular polynucleotide with a strand-displacing polymerase for about 15 minutes to about 2 hours. In embodiments, amplifying includes incubating the circular polynucleotide with a strand-displacing polymerase for about 30 minutes to about 60 minutes. In embodiments, amplifying includes binding an amplification primer to the primer binding sequence and extending the amplification primer with a strand-displacing polymerase.

In embodiments, amplifying the circular oligonucleotide includes incubating the circular oligonucleotide with the strand-displacing polymerase (a) for about 1 minute to about 2 hours, and/or (b) at a temperature of about 20° C. to about 50° C. In embodiments, amplifying the circular oligonucleotide includes incubating the circular oligonucleotide with the strand-displacing polymerase for about 1 minute to about 2 hours. In embodiments, amplifying the circular oligonucleotide includes incubating the circular oligonucleotide with the strand-displacing polymerase for about 5, about 10, about 20, about 30, about 40, about 45, about 50, about 55, or about 60 minutes. In embodiments, amplifying the circular oligonucleotide includes incubating the circular oligonucleotide with the strand-displacing polymerase for about 5 minutes. In embodiments, amplifying the circular oligonucleotide includes incubating the circular oligonucleotide with the strand-displacing polymerase for about 10 minutes. In embodiments, amplifying the circular oligonucleotide includes incubating the circular oligonucleotide with the strand-displacing polymerase for about 20 minutes. In embodiments, amplifying the circular oligonucleotide includes incubating the circular oligonucleotide with the strand-displacing polymerase for about 30 minutes. In embodiments, amplifying the circular oligonucleotide includes incubating the circular oligonucleotide with the strand-displacing polymerase for about 45 minutes. In embodiments, amplifying the circular oligonucleotide includes incubating the circular oligonucleotide with the strand-displacing polymerase for about 60 minutes.

In embodiments, amplifying the circular oligonucleotide includes incubating the circular oligonucleotide with the strand-displacing polymerase for about 1 hour to about 12 hours. In embodiments, amplifying includes incubation with the strand-displacing polymerase for about 60 seconds to about 60 minutes. In embodiments, amplifying includes incubation with the strand-displacing polymerase for about 10 minutes to about 60 minutes. In embodiments, amplifying includes incubation with the strand-displacing polymerase for about 10 minutes to about 30 minutes. In embodiments, amplifying the circular oligonucleotide includes incubating the circular oligonucleotide with the strand-displacing polymerase for about 1, about 2, about 3, about 4, about 5, about 6, about 7, about 8, about 9, about 10, about 11, about 12 hours, about 13 hours, about 14 hours, about 15 hours, about 16 hours, about 17 hours, about 18 hours, about 19, hours, or about 20 hours. In embodiments, amplifying the circular oligonucleotide includes incubating the circular oligonucleotide with the strand-displacing polymerase for more than 12 hours.

In embodiments, amplifying includes incubation in a buffer at about 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., or 65° C. In embodiments, amplifying includes incubation in a buffer at 30° C. to 40° C. In embodiments, amplifying includes incubation in a buffer at 40° C. to 50° C. In embodiments, amplifying the circular oligonucleotide includes incubating the circular oligonucleotide with the strand-displacing polymerase at a temperature of about 20° C. to about 50° C. In embodiments, incubation with the strand-displacing polymerase is at a temperature of about 20° C., about 25° C., about 30° C., about 35° C., about 40° C., about 45° C., or about 50° C. In embodiments, incubation with the strand-displacing polymerase is at a temperature of about 35° C. to 42° C. In embodiments, incubation with the strand-displacing polymerase is at a temperature of about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., or about 50° C. In embodiments, the strand-displacing polymerase is a phi29 polymerase, a SD polymerase, a Bst large fragment polymerase, phi29 mutant polymerase, a Thermus aquaticus polymerase, or a thermostable phi29 mutant polymerase.

In embodiments, amplifying includes rolling circle amplification (RCA) or rolling circle transcription (RCT) (see, e.g., Lizardi et al., Nat. Genet. 19:225-232 (1998), which is incorporated herein by reference in its entirety). Several suitable rolling circle amplification methods are known in the art. For example, RCA amplifies a circular polynucleotide (e.g., DNA) by polymerase extension of an amplification primer complementary to a portion of the template polynucleotide. This process generates copies of the circular polynucleotide template such that multiple complements of the template sequence arranged end to end in tandem are generated (i.e., a concatemer) locally preserved at the site of the circle formation. In embodiments, the amplifying occurs at isothermal conditions. In embodiments, the amplifying includes hybridization chain reaction (HCR). HCR uses a pair of complementary, kinetically trapped hairpin oligomers to propagate a chain reaction of hybridization events, as described in Dirks, R. M., & Pierce, N. A. (2004) PNAS USA, 101 (43), 15275-15278, which is incorporated herein by reference for all purposes. In embodiments, the amplifying includes branched rolling circle amplification (BRCA); e.g., as described in Fan T, Mao Y, Sun Q, et al. Cancer Sci. 2018; 109:2897-2906, which is incorporated herein by reference in its entirety. In embodiments, the amplifying includes hyberbranched rolling circle amplification (HRCA). Hyperbranched RCA uses a second primer complementary to the first amplification product. This allows products to be replicated by a strand-displacement mechanism, which yields drastic amplification within an isothermal reaction (Lage et al., Genome Research 13:294-307 (2003), which is incorporated herein by reference in its entirety). In embodiments, amplifying includes polymerase extension of an amplification primer. In embodiments, the polymerase is T4, T7, Sequenase, Taq, Klenow, and Pol I DNA polymerases. SD polymerase, Bst large fragment polymerase, or a phi29 polymerase or mutant thereof. In embodiments, the strand-displacing enzyme is an SD polymerase, Bst large fragment polymerase, or a phi29 polymerase or mutant thereof. In embodiments, the strand-displacing polymerase is Bst DNA Polymerase Large Fragment, Thermus aquaticus (Taq) polymerase, or a mutant thereof. In embodiments, the strand-displacing polymerase is a phi29 polymerase, a phi29 mutant polymerase or a thermostable phi29 mutant polymerase. In embodiments, amplifying includes rolling circle amplification for 15 minutes to 16 hours. In embodiments, amplifying includes rolling circle amplification for 15 minutes to 4 hours. In embodiments, amplifying includes rolling circle amplification for 15 minutes to 2 hours. In embodiments, amplifying includes rolling circle amplification for 15 minutes to 1 hour.

In embodiments, sequencing includes a plurality of sequencing cycles. In embodiments, sequencing includes a plurality of rounds of sequencing cycles (e.g., a first round of 10 sequencing cycles; followed by a second round of 10 sequencing cycles). In embodiments, sequencing includes a plurality of rounds of sequencing cycles (e.g., a first round of 1 sequencing cycle; followed by a second round of 1 sequencing cycle). In embodiments, sequencing includes 20 to 100 sequencing cycles. In embodiments, sequencing includes 50 to 100 sequencing cycles. In embodiments, sequencing includes 50 to 300 sequencing cycles. In embodiments, sequencing includes 50 to 150 sequencing cycles. In embodiments, sequencing includes at least 10, 20, 30 40, or 50 sequencing cycles. In embodiments, sequencing includes at least 10 sequencing cycles. In embodiments, sequencing includes 10 to 20 sequencing cycles. In embodiments, sequencing includes 10, 11, 12, 13, 14, or 15 sequencing cycles. In embodiments, sequencing includes (a) extending a sequencing primer by incorporating a labeled nucleotide, or labeled nucleotide analogue and (b) detecting the label to generate a signal for each incorporated nucleotide or nucleotide analogue. In embodiments, prior to initiating a next round of sequencing cycles, the first sequencing primer is terminated or removed. For example, termination may occur via incorporating a non-extendable nucleotide (e.g., a ddNTP) into the first sequencing primer.

In embodiments, the method includes sequencing the barcode (e.g., the barcode sequence or the barcode nucleotide). In embodiments, the method includes sequencing a plurality of barcodes in an optically resolved volume. A variety of sequencing methodologies can be used such as sequencing-by synthesis (SBS), pyrosequencing, sequencing by ligation (SBL), or sequencing by hybridization (SBH). Pyrosequencing detects the release of inorganic pyrophosphate (PPi) as particular nucleotides are incorporated into a nascent nucleic acid strand (Ronaghi, et al., Analytical Biochemistry 242 (1), 84-9 (1996); Ronaghi, Genome Res. 11 (1), 3-11 (2001); Ronaghi et al. Science 281 (5375), 363 (1998); U.S. Pat. Nos. 6,210,891; 6,258,568; and. 6,274,320, each of which is incorporated herein by reference in its entirety). In pyrosequencing, released PPi can be detected by being converted to adenosine triphosphate (ATP) by ATP sulfurylase, and the level of ATP generated can be detected via light produced by luciferase. In this manner, the sequencing reaction can be monitored via a luminescence detection system. In both SBL and SBH methods, target nucleic acids, and amplicons thereof, are subjected to repeated cycles of oligonucleotide delivery and detection. SBL methods, include those described in Shendure et al. Science 309:1728-1732 (2005); U.S. Pat. Nos. 5,599,675; and 5,750,341, each of which is incorporated herein by reference in its entirety; and the SBH methodologies are as described in Bains et al., Journal of Theoretical Biology 135 (3), 303-7 (1988); Drmanac et al., Nature Biotechnology 16, 54-58 (1998); Fodor et al., Science 251 (4995), 767-773 (1995); and WO 1989/10977, each of which is incorporated herein by reference in its entirety.

In SBS, extension of a nucleic acid primer along a nucleic acid template is monitored to determine the sequence of nucleotides in the template. The underlying chemical process can be catalyzed by a polymerase, wherein fluorescently labeled nucleotides are added to a primer (thereby extending the primer) in a template dependent fashion such that detection of the order and type of nucleotides added to the primer can be used to determine the sequence of the template. In embodiments, sequencing includes annealing and extending a sequencing primer to incorporate a detectable label that indicates the identity of a nucleotide in the target polynucleotide, detecting the detectable label, and repeating the extending and detecting of steps. In embodiments, the methods include sequencing one or more bases of a target nucleic acid by extending a sequencing primer hybridized to a target nucleic acid (e.g., an amplification product produced by the amplification methods described herein). In embodiments, sequencing may be accomplished by a sequencing-by-synthesis (SBS) process. In embodiments, sequencing comprises a sequencing by synthesis process, where individual nucleotides are identified iteratively, as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. Nos. 7,541,444 and 7,057,026. Once such a modified nucleotide has been incorporated into the growing polynucleotide chain complementary to the region of the template being sequenced, there is no free 3′-OH group available to direct further sequence extension and therefore the polymerase cannot add further nucleotides. Once the identity of the base incorporated into the growing chain has been determined, the 3′ reversible terminator may be removed to allow addition of the next successive nucleotide. By ordering the products derived using these modified nucleotides it is possible to deduce the DNA sequence of the oligonucleotide barcode.

In embodiments, sequencing includes extending a first sequencing primer to generate a sequencing read comprising the first barcode sequence, or a portion thereof. In embodiments, sequencing includes extending a first sequencing primer to generate a sequencing read comprising the first barcode sequence, or a portion thereof, and extending a second sequencing primer to generate a sequencing read comprising the second barcode sequence. In embodiments, sequencing includes sequentially extending a plurality of sequencing primers (e.g., sequencing a first region of a target nucleic acid followed by sequencing a second region of a target nucleic acid, followed by sequencing N regions, where Nis the number of sequencing primers in the known sequencing primer set). In embodiments, sequencing includes generating a plurality of sequencing reads.

In embodiments, sequencing includes sequentially sequencing a plurality of different targets by initiating sequencing with different sequencing primers. For example, a first circularizable probe includes a first primer binding site (a nucleic acid sequence complementary to a first sequencing primer) and optionally a first barcode sequence or barcode nucleotide. In a similar manner, a second and third padlock probe include a second primer binding site (a nucleic acid sequence complementary to a second, different, sequencing primer) and a third primer binding site (a nucleic acid sequence complementary to a third, different from both Primer 1 and Primer 2, sequencing primer), respectively. During the first round of sequencing (following probe circularization and amplification according to the methods described herein), using primer 1, the probe hybridized to the first nucleic acid molecule is detected. In the second round of sequencing, primer 2 can hybridize and sequence an identifying sequence of the probe (e.g., a barcode sequence or nucleotide) hybridized to a second nucleic acid molecule. Similarly, in the third round of sequencing, primer 3 can hybridize and sequence the probe hybridized to the third nucleic acid molecule.

In embodiments, sequencing includes encoding the sequencing read into a codeword. Useful encoding schemes include those developed for telecommunications, coding theory and information theory such as those set forth in Hamming, Coding and Information Theory, 2^{nd} Ed. Prentice Hall, Englewood Cliffs, N.J. (1986) and Moon T K. Error Correction Coding: Mathematical Methods and Algorithms. ed. 1st Wiley: 2005., each of which are incorporated herein by reference. A useful encoding scheme uses a Hamming code. A Hamming code can provide for signal (and therefore sequencing and barcode) distinction. In this scheme, signal states detected from a series of nucleotide incorporation and detection events (i.e., while sequencing the oligonucleotide barcode) can be represented as a series of the digits to form a codeword, the codeword having a length equivalent to the number incorporation/detection events. The digits can be binary (e.g. having a value of 1 for presence of signal and a value of 0 for absence of the signal) or digits can have a higher radix (e.g., a ternary digit having a value of 1 for fluorescence at a first wavelength, a value of 2 for fluorescence at a second wavelength, and a value of 0 for no fluorescence at those wavelengths, etc.). Barcode discrimination capabilities are provided when codewords can be quantified via Hamming distances between two codewords (i.e., barcode 1 having codeword 1, and barcode 2 having codeword 2, etc.).

In embodiments, prior to binding the polynucleotide probe, the method includes binding the specific binding agent to a target molecule in a cell or tissue. In embodiments, the targets are proteins or carbohydrates. In embodiments, the targets are proteins. In embodiments, the targets are carbohydrates. In embodiments when the target are proteins and/or carbohydrates, the method includes contacting the proteins with a specific binding reagent, wherein the specific binding reagent comprises an oligonucleotide barcode. In embodiments, the specific binding reagent comprises an antibody, single-chain Fv fragment (scFv), antibody fragment-antigen binding (Fab), or an aptamer. In embodiments, the specific binding reagent is a peptide, a cell penetrating peptide, an aptamer, a DNA aptamer, an RNA aptamer, an antibody, an antibody fragment, a light chain antibody fragment, a single-chain variable fragment (scFv), a lipid, a lipid derivative, a phospholipid, a fatty acid, a triglyceride, a glycerolipid, a glycerophospholipid, a sphingolipid, a saccharolipid, a polyketide, a polylysine, polyethyleneimine, diethylaminoethyl (DEAE)-dextran, cholesterol, or a sterol moiety. In embodiments, the specific binding reagent interacts (e.g., contacts, or binds) with one or more specific binding reagents on the cell surface. Carbohydrate-specific antibodies are known in the art, see for example Kappler, K., Hennet, T. Genes Immun 21, 224-239 (2020).

In embodiments, the oligonucleotide is at least partially double-stranded. In embodiments, the oligonucleotide includes a blocking polynucleotide hybridized to the sequence, and wherein the blocking polynucleotide is removed prior to binding the polynucleotide probe.

In embodiments, the polynucleotide has a first domain that is capable of hybridizing to a first target sequence domain, and a second domain capable of hybridizing to an adjacent second target sequence domain. In embodiments, the length of the first domain and second domain are the same length (e.g., both the first and the second domains are about 15 nucleotides). In embodiments, the length of the first domain and second domain are different lengths (e.g., the first domain is about 10 nucleotides and the second domain is about 20 nucleotides). In embodiments, an asymmetric polynucleotide (i.e., a polynucleotide having a first domain and second domain that are different lengths) may be advantageous in preventing non-specific hybridization. In embodiments, the total length of the first domain and second domain is about 25, 30, 35, or 40 nucleotides. In embodiments, the total length of the first domain and second domain is about 30 nucleotides.

In embodiments, the method further includes ligating the 5′ and 3′ ends of the polynucleotide described herein or the polynucleotide probe described herein to form a circular polynucleotide (i.e., a polynucleotide that is a continuous strand lacking free 5′ and 3′ ends). In embodiments, the method includes ligating the 5′ and 3′ ends of the polynucleotide to form a circular polynucleotide, wherein the circular polynucleotide includes the target nucleic acid. In embodiments, the method includes ligating the 5′ and 3′ ends of the polynucleotide to form a circular polynucleotide, wherein the circular polynucleotide includes the oligonucleotide barcode. In embodiments, the ligation includes enzymatic ligation. In embodiments, ligating includes enzymatic ligation including a ligation enzyme (e.g., CircLigase™ enzyme, Taq DNA Ligase, HiFi Taq DNA Ligase, T4 ligase, PBCV-1 DNA Ligase (also known as SplintR® ligase) or Ampligase™ DNA Ligase). Non-limiting examples of ligases include DNA ligases such as DNA Ligase I, DNA Ligase II, DNA Ligase III, DNA Ligase IV, T4 DNA ligase, T7 DNA ligase, T3 DNA Ligase, E. coli DNA Ligase, PBCV-1 DNA Ligase (also known as SplintR® ligase) or a Taq DNA Ligase. In embodiments, the ligase enzyme is a T4 DNA ligase, T4 RNA ligase 1, T4 RNA ligase 2, T3 DNA ligase or T7 DNA ligase. In embodiments, the enzymatic ligation is performed by a mixture of ligases. In embodiments, the ligation enzyme is selected from the group consisting of T4 DNA ligase, T4 RNA ligase 1, T4 RNA ligase 2, RtcB ligase, T3 DNA ligase, T7 DNA ligase, Taq DNA ligase, PBCV-1 DNA Ligase, a thermostable DNA ligase (e.g., 5′AppDNA/RNA ligase), an ATP dependent DNA ligase, an RNA-dependent DNA ligase (e.g., SplintR® ligase), and combinations thereof. In embodiments, prior to ligating the 5′ and 3′ ends of the polynucleotide probe to form a circular polynucleotide, the method includes extending the 3′ end of the polynucleotide probe (using a polymerase to incorporate one or more nucleotides) along the oligonucleotide to generate a complementary sequence and ligating the extended 3′ end of the polynucleotide probe to the 5′ end of the polynucleotide probe.

In embodiments, the method includes binding a sequencing primer to the amplification product. In embodiments, sequencing includes extending a sequencing primer to generate a sequencing read. In embodiments, sequencing includes extending a sequencing primer by incorporating a labeled nucleotide, or labeled nucleotide analogue and detecting the label to generate a signal for each incorporated nucleotide or nucleotide analogue. In embodiments, the labeled nucleotide or labeled nucleotide analogue further includes a reversible terminator moiety. In embodiments, sequencing includes binding a fluorescent moiety to the amplification product. In embodiments, the method includes binding a fluorescent moiety to the amplification product and detecting the fluorescent moiety, thereby detecting the target molecule. In embodiments, the fluorescent moiety is a fluorescently labeled oligonucleotide. In embodiments, the fluorescent moiety is a fluorescently labeled nucleotide. In embodiments, the method includes binding a sequencing primer to the amplification product and binding a fluorescently labeled nucleotide to the sequencing primer.

In embodiments, the labeled nucleotide or labeled nucleotide analogue further includes a reversible terminator moiety. In embodiments, the reversible terminator moiety is attached to the 3′ oxygen of the nucleotide and is independently

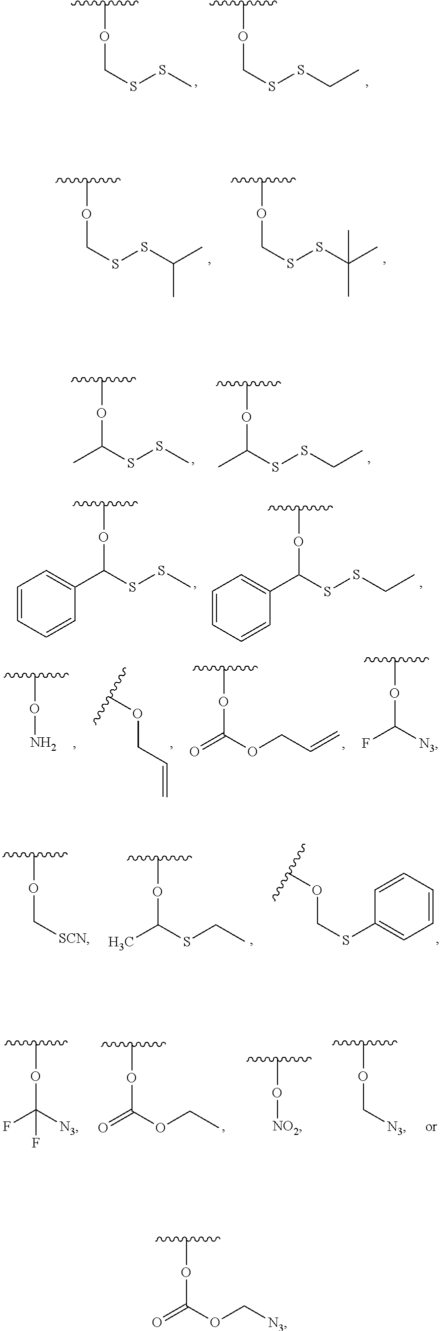

wherein the 3′ oxygen is explicitly depicted in the above formulae. Additional examples of reversible terminators may be found in U.S. Pat. No. 6,664,079, Ju J. et al. (2006) Proc Natl Acad Sci USA 103 (52): 19635-19640.; Ruparel H. et al. (2005) Proc Natl Acad Sci USA 102 (17): 5932-5937.; Wu J. et al. (2007) Proc Natl Acad Sci USA 104 (104): 16462-16467; Guo J. et al. (2008) Proc Natl Acad Sci USA 105 (27): 9145-9150 Bentley D. R. et al. (2008) Nature 456 (7218): 53-59; or Hutter D. et al. (2010) Nucleosides Nucleotides & Nucleic Acids 29:879-895, which are incorporated herein by reference in their entirety for all purposes. In embodiments, the reversible terminator moiety attached to the 3′ oxygen of the nucleotide is an allyl moiety. In embodiments, a polymerase-compatible cleavable moiety includes an azido moiety or a dithiol moiety.

A variety of suitable sequencing platforms are available for implementing methods disclosed herein (e.g., for performing the sequencing reaction). Non-limiting examples include SMRT (single-molecule real-time sequencing), ion semiconductor, pyrosequencing, sequencing by synthesis, sequencing by binding, combinatorial probe anchor synthesis, SOLID sequencing (sequencing by ligation), and nanopore sequencing. Sequencing platforms include those provided by Singular Genomics™ (e.g., the G4™ system) or Illumina™, Inc. (e.g., HiSeq™, MiSeq™, NextSeq™, or NovaSeq™ systems).

In embodiments, generating a sequencing read includes determining the identity of the nucleotides in the template polynucleotide (or complement thereof). In embodiments, a sequencing read, e.g., a first sequencing read or a second sequencing read, includes determining the identity of a portion (e.g., 1, 2, 5, 10, 20, 50 nucleotides) of the total template polynucleotide. In embodiments the first sequencing read determines the identity of 5-10 nucleotides and the second sequencing read determines the identity of more than 5-10 nucleotides (e.g., 11 to 200 nucleotides). In embodiments the first sequencing read determines the identity of more than 5-10 nucleotides (e.g., 11 to 200 nucleotides) and the second sequencing read determines the identity of 5-10 nucleotides. In embodiments, following the generation of a sequencing read, subsequent extension is performed using a plurality of standard (e.g., non-modified) dNTPs until the complementary strand is copied. In other embodiments, following the generation of a sequencing read, subsequent extension is performed using a plurality of dideoxy nucleotide triphosphates (ddNTPs) to prevent further extension of the first sequencing read product during a second sequencing read. In embodiments, following the identification of at least 5-10 (e.g., 11 to 200 nucleotides, or up to 1000 nucleotides), subsequent extension is performed using a plurality of standard (e.g., non-modified) dNTPs until the complementary strand is copied. In embodiments, following the identification of at least 5-10 (e.g., 11 to 200 nucleotides, or up to 1000 nucleotides), subsequent extension is performed using a plurality of dideoxy nucleotide triphosphates (ddNTPs) to prevent further extension of the sequencing read product.

In embodiments, the sequencing method relies on the use of modified nucleotides that can act as reversible reaction terminators. Once the modified nucleotide has been incorporated into the growing polynucleotide chain complementary to the region of the template being sequenced there is no free 3′-OH group available to direct further sequence extension and therefore the polymerase cannot add further nucleotides. Once the identity of the base incorporated into the growing chain has been determined, the 3′ reversible terminator may be removed to allow addition of the next successive nucleotide. These such reactions can be done in a single experiment if each of the modified nucleotides has attached a different label, known to correspond to the particular nucleobase, to facilitate discrimination between the bases added at each incorporation step. Alternatively, a separate reaction may be carried out containing each of the modified nucleotides separately.

The modified nucleotides may carry a label (e.g., a fluorescent label) to facilitate their detection. Each nucleotide type may carry a different fluorescent label. However, the detectable label need not be a fluorescent label. Any label can be used which allows the detection of an incorporated nucleotide. One method for detecting fluorescently labeled nucleotides includes using laser light of a wavelength specific for the labeled nucleotides, or the use of other suitable sources of illumination. The fluorescence from the label on the nucleotide may be detected (e.g., by a CCD camera or other suitable detection means).

In embodiments, the methods of sequencing a nucleic acid include extending a complementary polynucleotide (e.g., a primer) that is hybridized to the nucleic acid by incorporating a first nucleotide. In embodiments, the method includes a buffer exchange or wash step. In embodiments, the methods of sequencing a nucleic acid include a sequencing solution. The sequencing solution includes (a) an adenine nucleotide, or analog thereof; (b) (i) a thymine nucleotide, or analog thereof, or (ii) a uracil nucleotide, or analog thereof; (c) a cytosine nucleotide, or analog thereof; and (d) a guanine nucleotide, or analog thereof.

In embodiments, the method includes sequencing a plurality of target polynucleotides of a cell in situ within an optically resolved volume. In embodiments, the number of unique targets detected within an optically resolved volume of a sample is about 3, 10, 30, 50, or 100. In embodiments, the number of unique targets detected within an optically resolved volume of a sample is about 1 to 10. In embodiments, the number of unique targets detected within an optically resolved volume of a sample is about 5 to 10. In embodiments, the number of unique targets detected within an optically resolved volume of a sample is about 1 to 5. In embodiments, the number of unique targets detected within an optically resolved volume of a sample is at least 3, 10, 30, 50, or 100. In embodiments, the number of unique targets detected within an optically resolved volume of a sample is less than 3, 10, 30, 50, or 100. In embodiments, the number of unique targets detected within an optically resolved volume of a sample is about 1, 2, 3, 4, 5, 6, 7, 8, 9, 10, 20, 50, 100, 500, 1,000, 5,000, 10,000, or 200,000. In embodiments, the methods allow for detection of a single target of interest. In embodiments, the methods allow for multiplex detection of a plurality of targets of interest.

In embodiments, the optically resolved volume has an axial resolution (i.e., depth, or z) that is greater than the lateral resolution (i.e., xy plane). In embodiments, the optically resolved volume has an axial resolution that is greater than twice the lateral resolution. In embodiments, the dimensions (i.e., the x, y, and z dimensions) of the optically resolved volume are about 0.5 μm×0.5 μm×0.5 μm; 1 μm×1 μm×1 μm; 2 μm×2 μm×2μ; 0.5 μm×0.5 μm×1 μm; 0.5 μm×0.5 μm×2 μm; 2 μm×2 μm×1 μm; or 1 μm×1 μm×2 μm. In embodiments, the dimensions (i.e., the x, y, and z dimensions) of the optically resolved volume are about 1 μm×1 μm×2 μm; 1 μm×1 μm×3 μm; 1 μm×1 μm×4 μm; or about 1 μm×1 μm×5 μm. In embodiments, the dimensions (i.e., the x, y, and z dimensions) of the optically resolved volume are about 1 μm×1 μm×5 μm. In embodiments, the dimensions (i.e., the x, y, and z dimensions) of the optically resolved volume are about 1 μm×1 μm×6 μm. In embodiments, the dimensions (i.e., the x, y, and z dimensions) of the optically resolved volume are about 1 μm×1 μm×7 μm. In embodiments, the optically resolved volume is a cubic micron. In embodiments, the optically resolved volume has a lateral resolution from about 100 to 200 nanometers, from 200 to 300 nanometers, from 300 to 400 nanometers, from 400 to 500 nanometers, from 500 to 600 nanometers, or from 600 to 1000 nanometers. In embodiments, the optically resolved volume has a axial resolution from about 100 to 200 nanometers, from 200 to 300 nanometers, from 300 to 400 nanometers, from 400 to 500 nanometers, from 500 to 600 nanometers, or from 600 to 1000 nanometers. In embodiments, the optically resolved volume has a axial resolution from about 1 to 2 μm, from 2 to 3 μm, from 3 to 4 μm, from 4 to 5 μm, from 5 to 6 μm, or from 6 to 10 μm.

In embodiments, the method further includes an additional imaging modality, immunofluorescence (IF), or immunohistochemistry modality (e.g., immunostaining). In embodiments, the method includes ER staining (e.g., contacting the cell with a cell-permeable dye which localizes to the endoplasmic reticula), Golgi staining (e.g., contacting the cell with a cell-permeable dye which localizes to the Golgi), F-actin staining (e.g., contacting the cell with a phalloidin-conjugated dye that binds to actin filaments), lysosomal staining (e.g., contacting the cell with a cell-permeable dye that accumulates in the lysosome via the lysosome pH gradient), mitochondrial staining (e.g., contacting the cell with a cell-permeable dye which localizes to the mitochondria), nucleolar staining, or plasma membrane staining. For example, the method includes live cell imaging (e.g., obtaining images of the cell) prior to or during fixing, immobilizing, and permeabilizing the cell. Immunohistochemistry (IHC) is a powerful technique that exploits the specific binding between an antibody and antigen to detect and localize specific antigens in cells and tissue, commonly detected and examined with the light microscope. Known IHC modalities may be used, such as the protocols described in Magaki, S., Hojat, S. A., Wei, B., So, A., & Yong, W. H. (2019). Methods in molecular biology (Clifton, N.J.), 1897, 289-298, which is incorporated herein by reference. In embodiments, the additional imaging modality includes bright field microscopy, phase contrast microscopy, Nomarski differential-interference-contrast microscopy, or dark field microscopy. In embodiments, the method further includes determining the cell morphology (e.g., the cell boundary or cell shape) using known methods in the art. For example, to determining the cell boundary includes comparing the pixel values of an image to a single intensity threshold, which may be determined quickly using histogram-based approaches as described in Carpenter, A. et al Genome Biology 7, R100 (2006) and Arce, S., Sci Rep 3, 2266 (2013)). By “microscopic analysis” is meant the analysis of a specimen using techniques that provide for the visualization of aspects of a specimen that cannot be seen with the unaided eye, i.e., that are not within the resolution range of the normal human eye. Such techniques may include, without limitation, optical microscopy, e.g., bright field, oblique illumination, dark field, phase contrast, differential interference contrast, interference reflection, epifluorescence, confocal microscopy, CLARITY-optimized light sheet microscopy (COLM), light field microscopy, tissue expansion microscopy, etc., laser microscopy, such as, two photon microscopy, electron microscopy, and scanning probe microscopy. By “preparing a biological specimen for microscopic analysis” is generally meant rendering the specimen suitable for microscopic analysis at an unlimited depth within the specimen. In embodiments, the immobilized tissue section is imaged using “optical sectioning” techniques, such as laser scanning confocal microscopes, laser scanning 2-Photon microscopy, parallelized confocal (i.e. spinning disk), computational image deconvolution methods, and light sheet approaches. Optical sectioning microscopy methods provide information about single planes of a volume by minimizing contributions from other parts of the volume and do so without physical sectioning. The resulting “stack” of such optically sectioned images, represents a full reconstruction of the 3-dimensional features of a tissue volume. A typical confocal microscope includes a 10×/0.5 objective (dry; working distance, 2.0 mm) and/or a 20×/0.8 objective (dry; working distance, 0.55 mm), with a z-step interval of 1 to 5 μm. A typical light sheet fluorescence microscope includes an sCMOS camera, a 2×/0.5 objective lens, and zoom microscope body (magnification range of ×0.63 to ×6.3). For entire scanning of whole samples, the z-step interval is 5 or 10 μm, and for image acquisition in the regions of interest, an interval in the range of 2 to 5 μm may be used.

In embodiments, the imaging modality is capable of imaging an imaging area of about 1 cm² to about 10 cm², 1 cm² to about 5 cm², 5 cm² to about 10 cm², 10 cm² to about 30 cm², 30 cm² to about 60 cm², 60 cm² to about 90 cm², 90 cm² to about 120 cm², or more.

In embodiments, the collection of information (e.g., sequencing information and cell morphology) is referred to as a signature. The term “signature” may encompass any gene or genes, protein or proteins, or epigenetic element(s) whose expression profile or whose occurrence is associated with a specific cell type, subtype, or cell state of a specific cell type or subtype within a population of cells. It is to be understood that also when referring to proteins (e.g., differentially expressed proteins), such may fall within the definition of “gene” signature. Levels of expression or activity or prevalence may be compared between different cells in order to characterize or identify for instance signatures specific for cell (sub) populations. Increased or decreased expression or activity of signature genes may be compared between different cells in order to characterize or identify for instance specific cell (sub) populations.

In embodiments, the methods described herein may further include constructing a 3-dimensional pattern of abundance, expression, and/or activity of each target from spatial patterns of abundance, expression, and/or activity of each target of multiple samples. In embodiments, the multiple samples can be consecutive tissue sections of a 3-dimensional tissue sample.

In an aspect is provided a method of detecting a plurality of different nucleic acid sequences within an optically resolved volume of a cell in situ, wherein the method includes i) associating a different oligonucleotide barcode from a known set of barcodes with each of the plurality of targets, wherein associating an oligonucleotide barcode with each of the plurality of targets includes hybridizing a padlock probe to two adjacent nucleic acid sequences of the target, wherein the padlock probe is a single-stranded polynucleotide having a 5′ and a 3′ end, and wherein the padlock probe includes a primer binding sequence from a known set of primer binding sequences; ii) sequencing each barcode to obtain a multiplexed signal in the cell in situ; iii) demultiplexing the multiplexed signal by comparison with the known set of barcodes; and iv) detecting the plurality of targets by identifying the associated barcodes detected in the cell.

In another aspect is provided a method of detecting a plurality of carbohydrates within an optically resolved volume of a cell in situ, wherein the method includes i) associating a different oligonucleotide barcode from a known set of barcodes with each of the plurality of targets, wherein associating an oligonucleotide barcode with each of the plurality of targets includes contacting each of the targets with a specific binding reagent, wherein the specific binding reagent includes an oligonucleotide barcode; ii) hybridizing a padlock probe to two adjacent nucleic acid sequences of the barcode, wherein the padlock probe is a single-stranded polynucleotide having a 5′ and a 3′ end, and wherein the padlock probe includes a primer binding sequence from a known set of primer binding sequences; iii) sequencing each barcode to obtain a multiplexed signal in the cell in situ; iv) demultiplexing the multiplexed signal by comparison with the known set of barcodes; and v) detecting the plurality of targets by identifying the associated barcodes detected in the cell.

In another aspect is provided a method of detecting a plurality of proteins (e.g., different proteins) within an optically resolved volume of a cell in situ, wherein the method includes i) associating a different oligonucleotide barcode from a known set of barcodes with each of the plurality of targets, wherein associating an oligonucleotide barcode with each of the plurality of targets includes contacting each of the targets with a specific binding reagent, wherein the specific binding reagent includes an oligonucleotide barcode; ii) hybridizing a padlock probe to two adjacent nucleic acid sequences of the barcode, wherein the padlock probe is a single-stranded polynucleotide having a 5′ and a 3′ end, and wherein the padlock probe includes a primer binding sequence from a known set of primer binding sequences; iii) sequencing each barcode to obtain a multiplexed signal in the cell in situ; iv) demultiplexing the multiplexed signal by comparison with the known set of barcodes; and v) detecting the plurality of targets by identifying the associated barcodes detected in the cell. In another aspect is provided a method of detecting a plurality of proteins (e.g., different proteins) within an optically resolved volume of a cell in situ, wherein the method includes i) associating a different oligonucleotide barcode from a known set of barcodes with each of the plurality of targets, wherein associating an oligonucleotide barcode with each of the plurality of targets includes contacting each of the targets with a specific binding reagent, wherein the specific binding reagent includes an oligonucleotide barcode; ii) sequencing each barcode to obtain a multiplexed signal in the cell in situ; iii) demultiplexing the multiplexed signal by comparison with the known set of barcodes; and iv) detecting the plurality of targets by identifying the associated barcodes detected in the cell.

In embodiments, associating an oligonucleotide barcode with each of the plurality of targets includes hybridizing a padlock probe to two adjacent nucleic acid sequences of the target, wherein the padlock probe is a single-stranded polynucleotide having a 5′ and a 3′ end, and the padlock probe includes at least one oligonucleotide barcode, and at least one primer binding sequence. In embodiments, the oligonucleotide barcode includes at least two primer binding sequences. In embodiments, the oligonucleotide barcode includes an amplification primer binding sequence. In embodiments, the oligonucleotide barcode includes a sequencing primer binding sequence. The amplification primer binding sequence refers to a nucleotide sequence that is complementary to a primer useful in initiating amplification (i.e., an amplification primer). Likewise, a sequencing primer binding sequence is a nucleotide sequence that is complementary to a primer useful in initiating sequencing (i.e., a sequencing primer). Primer binding sequences usually have a length in the range of between 3 to 36 nucleotides, also 5 to 24 nucleotides, also from 14 to 36 nucleotides. In embodiments, an amplification primer and a sequencing primer are complementary to the same primer binding sequence, or overlapping primer binding sequences. In embodiments, an amplification primer and a sequencing primer are complementary to different primer binding sequences. In embodiments, the primer binding sequence is complementary to a fluorescent in situ hybridization (FISH) probe. FISH probes may be custom designed using known techniques in the art, see for example Gelali, E., Girelli, G., Matsumoto, M. et al. Nat Commun 10, 1636 (2019).

In embodiments, demultiplexing the multiplexed signal includes a linear decomposition of the multiplexed signal. Any of a variety of techniques may be employed for decomposition of the multiplexed signal. Examples include, but are not limited to, Zimmerman et al. Chapter 5: Clearing Up the Signal: Spectral Imaging and Linear Unmixing in Fluorescence Microscopy; Confocal Microscopy: Methods and Protocols, Methods in Molecular Biology, vol. 1075 (2014); Shirawaka H. et al.; Biophysical Journal Volume 86, Issue 3, March 2004, Pages 1739-1752; and S. Schlachter, et al, Opt. Express 17, 22747-22760 (2009); the content of each of which is incorporated herein by reference in its entirety. In embodiments, multiplexed signal includes overlap of a first signal and a second signal and is computationally resolved, for example, by imaging software.

In embodiments, the method further includes measuring an amount of one or more of the targets by counting the one or more associated sequences. In embodiments, the method further includes counting the one or more associated sequences in an optically resolved volume.

In embodiments, the number of unique targets detected within an optically resolved volume of a sample is about 3, 10, 30, 50, or 100. In embodiments, the number of unique targets detected within an optically resolved volume of a sample is about 1 to 10. In embodiments, the number of unique targets detected within an optically resolved volume of a sample is about 5 to 10. In embodiments, the number of unique targets detected within an optically resolved volume of a sample is about 1 to 5. In embodiments, the number of unique targets detected within an optically resolved volume of a sample is at least 3, 10, 30, 50, or 100. In embodiments, the number of unique targets detected within an optically resolved volume of a sample is less than 3, 10, 30, 50, or 100. In embodiments, the number of unique targets detected within an optically resolved volume of a sample is about 1, 2, 3, 4, 5, 6, 7, 8, 9, 10, 20, 50, 100, 500, 1,000, 5,000, 10,000, or 200,000. In embodiments, the methods allow for detection of a single target of interest. In embodiments, the methods allow for multiplex detection of a plurality of targets of interest. The use of oligonucleotide barcodes with unique identifier sequences as described herein allows for simultaneous detection of 2, 3, 4, 5, 6, 7, 8, 9, 10, 20, 30, 40, 50, 60, 70, 80, 90, 100, 200, 300, 400, 500, 600, 700, 800, 900, 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, 10,000 or more than 10,000 unique targets within a single cell. In contrast to existing in situ detection methods, the methods presented herein have the advantage of virtually limitless numbers of individually detected molecules in parallel and in situ.

In an aspect is provided a computer-implemented method for designing oligonucleotide sequences. In embodiments, the method includes generating a plurality of oligonucleotide sequences including 20 to 40 nucleotides; removing oligonucleotide sequences including a GC percentage greater than 60% over a portion of the oligonucleotide sequence; removing oligonucleotide sequences including five consecutive strong bases and or five consecutive weak bases; removing oligonucleotide sequences including a secondary structure; and removing oligonucleotide sequences including complementarity to the transcriptome.

In embodiments, removing oligonucleotide sequences including a GC percentage greater than 60% over a portion of the oligonucleotide sequence includes removing sequences with a GC percentage greater than 60%, 65%, 70%, 75%, 80%, 85%, 90%, 95%, or 100%. In embodiments, the oligonucleotide sequence includes a GC percentage of about 45%, 46%, 47%, 48%, 49%, 50%, 51%, 52%, 53%, 54%, 55%, 56%, 57%, 58%, 59%, or 60%.

In embodiments, the method includes removing oligonucleotide sequences including a secondary structure. In embodiments, secondary structure includes hairpin loop structures, stem-loop structures, bulges, internal loops, pseudoknots, G-quadruplex, or cruciform structures. Examples of sequences capable of forming DNA hairpins, pseudoknots, and cruciform are known in the art, and described in, e.g., Baker E et al. J. Phys. Chem. B. 2009; 113 (6): 1722-7, which is incorporated herein by reference in its entirety.

In embodiments, the method includes removing oligonucleotide sequences having complementarity to the transcriptome. In embodiments, the method includes removing oligonucleotide sequences having about 10%, 15%, 20%, 25%, 30%, 35%, 40%, 45%, 50%, 55%, 60%, 65%, 70%, 75%, 80%, 85%, 90%, or 95% complementarity to the transcriptome.

In embodiments, the method further includes removing oligonucleotide sequences having complementarity to the genome. In embodiments, the method includes removing oligonucleotide sequences having about 10%, 15%, 20%, 25%, 30%, 35%, 40%, 45%, 50%, 55%, 60%, 65%, 70%, 75%, 80%, 85%, 90%, or 95% complementarity to the genome.

In embodiments, the method further includes removing oligonucleotide sequences with a homopolymer sequence greater than 3 nucleotides. In embodiments, the method further includes removing oligonucleotide sequences with a homopolymer sequence greater than 4 nucleotides. In embodiments, the method further includes removing oligonucleotide sequences with a homopolymer sequence greater than 5 nucleotides. In embodiments, the method further includes removing oligonucleotide sequences with a homopolymer sequence greater than 6 nucleotides.

### EXAMPLES

### Example 1. Experimenting to Arrive at Effective Polynucleotide Sequences Useful for Target Detection

The field of cellular biology has recently been engaged in the intricate task of identifying and quantifying proteins within their native cellular environments. This endeavor, known as in situ protein detection, is pivotal for understanding the complex interplay of biomolecules within cells, thus offering insights into cellular functions, signaling pathways, and the underlying mechanisms of various diseases. Traditional methods of protein detection, such as immunohistochemistry and fluorescence microscopy, have provided substantial information. However, these techniques often face limitations in sensitivity, specificity, and the ability to simultaneously detect multiple proteins within the complex milieu of the cell. A significant limitation of current diagnostic tools is their capacity to perform multiple assays simultaneously. Typically, existing protein diagnostic assays can detect only 1-10 protein biomarkers at a time. For example, the prostate cancer PSA assay measures only the prostate-specific antigen protein, and the breast cancer Hercept Test detects only the Her2 receptor. However, cells undergo numerous interactions and pathways, many of which are altered in diseased states. To fully understand cellular function and the complex processes within and between healthy cells, as well as the changes in disease conditions, new technologies are needed to track and correlate a broader range of genetic, protein, and other cellular component changes. To address these limitations, introduced herein is a method that enhances sensitivity and specificity, particularly in detecting multiple proteins simultaneously, through the use of advanced antibody-oligonucleotide (Ab-O) conjugates. Ab-O conjugates have been utilized in various molecular biology applications, including biochemical and diagnostic assays, to improve assay sensitivity. For example, Ab-O conjugates have been used in immunoassays and sensitive nucleic acid-based diagnostic assays. These conjugates are prepared using methods such as glutaraldehyde crosslinking, maleimide-thiol coupling, isothiocyanate-amine coupling, hydrazone coupling, oxime coupling, and Schiff base formation/reduction.

The recent development of molecular techniques has revolutionized the landscape of in situ protein detection. Integrating nucleic acid-based methods (e.g., sequencing) with traditional protein detection strategies (e.g., antibody-oligo conjugates, or Ab-O conjugates) opens new avenues for enhancing sensitivity, specificity, and multiplexing. These advancements are crucial for high-throughput studies, enabling the simultaneous detection of multiple proteins, and is particularly important in the study of cellular heterogeneity and in diseases like cancer, where the expression levels of numerous proteins can provide vital diagnostic and prognostic information. The ability to accurately and efficiently detect a wide array of proteins in situ is, therefore, a cornerstone in both basic biological research and clinical diagnostics. The methods and compositions described herein further leverages this integration by offering a refined approach that combines the specificity of antibodies with the multiplexing capabilities of oligonucleotides for improved in situ protein detection.

Despite these advancements, challenges persist in optimizing molecular techniques for in situ protein detection. The need for high specificity and sensitivity necessitates the development of methods that can reliably distinguish between numerous protein targets in a complex cellular environment. This requires careful consideration of probe design, target accessibility, and signal amplification strategies. The method provided herein addresses these challenges by employing unique Ab-oligo conjugates designed for optimal target accessibility and employing advanced signal amplification techniques, thereby ensuring high fidelity in protein detection. Furthermore, the integration of these molecular tools into existing microscopy and imaging platforms is essential for the visualization and quantification of proteins at the cellular and subcellular levels. Addressing these challenges is critical for advancing our understanding of cellular processes and for the development of novel diagnostic and therapeutic approaches. Described herein is a solution to these and other problems in the art.

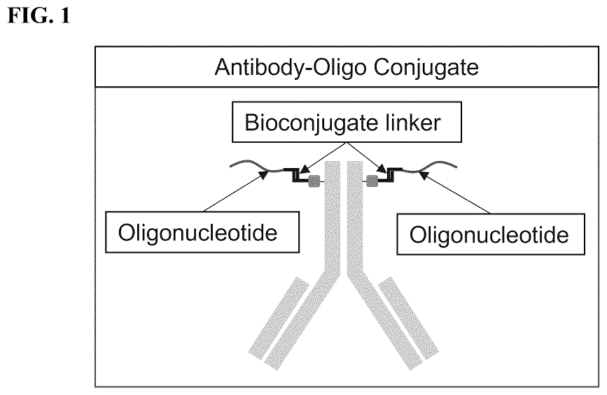

As further described herein, our method focuses on a methodology for protein detection employing specific oligonucleotide sequences attached to antibodies, referred to as Ab-oligo conjugates (e.g., alternatively referred as specific binding agents as described herein). This methodology surpasses existing methods by enhancing the detection of a broad spectrum of proteins with heightened sensitivity and specificity. See FIG. 1 for an embodiment of the Ab-oligo conjugate. This innovative technique leverages the specificity of antibodies to target proteins, for example proteins within cells or expressed on the surface of cells, coupled with the precision and diversity of oligonucleotide sequences for subsequent detection and multiplexing, respectively. The uniqueness of this method lies in its ability to detect a vast array of proteins, (e.g., greater than 100 different targets) within a sample without unintended non-specific interactions with other cellular components, particularly genomic DNA which exhibits a natural non-specific affinity for single stranded DNA. This is achieved through the design of highly orthogonal oligonucleotide sequences that ensure minimal cross-reactivity and high specificity in identifying distinct protein targets. The method includes binding a plurality of different specific binding reagents to a plurality of different targets and subsequently detecting the targets. This enhances the accuracy and reduces the likelihood of false positives in protein detection. Each binding reagent is specific for a particular target, and includes a sequence associated with that specific binding reagent. For example, a first specific binding reagent specific for protein PD-L1 includes an oligonucleotide having the sequence: “AGT.” A second specific binding reagent specific for protein CD15 includes an oligonucleotide having the sequence: “ACG.” When detecting both PD-L1 and CD15 in the same sample, every time the sequence “AGT” is detected, this corresponds to PD-L1 being present. Similarly, when “ACG” is detected, the presence of CD15 is determined.

Detection of the oligonucleotide sequences may occur directly. For example, fluorescent probes complementary to the oligonucleotides, may be bound to the Ab-oligo conjugates. Subsequently, the fluorescence emitted by the probes is observed and analyzed under a fluorescence microscope, enabling the detection and localization of specific nucleic acid sequences, typically within the cellular or tissue context. In embodiments, amplification of the oligonucleotide sequence is often pursued to enhance the sensitivity and specificity of molecular probes, such as Fluorescent In Situ Hybridization (FISH) probes. Amplifying an oligonucleotide sequence significantly enhancing the signal-to-noise ratio, markedly increases the intensity of the signal detected from target sequences, making them more discernible against background noise. Amplification of the oligonucleotide sequence is crucial when detecting sequences present in low abundance or alongside other independent readout modalities such as RNA, as it substantially improves the precision and reliability of localization and quantification within native cellular or tissue structure.

Many methods exist for the amplification of nucleic acids. These include the polymerase chain reaction (PCR), and the ligase chain reaction (LCR), both of which require thermal cycling, the transcription based amplification system (TAS), the nucleic acid sequence based amplification (NASBA), the strand displacement amplification (SDA), the invader assay, rolling circle amplification (RCA), and hyper-branched RCA (HRCA). In embodiments, Rolling Circle Amplification (RCA) is preferred for retaining the localization of amplification products, particularly in in situ applications. RCA, being an isothermal amplification process, generates a high number of localized amplification products that remain localized to the original site of the target sequence. RCA amplifies a circular polynucleotide (e.g., DNA) by polymerase extension of an oligonucleotide complementary to a portion of the circular polynucleotide. This process generates copies of the circular polynucleotide template such that multiple complements of the template sequence arranged end to end in tandem are generated (i.e., a concatemer) locally preserved at the site of the circle formation. RCA results in the generation of a long concatemeric nucleic acid product which comprises multiple tandemly repeated complementary copies of a circular RCA template, such as a circularized padlock probe, or other circular probe. Such a RCA product (RCP) coils into a bundle, or “blob”, of DNA which may readily be detected, for example by the hybridization to the RCP of labeled detection probes, and visualized by microscopy or flow cytometry. Alternatively, following amplification, the oligonucleotide may be detected using fluorescently labelled probes and/or sequencing.

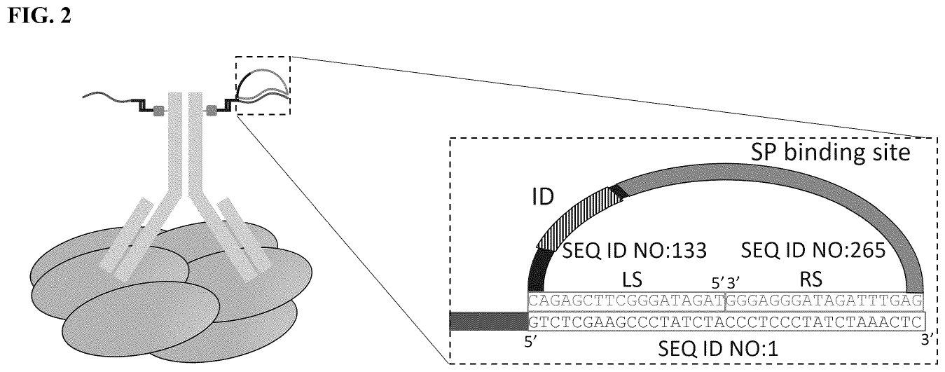

Implied in the name, a circular polynucleotide is required to perform RCA. Thus, preformed nucleic acid circles may be used as part of an RCA-based signal generation process. Accordingly, in one embodiment the circular probe may be a preformed circle. Alternatively, a circularizable probe may be used. A circularizable probe refers to one or more linear oligonucleotides which may be ligated together to form a circle. A typical example of a circularizable probe is a padlock probe. Padlock probes are specialized ligation probes, examples of which are known in the art, see for example Nilsson M, et al. Science. 1994; 265 (5181): 2085-2088), and has been applied to detect molecules in cells, see for example Christian A T, et al. PNAS USA. 2001; 98 (25): 14238-14243, both of which are incorporated herein by reference in their entireties. A padlock probe is typically a linear circularizable oligonucleotide which has free 5′ and 3′ ends which are available for ligation, to result in a circular conformation post ligation. It is understood that for circularization (ligation) to occur, the padlock probe has a free 5′ phosphate group. In embodiments, to permit juxtaposition of the ends of the padlock probe for ligation, the padlock probe is designed to have at its 5′ and 3′ ends, regions of complementarity to its target sequence. For example, the regions of the padlock probe are complementary to sequences provided by the oligonucleotide attached to the antibody. These regions of complementarity thus allow specific binding of the padlock probe to its target sequence by virtue of hybridization to specific sequences in the target, and by the hybridization the ends of the padlock probe are brought into juxtaposition for ligation. In embodiments, the ends of the padlock probe may be ligated directly to each other or they may be ligated to an intervening nucleic acid molecule, or sequence of nucleotides (for example, following gap-fill). As used herein, gap-fill refers to extending the 3′ end of the padlock probe described herein along the sequence of the target sequence using a polymerase to generate a complementary sequence. In embodiments, the padlock probe (e.g., the polynucleotide probe described herein) may be provided in 1, 2, 3 or more parts, and there may be more than one ligation reaction in order to circularize the polynucleotide. In an embodiment, the polynucleotide probe includes a hybridization sequence (i.e., a sequence complementary to the oligonucleotide attached to the specific binding reagent), a primer binding sequence, and optionally, an identifying portion such as a nucleotide unique to the polynucleotide probe or barcode sequence. For example, depicted in FIG. 2 is an Ab-oligo conjugate (e.g., a specific binding agent described herein) bound to a protein. The boxed portion shows a polynucleotide probe bound to the oligonucleotide, wherein the polynucleotide probe includes a first hybridization sequence on the left-side (LS), a second hybridization sequence on the right-side (RS), a sequencing primer binding sequence (SP binding site) and an optional barcode portion.

Delivering antibody-oligonucleotide (Ab-oligo) conjugates (e.g., a specific binding agent described herein) and subsequent amplification probes, such as padlock probes (e.g., a polynucleotide probe described herein), to a target within a cell presents several challenges. One primary complication arises from non-specific binding events, where the Ab-oligo conjugates and padlock probes may inadvertently bind to off-target molecules, leading to false positives and reduced specificity in the detection process. Additionally, the secondary structures formed by oligonucleotide sequences can impede efficient hybridization with the amplification probe sequence, as these structures may hinder the accessibility of the oligonucleotide portion to its complementary sequence. Furthermore, the cellular environment itself poses a barrier, as the delivery of these conjugates and probes into the cell must overcome cellular membranes and avoid degradation by cellular enzymes, while ensuring the functional integrity of the conjugates and probes for accurate target detection.

The oligonucleotide sequences are structured to avoid homopolymers, that is, consecutive nucleotides including the same nucleobase, thereby mitigating issues related to the amplification steps often encountered in nucleic acid-based amplification and detection methods. Avoiding the amplification of repeated stretches of consecutive nucleotides (e.g., a polyG sequence) is critical for maintaining the integrity and efficiency of the amplification process, which is valuable in enhancing the sensitivity of protein detection. Additionally, our approach takes into account the hybridization efficiencies of two distinct regions where the probes bind. This dual-region binding strategy not only ensures robustness in the detection process but also contributes to the high specificity and sensitivity of the technique in identifying and quantifying multiple proteins within the intricate environment of the cell.

Reliable oligonucleotide design is crucial for successful capture and detection, and given the diversity of targets and multiplexing, the design of oligonucleotides requires flexibility in the approach. Thus, a number of oligonucleotide design tools exist, for example PrimerSelect (Plasterer T N., Methods Mol. Biol., 1997, vol. 70 (pg. 291-302)), Primer Express (Applied BiosystemsPrimer Express® Software Version 3.0 Getting Started Guide, 2004), OLIGO 7 (Rychlik W., Methods Mol. Biol., 2007, vol. 402 (pg. 35-60)) and Primer3 (Untergasser, A. et al., Nucleic Acids Res 40, e115 (2012)). Additional online tools, for example the OligoAnalyzer™ Tool provided by Integrated DNA Technologies (accessible at www.sg.idtdna.com/pages/tools/oligoanalyzer) or PrimerROC (Johnston, A. D., Lu, J., Ru, Kl. et al. Sci Rep 9, 209 (2019)) sheds additional insight into the secondary structure of oligonucleotides and the resulting amplification products, as well as the self- and heterodimerization tendencies of each primer set. Primer-BLAST is another web service that supports the selection of effective primers by considering opportunities for mispriming across an entire genome or transcriptome (Sayers E W et al., Nucleic Acids Res., 2012, vol. 40 (pg. D13-D25)). Additionally, RNAFold, provided with ViennaRNA Package 2.0 (LorenzR, et al. Algorithms Mol Biol. 2011 Nov. 24; 6:26.) utilizes thermodynamic principles to determine the most stable structure based on the sequence provided, offering insights into the functional properties and cross-interactions. These tools are useful starting points for generating effective oligonucleotide sequences.

A goal of effective oligonucleotide design is to maximize detection and minimize off-target amplification, without introducing any biases (e.g., skewing the amplification products to over- or under-represent targets). Primer3 allows for the selection of the primer on the basis of melting temperature (Tm), primer length, and 3′-end stability, which was considered when designing each primer set. Calculating the melting temperature and performing thermodynamic modelling for estimating the propensity of primers to hybridize with other primers or to hybridize at unintended sites in the template offer an accurate approach for predicting the energetic stability of DNA structures. For example, because of electronic effects of nucleobase stacking, the stability of 5′-CT-3′ hybridized to 3′-GA-5′ is different from that of 5′-CA-3′ hybridized to 3′-GT-5′, despite the base pairings C:G and T:A are the same. It is recommended to perform primer analysis with sophisticated modelling capabilities to capture such electronic effects. Additionally, in silico validation of primer and amplicon sequences was performed. The online software OligoAnalyzer™ Tool provides information on amplicon secondary structure and the possibility of self- or heterodimer formation by the primer sequence itself by calculating the Gibbs free energy (ΔG).

While software and online tools can provide an initial set of primer sequences, proper confirmatory validation occurs ex silico. We performed a series of experiments varying reaction conditions (e.g., temperature, additives, incubation duration) and detecting the resulting amplicons to derive an optimized oligonucleotide sequence.

The oligonucleotides described herein include one or more binding sequences, which enable specific hybridization of corresponding circular or circularizable probes. The sequence of these oligonucleotides is advantageously selected to ideally avoid, or at least minimize, binding of the oligonucleotides to off-target sequences (e.g., sequences other than their partner circular or circularizable probe). By way of example, the sequences of the oligonucleotides were selected to minimize non-specific binding to any human genomic sequence.

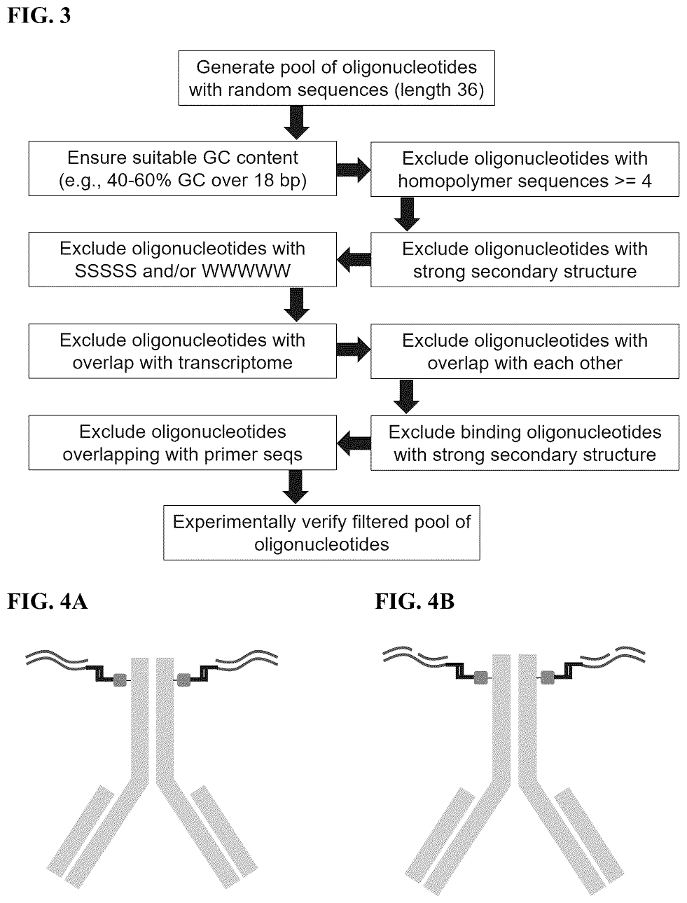

An example workflow is provided in FIG. 3. First, to generate effective oligonucleotide sequences, we generated 5,000,000 random oligonucleotide sequences with a target GC content of approximately 50-55% GC having a length of 36 bp. Sequences containing elevated amounts of GC can bind non-specifically to off target templates, and secondary structures are more likely in high GC content target DNA and can cause inefficient binding. Next, to ensure the target GC content remains within 50-55% for each hybridization sequence of the circularizable probe, we selected oligonucleotides containing 8-11 GC per hybridization sequence, resulting in 44-60% GC over the range of 18 bp. Oligonucleotides containing homopolymer sequences of greater than 4 consecutive nucleotides were omitted. We further excluded oligonucleotides containing SSSSS and/or WWWWW to further control both the global and local GC content, wherein S=Strong bases (G or C) and W=weak bases (A or T) according to IUPAC naming convention for DNA beyond just the standard nucleotides. We filtered oligonucleotides to retain sequences with i) limited sequence overlap (i.e., minimal overlap so as not to form dimers) and ii) minimal secondary structure. From this filtered set we performed a BLAST alignment to determine the % identity against all available sequences in GenBank and removed sequences with any significant matches (e.g., removed sequences with 10 nucleotide match to a known sequence in the human transcriptome). We manually filtered the oligonucleotides to discard any sequences with a 10 nucleotide match to any oligonucleotides within the set (i.e., removing redundant sequences) and to any amplification primers and sequencing primers. It is understood that the order of the filtering steps may occur in any order, and the particular outline provided above and in FIG. 3 may be modified.

The resulting orthogonal sequences are provided in Table 1, absent any linking spacer nucleotides or phosphorothioate modifications. In the development of antibody-oligo conjugates (e.g., specific binding agents described herein) for cellular and tissue applications, careful consideration is given to the choice of oligonucleotide sequences to minimize non-specific interactions. Typically, mRNA molecules in eukaryotic cells are characterized by polyadenylation, a feature that includes stretches of polyT sequences. To circumvent non-specific binding to these mRNA sequences, poly A linkers are useful for attaching oligonucleotides to antibodies. This approach leverages the specificity of polyA sequences in hybridizing with complementary oligos, while significantly reducing the likelihood of cross-reactivity with endogenous mRNA. In embodiments, the oligonucleotide includes a poly-A linker i.e., two or more consecutive adenine nucleotides spacers, although other nucleotides and combinations thereof can be used. In embodiments, the linker includes 6, 7, 8, 9, 10, 11, 12, 13, 14, or 15 dATP spacer nucleotides. In embodiments, the linker includes 6-8 dATP spacer nucleotides. In embodiments, the linker includes 7 dATP spacer nucleotides. For example, the oligonucleotide may include, from 5′ to 3′, a bioconjugate reactive moiety, a linker (e.g., a poly A sequence), and the sequence provided in Table 1.

The enzymes used in amplification (e.g., rolling circle amplification) pose a significant challenge when working with oligonucleotides, particularly due to its pronounced exonuclease activity. This enzymatic activity leads to the degradation of free ends of oligonucleotides, which can compromise their stability and integrity, thus impacting their efficacy. Additionally, addressing the susceptibility of antibody-oligonucleotide conjugates to nuclease degradation during storage is critical. Nucleases, which are ubiquitous in laboratory and storage environments, may compromise the integrity of these conjugates. Nuclease contamination can lead to the unwarranted cleavage of oligonucleotides, particularly at their unmodified ends, thereby limiting their functional utility and shelf-life.

To mitigate these issues, one may include phosphorothioate modifications at the terminal ends of the oligonucleotides. These modifications entail substituting a non-bridging oxygen for a sulfur atom within the phosphate backbone, rendering the oligonucleotides resistant to the exonuclease activity of phi29 DNA polymerase and confer increased resistance to nuclease activity. By incorporating phosphorothioate bonds, particularly at the 5′ and 3′ ends of the oligonucleotides, one can significantly enhance the nuclease resistance of the antibody-oligonucleotide conjugates. Phosphorothioate modifications are not explicitly shown in the sequences provided herein, but it is understood that such modifications may be included on vulnerable regions, such as the 3′ ends of single-stranded oligonucleotides. In embodiments, the oligonucleotide includes one or more phosphorothioate nucleotides.

TABLE 1
Effective oligonucleotide sequences. It is understood that white space, line
breaks, and text formatting are not indicative of separate sequences or
structural implications. Phosphorothioate nucleotides are not specifically
identified in the table, yet in embodiments, the 3′ end includes 1, 2, or 3
phosphorothioate nucleotides which may prevent nuclease digestion or
enzyme digestion.

| Ab Oligo | Sequence (5′-3′) |
|---|---|
| AbO-1 | GTCTCGAAGCCCTATCTACCCTCCCTATCTAAACTC; SEQ ID NO: 1 |
| AbO-2 | GCTCCACTCAACCAGACTCCCATACCATTCTACAGC; SEQ ID NO: 2 |
| AbO-3 | AGTCCGCTGCAATCCTAATCCGATCATACAACACCG; SEQ ID NO: 3 |
| AbO-4 | TACCTGCCGATCCCACACACCGTTATCACCATTCTC; SEQ ID NO: 4 |
| AbO-5 | GGAAGTTCAGGTCGGATATGGGTAGGGTTCAGTGTC; SEQ ID NO: 5 |
| AbO-6 | GTAAAGTGCCGTGTACCCTCTCCCTTCGACCCTTAT; SEQ ID NO: 6 |
| AbO-7 | TTCCTCGAGCCTTAACCGACCTATCTCAAACACCGC; SEQ ID NO: 7 |
| AbO-8 | GGCAGTCTTACGCTAACCCAACCAATCTACCCAACG; SEQ ID NO: 8 |
| AbO-9 | TAATGCTCGGTCCGCAACACTACATACAACCAACGC; SEQ ID NO: 9 |
| AbO-10 | CTGCCCACTTAACCGCTCTCTGTCGTTCATCACCAT; SEQ ID NO: 10 |
| AbO-11 | GAGTAAGAGCCCAACCAAGCATTCCCTAAACGAGTC; SEQ ID NO: 11 |
| AbO-12 | CCTCCGACATACCATACCGATTTGACTAACCGAACC; SEQ ID NO: 12 |
| AbO-13 | CCACCGCTCTCATCTAATCAGAAACACGACGCCAGA; SEQ ID NO: 13 |
| AbO-14 | CCATCACACGACCAGTTACGGCAATCTCACCACAAG; SEQ ID NO: 14 |
| AbO-15 | CTGTAGCCACCGATCAAAGACGCAACCACCTAATCC; SEQ ID NO: 15 |
| AbO-16 | AAGGGAGGTCGGTAGGATTCGGAGACAAGGTTCAGG; SEQ ID NO: 16 |
| AbO-17 | ATCCCACGCTAGTCACACACCCTTCTCTATGCCGTT; SEQ ID NO: 17 |
| AbO-18 | CGGGATAGTGCAAGTACGGACAGATAGGGTAAGATC; SEQ ID NO: 18 |
| AbO-19 | AAAGGGCTGTTCGGTTCATCTACTTATCGGCTGCAG; SEQ ID NO: 19 |
| AbO-20 | GGGAGTATGGTGCGTCAGGTCCGTATCGATGTCTTA; SEQ ID NO: 20 |
| AbO-21 | ATCCGACTAGCTGCCTAACACATGAACCCACTATGC; SEQ ID NO: 21 |
| AbO-22 | CAGAATCAACGAGCTCCACGACACGCACAACCAAGA; SEQ ID NO: 22 |
| AbO-23 | CTATCCACTCCGATCCTAAACTGCCTGCGAGCGAAT; SEQ ID NO: 23 |
| AbO-24 | GTGTGGGTGGTCTACATATGGTTGCGAGTCTTGGAG; SEQ ID NO: 24 |
| AbO-25 | CTCCACTCCTGTTCGACGACTTACCAATCCAACCGC; SEQ ID NO: 25 |
| AbO-26 | TACCTCTTCGCAACACCACTACCCTCGAACAAGTCT; SEQ ID NO: 26 |
| AbO-27 | GTTGTATTGGGTGTCGCGAATAGGTAGTGGAGATCG; SEQ ID NO: 27 |
| AbO-28 | GGTGAGATGCGGTAGAACTACGTGTTGGAATTGGGC; SEQ ID NO: 28 |
| AbO-29 | AACACTCTCTTGGTCCCGTTTACTCCGATTTCACCG; SEQ ID NO: 29 |
| AbO-30 | TAGGGAGGAGATAGTCGAACACGAGAGGCTGGATGA; SEQ ID NO: 30 |
| AbO-31 | CATCCCTTCCTCGCATATGATCACGCACTTCCAGAG; SEQ ID NO: 31 |
| AbO-32 | CCGAAGATCCACCGAACGCAACCTAAGACCCTTCCA; SEQ ID NO: 32 |
| AbO-33 | GATTAGGGTTGAGCTCGTGTCAGTATCAGGTTCGTG; SEQ ID NO: 33 |
| AbO-34 | AGCCATATCCCACGCCACTTCGGAACCTAGATTTGC; SEQ ID NO: 34 |
| AbO-35 | TCTCGTGCCCATTCCATCCTGCATACTTCTCGAAAC; SEQ ID NO: 35 |
| AbO-36 | GCTACGACTATCCATGCGACTCACCAACCTTCTGCA; SEQ ID NO: 36 |
| AbO-37 | CCATATCGTGCCATCCAGTACGCGAATCCACAACTT; SEQ ID NO: 37 |
| AbO-38 | TGCCACCGACCTGTATCAACCTAGCTGACGCGAATT; SEQ ID NO: 38 |
| AbO-39 | AGGGTAGGTGTCAGCGAAGGTCTAGAAGCGTGCAGA; SEQ ID NO: 39 |
| AbO-40 | AACCCTTACCGCTTCTGTCATGCCGTCCTAGCTTTG; SEQ ID NO: 40 |
| AbO-41 | CTCTAGCACACGCGATAGTAACACCACTTCCTTCCA; SEQ ID NO: 41 |
| AbO-42 | CCTGTCTATACCTCGGCAAGCTTCTCGTCGCATCTG; SEQ ID NO: 42 |
| AbO-43 | CTTGCTGCCGTACCTTGATCGAACTCTGTGCCAATG; SEQ ID NO: 43 |
| AbO-44 | GAGCTGCAATACCAACCCGACGAACAACACGAAAGA; SEQ ID NO: 44 |
| AbO-45 | GCATATCCTCCTAGTCCGGTTCCATCCTAGATCACC; SEQ ID NO: 45 |
| AbO-46 | CCCTGCAACGACCGATAACGCCTACTAGTAACCACC; SEQ ID NO: 46 |
| AbO-47 | GCGCAGATCCAACTATCAACGACAAACTCCCAACAC; SEQ ID NO: 47 |
| AbO-48 | CCTCAGTAACAGCACGCTAGACCACGGACGACTAAC; SEQ ID NO: 48 |
| AbO-49 | CAACCCAATGCCACGTATGCTACTAGAACCGCACCT; SEQ ID NO: 49 |
| AbO-50 | CTAACCACGACCCGAACTTATCCGCTAGATTCCACC; SEQ ID NO: 50 |
| AbO-51 | GGGTGAATAGGAAGGTCGATGTGATGAGACACGGGA; SEQ ID NO: 51 |
| AbO-52 | TATGCCCTTGAGCGTACCTAGACAAACCGGAACCAC; SEQ ID NO: 52 |
| AbO-53 | ACATGTCAGTCGTGGGTGTGATCTTTAGTGTGCCAG; SEQ ID NO: 53 |
| AbO-54 | GCCGTCCAGATCACATTGAAGCGACTACAAATCTCC; SEQ ID NO: 54 |
| AbO-55 | GCCCTCATACGACGTATCGCAACTACACACCTTACT; SEQ ID NO: 55 |
| AbO-56 | TGTGGGTATTGAGGGTCGCGTGTTGTTTGCATCGTA; SEQ ID NO: 56 |
| AbO-57 | GTACACCTAGCGTAAGGACCAATCAAACTCCAACCC; SEQ ID NO: 57 |
| AbO-58 | AGGCACCACTCATCATGTCATCGCTCAGACTTCAAG; SEQ ID NO: 58 |
| AbO-59 | GCCGAAAGAGAACACCGTACCCACCAACAAGCTCTT; SEQ ID NO: 59 |
| AbO-60 | GCGAAGGTGCGAGGATATGGTTTGCGGTTGTCTGGT; SEQ ID NO: 60 |
| AbO-61 | AACTAGCTCGTGTGGGTAATCGTTTGTGGAGGTGCA; SEQ ID NO: 61 |
| AbO-62 | AGTGGGTATGGACGTCGGAATGCACGGGAAGTTTAG; SEQ ID NO: 62 |
| AbO-63 | GCTGAGTAGTACGTTCGGTTCTGGGTGCATTGAGTA; SEQ ID NO: 63 |
| AbO-64 | CACCTCTTCCGTTCAGTCTCGATCACAGTGGAATCT; SEQ ID NO: 64 |
| AbO-65 | ACCATCCTAGCCTTGCACCTGTTATCGTTGGACTGA; SEQ ID NO: 65 |
| AbO-66 | GTATCATCGGTGGCAGTAGTCGTGTATTCGGTGTAG; SEQ ID NO: 66 |
| AbO-67 | AGCGAGGTATCACGACATCTACACATAAGCGGATCC; SEQ ID NO: 67 |
| AbO-68 | GTGGCTGTCAGATAGGCTCGGTAAAGTTGTAGGAGG; SEQ ID NO: 68 |
| AbO-69 | GTGCGTACAGGTTATCGTTCCAAAGCCGTCCATTAC; SEQ ID NO: 69 |
| AbO-70 | GGAGATGTCGGACGTTTGCAGTGTATAGACGGTGTG; SEQ ID NO: 70 |
| AbO-71 | GCCCACTTGATGATTCCTACCGATGTTGCCCTGATC; SEQ ID NO: 71 |
| AbO-72 | GCATCTAGTGTCCATCCAGAATACGCTCCTCCCTGA; SEQ ID NO: 72 |
| AbO-73 | CCGTTTCCTGCCCTGTTAACCCTACCAAGCTACAAG; SEQ ID NO: 73 |
| AbO-74 | GCTGCACGATCTCTACATGACGACGGTTCTGAGTTA; SEQ ID NO: 74 |
| AbO-75 | AGTGGAGCGGAAGTTTGGGATAGCGTGCATAACAGA; SEQ ID NO: 75 |
| AbO-76 | ACCCATGCTCTTGGTACGGCTCTATACATGACTCTC; SEQ ID NO: 76 |
| AbO-77 | GTCCATGTACGTATCGGTCTAGCACGGGTCTGAGTA; SEQ ID NO: 77 |
| AbO-78 | CACGTTAACCAAGGCGAGCTATTGAGGGAGAGTATG; SEQ ID NO: 78 |
| AbO-79 | CACAAACCAAGAGCGCAACATCGTCGCCACATACCT; SEQ ID NO: 79 |
| AbO-80 | AAAGGGAGTGGACGCTAGTGTGTCAAATCGATGAGC; SEQ ID NO: 80 |
| AbO-81 | GAACCGCAAGCATCCAACCGGACAACTACTCATCAT; SEQ ID NO: 81 |
| AbO-82 | TCCTCATAACGCTCGACTTACATGTGACGCTTGGGT; SEQ ID NO: 82 |
| AbO-83 | GCCGACTCAATACTCCACGCTAGCCGAACAAATGAC; SEQ ID NO: 83 |
| AbO-84 | GGAAGGTTAAGGCGTGTAGGAGTTACTCAATGGAGC; SEQ ID NO: 84 |
| AbO-85 | AGAGTTGGAGTGTGCCGATTCGACGTGTACAAGGAG; SEQ ID NO: 85 |
| AbO-86 | TATAGGCGAACGGGTTGGATTAGCTACGGACACTCG; SEQ ID NO: 86 |
| AbO-87 | GTCTGGTACGATGGTCATTACGTTGTTAGTGCTCCG; SEQ ID NO: 87 |
| AbO-88 | CCTAACGCTATCCGGAACCGACTGATCACCTACAAC; SEQ ID NO: 88 |
| AbO-89 | GCTCTGATAGGTGTGTCGTAACGTAGATGGTAGTGG; SEQ ID NO: 89 |
| AbO-90 | CCGAGGATGATAGGACCCGACAAGTTACCCGACCAA; SEQ ID NO: 90 |
| AbO-91 | CCCGAGGCCTATACATAACCTACTCACGCACAGCAA; SEQ ID NO: 91 |
| AbO-92 | CCAAGAGACCGCTACACTGAAACGATCCCAACGAGT; SEQ ID NO: 92 |
| AbO-93 | TGTTGACGGTCGGTAAGTGTATAGTCCCATCGAGTC; SEQ ID NO: 93 |
| AbO-94 | GTACGGTCGTCACTATCTTAACGCTATGCCACTGAC; SEQ ID NO: 94 |
| AbO-95 | ATGCGTGTTGGTTCGTGTTCTAGTTGGGACGTTCTG; SEQ ID NO: 95 |
| AbO-96 | AGCAAGATAGGACCGGAGAACTCGTTAAGCTGTCAC; SEQ ID NO: 96 |
| AbO-97 | GGTCAGTGACGGCTTGAGATATGAGGTTCGGCTTGT; SEQ ID NO: 97 |
| AbO-98 | CGGTGGGTGGGTAGTTACATGGTTGCCTGATATCGA; SEQ ID NO: 98 |
| AbO-99 | CCGCAAGCTATCCATGTAGTCCGTCGAATTCTGTGA; SEQ ID NO: 99 |
| AbO-100 | CACCAGCAACCTTACCCGTCCTACAGACCATGATGC; SEQ ID NO: 100 |
| AbO-101 | AACTCTAGCTCCACGGTTAGGCCAACGAACTGATCC; SEQ ID NO: 101 |
| AbO-102 | GGCTAACTCTACTCCCATAACCTAGCGTGAGACTGC; SEQ ID NO: 102 |
| AbO-103 | GCTCTGATCCTAACGACGGTGGACTCGAATGTTACT; SEQ ID NO: 103 |
| AbO-104 | GACACTCCCATCGAACAACCAATCGTCCATCCTTTG; SEQ ID NO: 104 |
| AbO-105 | CTTGAGACAGCGTGCATGCTACTAACTCCCTAACCG; SEQ ID NO: 105 |
| AbO-106 | GGATAAGGTCTGGCGAAGATAGGGTCCGAACTAAGT; SEQ ID NO: 106 |
| AbO-107 | GACCGATCCTAGATGACGACGCGAAACAACCTTCCA; SEQ ID NO: 107 |
| AbO-108 | GGTGGTCTATGGCGACTGTATGAGGAGCAAGGTAGG; SEQ ID NO: 108 |
| AbO-109 | GCAGGATCGAAACTCAGTATGGGAGGTCTATGCGTA; SEQ ID NO: 109 |
| AbO-110 | GAGGTCGCTGTGGTATAGTTAGACGGAATAGGTCAG; SEQ ID NO: 110 |
| AbO-111 | AGGTCTAATGGAGGAGGACGTAATCACATCAGTCGC; SEQ ID NO: 111 |
| AbO-112 | GAACAAGGCCTACCGTTAGAGAGTTGACAGTCCAAG; SEQ ID NO: 112 |
| AbO-113 | CGTAGGTGCGAGAATTGAGCGTGTATGTTTGCCTGA; SEQ ID NO: 113 |
| AbO-114 | ATGGTATGCTGTTCGGGATGTAATGGGAGGACCTGA; SEQ ID NO: 114 |
| AbO-115 | CCACCTTCCACGAGTGAGTTGACCATAGCTAGAACG; SEQ ID NO: 115 |
| AbO-116 | GCCAAGACAAGACACGAAGCCATCCCACCGTACTTA; SEQ ID NO: 116 |
| AbO-117 | AGCCCTAGTTCTGTCGAAAGTATGGATGGGAGCGTT; SEQ ID NO: 117 |
| AbO-118 | GGGAGAGTGACTTAAGCCGAAGTGAGATCTAGGAGG; SEQ ID NO: 118 |
| AbO-119 | TATCGGCTGAGTGGAGAGTACCTAGATTCGTCGGTT; SEQ ID NO: 119 |
| AbO-120 | GTAGCTGATCCTGCGATACGGTGAGATAGACATTCC; SEQ ID NO: 120 |
| AbO-121 | TAGAGTCCCGATGAGCAAGTCAGAGCGAGATTGTGT; SEQ ID NO: 121 |
| AbO-122 | CCGCAAAGTAGGTCCAACATAGCAGTCGTGATCAAC; SEQ ID NO: 122 |
| AbO-123 | GAAGTGTGGAGGTTCGTCTAGTGACCGGTTGAGTTG; SEQ ID NO: 123 |
| AbO-124 | GTCGGATGTATTGGACGTCGATGATATGGAGTCTGG; SEQ ID NO: 124 |
| AbO-125 | CTACCACAGACCTCGAGCACCGTTCAACACTACCAG; SEQ ID NO: 125 |
| AbO-126 | TTTCCCGTCCCATTACCGTAATCCTCGCTGACAACT; SEQ ID NO: 126 |
| AbO-127 | GCACTCCTTAACACCGATCTGGCCACGCATACTAGA; SEQ ID NO: 127 |
| AbO-128 | GGCGTGTTTGGTCCCTATGTCCTAGCCGAAATCTTG; SEQ ID NO: 128 |
| AbO-129 | TCTAGTTAGGTGGCCAGTATACTCATGCGACGGTTC; SEQ ID NO: 129 |
| AbO-130 | CCAAACGAAGCGGTACTCAGGACTCACAAGCAATCT; SEQ ID NO: 130 |
| AbO-131 | CTAGAGGGAGGCGTGAAATGTAGAGCGAACGGATCA; SEQ ID NO: 131 |
| AbO-132 | ATCGTGGTTGCTAGGTCTTAGGCGTGGCTAGTTTCA; SEQ ID NO: 132 |
| AbO-133 | CGTCTCTACCCGAACGGACGGCGCCCGTTAGATTGT; SEQ ID NO: 397 |
| AbO-134 | GTCTATCGCCCCGACGGAGCTAGTCGACGACATGCG; SEQ ID NO: 398 |
| AbO-135 | ATGATCGCGTATCGCGGTAGCTCGTCGTTACGGGCG; SEQ ID NO: 399 |
| AbO-136 | CGATTGTTTACGGCGCCATGCTCGACGCGAACCGAC; SEQ ID NO: 400 |
| AbO-137 | ACGTCCGCGCATCGGATAAGCGCGATATGCGCGTAC; SEQ ID NO: 401 |
| AbO-138 | AGCGCGCTAACTCGATATTCGAACGGCTGGTCGGAC; SEQ ID NO: 402 |
| AbO-139 | ACCAACCGGCGTTACGGTAGTCGGGACGTGACTACC; SEQ ID NO: 403 |
| AbO-140 | TCGTTCTACTACGCGGTCTCGTTCGACCGTGCGGTT; SEQ ID NO: 404 |
| AbO-141 | CGCGCGTAACGACCATCGAAGTGTCGACGATCGACC; SEQ ID NO: 405 |
| AbO-142 | CGTAGGAGACTATCCCGCTCGCGACTCAAATCGCGG; SEQ ID NO: 406 |
| AbO-143 | GATTTACGCCGCCGCGGTATGAGGCGAATTGCGCCG; SEQ ID NO: 407 |
| AbO-144 | ACGGAGATCGCGTTAGCTCTCGAACGATGCGCGGTG; SEQ ID NO: 408 |
| AbO-145 | TCTGTAACGCCCGAAACGGCGCGATTCGAGTCTAGG; SEQ ID NO: 409 |
| AbO-146 | AAGCGTGCGGTAGCGTAAGTTCAATGCGCGTAGTCG; SEQ ID NO: 410 |
| AbO-147 | CGGGCTTGCACGACGATTAAGTGTCGACGATCGACC; SEQ ID NO: 411 |
| AbO-148 | GTGCGTCATCGTCGTAAAGACCCGATATCGCGTCCG; SEQ ID NO: 412 |
| AbO-149 | GACGCGCAACGGATCGATGATCGACGAGTCGGAGTC; SEQ ID NO: 413 |
| AbO-150 | GCTCGACACGCGCAATGACGACCAAACGTTAGCACG; SEQ ID NO: 414 |
| AbO-151 | CGACCCGCGGAGCAACTAAGTACGGATTCGACCGTC; SEQ ID NO: 415 |
| AbO-152 | GCTCGACACGCGCAATGACGACCAAACGTTAGCACG; SEQ ID NO: 416 |
| AbO-153 | GTATTCGGACGTAGCGGTTTGCCGGGCGATCGGTTG; SEQ ID NO: 417 |
| AbO-154 | ATGCCTATCGGGTCGCGACCACGAGACGTCTAACGG; SEQ ID NO: 418 |
| AbO-155 | CGTACGTGCGGTTCAGCTGTCGGTAGTCGCTCTCGC; SEQ ID NO: 419 |
| AbO-156 | CGATGCTGTGCGGGCGTAGTCAATTGCGCCGTACGC; SEQ ID NO: 420 |
| AbO-157 | TGGAGCCGACGGGCAGACGCCGTTCCGCTACTATAT; SEQ ID NO: 421 |
| AbO-158 | GTGGCTCCAGCGTGTTCTCGAAGTGCGAGTACGCGA; SEQ ID NO: 422 |
| AbO-159 | CAGAGTGCACTCACGAGGCTGTATCGCATGGTCGCG; SEQ ID NO: 423 |
| AbO-160 | CCGGACCGTACATTGCCGGGTTTCTCTCGGCGAGGA; SEQ ID NO: 424 |
| AbO-161 | ACATCTGCACGCCGGTCGCAGACGAAACGCTTCTCA; SEQ ID NO: 425 |
| AbO-162 | ATCGTCGCGCGGATTCGCTACGTTCGTCGTAGTGGA; SEQ ID NO: 426 |
| AbO-163 | AGAACACTGTGTGGCGCCGCCCGATCCGGGTACCGC; SEQ ID NO: 427 |
| AbO-164 | CTGCCGCGACCTGTTGCGGAGGTCGACGGAGCGCCG; SEQ ID NO: 428 |
| AbO-165 | GCGCTTCAAGTGGCGTCTGGCTCCGGCGAGTTAGAG; SEQ ID NO: 429 |
| AbO-166 | GGCCGCCATTCACGCCTTGCGCCGTTAATCTTGGAC; SEQ ID NO: 430 |
| AbO-167 | ATGTGGTCCGTGGACTATGCGGTCATTCCCGCCGTC; SEQ ID NO: 431 |
| AbO-168 | ACCAGTTCACGCTGGGTCGCGCGAACCCTTATCTGT; SEQ ID NO: 432 |
| AbO-169 | CTCCGGCGGGCTTTAATGGCCCGGACCGCCAATCAT; SEQ ID NO: 433 |
| AbO-170 | ACCTGGCCAAGCGGGAACGTCTATCTCGCTGCTGAA; SEQ ID NO: 434 |
| AbO-171 | CGAGTTGGGCGTCAATGTCGGCCGTCATGATGCACC; SEQ ID NO: 435 |
| AbO-172 | GCCGTCGTTGGTACCTCGATCGGTCCCGGAAGCGGC; SEQ ID NO: 436 |
| AbO-173 | GCGGATCGGCCCGCGGGAGTCGGTGACAGATTCTTG; SEQ ID NO: 437 |
| AbO-174 | GAAGCCTGGCGGATTGGTAGGCCGCTTTCCGGACTC; SEQ ID NO: 438 |
| AbO-175 | CCAGGCCGTCAAACGTCGGAGGTCCGGCTGCGACAT; SEQ ID NO: 439 |
| AbO-176 | CACCGTCGAGCAGGCCGTCCTTAAGGCTCTAGGCTG; SEQ ID NO: 440 |
| AbO-177 | GGCCGCGCGCTCGCTAGAGGTTCACGAACCTTGTGC; SEQ ID NO: 441 |
| AbO-178 | GGATGCGAAAGCCCTCGCGCCGAAATCGTGGATTCT; SEQ ID NO: 442 |
| AbO-179 | CGCCCGTTATCATACGAGCCCTAGGCGTTGGTCCGG; SEQ ID NO: 443 |
| AbO-180 | GGCGAAATCCCGTGTGCGAGAGACGCGAAGGCAGAT; SEQ ID NO: 444 |
| AbO-181 | TGCCGCAGTGCGTAAGCTACGCGCTCGTTCCATACT; SEQ ID NO: 445 |
| AbO-182 | TGCCAGGGCTCGACCAGTTGGACTCGCAGGACATCA; SEQ ID NO: 446 |
| AbO-183 | GGCGGGTCCAAGTATGCCGACTGCGATCGACAGGAT; SEQ ID NO: 447 |
| AbO-184 | CCTCTCAGCGGTCCGATCGGCTGGAATTCGCCAGCT; SEQ ID NO: 448 |
| AbO-185 | ACATGCGTACTGCGTTCCTTCCGGTCCGCGACGAGT; SEQ ID NO: 449 |
| AbO-186 | CTAGCCTCACGCCCTGCGAACCGACCGTTGCCATCG; SEQ ID NO: 450 |
| AbO-187 | AGATAGTGGGCCATCGGCACTCCGGGCTAAGCCGGT; SEQ ID NO: 451 |
| AbO-188 | CCACGTGCGCCATACACTCCCGACGCCTGTCCTCTC; SEQ ID NO: 452 |
| AbO-189 | AGCTGCCGAATCTACAAGGGCCGACTAGCAGCCTGC; SEQ ID NO: 453 |
| AbO-190 | AGCAGCCTAGGCGCCTTTGTCGGTTTGACTCACGTG; SEQ ID NO: 454 |
| AbO-191 | ATCGCGCCTTGTGTCGTTCCGTGGATCAGCACCGCA; SEQ ID NO: 455 |
| AbO-192 | GACCTACGACTCGTGTGCCGCGCATGTCATGGTGGA; SEQ ID NO: 456 |
| AbO-193 | AACCCTAAGGGCGTTGCGCGATGCACGCGCATGGAG; SEQ ID NO: 457 |
| AbO-194 | CACGGCCTCGGTGGTCGGCCGGAGCATCAGACCGTA; SEQ ID NO: 458 |
| AbO-195 | CGCGTATCCCTTCGGCCTTTCGGTTCGTGTGGTGTT; SEQ ID NO: 459 |
| AbO-196 | TCAGGGTCCCGGTTACGCGACTATAGGAGGAGCCGA; SEQ ID NO: 460 |
| AbO-197 | GAGCCCGCGGTTGTTGATACCGGGACTATGCGCGAC; SEQ ID NO: 461 |
| AbO-198 | AGGGAGCGCGTGGTAGATCCAATTCGATACGGCCGT; SEQ ID NO: 462 |
| AbO-199 | GCACGCAGAGCAATCAACCGATAAGGCACGCCGAGT; SEQ ID NO: 463 |
| AbO-200 | GCCACAGGGCGACTCGGAGGGACTCTGTTCGGATGA; SEQ ID NO: 464 |
| AbO-201 | AGTGTGGCGGACAAGCAGTGGCTCGCGAGGCAGTTC; SEQ ID NO: 465 |
| AbO-202 | AGGTCGACCGGATTCGTTATCCAGCGCAGCGTGGTT; SEQ ID NO: 466 |
| AbO-203 | TCAAATAGCGCTCCCGTGCACAAGGCCGCTCCTAGG; SEQ ID NO: 467 |
| AbO-204 | ACCTGGCCGTTCCGCGACACAGGCACGGGCTACTCT; SEQ ID NO: 468 |
| AbO-205 | GCGGGCTAGCTGAACGTCTTTCCCGGCCACTGCCTA; SEQ ID NO: 469 |
| AbO-206 | AGGCAAGTGGGTACGCGTTAACAGTGAGCGCGGCTC; SEQ ID NO: 470 |
| AbO-207 | TGCTCCTAGGTGGATACATCGCCGCTCGGTCCACCT; SEQ ID NO: 471 |
| AbO-208 | GGCGATAGCGCGTTTACTTTGGTCCGCACGACACGT; SEQ ID NO: 472 |
| AbO-209 | ACGTCGAATGGGTACCGGCGCGCCTACTTCGCTACT; SEQ ID NO: 473 |
| AbO-210 | AAGTGCGATATGCGAGCGCTGTTCCGCATGCCGCAC; SEQ ID NO: 474 |
| AbO-211 | GGCAGGCTGATCGGGACCGGCCCGTAGGTTCGCTTC; SEQ ID NO: 475 |
| AbO-212 | CATGAGCGTGTTGCGTCGGACAAGGAGGGAGTCGCT; SEQ ID NO: 476 |
| AbO-213 | GCTAGGGTGAATCGGGCACGAGGCGTCGCATGTCAT; SEQ ID NO: 477 |
| AbO-214 | AGAGGGCCTACTCGTTGACAGCGCGACGCCACAAGC; SEQ ID NO: 478 |
| AbO-215 | CGGCGTCGGCACGTCGGTCGAACACTATGCGAACAC; SEQ ID NO: 479 |
| AbO-216 | ACTGGTGCTGTAGTTCGGCCCGGCTCCCAGCAAGAA; SEQ ID NO: 480 |
| AbO-217 | AGACGGTCGAATAAGGTAGCGGCGGCCGACTCCGTC; SEQ ID NO: 481 |
| AbO-218 | TATCACATGGCGGACGGGTGCATACGCCCTGCGTTC; SEQ ID NO: 482 |
| AbO-219 | AGGCTCGCGATGAACGGATGCGCTACCGACATGGCT; SEQ ID NO: 483 |
| AbO-220 | GCGCAGTAGCTAAGACGCGCGTTACCATGCCGCCAG; SEQ ID NO: 484 |
| AbO-221 | AGTTGGATCGCGCACGACCGACCGAGGATCGCAGAC; SEQ ID NO: 485 |
| AbO-222 | CGCGGAGCACGGCGTCCACTTAATCGCACGCAGCAA; SEQ ID NO: 486 |
| AbO-223 | TCCCGTTGTCGATCCTACCCGCCGGTGCGGATTATG; SEQ ID NO: 487 |
| AbO-224 | GGTTCGCTGTTATCGGGTGGGAACGACCAGCGGTGC; SEQ ID NO: 488 |
| AbO-225 | ACCACCAACACTCGGATAGTTCTCGCCGTTCCGCCT; SEQ ID NO: 489 |
| AbO-226 | GGTAGGGAGCGCGAGGCATGATAGGAGCGATGTGGT; SEQ ID NO: 490 |
| AbO-227 | GGCAGCTGATGCATTGGCGTGCGCGCCATTACCTCC; SEQ ID NO: 491 |
| AbO-228 | CAAGGCACGCCGCGTTCGGCACGCGACCTACCAGCA; SEQ ID NO: 492 |
| AbO-229 | TTGAGGTCGCCGGAACTACGCGAGTCCGCGAAGAGT; SEQ ID NO: 493 |
| AbO-230 | GCCCTGAAACGTGTAAGCTCCTGCCGCGAACCGTAA; SEQ ID NO: 494 |
| AbO-231 | TATTCCCGCCCGAGACTCACGAGGTCGGTTGCCGTG; SEQ ID NO: 495 |
| AbO-232 | TCAGCCAGCGGAATGGGCGGACCGTTACGGTCAGTA; SEQ ID NO: 496 |
| AbO-233 | CGGCGGAGCGTTCGAGTACAACCTGTCTTGAGCGCG; SEQ ID NO: 497 |
| AbO-234 | CTCATAGTGACAGCGGATCCGCTCACGGCTCCGTGA; SEQ ID NO: 498 |
| AbO-235 | GTAGTCTGCGTACGAGTCCGGGCGGTGCATGTTCGT; SEQ ID NO: 499 |
| AbO-236 | TGACCATGAGCACGTCACCCACGCGCATGCAGTATC; SEQ ID NO: 500 |
| AbO-237 | TGTTTCGGCCTGTGAGCTGCCGGCCCTGTATGCTAG; SEQ ID NO: 501 |
| AbO-238 | CTGCGGACGTTGAGGCGGCTAATGGGAAGACGACCT; SEQ ID NO: 502 |
| AbO-239 | GACAAATTAAGAGTCGCGGCGGACCGCGCCTTGACT; SEQ ID NO: 503 |
| AbO-240 | CAGCAGGGTTAATGCGCGCGCTGGCAACGTTAGCTC; SEQ ID NO: 504 |
| AbO-241 | CCACGAGCCCTCAATGAGGCTGGCCGTCCGAAGATC; SEQ ID NO: 505 |
| AbO-242 | ATCGACGCATTCGGTGTGGCCCGTCTGGTGGTAGCT; SEQ ID NO: 506 |
| AbO-243 | CCTGATGGCGAGGCTTGCGACGGCATTGCTCAGGCT; SEQ ID NO: 507 |
| AbO-244 | CGCCTAGCGCGTGGAGCTGCGCAAAGTGTCACGAAG; SEQ ID NO: 508 |
| AbO-245 | CGGGCCCGACCACGGCATGTTCCATCGTCGGTCACT; SEQ ID NO: 509 |
| AbO-246 | TACATCGCTGGTGTTCACCACGCAGCGAGTACGGCG; SEQ ID NO: 510 |
| AbO-247 | TCCCGAGTCTCTTCAAGGGCGTGGGCGCTTCCAGTT; SEQ ID NO: 511 |
| AbO-248 | CGAAGTCAGCGCTGAATGAGCGGGACCGTTACCGGT; SEQ ID NO: 512 |
| AbO-249 | CGAGTGCTGCCAGCCTGGCTGGCCTAGCGCTTCCAG; SEQ ID NO: 513 |
| AbO-250 | ACGTCAGAATGCTGGGCAGCCTCAAGGCTCGGAGCC; SEQ ID NO: 514 |
| AbO-251 | TACCGTTCACAACCACAGGGCGCGAGCGCAACCGCG; SEQ ID NO: 515 |
| AbO-252 | TCGACACCGAGTTTGCCCTCCTATTTGCTGGCGGCG; SEQ ID NO: 516 |
| AbO-253 | GTCCGTTAGATTGGAGTTCTGTCAGCGCCCTCGCGC; SEQ ID NO: 517 |
| AbO-254 | GTCGCCGAACACCCTGACTTCCACCGTGTGTTCCGA; SEQ ID NO: 518 |
| AbO-255 | CCGGGATAGCTCCACCTGAGTAGCCGCGCCATCTAC; SEQ ID NO: 519 |
| AbO-256 | TGCATGTTTGGTGTGGATGCCACAGCTCAGCGCGAG; SEQ ID NO: 520 |
| AbO-257 | GGTCAGGGAGCTCGTGCCAAGACCCACAGACCGTCC; SEQ ID NO: 521 |
| AbO-258 | TACGGTCAAGATCACCAGGCCGGCTGCGTCTTGTCA; SEQ ID NO: 522 |
| AbO-259 | AAAGACTATTGCCCAGCGGGCGGGCTGACTGAAGGC; SEQ ID NO: 523 |
| AbO-260 | TGGCTGCCACGCATTGAAGCAAAGACGCGAAGACGG; SEQ ID NO: 524 |
| AbO-261 | TTGGCTGGATCCCAAGTCTGGCCCGAGGGACGGTAC; SEQ ID NO: 525 |
| AbO-262 | ACCGGCCTGCATTTCAGCTCTCGGAGCCTGAAGCTG; SEQ ID NO: 526 |
| AbO-263 | GTTGAACGCACCCGGGCTTCCTCTGGGAATCGCAGA; SEQ ID NO: 527 |
| AbO-264 | ATCACGGACACCCAAGTGGCGCGCCTCTTACCACGC; SEQ ID NO: 528 |
| AbO-265 | CCGAGACAGTGCACGCCTCACGTGGACAGCTGCCAT; SEQ ID NO: 529 |
| AbO-266 | TGCCAGGGCTCGACCAGTTGGACTCGCAGGACATCA; SEQ ID NO: 584 |
| AbO-267 | CGCCTAGCGCGTGGAGCTGCGCAAAGTGTCACGAAG; SEQ ID NO: 585 |
| AbO-268 | CGAGTTGGGCGTCAATGTCGGCCGTCATGATGCACC; SEQ ID NO: 586 |
| AbO-269 | TGACCATGAGCACGTCACCCACGCGCATGCAGTATC; SEQ ID NO: 587 |

TABLE 2
Effective probe oligonucleotide sequences. It is understood that white space,
line breaks, and text formatting are not indicative of separate sequences or
structural implications.

| Probe Oligo | LS Sequence (3′-5′) | RS Sequence (3′-5′) |
|---|---|---|
| ProProbe-1 | CAGAGCTTCGGGATAGAT; | GGGAGGGATAGATTTGAG; |
| | SEQ ID NO: 133 | SEQ ID NO: 265 |
| ProProbe-2 | CGAGGTGAGTTGGTCTGA; | GGGTATGGTAAGATGTCG; |
| | SEQ ID NO: 134 | SEQ ID NO: 266 |
| ProProbe-3 | TCAGGCGACGTTAGGATT; | AGGCTAGTATGTTGTGGC; |
| | SEQ ID NO: 135 | SEQ ID NO: 267 |
| ProProbe-4 | ATGGACGGCTAGGGTGTG; | TGGCAATAGTGGTAAGAG; |
| | SEQ ID NO: 136 | SEQ ID NO: 268 |
| ProProbe-5 | CCTTCAAGTCCAGCCTAT; | ACCCATCCCAAGTCACAG; |
| | SEQ ID NO: 137 | SEQ ID NO: 269 |
| ProProbe-6 | CATTTCACGGCACATGGG; | AGAGGGAAGCTGGGAATA; |
| | SEQ ID NO: 138 | SEQ ID NO: 270 |
| ProProbe-7 | AAGGAGCTCGGAATTGGC; | TGGATAGAGTTTGTGGCG; |
| | SEQ ID NO: 139 | SEQ ID NO: 271 |
| ProProbe-8 | CCGTCAGAATGCGATTGG; | GTTGGTTAGATGGGTTGC; |
| | SEQ ID NO: 140 | SEQ ID NO: 272 |
| ProProbe-9 | ATTACGAGCCAGGCGTTG; | TGATGTATGTTGGTTGCG; |
| | SEQ ID NO: 141 | SEQ ID NO: 273 |
| ProProbe-10 | GACGGGTGAATTGGCGAG; | AGACAGCAAGTAGTGGTA; |
| | SEQ ID NO: 142 | SEQ ID NO: 274 |
| ProProbe-11 | CTCATTCTCGGGTTGGTT; | CGTAAGGGATTTGCTCAG; |
| | SEQ ID NO: 143 | SEQ ID NO: 275 |
| ProProbe-12 | GGAGGCTGTATGGTATGG; | CTAAACTGATTGGCTTGG; |
| | SEQ ID NO: 144 | SEQ ID NO: 276 |
| ProProbe-13 | GGTGGCGAGAGTAGATTA; | GTCTTTGTGCTGCGGTCT; |
| | SEQ ID NO: 145 | SEQ ID NO: 277 |
| ProProbe-14 | GGTAGTGTGCTGGTCAAT; | GCCGTTAGAGTGGTGTTC; |
| | SEQ ID NO: 146 | SEQ ID NO: 278 |
| ProProbe-15 | GACATCGGTGGCTAGTTT; | CTGCGTTGGTGGATTAGG; |
| | SEQ ID NO: 147 | SEQ ID NO: 279 |
| ProProbe-16 | TTCCCTCCAGCCATCCTA; | AGCCTCTGTTCCAAGTCC; |
| | SEQ ID NO: 148 | SEQ ID NO: 280 |
| ProProbe-17 | TAGGGTGCGATCAGTGTG; | TGGGAAGAGATACGGCAA; |
| | SEQ ID NO: 149 | SEQ ID NO: 281 |
| ProProbe-18 | GCCCTATCACGTTCATGC; | CTGTCTATCCCATTCTAG; |
| | SEQ ID NO: 150 | SEQ ID NO: 282 |
| ProProbe-19 | TTTCCCGACAAGCCAAGT; | AGATGAATAGCCGACGTC; |
| | SEQ ID NO: 151 | SEQ ID NO: 283 |
| ProProbe-20 | CCCTCATACCACGCAGTC; | CAGGCATAGCTACAGAAT; |
| | SEQ ID NO: 152 | SEQ ID NO: 284 |
| ProProbe-21 | TAGGCTGATCGACGGATT; | GTGTACTTGGGTGATACG; |
| | SEQ ID NO: 153 | SEQ ID NO: 285 |
| ProProbe-22 | GTCTTAGTTGCTCGAGGT; | GCTGTGCGTGTTGGTTCT; |
| | SEQ ID NO: 154 | SEQ ID NO: 286 |
| ProProbe-23 | GATAGGTGAGGCTAGGAT; | TTGACGGACGCTCGCTTA; |
| | SEQ ID NO: 155 | SEQ ID NO: 287 |
| ProProbe-24 | CACACCCACCAGATGTAT; | ACCAACGCTCAGAACCTC; |
| | SEQ ID NO: 156 | SEQ ID NO: 288 |
| ProProbe-25 | GAGGTGAGGACAAGCTGC; | TGAATGGTTAGGTTGGCG; |
| | SEQ ID NO: 157 | SEQ ID NO: 289 |
| ProProbe-26 | ATGGAGAAGCGTTGTGGT; | GATGGGAGCTTGTTCAGA; |
| | SEQ ID NO: 158 | SEQ ID NO: 290 |
| ProProbe-27 | CAACATAACCCACAGCGC; | TTATCCATCACCTCTAGC; |
| | SEQ ID NO: 159 | SEQ ID NO: 291 |
| ProProbe-28 | CCACTCTACGCCATCTTG; | ATGCACAACCTTAACCCG; |
| | SEQ ID NO: 160 | SEQ ID NO: 292 |
| ProProbe-29 | TTGTGAGAGAACCAGGGC; | AAATGAGGCTAAAGTGGC; |
| | SEQ ID NO: 161 | SEQ ID NO: 293 |
| ProProbe-30 | ATCCCTCCTCTATCAGCT; | TGTGCTCTCCGACCTACT; |
| | SEQ ID NO: 162 | SEQ ID NO: 294 |
| ProProbe-31 | GTAGGGAAGGAGCGTATA; | CTAGTGCGTGAAGGTCTC; |
| | SEQ ID NO: 163 | SEQ ID NO: 295 |
| ProProbe-32 | GGCTTCTAGGTGGCTTGC; | GTTGGATTCTGGGAAGGT; |
| | SEQ ID NO: 164 | SEQ ID NO: 296 |
| ProProbe-33 | CTAATCCCAACTCGAGCA; | CAGTCATAGTCCAAGCAC; |
| | SEQ ID NO: 165 | SEQ ID NO: 297 |
| ProProbe-34 | TCGGTATAGGGTGCGGTG; | AAGCCTTGGATCTAAACG; |
| | SEQ ID NO: 166 | SEQ ID NO: 298 |
| ProProbe-35 | AGAGCACGGGTAAGGTAG; | GACGTATGAAGAGCTTTG; |
| | SEQ ID NO: 167 | SEQ ID NO: 299 |
| ProProbe-36 | CGATGCTGATAGGTACGC; | TGAGTGGTTGGAAGACGT; |
| | SEQ ID NO: 168 | SEQ ID NO: 300 |
| ProProbe-37 | GGTATAGCACGGTAGGTC; | ATGCGCTTAGGTGTTGAA; |
| | SEQ ID NO: 169 | SEQ ID NO: 301 |
| ProProbe-38 | ACGGTGGCTGGACATAGT; | TGGATCGACTGCGCTTAA; |
| | SEQ ID NO: 170 | SEQ ID NO: 302 |
| ProProbe-39 | TCCCATCCACAGTCGCTT; | CCAGATCTTCGCACGTCT; |
| | SEQ ID NO: 171 | SEQ ID NO: 303 |
| ProProbe-40 | TTGGGAATGGCGAAGACA; | GTACGGCAGGATCGAAAC; |
| | SEQ ID NO: 172 | SEQ ID NO: 304 |
| ProProbe-41 | GAGATCGTGTGCGCTATC; | ATTGTGGTGAAGGAAGGT; |
| | SEQ ID NO: 173 | SEQ ID NO: 305 |
| ProProbe-42 | GGACAGATATGGAGCCGT; | TCGAAGAGCAGCGTAGAC; |
| | SEQ ID NO: 174 | SEQ ID NO: 306 |
| ProProbe-43 | GAACGACGGCATGGAACT; | AGCTTGAGACACGGTTAC; |
| | SEQ ID NO: 175 | SEQ ID NO: 307 |
| ProProbe-44 | CTCGACGTTATGGTTGGG; | CTGCTTGTTGTGCTTTCT; |
| | SEQ ID NO: 176 | SEQ ID NO: 308 |
| ProProbe-45 | CGTATAGGAGGATCAGGC; | CAAGGTAGGATCTAGTGG; |
| | SEQ ID NO: 177 | SEQ ID NO: 309 |
| ProProbe-46 | GGGACGTTGCTGGCTATT; | GCGGATGATCATTGGTGG; |
| | SEQ ID NO: 178 | SEQ ID NO: 310 |
| ProProbe-47 | CGCGTCTAGGTTGATAGT; | TGCTGTTTGAGGGTTGTG; |
| | SEQ ID NO: 179 | SEQ ID NO: 311 |
| ProProbe-48 | GGAGTCATTGTCGTGCGA; | TCTGGTGCCTGCTGATTG; |
| | SEQ ID NO: 180 | SEQ ID NO: 312 |
| ProProbe-49 | GTTGGGTTACGGTGCATA; | CGATGATCTTGGCGTGGA; |
| | SEQ ID NO: 181 | SEQ ID NO: 313 |
| ProProbe-50 | GATTGGTGCTGGGCTTGA; | ATAGGCGATCTAAGGTGG; |
| | SEQ ID NO: 182 | SEQ ID NO: 314 |
| ProProbe-51 | CCCACTTATCCTTCCAGC; | TACACTACTCTGTGCCCT; |
| | SEQ ID NO: 183 | SEQ ID NO: 315 |
| ProProbe-52 | ATACGGGAACTCGCATGG; | ATCTGTTTGGCCTTGGTG; |
| | SEQ ID NO: 184 | SEQ ID NO: 316 |
| ProProbe-53 | TGTACAGTCAGCACCCAC; | ACTAGAAATCACACGGTC; |
| | SEQ ID NO: 185 | SEQ ID NO: 317 |
| ProProbe-54 | CGGCAGGTCTAGTGTAAC; | TTCGCTGATGTTTAGAGG; |
| | SEQ ID NO: 186 | SEQ ID NO: 318 |
| ProProbe-55 | CGGGAGTATGCTGCATAG; | CGTTGATGTGTGGAATGA; |
| | SEQ ID NO: 187 | SEQ ID NO: 319 |
| ProProbe-56 | ACACCCATAACTCCCAGC; | GCACAACAAACGTAGCAT; |
| | SEQ ID NO: 188 | SEQ ID NO: 320 |
| ProProbe-57 | CATGTGGATCGCATTCCT; | GGTTAGTTTGAGGTTGGG; |
| | SEQ ID NO: 189 | SEQ ID NO: 321 |
| ProProbe-58 | TCCGTGGTGAGTAGTACA; | GTAGCGAGTCTGAAGTTC; |
| | SEQ ID NO: 190 | SEQ ID NO: 322 |
| ProProbe-59 | CGGCTTTCTCTTGTGGCA; | TGGGTGGTTGTTCGAGAA; |
| | SEQ ID NO: 191 | SEQ ID NO: 323 |
| ProProbe-60 | CGCTTCCACGCTCCTATA; | CCAAACGCCAACAGACCA; |
| | SEQ ID NO: 192 | SEQ ID NO: 324 |
| ProProbe-61 | TTGATCGAGCACACCCAT; | TAGCAAACACCTCCACGT; |
| | SEQ ID NO: 193 | SEQ ID NO: 325 |
| ProProbe-62 | TCACCCATACCTGCAGCC; | TTACGTGCCCTTCAAATC; |
| | SEQ ID NO: 194 | SEQ ID NO: 326 |
| ProProbe-63 | CGACTCATCATGCAAGCC; | AAGACCCACGTAACTCAT; |
| | SEQ ID NO: 195 | SEQ ID NO: 327 |
| ProProbe-64 | GTGGAGAAGGCAAGTCAG; | AGCTAGTGTCACCTTAGA; |
| | SEQ ID NO: 196 | SEQ ID NO: 328 |
| ProProbe-65 | TGGTAGGATCGGAACGTG; | GACAATAGCAACCTGACT; |
| | SEQ ID NO: 197 | SEQ ID NO: 329 |
| ProProbe-66 | CATAGTAGCCACCGTCAT; | CAGCACATAAGCCACATC; |
| | SEQ ID NO: 198 | SEQ ID NO: 330 |
| ProProbe-67 | TCGCTCCATAGTGCTGTA; | GATGTGTATTCGCCTAGG; |
| | SEQ ID NO: 199 | SEQ ID NO: 331 |
| ProProbe-68 | CACCGACAGTCTATCCGA; | GCCATTTCAACATCCTCC; |
| | SEQ ID NO: 200 | SEQ ID NO: 332 |
| ProProbe-69 | CACGCATGTCCAATAGCA; | AGGTTTCGGCAGGTAATG; |
| | SEQ ID NO: 201 | SEQ ID NO: 333 |
| ProProbe-70 | CCTCTACAGCCTGCAAAC; | GTCACATATCTGCCACAC; |
| | SEQ ID NO: 202 | SEQ ID NO: 334 |
| ProProbe-71 | CGGGTGAACTACTAAGGA; | TGGCTACAACGGGACTAG; |
| | SEQ ID NO: 203 | SEQ ID NO: 335 |
| ProProbe-72 | CGTAGATCACAGGTAGGT; | CTTATGCGAGGAGGGACT; |
| | SEQ ID NO: 204 | SEQ ID NO: 336 |
| ProProbe-73 | GGCAAAGGACGGGACAAT; | TGGGATGGTTCGATGTTC; |
| | SEQ ID NO: 205 | SEQ ID NO: 337 |
| ProProbe-74 | CGACGTGCTAGAGATGTA; | CTGCTGCCAAGACTCAAT; |
| | SEQ ID NO: 206 | SEQ ID NO: 338 |
| ProProbe-75 | TCACCTCGCCTTCAAACC; | CTATCGCACGTATTGTCT; |
| | SEQ ID NO: 207 | SEQ ID NO: 339 |
| ProProbe-76 | TGGGTACGAGAACCATGC; | CGAGATATGTACTGAGAG; |
| | SEQ ID NO: 208 | SEQ ID NO: 340 |
| ProProbe-77 | CAGGTACATGCATAGCCA; | GATCGTGCCCAGACTCAT; |
| | SEQ ID NO: 209 | SEQ ID NO: 341 |
| ProProbe-78 | GTGCAATTGGTTCCGCTC; | GATAACTCCCTCTCATAC; |
| | SEQ ID NO: 210 | SEQ ID NO: 342 |
| ProProbe-79 | GTGTTTGGTTCTCGCGTT; | GTAGCAGCGGTGTATGGA; |
| | SEQ ID NO: 211 | SEQ ID NO: 343 |
| ProProbe-80 | TTTCCCTCACCTGCGATC; | ACACAGTTTAGCTACTCG; |
| | SEQ ID NO: 212 | SEQ ID NO: 344 |
| ProProbe-81 | CTTGGCGTTCGTAGGTTG; | GCCTGTTGATGAGTAGTA; |
| | SEQ ID NO: 213 | SEQ ID NO: 345 |
| ProProbe-82 | AGGAGTATTGCGAGCTGA; | ATGTACACTGCGAACCCA; |
| | SEQ ID NO: 214 | SEQ ID NO: 346 |
| ProProbe-83 | CGGCTGAGTTATGAGGTG; | CGATCGGCTTGTTTACTG; |
| | SEQ ID NO: 215 | SEQ ID NO: 347 |
| ProProbe-84 | CCTTCCAATTCCGCACAT; | CCTCAATGAGTTACCTCG; |
| | SEQ ID NO: 216 | SEQ ID NO: 348 |
| ProProbe-85 | TCTCAACCTCACACGGCT; | AAGCTGCACATGTTCCTC; |
| | SEQ ID NO: 217 | SEQ ID NO: 349 |
| ProProbe-86 | ATATCCGCTTGCCCAACC; | TAATCGATGCCTGTGAGC; |
| | SEQ ID NO: 218 | SEQ ID NO: 350 |
| ProProbe-87 | CAGACCATGCTACCAGTA; | ATGCAACAATCACGAGGC; |
| | SEQ ID NO: 219 | SEQ ID NO: 351 |
| ProProbe-88 | GGATTGCGATAGGCCTTG; | GCTGACTAGTGGATGTTG; |
| | SEQ ID NO: 220 | SEQ ID NO: 352 |
| ProProbe-89 | CGAGACTATCCACACAGC; | ATTGCATCTACCATCACC; |
| | SEQ ID NO: 221 | SEQ ID NO: 353 |
| ProProbe-90 | GGCTCCTACTATCCTGGG; | CTGTTCAATGGGCTGGTT; |
| | SEQ ID NO: 222 | SEQ ID NO: 354 |
| ProProbe-91 | GGGCTCCGGATATGTATT; | GGATGAGTGCGTGTCGTT; |
| | SEQ ID NO: 223 | SEQ ID NO: 355 |
| ProProbe-92 | GGTTCTCTGGCGATGTGA; | CTTTGCTAGGGTTGCTCA; |
| | SEQ ID NO: 224 | SEQ ID NO: 356 |
| ProProbe-93 | ACAACTGCCAGCCATTCA; | CATATCAGGGTAGCTCAG; |
| | SEQ ID NO: 225 | SEQ ID NO: 357 |
| ProProbe-94 | CATGCCAGCAGTGATAGA; | ATTGCGATACGGTGACTG; |
| | SEQ ID NO: 226 | SEQ ID NO: 358 |
| ProProbe-95 | TACGCACAACCAAGCACA; | AGATCAACCCTGCAAGAC; |
| | SEQ ID NO: 227 | SEQ ID NO: 359 |
| ProProbe-96 | TCGTTCTATCCTGGCCTC; | TTGAGCAATTCGACAGTG; |
| | SEQ ID NO: 228 | SEQ ID NO: 360 |
| ProProbe-97 | CCAGTCACTGCCGAACTC; | TATACTCCAAGCCGAACA; |
| | SEQ ID NO: 229 | SEQ ID NO: 361 |
| ProProbe-98 | GCCACCCACCCATCAATG; | TACCAACGGACTATAGCT; |
| | SEQ ID NO: 230 | SEQ ID NO: 362 |
| ProProbe-99 | GGCGTTCGATAGGTACAT; | CAGGCAGCTTAAGACACT; |
| | SEQ ID NO: 231 | SEQ ID NO: 363 |
| ProProbe-100 | GTGGTCGTTGGAATGGGC; | AGGATGTCTGGTACTACG; |
| | SEQ ID NO: 232 | SEQ ID NO: 364 |
| ProProbe-101 | TTGAGATCGAGGTGCCAA; | TCCGGTTGCTTGACTAGG; |
| | SEQ ID NO: 233 | SEQ ID NO: 365 |
| ProProbe-102 | CCGATTGAGATGAGGGTA; | TTGGATCGCACTCTGACG; |
| | SEQ ID NO: 234 | SEQ ID NO: 366 |
| ProProbe-103 | CGAGACTAGGATTGCTGC; | CACCTGAGCTTACAATGA; |
| | SEQ ID NO: 235 | SEQ ID NO: 367 |
| ProProbe-104 | CTGTGAGGGTAGCTTGTT; | GGTTAGCAGGTAGGAAAC; |
| | SEQ ID NO: 236 | SEQ ID NO: 368 |
| ProProbe-105 | GAACTCTGTCGCACGTAC; | GATGATTGAGGGATTGGC; |
| | SEQ ID NO: 237 | SEQ ID NO: 369 |
| ProProbe-106 | CCTATTCCAGACCGCTTC; | TATCCCAGGCTTGATTCA; |
| | SEQ ID NO: 238 | SEQ ID NO: 370 |
| ProProbe-107 | CTGGCTAGGATCTACTGC; | TGCGCTTTGTTGGAAGGT; |
| | SEQ ID NO: 239 | SEQ ID NO: 371 |
| ProProbe-108 | CCACCAGATACCGCTGAC; | ATACTCCTCGTTCCATCC; |
| | SEQ ID NO: 240 | SEQ ID NO: 372 |
| ProProbe-109 | CGTCCTAGCTTTGAGTCA; | TACCCTCCAGATACGCAT; |
| | SEQ ID NO: 241 | SEQ ID NO: 373 |
| ProProbe-110 | CTCCAGCGACACCATATC; | AATCTGCCTTATCCAGTC; |
| | SEQ ID NO: 242 | SEQ ID NO: 374 |
| ProProbe-111 | TCCAGATTACCTCCTCCT; | GCATTAGTGTAGTCAGCG; |
| | SEQ ID NO: 243 | SEQ ID NO: 375 |
| ProProbe-112 | CTTGTTCCGGATGGCAAT; | CTCTCAACTGTCAGGTTC; |
| | SEQ ID NO: 244 | SEQ ID NO: 376 |
| ProProbe-113 | GCATCCACGCTCTTAACT; | CGCACATACAAACGGACT; |
| | SEQ ID NO: 245 | SEQ ID NO: 377 |
| ProProbe-114 | TACCATACGACAAGCCCT; | ACATTACCCTCCTGGACT; |
| | SEQ ID NO: 246 | SEQ ID NO: 378 |
| ProProbe-115 | GGTGGAAGGTGCTCACTC; | AACTGGTATCGATCTTGC; |
| | SEQ ID NO: 247 | SEQ ID NO: 379 |
| ProProbe-116 | CGGTTCTGTTCTGTGCTT; | CGGTAGGGTGGCATGAAT; |
| | SEQ ID NO: 248 | SEQ ID NO: 380 |
| ProProbe-117 | TCGGGATCAAGACAGCTT; | TCATACCTACCCTCGCAA; |
| | SEQ ID NO: 249 | SEQ ID NO: 381 |
| ProProbe-118 | CCCTCTCACTGAATTCGG; | CTTCACTCTAGATCCTCC; |
| | SEQ ID NO: 250 | SEQ ID NO: 382 |
| ProProbe-119 | ATAGCCGACTCACCTCTC; | ATGGATCTAAGCAGCCAA; |
| | SEQ ID NO: 251 | SEQ ID NO: 383 |
| ProProbe-120 | CATCGACTAGGACGCTAT; | GCCACTCTATCTGTAAGG; |
| | SEQ ID NO: 252 | SEQ ID NO: 384 |
| ProProbe-121 | ATCTCAGGGCTACTCGTT; | CAGTCTCGCTCTAACACA; |
| | SEQ ID NO: 253 | SEQ ID NO: 385 |
| ProProbe-122 | GGCGTTTCATCCAGGTTG; | TATCGTCAGCACTAGTTG; |
| | SEQ ID NO: 254 | SEQ ID NO: 386 |
| ProProbe-123 | CTTCACACCTCCAAGCAG; | ATCACTGGCCAACTCAAC; |
| | SEQ ID NO: 255 | SEQ ID NO: 387 |
| ProProbe-124 | CAGCCTACATAACCTGCA; | GCTACTATACCTCAGACC; |
| | SEQ ID NO: 256 | SEQ ID NO: 388 |
| ProProbe-125 | GATGGTGTCTGGAGCTCG; | TGGCAAGTTGTGATGGTC; |
| | SEQ ID NO: 257 | SEQ ID NO: 389 |
| ProProbe-126 | AAAGGGCAGGGTAATGGC; | ATTAGGAGCGACTGTTGA; |
| | SEQ ID NO: 258 | SEQ ID NO: 390 |
| ProProbe-127 | CGTGAGGAATTGTGGCTA; | GACCGGTGCGTATGATCT; |
| | SEQ ID NO: 259 | SEQ ID NO: 391 |
| ProProbe-128 | CCGCACAAACCAGGGATA; | CAGGATCGGCTTTAGAAC; |
| | SEQ ID NO: 260 | SEQ ID NO: 392 |
| ProProbe-129 | AGATCAATCCACCGGTCA; | TATGAGTACGCTGCCAAG; |
| | SEQ ID NO: 261 | SEQ ID NO: 393 |
| ProProbe-130 | GGTTTGCTTCGCCATGAG; | TCCTGAGTGTTCGTTAGA; |
| | SEQ ID NO: 262 | SEQ ID NO: 394 |
| ProProbe-131 | GATCTCCCTCCGCACTTT; | ACATCTCGCTTGCCTAGT; |
| | SEQ ID NO: 263 | SEQ ID NO: 395 |
| ProProbe-132 | TAGCACCAACGATCCAGA; | ATCCGCACCGATCAAAGT; |
| | SEQ ID NO: 264 | SEQ ID NO: 396 |
| ProProbe-133 | ACGGTCCCGAGCTGGTCA; | ACCTGAGCGTCCTGTAGT; |
| | SEQ ID NO: 588 | SEQ ID NO: 592 |
| ProProbe-134 | GCGGATCGCGCACCTCGA; | CGCGTTTCACAGTGCTTC; |
| | SEQ ID NO: 589 | SEQ ID NO: 593 |
| ProProbe-135 | GCTCAACCCGCAGTTACA; | GCCGGCAGTACTACGTGG; |
| | SEQ ID NO: 590 | SEQ ID NO: 594 |
| ProProbe-136 | ACTGGTACTCGTGCAGTG; | GGTGCGCGTACGTCATAG; |
| | SEQ ID NO: 591 | SEQ ID NO: 595 |

Out of the initial 5,000,000 random oligonucleotide sequences, only a selection of sequences satisfied the selection criteria (e.g., melting temps, GC content, secondary structure, etc.) and performed well during initial studies, resulting in a significant amount of detectable clusters under the tested conditions. It is understood that the complementary and reverse complementary sequences of the sequences identified in Tables 1 and 2 perform equally as well. Those not explicitly depicted in the Table(s), the 5′ end, for example, the 5′ end of the LS sequence may be phosphorylated. The polynucleotide may be phosphorylated prior to or post hybridization using common phosphorylation techniques known in the art. (e.g., T4 PNK phosphorylation). In embodiments, a phosphate group is present at the 5′ end of one strand and a hydroxyl group at the 3′ end of the adjacent strand, for example, when using enzymatic ligation. The DNA ligase enzyme acts by forming a covalent bond between these two groups. In many cases, DNA strands, particularly synthetic oligonucleotides, are not naturally equipped with a 5′ phosphate group. Therefore, they must be phosphorylated to provide the necessary substrate for the ligase enzyme. Alternatively, ligation of the two ends together may include chemical ligation (e.g., enzyme-free, click-mediated ligation). In embodiments, the polynucleotide probe includes a first bioconjugate reactive moiety on one end that is capable of bonding upon contact with a second (complementary) bioconjugate reactive moiety provided on the second end. For example, the polynucleotide probe includes an alkynyl moiety at the 3′ and an azide moiety at the 5′ end that, upon hybridization to the target nucleic acid react to form a triazole linkage during suitable reaction conditions. Reaction conditions and protocols for chemical ligation techniques that are compatible with nucleic acid amplification methods are known in the art, for example El-Sagheer, A. H., & Brown, T. (2012). Accounts of chemical research, 45 (8), 1258-1267; Manuguerra I. et al. Chem Commun (Camb). 2018; 54 (36): 4529-4532; and Odeh, F., et al. (2019). Molecules (Basel, Switzerland), 25 (1), 3, each of which is incorporated herein by reference in their entirety.

The polynucleotides described herein or polynucleotide probes described herein may further include a primer binding sequence. For example, a sequencing primer binding sequence may be included to enable subsequent hybridization of a sequencing or amplification primer. Provided in Table 3 are some effective primer binding sequences useful for including in the polynucleotide backbone of a polynucleotide probe described herein.

| Internal Name | Sequence (5′->3′) | SEQ ID Num. |
|---|---|---|
| S1 | ACAAAGGCAGCCACG CACTCCTTCCCTGT | SEQ ID NO: 530 |
| SP1 | ACACTCTTTCCCTACACGACGCTCTTCCGATC | SEQ ID NO: 531 |
| S2 | CTCCAGCGAGATGACC CTCACCAACCACT | SEQ ID NO: 532 |
| SP2 | GTGACTGGAGTTCAGA CGTGTGCTCTTCCGATC | SEQ ID NO: 533 |
| P5 | AATGATACGGCGACCACCG | SEQ ID NO: 534 |
| P7 | CAAGCAGAAGACGGCATACGAGAT | SEQ ID NO: 535 |
| MIA | AACGCCAAACCTACGGCTTTACTTCCTGTGGC | SEQ ID NO: 536 |
| M2A | TCTTGAGTCATTCGCAGGGCATGTGCCAGACC | SEQ ID NO: 537 |
| M3A | TCGGCGTTGTCTGCTATCGTTCTTGGCACTCC | SEQ ID NO: 538 |
| M4A | GGAGCAATAACCATAAGGCCGTTGACAAGCCC | SEQ ID NO: 539 |
| M5A | GGCGTATTGCCTTGGTTCTGGCAGCCTCATTG | SEQ ID NO: 540 |
| M1B | CAGCAGAGGGAACGATTTCAACTTCCTGTGGC | SEQ ID NO: 541 |
| M2B | CTACTGCAAGGGTGTCTAGAATGTGCCAGACC | SEQ ID NO: 542 |
| M3B | GACCGACTCGTGAAACGTAATCTTGGCACTCC | SEQ ID NO: 543 |
| M4B | ACACATTCTTTGCGCCCAGAGTTGACAAGCCC | SEQ ID NO: 544 |
| M5B | ATTTCATTCGACACCCGGTCGCAGCCTCATTG | SEQ ID NO: 545 |
| M1A_RC | AGCCACAGGAAGTAAAGCCGTAGGTTTGGCGT | SEQ ID NO: 546 |
| M2A_RC | AGGTCTGGCACATGCCCTGCGAATGACTCAAGA | SEQ ID NO: 547 |
| M3A_RC | AGGAGTGCCAAGAACGATAGCAGACAACGCCGA | SEQ ID NO: 548 |
| M4A_RC | AGGGCTTGTCAACGGCCTTATGGTTATTGCTCC | SEQ ID NO: 549 |
| M5A_RC | ACAATGAGGCTGCCAGAACCAAGGCAATACGCC | SEQ ID NO: 550 |
| MIB_RC | AGCCACAGGAAGTTGAAATCGTTCCCTCTGCTG | SEQ ID NO: 551 |
| M2B_RC | AGGTCTGGCACATTCTAGACACCCTTGCAGTAG | SEQ ID NO: 552 |
| M3B_RC | AGGAGTGCCAAGATTACGTTTCACGAGTCGGTC | SEQ ID NO: 553 |
| M4B_RC | AGGGCTTGTCAACTCTGGGCGCAAAGAATGTGT | SEQ ID NO: 554 |
| M5B_RC | ACAATGAGGCTGCGACCGGGTGTCGAATGAAAT | SEQ ID NO: 555 |
| M6A | TGTTGCATCTCCACCCGGATTGAGCCTTCAGC | SEQ ID NO: 556 |
| M7A | CACAACGGGAGCTGTGGAATTGGTTCACCTGG | SEQ ID NO: 557 |
| M8A | TGGACTAAGACTCGTCCTCCAGCGGACCTAAG | SEQ ID NO: 558 |
| M9A | GTATGATGGTGTTGCGGCTTCTCGCTTAACGC | SEQ ID NO: 559 |
| M10A | TCTGAGTGCCAGTGACTTCACGCATTCGCTTG | SEQ ID NO: 560 |
| M11A | TACGACACACTCGGGCTCTATGGGCTTCATGG | SEQ ID NO: 561 |
| M12A | GTTTGAGTGAAGGCGGTCCAACCCTTAGTGCG | SEQ ID NO: 562 |
| M6B | CTATAAGTTTGTCGTGCCCGTGAGCCTTCAGC | SEQ ID NO: 563 |
| M7B | GGAGTGACACTGACTACGTTTGGTTCACCTGG | SEQ ID NO: 564 |
| M8B | GTCAACGCCCTAGCAGACATAGCGGACCTAAG | SEQ ID NO: 565 |
| M9B | CCAGAACCTATTGAGCCTGACTCGCTTAACGC | SEQ ID NO: 566 |
| M10B | AGGTGTTCGTACAATGAGGCCGCATTCGCTTG | SEQ ID NO: 567 |
| M11B | TGGTCAAGGGCAACTAATCCTGGGCTTCATGG | SEQ ID NO: 568 |
| M12B | ACAATTACCCGTTTACCGGCACCCTTAGTGCG | SEQ ID NO: 569 |
| M6A_RC | AGCTGAAGGCTCAATCCGGGTGGAGATGCAACA | SEQ ID NO: 570 |
| M7A_RC | ACCAGGTGAACCAATTCCACAGCTCCCGTTGTG | SEQ ID NO: 571 |
| M8A_RC | ACTTAGGTCCGCTGGAGGACGAGTCTTAGTCCA | SEQ ID NO: 572 |
| M9A_RC | AGCGTTAAGCGAGAAGCCGCAACACCATCATAC | SEQ ID NO: 573 |
| M10A_RC | ACAAGCGAATGCGTGAAGTCACTGGCACTCAGA | SEQ ID NO: 574 |
| M11A_RC | ACCATGAAGCCCATAGAGCCCGAGTGTGTCGTA | SEQ ID NO: 575 |
| M12A_RC | ACGCACTAAGGGTTGGACCGCCTTCACTCAAAC | SEQ ID NO: 576 |
| M6B_RC | AGCTGAAGGCTCACGGGCACGACAAACTTATAG | SEQ ID NO: 577 |
| M7B_RC | ACCAGGTGAACCAAACGTAGTCAGTGTCACTCC | SEQ ID NO: 578 |
| M8B_RC | ACTTAGGTCCGCTATGTCTGCTAGGGCGTTGAC | SEQ ID NO: 579 |
| M9B_RC | AGCGTTAAGCGAGTCAGGCTCAATAGGTTCTGG | SEQ ID NO: 580 |
| M10B_RC | ACAAGCGAATGCGGCCTCATTGTACGAACACCT | SEQ ID NO: 581 |
| M11B_RC | ACCATGAAGCCCAGGATTAGTTGCCCTTGACCA | SEQ ID NO: 582 |
| M12B_RC | ACGCACTAAGGGTGCCGGTAAACGGGTAATTGT | SEQ ID NO: 583 |

### Example 2. Protein-Specific Binding Agents

As described herein, the proteins of interest are targeted using probes specific for their intended target. The probe includes a binding moiety capable of binding the target biomolecule, wherein the binding moiety is a specific binding reagent. For example, the binding moiety may be an antibody, single-chain Fv fragment (scFv), antibody fragment-antigen binding (Fab), affimer, or an aptamer. An embodiment contemplated herein uses specific oligonucleotide sequences attached to antibodies, referred to as Ab-oligo conjugates. See FIG. 1 for an illustration of the Ab-oligo conjugate (e.g., specific binding agent described herein).

Monoclonal antibodies are synthesized by injecting an antigen into an animal, triggering an immune response. The animal spleen cells are fused with malignant myeloma cells to form hybridomas, which are both immortal and capable of producing homogeneous antibodies targeting a single epitope. Monoclonal antibodies are specific to that epitope and provide a stable, long-term supply, differentiating them from polyclonal antibodies. Polyclonal antibodies are typically produced by the inoculation of a suitable mammal, such as a mouse, rabbit, or goat. An antigen is injected into the mammal, typically over several weeks, inducing the B-lymphocytes to produce immunoglobulins (IgG) specific for the antigen, which is then isolated and purified.

Scientists are using engineered antibodies in creative ways to solve challenges in antibody therapeutic research and development. In some cases, they are using VHH fragments, which are single-domain antibodies that can be tuned to bind to a variety of biomolecules. Conventional antibodies have two copies of two different protein chains, a heavy chain and a light chain. In the late 1980s, an atypical variety of antibodies was discovered that contained only a heavy chain. The VHH domain of these heavy-chain-only antibodies recognizes a binding partner. This VHH domain can be engineered as an antibody fragment, producing single-domain antibodies with tunable binding sites. Single-domain antibodies offer several advantages over conventional antibodies when used in research applications: Conventional antibodies require two heavy chains and two light chains to function, while single-domain antibodies require only a single protein. This simplifies production compared with conventional antibodies, which in turn reduces costs. Single-domain antibodies are more pH and heat tolerant, which allows for their use in a wider range of conditions. Additionally, the molecular weight of a single-domain antibody is 12-15 kDa, about one-tenth the weight of a standard antibody. Its small size improves the single-domain antibody's ability to bind to antigen sites that are difficult for conventional antibodies to reach.

The number of different proteins in a cell can vary widely depending on the type of cell and organism. In human cells, for instance, scientists estimate there can be around 20,000 different types of proteins existing simultaneously, corresponding to the number of protein-coding genes in the human genome. However, this number does not account for the variations due to post-translational modifications or alternative splicing, which can significantly increase the diversity of proteins. For example, in a typical mammalian cell, there can be approximately 10 million to 1 billion protein molecules, depending on the cell type and size. Identified in Table 4 is a set of protein targets useful in providing information about the cell type.

TABLE 4
Protein Targets for Immune Cells. The table provides a concise description
of a function of each target, however, it is important to note that
many of these molecules have multifaceted roles in the immune system
and can have varying functions depending on the context.

| Target | Function Description |
|---|---|
| PD-L1 | Programmed death-ligand 1, a protein that plays a major role in suppressing |
| (CD274) | the immune system; often expressed in tumor cells. |
| CD8 | Found on cytotoxic T cells and functions in cell-mediated immunity. |
| CD3 | A protein complex and T-cell co-receptor involved in activating both the |
| | cytotoxic T-Cell and T-Helper cells. |
| PD-1 | Programmed cell death protein 1, a cell surface receptor that plays an |
| | important role in down-regulating the immune system and promoting self- |
| | tolerance by suppressing T cell inflammatory activity. |
| CD45 | A cell surface protein found on all white blood cells and is involved in |
| | activation and differentiation of lymphocytes. |
| CD4 | Found on helper T cells and plays a role in cell-mediated immunity. |
| CD68 | A marker for macrophages and is involved in the immune response. |
| CD11c | A type of integrin found on dendritic cells and is involved in antigen |
| | presentation. |
| FoxP3 | A transcription factor crucial for the development and function of regulatory T |
| | cells which are involved in immune tolerance. |
| α-SMA | Alpha-smooth muscle actin, a marker for myofibroblasts and smooth muscle |
| | cells. |
| CD20 | Found on B cells and is involved in B cell activation and development. |
| Ki67 | A nuclear protein associated with cellular proliferation and is a marker for |
| | dividing cells. |
| CD56 | Also known as Neural Cell Adhesion Molecule (NCAM); found on natural |
| | killer cells. |
| CD31 | Also known as PECAM-1, found on endothelial cells and involved in |
| | angiogenesis. |
| CTLA-4 | Cytotoxic T-lymphocyte-associated protein 4, plays a role in down-regulating |
| | the immune response. |
| PanCK | Pan Cytokeratin, markers for epithelial cells. |
| CD45RO | Memory T cell marker, indicating past exposure to an antigen. |
| CD45RA | Naive T cell marker, indicating no prior exposure to an antigen. |
| HLA-DR | Major histocompatibility complex class II cell surface receptor, involved in |
| | antigen presentation to T cells. |

Additional protein targets include ATPase, Pan-Cadherin, Vimentin, and/or Beta-2-microglobulin, which provides useful information on the structure and boundary of the cell. Additional targets may include pan-cytokeratin (PAN-CK) associated with epithelial cells, Smooth Muscle Actin (SMActin) associated with muscle cells, CD31 and/or LYVE-1, each of which are associated with lymphatic and vessel endothelial cells, CD45 associated with immune cells of hematopoietic lineage, CD20 associated with B cells, CD3 associated with T cells, FOXP marker for regulatory T cells3, CD11c and/or CD68 each of which are associated with myeloid cells.

Means for attaching the oligonucleotides to the probe are known in the art. Typically, the oligonucleotide includes a first bioconjugate reactive moiety which reacts with a second bioconjugate reactive moiety on the probe (e.g., the antibody). The two bioconjugate reactive moieties react, thereby forming a bioconjugate reactive linker. For example, the oligonucleotide moiety includes a DBCO bioconjugate reactive moiety that reacts with an azide bioconjugate reactive moiety on the antibody and forms a bioconjugate linker that covalently links the oligonucleotide moiety to the antibody, for example according to the following scheme:

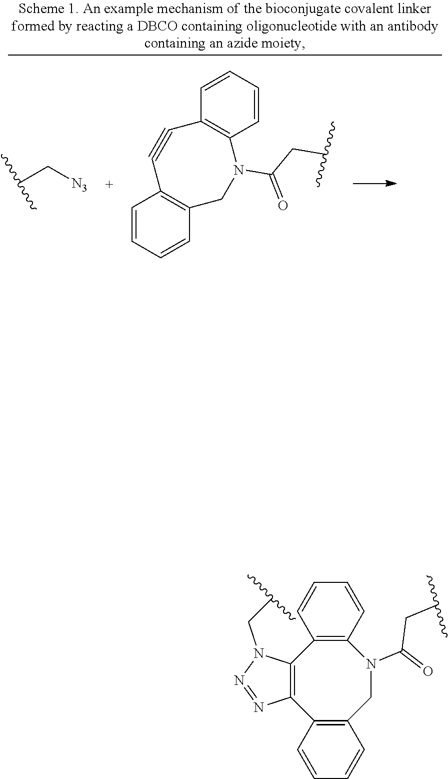 [SMILES: *CN=[N+]=[N-] ; *CC(=O)N1Cc2ccccc2C#Cc2ccccc21 ; *CC(=O)N1Cc2ccccc2-c2nnn(C*)c2-c2ccccc21]

wherein the “ [symbol] ” refers to the attachment point to the oligonucleotide moiety and antibody, respectively.

In embodiments, the linker is a linker as described in Bioconjugate Techniques (Second Edition) by Greg T. Hermanson (2008), which is herein incorporated by referenced in its entirety for all purposes. In embodiments, the linker is a linker as described in Flygare J A, Pillow T H, Aristoff P., Antibody-drug conjugates for the treatment of cancer. Chemical Biology and Drug Design. 2013 January; 81 (1): 113-21, which is herein incorporated by referenced in its entirety for all purposes. In embodiments, the linker is a linker as described in Drachman J G, Senter P D., Antibody-drug conjugates: the chemistry behind empowering antibodies to fight cancer. Hematology Am Soc Hematol Educ Program. 2013; 2013:306-10, which is herein incorporated by referenced in its entirety for all purposes.

One effective method to attach an oligonucleotide to an antibody is through amine conjugation. To start, an antibody with known amino groups, primarily from lysine side chains, is selected. The amino groups of the antibody are then activated using a suitable cross-linking agent. Subsequently, an oligonucleotide with a complementary reactive group is introduced to the activated antibody. The reaction is allowed to proceed, resulting in a stable bond formation between the oligonucleotide and the antibody. Another method involves the use of sulfhydryl conjugation. In this approach, an antibody containing disulfide bridges is chosen. These disulfide bridges are then reduced to expose reactive thiol groups using a specific reducing agent. Once exposed, an oligonucleotide that has been modified to contain a maleimide group or another thiol-reactive group is introduced. The reaction between the thiol and maleimide groups is allowed to proceed, leading to the establishment of a stable thioether bond between the oligonucleotide and the antibody. Carbohydrate conjugation offers yet another method. An antibody containing carbohydrate residues, especially in the Fc region, is selected for this procedure. These carbohydrate residues are oxidized to aldehyde groups. Following this, an oligonucleotide modified to contain a hydrazine or aminooxy group is introduced. The reaction between the aldehyde and the hydrazine/aminooxy group is allowed to unfold, ensuring a stable bond formation between the oligonucleotide and the antibody. This approach is particularly beneficial as the carbohydrate residues are distant from the antigen-binding sites, minimizing interference with the antibody's binding capability.

GlyCLICK™ conjugation is a technology specifically designed for attaching oligonucleotides to antibodies. The process involves a few key steps: 1. Enzymatic Remodeling of the Antibody: GlyCLICK uses an enzyme to specifically target and modify the Fc glycan structures present on antibodies. 2. Introduction of a Click Chemistry Functional Group: Once the Fc glycans are modified, a functional group that facilitates click chemistry, typically azide or alkyne groups, is introduced to the antibody. 3. Conjugation with the Oligonucleotide: The oligonucleotide, which is pre-modified with a complementary click chemistry group (azide if alkyne or DBCO moiety was used on the antibody and vice versa), is then mixed with the modified antibody. The click chemistry reaction between these groups is highly efficient and specific, leading to the formation of a stable covalent bond between the antibody and the oligonucleotide. Optionally, the resulting antibody-oligonucleotide conjugate is purified to remove any unreacted components.

Initial approaches of binding the antibody-oligo conjugates to a cell or tissue, where the antibody is covalently bound to a single-stranded oligonucleotide, has shown a propensity for nonspecific binding to various structures within the tissue samples. Such nonspecific interactions are undesirable as they can lead to aberrant artifacts and result in false positive detections, thus compromising the accuracy and reliability of the diagnostic procedure. A subtle modification introduces a significant enhancement to this method. Specifically, the antibody-oligo conjugates utilized herein are initially deposited in a double-stranded format; see FIGS. 4A and 4B for illustrative embodiments. This configuration offers a key advantage: the additional strand, referred to as the “blocking oligonucleotide(s),” serves to mitigate the aforementioned issue of nonspecific binding. Following the deposition of these double-stranded conjugates, the blocking strand(s) are strategically removed prior to the detection phase. This removal is a critical step, as it exposes the single-stranded oligonucleotide that is covalently bound to the antibody, now devoid of the blocking strand, thereby ensuring that the active site is available for specific binding to the target structure within the tissue sample. The oligonucleotides may include a blocking strand that is substantially the same length as the oligonucleotide as illustrated in FIG. 4A. For example, if the oligonucleotide provided by the Ab-O conjugate includes SEQ ID NO:1, the single blocking strand includes both the LS (SEQ ID NO:133) and RS (SEQ ID NO:265) sequences. Alternatively, as illustrated in FIG. 4B, the blocking oligonucleotide may be provided in two (or more) parts. In this embodiment, a first blocking oligonucleotide includes the LS sequence (SEQ ID NO:133) and a second blocking oligonucleotide includes the RS sequence (SEQ ID NO:265). The significance of this lies in its potential to enhance the specificity and accuracy of antibody-oligo conjugate-based diagnostic procedures. By implementing the double-stranded configuration followed by the removal of the blocking strand(s), this method effectively reduces the incidence of nonspecific binding and the consequent false positive detections. This advancement holds substantial promise for improving the reliability of tissue sample analysis, particularly in the fields of pathology and molecular diagnostics.

Sample prep includes fixation of a cell or tissue (i.e., the sample) to a suitable solid support. Permeabilization includes adding of 0.1% Triton X-100 in 1×PBS and incubating the sample at room temperature for 15 min. The sample is washed at least three times with 500 μL of 1×PBS. Blocking includes flowing 2% BSA in 1×PBS and incubating the cells at room temperature for 60 min. Alternately, the sample can be incubated overnight in 2% BSA or 1×PBS. Binding the Ab-oligo probes includes adding the desired concentration in 0.1% BSA to the sample and incubate for at least 3 hours at room temperature or overnight at 4° C. The sample is washed at least three times with 1×PBS.

Padlock probes (PLPs) are added at a final concentration of 100 nM each in hybridization buffer. PLPs then hybridize overnight at 45° C. The sample is then washed 1× with hybridization buffer for 5 min at 45° C. and 2× with 1×PBS for 5 min each at 37° C. After the washes, a ligase (for example, T4 DNA ligase (New England Biolabs Catalog #M0202S)) is added at a final concentration of 2.5 U/μL with 0.2 U/μL in 1× ligase buffer and incubates for 30-60 min at 37° C. The sample is then washed 1× with 1×PBS and 2× with hybridization buffer. A phosphorothioated amplification primer is added at a final concentration of 0.5 μM in hybridization buffer and incubates for 1 hr at 37° C. Alternatively, the nucleic acid molecule attached to the protein-specific binding agent may serve as the primer and may be extended. The sample is washed 1× with hybridization buffer and 2× with 1×PBS. A mutant version of phi29 DNA polymerase is then added at a final concentration of 1.8 μM with 1 M betaine, dNTPs (0.5 mM each), 0.2 mg/mL BSA, and 4 mM DTT in DEPC-treated water and incubates for 1 hr at 37° C. A Sequencing primer is then added at a final concentration of 0.5 μM in hybridization buffer and incubated for 30 min at 37° C. The sample is washed 3× with flow cell wash buffer, and sequencing-by-synthesis with detectable nucleotides was performed for a suitable amount of cycles (e.g., 1-5 cycles) so as to determine the identity of the PLP and thus the protein.

FIG. 5 provides fluorescent images of proteins detected using the Ab-O conjugates described herein (e.g., specific binding agents). The proteins include PD-1, PD-L1, CD56, CD8, HLA-DR, CD4, CD3, Ki-67, CD20, ATPase, CD45RA, and PanCk shown in individual channels and the composite image (left) for a tonsil tissue section. The scale bar is shown as 1000 μm.

### Example 3. Serial Revealing Cell Paints

The detection and analysis of multiple biomolecules within the same cell or tissue section is crucial for understanding the phenotypic and functional architecture of healthy and diseased states. Traditional single-plex techniques, such as enzyme-linked immunosorbent assays (ELISA), are limited in their ability to provide comprehensive insights due to their focus on single analytes. In contrast, multiplexed detection methods offer the potential to simultaneously analyze multiple biomarkers, thereby providing a more holistic view of cellular and tissue states. However, existing multiplexed antibody-based techniques, including those employing fluorophores, metal markers, and DNA barcodes, face significant challenges. These challenges include the need for meticulous antibody validation, issues with spectral overlap, and the complex and time-consuming nature of sequential staining and bleaching protocols.

Fluorescent multiplexing techniques, such as multiplex immunofluorescence and tissue-based circular immunofluorescence, rely on labeling biomolecules with distinct fluorophores. While these methods can provide sensitive and specific detection, they are constrained by the limited spectral range available for fluorescence detection. Spectral overlap occurs when fluorophores emit light at similar wavelengths, making it difficult to distinguish between different targets. This overlap necessitates the use of dyes with minimal emission overlap, often restricting the number of biomarkers that can be simultaneously detected to four or five. Additionally, methods for removing or inactivating fluorophores after each round of staining, such as enzymatic digestion or chemical bleaching, can damage the sample and prolong the imaging process. As a result, detecting a large number of targets can become impractically lengthy and complex.

To address these limitations, advanced techniques like DNA barcoding have been developed, enabling higher multiplexing capabilities by avoiding spectral limitations. However, these methods typically require complex probe design and hybridization protocols, which can be cumbersome and expensive. Additionally, despite these advances, the sole reliance on antibodies remains a hurdle, as it requires rigorous validation to ensure accuracy and reproducibility. Consequently, there is a pressing need for innovative approaches that can overcome these challenges, enabling efficient and accurate multiplex detection of biomolecules in situ, without the drawbacks associated with current technologies.

The present disclosure addresses the limitations of existing multiplexed biomolecule detection methods by introducing an advanced cell painting technique, capable of being used existing spatial biology platforms (e.g., the G4X™ Platform or ImageXpress® Confocal HT.ai system). This innovative approach combines the principles of traditional cell painting with the enhanced capabilities of cleavable linkers and sequential staining cycles. By integrating these elements, the invention enables the detection of a significantly larger number of cellular structures and biomarkers within the same sample, overcoming the spectral overlap and optical cross-talk issues inherent in conventional fluorescence-based methods.

Described herein is the use of an oligonucleotide described herein covalently attached to cell paint targeting molecules, such as phalloidin or wheat germ agglutinin (WGA), to fluorescent dyes. These oligonucleotides include a first blocking oligonucleotide hybridized to a first sequence of the oligonucleotide and a second blocking oligonucleotide hybridized to a second sequence of the oligonucleotide prior to removing the blocking oligonucleotides (e.g., the first blocking oligonucleotide and second blocking oligonucleotide) and hybridizing a polynucleotide probe described herein to the oligonucleotide to allow detecting a target molecule in or on a cell or tissue.

The mushroom toxin phalloidin is a small bicyclic peptide consisting of seven amino acids with a molecular weight of 789. Phalloidin binds to both large and small filamentous actin (F-actin) with high affinity, and compared to actin-specific antibodies, the non-specific binding of phalloidin is negligible, thus providing minimal background and high contrast during cellular imaging. Phalloidin-dye conjugates have been described previously, for example Capani et al Journal of Histochemistry & Cytochemistry. 2001; 49 (11): 1351-1361, and including a cleavable site in the linker to the fluorophore enables the conjugate to be used in the method described herein. For example, the probe may have the structure:

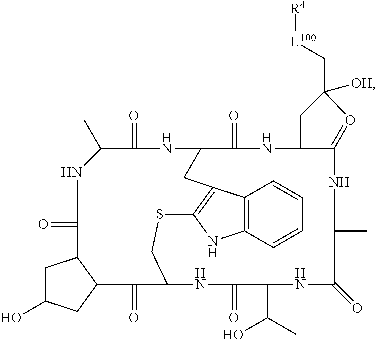

where L¹⁰⁰ is the cleavable linker and R⁴ is a fluorophore moiety.

The method also incorporates automated imaging and image analysis software, enhancing the efficiency and reproducibility of the staining and imaging process. This automation reduces manual intervention, minimizes potential errors, and facilitates large-scale studies. The resulting high-dimensional data can be integrated and analyzed to provide comprehensive profiles of cellular phenotypes, enabling detailed studies of cellular behavior, disease mechanisms, and treatment responses.

The phenotypic profile of a cell reveals the biological state of a cell. More specifically, the phenotypic profile can be used to interrogate biological perturbations because the cellular morphology is influenced by factors such as metabolism, genetic and epigenetic state of the cell, and environmental cues. In addition, it can be used to characterize healthy cells from diseased cells. Because a phenotypic profile is an aggregation of a large number of measurements, it is sensitive to deviations or changes to those features extracted using cellular paints. To create a profile of the cells, all of the features from the different organelles that are imaged and analyzed using commercially available cell imaging software (e.g., CellProfiler™). In morphological profiling, measured features include staining intensities, textural patterns, size, and shape of the labeled cellular structures, as well as correlations between stains across channels, and adjacency relationships between cells and among intracellular structures.

Existing cell paints, described in Table 5, are employed to target specific biomolecules. In current cell painting approaches, fluorescent dyes are conjugated to targeting molecules through covalent bonding, ensuring specific and stable labeling of cellular structures. The attachment process typically involves the use of chemical linkers that form a stable covalent bond between the dye and the targeting molecule. For example, phalloidin, which binds specifically to actin filaments, is covalently linked to a fluorescent dye like Alexa Fluor® 488 using a reactive group on the dye that reacts with a functional group on phalloidin. Similarly, wheat germ agglutinin (WGA), which targets the plasma membrane, is conjugated to a fluorescent dye through a linker that attaches to its glycoprotein-binding sites. This covalent linkage ensures that the dye remains firmly attached to the targeting molecule during the staining, imaging, and any subsequent washing steps, providing consistent and reliable 10 fluorescence labeling of the intended cellular structure.

TABLE 5
Commercially available cell paints

| Targeting Molecule | Fluorescent Dye | Cell Structure Targeted |
|---|---|---|
| Phalloidin | Various (e.g., Alexa Fluor ® 488, | Actin filaments |
| | Alexa Fluor ® 568) | |
| Wheat Germ | Various (e.g., Alexa Fluor ® 488, | Plasma membrane |
| Agglutinin (WGA) | Alexa Fluor ® 594) | |
| MitoTracker ® | Various (e.g., MitoTracker ® Red | Mitochondria |
| | CMXRos, MitoTracker ® Green | |
| | FM) | |
| ER-Tracker ™ | Various (e.g., ER-Tracker ™ Red, | Endoplasmic reticulum |
| | ER-Tracker ™ Green) | |
| Concanavalin A | Various (e.g., Alexa Fluor ® 350) | Endoplasmic reticulum |
| Golgi-Tracker ™ | Various (e.g., BODIPY ® FL C5- | Golgi apparatus |
| | Ceramide) | |
| LysoTracker ® | Various (e.g., LysoTracker ® | Lysosomes |
| | Green DND-26, LysoTracker ® | |
| | Red DND-99) | |
| | CytoFix ™ Red | |
| Annexin V | Various (e.g., Annexin V Alexa | Phosphatidylserine (apoptosis |
| | Fluor ® 488, Annexin V FITC) | marker) |
| Concanavalin A | Various (e.g., Alexa Fluor ® 488, | Cell surface carbohydrates |
| (ConA) | Alexa Fluor ® 594) | |
| Transferrin | Various (e.g., Alexa Fluor ® 488, | Transferrin receptors |
| | Alexa Fluor ® 568) | |
| Lectins (e.g., PNA, | Various (e.g., Alexa Fluor ® 488, | Specific carbohydrate |
| UEA-1) | Alexa Fluor ® 594) | structures |

The method may be useful in detecting biomolecules such as proteins and nucleic acid molecules, organelle structures such as the Golgi Apparatus, and also the cytoskeleton. The cytoskeleton is a network of different protein fibers (e.g., actin and myosin) that maintains the shape and position of the organelles within a cell. The cytoplasm, a fluid which can be rather gel-like, surrounds the nucleus, is considered an organelle.

Additional organelles detectable using the methods and compositions described herein include the Endoplasmic Reticulum (ER), which is a network of membranes that forms channels that cris-crosses the cytoplasm utilizing its tubular and vesicular structures to manufacture various molecules. The ER includes small granular structures called ribosomes useful for the synthesis of proteins. Smooth ER makes fat compounds and deactivates certain chemicals like alcohol or detected undesirable chemicals such as pesticides. Rough ER makes and modifies proteins and stores them until notified by the cell communication system to send them to organelles that require the substances. Typically, all healthy cells in humans, except erythrocytes (red blood cells) and spermatozoa, are equipped with endoplasmic reticulum. The Golgi apparatus (also referred to as a Golgi complex) consists of one or more Golgi bodies which are located close to the nucleus and consist of flattened membranes stacked atop one another like a stack of coins. The Golgi apparatus prepares proteins and lipid (fat) molecules for use in other places inside and outside the cell. Lysosomes are membrane-enclosed organelles that have an acidic interior (pH˜4.8) and can vary in size from 0.1 to 1.2 μm. Lysosomes house various hydrolytic enzymes responsible for digesting biopolymers such as proteins, peptides, nucleic acids, carbohydrates and lipids. Ribosomes are tiny spherical organelles distributed around the cell in large numbers to synthesize cell proteins. They also create amino acid chains for protein manufacture. Ribosomes are created within the nucleus at the level of the nucleolus and then released into the cytoplasm.

### Example 4. Imaging a Multiplex Tonsil Tissue Sample

To image and analyze a multiplex tonsil tissue sample using a combination of intrinsic (e.g., Hoescht 33342) and non-intrinsic ([targeting molecule]-[cleavable linker (CL)]-[fluorophore]) cell paints as the specific binding agents, cleavable linkers for sequential staining and imaging cycles may be employed. By spatially separating the dyes, we minimize optical cross-talk and maximize detection clarity. To begin, the fixed and prepared tonsil tissue sample is subjected to an initial round of staining using a set of cell paints and immunostains designed to target specific cellular components. The first set includes:
Endoplasmic Reticulum: Concanavalin A (ConA)-CL-Alexa Fluor® 532 (emission: 532 nm)
Golgi Apparatus: Wheat germ agglutinin (WGA)-CL-Alexa Fluor® 594 (emission: 594 nm)
F-Actin: Phalloidin-CL-Alexa Fluor® 647 (emission: 647 nm)
Lysosomes: Lyso Tracker-CL-Alexa Fluor® 680 (emission: 680 nm)

Once the tissue is stained, it is imaged to capture the fluorescence signals from each dye. Following the initial imaging, the tissue sample undergoes treatment with specific cleavage reagents designed to remove the fluorescent dyes linked through cleavable linkers. The sample is then thoroughly washed to ensure complete removal of the cleaved dyes, preparing it for the next cycle of staining. In the second cycle, the tissue is stained with a new set of cell paints targeting additional structures, each conjugated with non-overlapping dyes to avoid optical cross-talk. This second set includes:
Nucleus: Hoechst 33342 (intrinsic, excitation/emission: 387/447 nm)
Nucleoli: SYTO 14 green fluorescent nucleic acid stain (intrinsic, emission: 531/593 nm)
Mitochondria: MitoTracker™ Deep Red (intrinsic, emission: 628/692 nm)
Transferrin Receptors: Transferrin-CL-Alexa Fluor 532 (emission: 532 nm)
Nuclear Envelope: Anti-Lamin A/C-CL-Alexa Fluor 594 (emission: 594 nm)
Cell Surface Receptors: Anti-CD3-CL-Alexa Fluor 422 (emission: 422 nm)

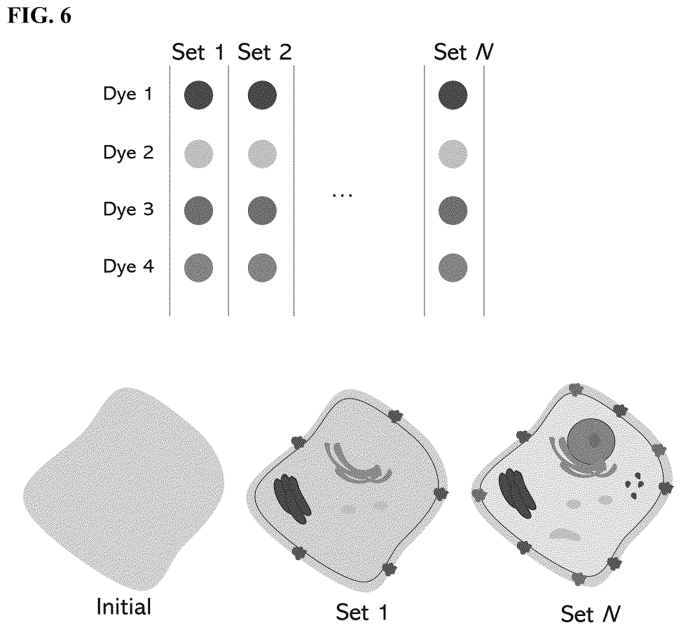

The tissue is then imaged again. After imaging, the dyes are cleaved, and the tissue is prepared for additional cycles, or detection modes, if necessary. This process of staining, imaging, and cleavage is repeated for subsequent cycles, each time introducing new cell paints to target different cellular components as illustrated in FIG. 6. Note, intrinsic stains such as Hoechst 33342 and SYTO 14, should be included in the final set so as not to interfere with detection in intervening staining cycles.

Each cycle ensures that only non-overlapping dyes are used to maintain clear separation of signals. For example, following one or more cycles using the cleavable conjugates described supra one can use traditional (i.e., non-cleavable) staining agents, such as primary antibodies (e.g., beta tubulin monoclonal antibody (ThermoFisher Scientific, 32-2600), anti-clathrin heavy chain antibody (abcam, ab21679), and anti-caveolin-1 antibody (abcam, ab2910) coupled with secondary antibody-oligonucleotide conjugates. For example, protocols for traditional immunostaining may be found Civitci, F. et al. Protoc. Exch. doi.org/10.21203/rs.3.pex-1069/v1 (2020).

In addition, the aforementioned cell paints may be covalently attached to an oligonucleotide described herein. These oligonucleotides include a first blocking oligonucleotide hybridized to a first sequence of the oligonucleotide and a second blocking oligonucleotide hybridized to a second sequence of the oligonucleotide prior to removing the blocking oligonucleotides (e.g., the first blocking oligonucleotide and second blocking oligonucleotide) and hybridizing a polynucleotide probe described herein to the oligonucleotide. In embodiments, the polynucleotide probe includes a first binding sequence and a second binding sequence to the oligonucleotide covalently attached to the cell paint. Following hybridizing the polynucleotide probe to the oligonucleotide, the polynucleotide probe be ligated and amplified to form an amplification product including more than one copy of the first binding sequence and second binding sequence of the polynucleotide probe to allow detecting a target molecule in or on a cell or tissue. In embodiments, detecting the target molecule includes sequencing the amplification product.

After all cycles are completed, the imaging data from each cycle are integrated using commercially available image analysis software. This software aligns the images from different cycles to create a comprehensive map of the cellular structures and biomarkers within the tonsil tissue. The data are then analyzed to quantify the expression and spatial distribution of the targeted components. By sequentially applying cell paints and utilizing cleavable linkers, this method allows for the imaging of a tonsil tissue sample, providing detailed and comprehensive visualization of various cellular components without the limitations of spectral overlap. The high-content imaging system captures high-resolution images, and the integrated data analysis offers insights into the cellular architecture and biomarker distribution within the tissue, facilitating a deeper understanding of tonsil tissue structure and function.

To facilitate the visualization of organelle and related target data commercially available software (e.g., TissueMaker®, TissueFAXS™, THUNDER™) can allow users to dynamically generate a visual interpretation of data. For example, a typical software may present a user interface with a three-dimensional representation of the cell and/or tissue. For example, the method may further include stitching. Stitching combines multiple field of view (FOV) into a single image. Stitching can be performed using a variety of techniques. For example, one approach is, for each row of FOV that together will form the combined image of the sample and each FOV within the row, determine a horizontal shift for each FOV. Once the horizontal shifting is calculated, a vertical shift is calculated for each row of FOV. The horizontal and vertical shifts can be calculated based on cross-correlation, e.g., phase correlation. With the horizontal and vertical shift for each FOV, a single combined image can be generated, and target biomolecule coordinates can be transferred to the combined image based on the horizontal and vertical shift. For the reconstruction of 3D tissues, several computational methods such as PASTE, PASTE2, SLAT, and SPACEL can be utilized. These methods and algorithms typically involve aligning detected targets between different slices and performing coordinate transformation and rotation of different slices to achieve a 3D structure composed of multiple slices. Thus, the use of cell paints as specific binding agents enable targeting organelles and/or cellular components of interest, while the sequencing the amplification product resulting the amplification and hybridization of the polynucleotide probe to the oligonucleotide covalently attached to the cell paint enables detecting the target molecule.

### EMBODIMENTS

Embodiment P1. A composition comprising: a specific binding agent covalently attached to an oligonucleotide, wherein the oligonucleotide comprises a sequence at least 80% identical to a sequence selected from SEQ ID NO:1 to SEQ ID NO:132.

Embodiment P2. The composition of Embodiment P1, wherein the specific binding agent is an antibody, single-chain Fv fragment (scFv), antibody fragment-antigen binding (Fab), affimer, or an aptamer.

Embodiment P3. The composition of Embodiment P1, wherein the specific binding agent is an antibody, single-chain Fv fragment (scFv), or antibody fragment-antigen binding (Fab).

Embodiment P4. The composition of Embodiment P1, wherein the specific binding agent is an enzyme, enzyme mutant, peptide, Molecular Imprinted Polymer (MIP), DARPin (Designed Ankyrin Repeat Protein), peptoid, lectin, siRNA, or miRNA molecule.

Embodiment P5. The composition of any one of Embodiments P1 to P4, further comprising a polynucleotide hybridized to the oligonucleotide.

Embodiment P6. The composition of Embodiment P5, wherein said polynucleotide comprises a fluorophore.

Embodiment P7. The composition of Embodiment P5, wherein said polynucleotide comprises a primer binding sequence.

Embodiment P8. The composition of any one of Embodiments P5 to P7, wherein said polynucleotide comprises a barcode sequence.

Embodiment P9. The composition of any one of Embodiments P5 to P7, wherein said polynucleotide comprises a barcode nucleotide.

Embodiment P10. The composition of any one of Embodiments P1 to P9, wherein the oligonucleotide comprises a sequence at least 90%, 95%, or 98% identical to a sequence selected from SEQ ID NO: 1 to SEQ ID NO:132.

Embodiment P11. The composition of any one of Embodiments P1 to P9, wherein the oligonucleotide comprises a sequence at least a sequence selected from SEQ ID NO:1 to SEQ ID NO: 132.

Embodiment P12. The composition of any one of Embodiments P1 to P11, further comprising a first polynucleotide and a second polynucleotide hybridized to the oligonucleotide.

Embodiment P13. The composition of any one of Embodiments P1 to P12, wherein the polynucleotide comprises a sequence at least 80% identical to a sequence selected from SEQ ID NO: 133 to SEQ ID NO:264.

Embodiment P14. The composition of any one of Embodiments P1 to P12, wherein the polynucleotide comprises a sequence at least 80% identical to a sequence selected from SEQ ID NO: 265 to SEQ ID NO:396.

Embodiment P15. The composition of Embodiment P12, wherein the first polynucleotide comprises a sequence at least 80% identical to a sequence selected from SEQ ID NO: 133 to SEQ ID NO:264 and the second polynucleotide comprises a sequence at least 80% identical to a sequence selected from SEQ ID NO:265 to SEQ ID NO:396.

Embodiment P16. The composition of any one of Embodiments P1 to P12, wherein the polynucleotide comprises a first sequence at least 80% identical to a sequence selected from SEQ ID NO:133 to SEQ ID NO:264 and a second sequence at least 80% identical to a sequence selected from SEQ ID NO:265 to SEQ ID NO:396.

Embodiment P17. The composition of any one of Embodiments P1 to P12, wherein the oligonucleotide comprises a sequence at least 80% identical to SEQ ID NO:1 and the polynucleotide comprises a sequence at least 80% identical to SEQ ID NO:133; the oligonucleotide comprises a sequence at least 80% identical to SEQ ID NO:2 and the polynucleotide comprises a sequence at least 80% identical to SEQ ID NO:134; the oligonucleotide comprises a sequence at least 80% identical to SEQ ID NO:3 and the polynucleotide comprises a sequence at least 80% identical to SEQ ID NO:135; the oligonucleotide comprises a sequence at least 80% identical to SEQ ID NO:4 and the polynucleotide comprises a sequence at least 80% identical to SEQ ID NO:136; the oligonucleotide comprises a sequence at least 80% identical to SEQ ID NO:5 and the polynucleotide comprises a sequence at least 80% identical to SEQ ID NO:137; the oligonucleotide comprises a sequence at least 80% identical to SEQ ID NO:6 and the polynucleotide comprises a sequence at least 80% identical to SEQ ID NO:138; the oligonucleotide comprises a sequence at least 80% identical to SEQ ID NO:7 and the polynucleotide comprises a sequence at least 80% identical to SEQ ID NO:139; the oligonucleotide comprises a sequence at least 80% identical to SEQ ID NO:8 and the polynucleotide comprises a sequence at least 80% identical to SEQ ID NO:140; the oligonucleotide comprises a sequence at least 80% identical to SEQ ID NO:9 and the polynucleotide comprises a sequence at least 80% identical to SEQ ID NO:141; the oligonucleotide comprises a sequence at least 80% identical to SEQ ID NO:10 and the polynucleotide comprises a sequence at least 80% identical to SEQ ID NO:142; the oligonucleotide comprises a sequence at least 80% identical to SEQ ID NO:11 and the polynucleotide comprises a sequence at least 80% identical to SEQ ID NO:143; the oligonucleotide comprises a sequence at least 80% identical to SEQ ID NO:12 and the polynucleotide comprises a sequence at least 80% identical to SEQ ID NO:144; the oligonucleotide comprises a sequence at least 80% identical to SEQ ID NO:13 and the polynucleotide comprises a sequence at least 80% identical to SEQ ID NO:145; the oligonucleotide comprises a sequence at least 80% identical to SEQ ID NO:14 and the polynucleotide comprises a sequence at least 80% identical to SEQ ID NO:146; the oligonucleotide comprises a sequence at least 80% identical to SEQ ID NO: 15 and the polynucleotide comprises a sequence at least 80% identical to SEQ ID NO:147; the oligonucleotide comprises a sequence at least 80% identical to SEQ ID NO: 16 and the polynucleotide comprises a sequence at least 80% identical to SEQ ID NO:148; the oligonucleotide comprises a sequence at least 80% identical to SEQ ID NO: 17 and the polynucleotide comprises a sequence at least 80% identical to SEQ ID NO:149; the oligonucleotide comprises a sequence at least 80% identical to SEQ ID NO: 18 and the polynucleotide comprises a sequence at least 80% identical to SEQ ID NO:150; the oligonucleotide comprises a sequence at least 80% identical to SEQ ID NO:19 and the polynucleotide comprises a sequence at least 80% identical to SEQ ID NO:151; the oligonucleotide comprises a sequence at least 80% identical to SEQ ID NO:20 and the polynucleotide comprises a sequence at least 80% identical to SEQ ID NO:152; the oligonucleotide comprises a sequence at least 80% identical to SEQ ID NO:21 and the polynucleotide comprises a sequence at least 80% identical to SEQ ID NO:153; the oligonucleotide comprises a sequence at least 80% identical to SEQ ID NO:22 and the polynucleotide comprises a sequence at least 80% identical to SEQ ID NO: 154; the oligonucleotide comprises a sequence at least 80% identical to SEQ ID NO:23 and the polynucleotide comprises a sequence at least 80% identical to SEQ ID NO:155; the oligonucleotide comprises a sequence at least 80% identical to SEQ ID NO:24 and the polynucleotide comprises a sequence at least 80% identical to SEQ ID NO:156; the oligonucleotide comprises a sequence at least 80% identical to SEQ ID NO:25 and the polynucleotide comprises a sequence at least 80% identical to SEQ ID NO:157; the oligonucleotide comprises a sequence at least 80% identical to SEQ ID NO:26 and the polynucleotide comprises a sequence at least 80% identical to SEQ ID NO:158; the oligonucleotide comprises a sequence at least 80% identical to SEQ ID NO:27 and the polynucleotide comprises a sequence at least 80% identical to SEQ ID NO:159; the oligonucleotide comprises a sequence at least 80% identical to SEQ ID NO:28 and the polynucleotide comprises a sequence at least 80% identical to SEQ ID NO:160; the oligonucleotide comprises a sequence at least 80% identical to SEQ ID NO:29 and the polynucleotide comprises a sequence at least 80% identical to SEQ ID NO:161; the oligonucleotide comprises a sequence at least 80% identical to SEQ ID NO:30 and the polynucleotide comprises a sequence at least 80% identical to SEQ ID NO:162; the oligonucleotide comprises a sequence at least 80% identical to SEQ ID NO:31 and the polynucleotide comprises a sequence at least 80% identical to SEQ ID NO:163; the oligonucleotide comprises a sequence at least 80% identical to SEQ ID NO:32 and the polynucleotide comprises a sequence at least 80% identical to SEQ ID NO:164; the oligonucleotide comprises a sequence at least 80% identical to SEQ ID NO:33 and the polynucleotide comprises a sequence at least 80% identical to SEQ ID NO:165; the oligonucleotide comprises a sequence at least 80% identical to SEQ ID NO:34 and the polynucleotide comprises a sequence at least 80% identical to SEQ ID NO:166; the oligonucleotide comprises a sequence at least 80% identical to SEQ ID NO:35 and the polynucleotide comprises a sequence at least 80% identical to SEQ ID NO:167; the oligonucleotide comprises a sequence at least 80% identical to SEQ ID NO:36 and the polynucleotide comprises a sequence at least 80% identical to SEQ ID NO:168; the oligonucleotide comprises a sequence at least 80% identical to SEQ ID NO:37 and the polynucleotide comprises a sequence at least 80% identical to SEQ ID NO:169; the oligonucleotide comprises a sequence at least 80% identical to SEQ ID NO:38 and the polynucleotide comprises a sequence at least 80% identical to SEQ ID NO:170; the oligonucleotide comprises a sequence at least 80% identical to SEQ ID NO:39 and the polynucleotide comprises a sequence at least 80% identical to SEQ ID NO:171; the oligonucleotide comprises a sequence at least 80% identical to SEQ ID NO:40 and the polynucleotide comprises a sequence at least 80% identical to SEQ ID NO:172; the oligonucleotide comprises a sequence at least 80% identical to SEQ ID NO:41 and the polynucleotide comprises a sequence at least 80% identical to SEQ ID NO:173; the oligonucleotide comprises a sequence at least 80% identical to SEQ ID NO:42 and the polynucleotide comprises a sequence at least 80% identical to SEQ ID NO:174; the oligonucleotide comprises a sequence at least 80% identical to SEQ ID NO:43 and the polynucleotide comprises a sequence at least 80% identical to SEQ ID NO:175; the oligonucleotide comprises a sequence at least 80% identical to SEQ ID NO:44 and the polynucleotide comprises a sequence at least 80% identical to SEQ ID NO:176; the oligonucleotide comprises a sequence at least 80% identical to SEQ ID NO:45 and the polynucleotide comprises a sequence at least 80% identical to SEQ ID NO:177; the oligonucleotide comprises a sequence at least 80% identical to SEQ ID NO:46 and the polynucleotide comprises a sequence at least 80% identical to SEQ ID NO:178; the oligonucleotide comprises a sequence at least 80% identical to SEQ ID NO:47 and the polynucleotide comprises a sequence at least 80% identical to SEQ ID NO:179; the oligonucleotide comprises a sequence at least 80% identical to SEQ ID NO:48 and the polynucleotide comprises a sequence at least 80% identical to SEQ ID NO:180; the oligonucleotide comprises a sequence at least 80% identical to SEQ ID NO:49 and the polynucleotide comprises a sequence at least 80% identical to SEQ ID NO:181; the oligonucleotide comprises a sequence at least 80% identical to SEQ ID NO:50 and the polynucleotide comprises a sequence at least 80% identical to SEQ ID NO: 182; the oligonucleotide comprises a sequence at least 80% identical to SEQ ID NO:51 and the polynucleotide comprises a sequence at least 80% identical to SEQ ID NO:183; the oligonucleotide comprises a sequence at least 80% identical to SEQ ID NO:52 and the polynucleotide comprises a sequence at least 80% identical to SEQ ID NO:184; the oligonucleotide comprises a sequence at least 80% identical to SEQ ID NO:53 and the polynucleotide comprises a sequence at least 80% identical to SEQ ID NO:185; the oligonucleotide comprises a sequence at least 80% identical to SEQ ID NO:54 and the polynucleotide comprises a sequence at least 80% identical to SEQ ID NO:186; the oligonucleotide comprises a sequence at least 80% identical to SEQ ID NO:55 and the polynucleotide comprises a sequence at least 80% identical to SEQ ID NO:187; the oligonucleotide comprises a sequence at least 80% identical to SEQ ID NO:56 and the polynucleotide comprises a sequence at least 80% identical to SEQ ID NO:188; the oligonucleotide comprises a sequence at least 80% identical to SEQ ID NO:57 and the polynucleotide comprises a sequence at least 80% identical to SEQ ID NO:189; the oligonucleotide comprises a sequence at least 80% identical to SEQ ID NO:58 and the polynucleotide comprises a sequence at least 80% identical to SEQ ID NO:190; the oligonucleotide comprises a sequence at least 80% identical to SEQ ID NO:59 and the polynucleotide comprises a sequence at least 80% identical to SEQ ID NO:191; the oligonucleotide comprises a sequence at least 80% identical to SEQ ID NO:60 and the polynucleotide comprises a sequence at least 80% identical to SEQ ID NO:192; the oligonucleotide comprises a sequence at least 80% identical to SEQ ID NO:61 and the polynucleotide comprises a sequence at least 80% identical to SEQ ID NO:193; the oligonucleotide comprises a sequence at least 80% identical to SEQ ID NO:62 and the polynucleotide comprises a sequence at least 80% identical to SEQ ID NO:194; the oligonucleotide comprises a sequence at least 80% identical to SEQ ID NO:63 and the polynucleotide comprises a sequence at least 80% identical to SEQ ID NO:195; the oligonucleotide comprises a sequence at least 80% identical to SEQ ID NO:64 and the polynucleotide comprises a sequence at least 80% identical to SEQ ID NO:196; the oligonucleotide comprises a sequence at least 80% identical to SEQ ID NO:65 and the polynucleotide comprises a sequence at least 80% identical to SEQ ID NO:197; the oligonucleotide comprises a sequence at least 80% identical to SEQ ID NO:66 and the polynucleotide comprises a sequence at least 80% identical to SEQ ID NO:198; the oligonucleotide comprises a sequence at least 80% identical to SEQ ID NO:67 and the polynucleotide comprises a sequence at least 80% identical to SEQ ID NO:199; the oligonucleotide comprises a sequence at least 80% identical to SEQ ID NO:68 and the polynucleotide comprises a sequence at least 80% identical to SEQ ID NO:200; the oligonucleotide comprises a sequence at least 80% identical to SEQ ID NO:69 and the polynucleotide comprises a sequence at least 80% identical to SEQ ID NO:201; the oligonucleotide comprises a sequence at least 80% identical to SEQ ID NO:70 and the polynucleotide comprises a sequence at least 80% identical to SEQ ID NO:202; the oligonucleotide comprises a sequence at least 80% identical to SEQ ID NO:71 and the polynucleotide comprises a sequence at least 80% identical to SEQ ID NO:203; the oligonucleotide comprises a sequence at least 80% identical to SEQ ID NO:72 and the polynucleotide comprises a sequence at least 80% identical to SEQ ID NO:204; the oligonucleotide comprises a sequence at least 80% identical to SEQ ID NO:73 and the polynucleotide comprises a sequence at least 80% identical to SEQ ID NO:205; the oligonucleotide comprises a sequence at least 80% identical to SEQ ID NO:74 and the polynucleotide comprises a sequence at least 80% identical to SEQ ID NO:206; the oligonucleotide comprises a sequence at least 80% identical to SEQ ID NO:75 and the polynucleotide comprises a sequence at least 80% identical to SEQ ID NO:207; the oligonucleotide comprises a sequence at least 80% identical to SEQ ID NO:76 and the polynucleotide comprises a sequence at least 80% identical to SEQ ID NO:208; the oligonucleotide comprises a sequence at least 80% identical to SEQ ID NO:77 and the polynucleotide comprises a sequence at least 80% identical to SEQ ID NO:209; the oligonucleotide comprises a sequence at least 80% identical to SEQ ID NO:78 and the polynucleotide comprises a sequence at least 80% identical to SEQ ID NO:210; the oligonucleotide comprises a sequence at least 80% identical to SEQ ID NO:79 and the polynucleotide comprises a sequence at least 80% identical to SEQ ID NO:211; the oligonucleotide comprises a sequence at least 80% identical to SEQ ID NO:80 and the polynucleotide comprises a sequence at least 80% identical to SEQ ID NO:212; the oligonucleotide comprises a sequence at least 80% identical to SEQ ID NO:81 and the polynucleotide comprises a sequence at least 80% identical to SEQ ID NO:213; the oligonucleotide comprises a sequence at least 80% identical to SEQ ID NO:82 and the polynucleotide comprises a sequence at least 80% identical to SEQ ID NO:214; the oligonucleotide comprises a sequence at least 80% identical to SEQ ID NO:83 and the polynucleotide comprises a sequence at least 80% identical to SEQ ID NO:215; the oligonucleotide comprises a sequence at least 80% identical to SEQ ID NO:84 and the polynucleotide comprises a sequence at least 80% identical to SEQ ID NO:216; the oligonucleotide comprises a sequence at least 80% identical to SEQ ID NO:85 and the polynucleotide comprises a sequence at least 80% identical to SEQ ID NO:217; the oligonucleotide comprises a sequence at least 80% identical to SEQ ID NO:86 and the polynucleotide comprises a sequence at least 80% identical to SEQ ID NO:218; the oligonucleotide comprises a sequence at least 80% identical to SEQ ID NO:87 and the polynucleotide comprises a sequence at least 80% identical to SEQ ID NO:219; the oligonucleotide comprises a sequence at least 80% identical to SEQ ID NO:88 and the polynucleotide comprises a sequence at least 80% identical to SEQ ID NO:220; the oligonucleotide comprises a sequence at least 80% identical to SEQ ID NO:89 and the polynucleotide comprises a sequence at least 80% identical to SEQ ID NO:221; the oligonucleotide comprises a sequence at least 80% identical to SEQ ID NO:90 and the polynucleotide comprises a sequence at least 80% identical to SEQ ID NO:222; the oligonucleotide comprises a sequence at least 80% identical to SEQ ID NO:91 and the polynucleotide comprises a sequence at least 80% identical to SEQ ID NO:223; the oligonucleotide comprises a sequence at least 80% identical to SEQ ID NO:92 and the polynucleotide comprises a sequence at least 80% identical to SEQ ID NO:224; the oligonucleotide comprises a sequence at least 80% identical to SEQ ID NO:93 and the polynucleotide comprises a sequence at least 80% identical to SEQ ID NO:225; the oligonucleotide comprises a sequence at least 80% identical to SEQ ID NO:94 and the polynucleotide comprises a sequence at least 80% identical to SEQ ID NO:226; the oligonucleotide comprises a sequence at least 80% identical to SEQ ID NO:95 and the polynucleotide comprises a sequence at least 80% identical to SEQ ID NO:227; the oligonucleotide comprises a sequence at least 80% identical to SEQ ID NO:96 and the polynucleotide comprises a sequence at least 80% identical to SEQ ID NO:228; the oligonucleotide comprises a sequence at least 80% identical to SEQ ID NO:97 and the polynucleotide comprises a sequence at least 80% identical to SEQ ID NO:229; the oligonucleotide comprises a sequence at least 80% identical to SEQ ID NO:98 and the polynucleotide comprises a sequence at least 80% identical to SEQ ID NO:230; the oligonucleotide comprises a sequence at least 80% identical to SEQ ID NO:99 and the polynucleotide comprises a sequence at least 80% identical to SEQ ID NO:231; the oligonucleotide comprises a sequence at least 80% identical to SEQ ID NO: 100 and the polynucleotide comprises a sequence at least 80% identical to SEQ ID NO:232; the oligonucleotide comprises a sequence at least 80% identical to SEQ ID NO: 101 and the polynucleotide comprises a sequence at least 80% identical to SEQ ID NO:233; the oligonucleotide comprises a sequence at least 80% identical to SEQ ID NO: 102 and the polynucleotide comprises a sequence at least 80% identical to SEQ ID NO:234; the oligonucleotide comprises a sequence at least 80% identical to SEQ ID NO:103 and the polynucleotide comprises a sequence at least 80% identical to SEQ ID NO:235; the oligonucleotide comprises a sequence at least 80% identical to SEQ ID NO: 104 and the polynucleotide comprises a sequence at least 80% identical to SEQ ID NO:236; the oligonucleotide comprises a sequence at least 80% identical to SEQ ID NO:105 and the polynucleotide comprises a sequence at least 80% identical to SEQ ID NO:237; the oligonucleotide comprises a sequence at least 80% identical to SEQ ID NO: 106 and the polynucleotide comprises a sequence at least 80% identical to SEQ ID NO:238; the oligonucleotide comprises a sequence at least 80% identical to SEQ ID NO: 107 and the polynucleotide comprises a sequence at least 80% identical to SEQ ID NO:239; the oligonucleotide comprises a sequence at least 80% identical to SEQ ID NO: 108 and the polynucleotide comprises a sequence at least 80% identical to SEQ ID NO:240; the oligonucleotide comprises a sequence at least 80% identical to SEQ ID NO: 109 and the polynucleotide comprises a sequence at least 80% identical to SEQ ID NO:241; the oligonucleotide comprises a sequence at least 80% identical to SEQ ID NO:110 and the polynucleotide comprises a sequence at least 80% identical to SEQ ID NO:242; the oligonucleotide comprises a sequence at least 80% identical to SEQ ID NO: 111 and the polynucleotide comprises a sequence at least 80% identical to SEQ ID NO:243; the oligonucleotide comprises a sequence at least 80% identical to SEQ ID NO:112 and the polynucleotide comprises a sequence at least 80% identical to SEQ ID NO:244; the oligonucleotide comprises a sequence at least 80% identical to SEQ ID NO:113 and the polynucleotide comprises a sequence at least 80% identical to SEQ ID NO:245; the oligonucleotide comprises a sequence at least 80% identical to SEQ ID NO:114 and the polynucleotide comprises a sequence at least 80% identical to SEQ ID NO:246; the oligonucleotide comprises a sequence at least 80% identical to SEQ ID NO:115 and the polynucleotide comprises a sequence at least 80% identical to SEQ ID NO:247; the oligonucleotide comprises a sequence at least 80% identical to SEQ ID NO: 116 and the polynucleotide comprises a sequence at least 80% identical to SEQ ID NO:248; the oligonucleotide comprises a sequence at least 80% identical to SEQ ID NO:117 and the polynucleotide comprises a sequence at least 80% identical to SEQ ID NO:249; the oligonucleotide comprises a sequence at least 80% identical to SEQ ID NO: 118 and the polynucleotide comprises a sequence at least 80% identical to SEQ ID NO:250; the oligonucleotide comprises a sequence at least 80% identical to SEQ ID NO: 119 and the polynucleotide comprises a sequence at least 80% identical to SEQ ID NO:251; the oligonucleotide comprises a sequence at least 80% identical to SEQ ID NO: 120 and the polynucleotide comprises a sequence at least 80% identical to SEQ ID NO:252; the oligonucleotide comprises a sequence at least 80% identical to SEQ ID NO:121 and the polynucleotide comprises a sequence at least 80% identical to SEQ ID NO:253; the oligonucleotide comprises a sequence at least 80% identical to SEQ ID NO: 122 and the polynucleotide comprises a sequence at least 80% identical to SEQ ID NO:254; the oligonucleotide comprises a sequence at least 80% identical to SEQ ID NO:123 and the polynucleotide comprises a sequence at least 80% identical to SEQ ID NO:255; the oligonucleotide comprises a sequence at least 80% identical to SEQ ID NO:124 and the polynucleotide comprises a sequence at least 80% identical to SEQ ID NO:256; the oligonucleotide comprises a sequence at least 80% identical to SEQ ID NO:125 and the polynucleotide comprises a sequence at least 80% identical to SEQ ID NO:257; the oligonucleotide comprises a sequence at least 80% identical to SEQ ID NO:126 and the polynucleotide comprises a sequence at least 80% identical to SEQ ID NO:258; the oligonucleotide comprises a sequence at least 80% identical to SEQ ID NO:127 and the polynucleotide comprises a sequence at least 80% identical to SEQ ID NO:259; the oligonucleotide comprises a sequence at least 80% identical to SEQ ID NO:128 and the polynucleotide comprises a sequence at least 80% identical to SEQ ID NO:260; the oligonucleotide comprises a sequence at least 80% identical to SEQ ID NO:129 and the polynucleotide comprises a sequence at least 80% identical to SEQ ID NO:261; the oligonucleotide comprises a sequence at least 80% identical to SEQ ID NO:130 and the polynucleotide comprises a sequence at least 80% identical to SEQ ID NO:262; the oligonucleotide comprises a sequence at least 80% identical to SEQ ID NO:131 and the polynucleotide comprises a sequence at least 80% identical to SEQ ID NO:263; or the oligonucleotide comprises a sequence at least 80% identical to SEQ ID NO:132 and the polynucleotide comprises a sequence at least 80% identical to SEQ ID NO:264.

Embodiment P18. The composition of any one of Embodiments P1 to P12, wherein the oligonucleotide comprises a sequence at least 80% identical to SEQ ID NO:1 and the polynucleotide comprises a sequence at least 80% identical to SEQ ID NO:265; the oligonucleotide comprises a sequence at least 80% identical to SEQ ID NO:2 and the polynucleotide comprises a sequence at least 80% identical to SEQ ID NO:266; the oligonucleotide comprises a sequence at least 80% identical to SEQ ID NO:3 and the polynucleotide comprises a sequence at least 80% identical to SEQ ID NO:267; the oligonucleotide comprises a sequence at least 80% identical to SEQ ID NO:4 and the polynucleotide comprises a sequence at least 80% identical to SEQ ID NO:268; the oligonucleotide comprises a sequence at least 80% identical to SEQ ID NO:5 and the polynucleotide comprises a sequence at least 80% identical to SEQ ID NO:269; the oligonucleotide comprises a sequence at least 80% identical to SEQ ID NO:6 and the polynucleotide comprises a sequence at least 80% identical to SEQ ID NO:270; the oligonucleotide comprises a sequence at least 80% identical to SEQ ID NO:7 and the polynucleotide comprises a sequence at least 80% identical to SEQ ID NO:271; the oligonucleotide comprises a sequence at least 80% identical to SEQ ID NO:8 and the polynucleotide comprises a sequence at least 80% identical to SEQ ID NO:272; the oligonucleotide comprises a sequence at least 80% identical to SEQ ID NO:9 and the polynucleotide comprises a sequence at least 80% identical to SEQ ID NO:273; the oligonucleotide comprises a sequence at least 80% identical to SEQ ID NO:10 and the polynucleotide comprises a sequence at least 80% identical to SEQ ID NO:274; the oligonucleotide comprises a sequence at least 80% identical to SEQ ID NO:11 and the polynucleotide comprises a sequence at least 80% identical to SEQ ID NO:275; the oligonucleotide comprises a sequence at least 80% identical to SEQ ID NO:12 and the polynucleotide comprises a sequence at least 80% identical to SEQ ID NO:276; the oligonucleotide comprises a sequence at least 80% identical to SEQ ID NO:13 and the polynucleotide comprises a sequence at least 80% identical to SEQ ID NO:277; the oligonucleotide comprises a sequence at least 80% identical to SEQ ID NO:14 and the polynucleotide comprises a sequence at least 80% identical to SEQ ID NO:278; the oligonucleotide comprises a sequence at least 80% identical to SEQ ID NO:15 and the polynucleotide comprises a sequence at least 80% identical to SEQ ID NO:279; the oligonucleotide comprises a sequence at least 80% identical to SEQ ID NO:16 and the polynucleotide comprises a sequence at least 80% identical to SEQ ID NO:280; the oligonucleotide comprises a sequence at least 80% identical to SEQ ID NO:17 and the polynucleotide comprises a sequence at least 80% identical to SEQ ID NO:281; the oligonucleotide comprises a sequence at least 80% identical to SEQ ID NO:18 and the polynucleotide comprises a sequence at least 80% identical to SEQ ID NO:282; the oligonucleotide comprises a sequence at least 80% identical to SEQ ID NO: 19 and the polynucleotide comprises a sequence at least 80% identical to SEQ ID NO:283; the oligonucleotide comprises a sequence at least 80% identical to SEQ ID NO:20 and the polynucleotide comprises a sequence at least 80% identical to SEQ ID NO:284; the oligonucleotide comprises a sequence at least 80% identical to SEQ ID NO:21 and the polynucleotide comprises a sequence at least 80% identical to SEQ ID NO:285; the oligonucleotide comprises a sequence at least 80% identical to SEQ ID NO:22 and the polynucleotide comprises a sequence at least 80% identical to SEQ ID NO:286; the oligonucleotide comprises a sequence at least 80% identical to SEQ ID NO:23 and the polynucleotide comprises a sequence at least 80% identical to SEQ ID NO:287; the oligonucleotide comprises a sequence at least 80% identical to SEQ ID NO:24 and the polynucleotide comprises a sequence at least 80% identical to SEQ ID NO:288; the oligonucleotide comprises a sequence at least 80% identical to SEQ ID NO:25 and the polynucleotide comprises a sequence at least 80% identical to SEQ ID NO:289; the oligonucleotide comprises a sequence at least 80% identical to SEQ ID NO:26 and the polynucleotide comprises a sequence at least 80% identical to SEQ ID NO:290; the oligonucleotide comprises a sequence at least 80% identical to SEQ ID NO:27 and the polynucleotide comprises a sequence at least 80% identical to SEQ ID NO:291; the oligonucleotide comprises a sequence at least 80% identical to SEQ ID NO:28 and the polynucleotide comprises a sequence at least 80% identical to SEQ ID NO:292; the oligonucleotide comprises a sequence at least 80% identical to SEQ ID NO:29 and the polynucleotide comprises a sequence at least 80% identical to SEQ ID NO:293; the oligonucleotide comprises a sequence at least 80% identical to SEQ ID NO:30 and the polynucleotide comprises a sequence at least 80% identical to SEQ ID NO:294; the oligonucleotide comprises a sequence at least 80% identical to SEQ ID NO:31 and the polynucleotide comprises a sequence at least 80% identical to SEQ ID NO:295; the oligonucleotide comprises a sequence at least 80% identical to SEQ ID NO:32 and the polynucleotide comprises a sequence at least 80% identical to SEQ ID NO:296; the oligonucleotide comprises a sequence at least 80% identical to SEQ ID NO:33 and the polynucleotide comprises a sequence at least 80% identical to SEQ ID NO:297; the oligonucleotide comprises a sequence at least 80% identical to SEQ ID NO:34 and the polynucleotide comprises a sequence at least 80% identical to SEQ ID NO:298; the oligonucleotide comprises a sequence at least 80% identical to SEQ ID NO:35 and the polynucleotide comprises a sequence at least 80% identical to SEQ ID NO:299; the oligonucleotide comprises a sequence at least 80% identical to SEQ ID NO:36 and the polynucleotide comprises a sequence at least 80% identical to SEQ ID NO:300; the oligonucleotide comprises a sequence at least 80% identical to SEQ ID NO:37 and the polynucleotide comprises a sequence at least 80% identical to SEQ ID NO:301; the oligonucleotide comprises a sequence at least 80% identical to SEQ ID NO:38 and the polynucleotide comprises a sequence at least 80% identical to SEQ ID NO:302; the oligonucleotide comprises a sequence at least 80% identical to SEQ ID NO:39 and the polynucleotide comprises a sequence at least 80% identical to SEQ ID NO:303; the oligonucleotide comprises a sequence at least 80% identical to SEQ ID NO:40 and the polynucleotide comprises a sequence at least 80% identical to SEQ ID NO:304; the oligonucleotide comprises a sequence at least 80% identical to SEQ ID NO:41 and the polynucleotide comprises a sequence at least 80% identical to SEQ ID NO:305; the oligonucleotide comprises a sequence at least 80% identical to SEQ ID NO:42 and the polynucleotide comprises a sequence at least 80% identical to SEQ ID NO:306; the oligonucleotide comprises a sequence at least 80% identical to SEQ ID NO:43 and the polynucleotide comprises a sequence at least 80% identical to SEQ ID NO:307; the oligonucleotide comprises a sequence at least 80% identical to SEQ ID NO:44 and the polynucleotide comprises a sequence at least 80% identical to SEQ ID NO:308; the oligonucleotide comprises a sequence at least 80% identical to SEQ ID NO:45 and the polynucleotide comprises a sequence at least 80% identical to SEQ ID NO:309; the oligonucleotide comprises a sequence at least 80% identical to SEQ ID NO:46 and the polynucleotide comprises a sequence at least 80% identical to SEQ ID NO:310; the oligonucleotide comprises a sequence at least 80% identical to SEQ ID NO:47 and the polynucleotide comprises a sequence at least 80% identical to SEQ ID NO:311; the oligonucleotide comprises a sequence at least 80% identical to SEQ ID NO:48 and the polynucleotide comprises a sequence at least 80% identical to SEQ ID NO:312; the oligonucleotide comprises a sequence at least 80% identical to SEQ ID NO:49 and the polynucleotide comprises a sequence at least 80% identical to SEQ ID NO:313; the oligonucleotide comprises a sequence at least 80% identical to SEQ ID NO:50 and the polynucleotide comprises a sequence at least 80% identical to SEQ ID NO:314; the oligonucleotide comprises a sequence at least 80% identical to SEQ ID NO:51 and the polynucleotide comprises a sequence at least 80% identical to SEQ ID NO:315; the oligonucleotide comprises a sequence at least 80% identical to SEQ ID NO:52 and the polynucleotide comprises a sequence at least 80% identical to SEQ ID NO:316; the oligonucleotide comprises a sequence at least 80% identical to SEQ ID NO:53 and the polynucleotide comprises a sequence at least 80% identical to SEQ ID NO:317; the oligonucleotide comprises a sequence at least 80% identical to SEQ ID NO:54 and the polynucleotide comprises a sequence at least 80% identical to SEQ ID NO:318; the oligonucleotide comprises a sequence at least 80% identical to SEQ ID NO:55 and the polynucleotide comprises a sequence at least 80% identical to SEQ ID NO:319; the oligonucleotide comprises a sequence at least 80% identical to SEQ ID NO:56 and the polynucleotide comprises a sequence at least 80% identical to SEQ ID NO:320; the oligonucleotide comprises a sequence at least 80% identical to SEQ ID NO:57 and the polynucleotide comprises a sequence at least 80% identical to SEQ ID NO:321; the oligonucleotide comprises a sequence at least 80% identical to SEQ ID NO:58 and the polynucleotide comprises a sequence at least 80% identical to SEQ ID NO:322; the oligonucleotide comprises a sequence at least 80% identical to SEQ ID NO:59 and the polynucleotide comprises a sequence at least 80% identical to SEQ ID NO:323; the oligonucleotide comprises a sequence at least 80% identical to SEQ ID NO:60 and the polynucleotide comprises a sequence at least 80% identical to SEQ ID NO:324; the oligonucleotide comprises a sequence at least 80% identical to SEQ ID NO:61 and the polynucleotide comprises a sequence at least 80% identical to SEQ ID NO:325; the oligonucleotide comprises a sequence at least 80% identical to SEQ ID NO:62 and the polynucleotide comprises a sequence at least 80% identical to SEQ ID NO:326; the oligonucleotide comprises a sequence at least 80% identical to SEQ ID NO:63 and the polynucleotide comprises a sequence at least 80% identical to SEQ ID NO:327; the oligonucleotide comprises a sequence at least 80% identical to SEQ ID NO:64 and the polynucleotide comprises a sequence at least 80% identical to SEQ ID NO:328; the oligonucleotide comprises a sequence at least 80% identical to SEQ ID NO:65 and the polynucleotide comprises a sequence at least 80% identical to SEQ ID NO:329; the oligonucleotide comprises a sequence at least 80% identical to SEQ ID NO:66 and the polynucleotide comprises a sequence at least 80% identical to SEQ ID NO:330; the oligonucleotide comprises a sequence at least 80% identical to SEQ ID NO:67 and the polynucleotide comprises a sequence at least 80% identical to SEQ ID NO:331; the oligonucleotide comprises a sequence at least 80% identical to SEQ ID NO:68 and the polynucleotide comprises a sequence at least 80% identical to SEQ ID NO:332; the oligonucleotide comprises a sequence at least 80% identical to SEQ ID NO:69 and the polynucleotide comprises a sequence at least 80% identical to SEQ ID NO:333; the oligonucleotide comprises a sequence at least 80% identical to SEQ ID NO:70 and the polynucleotide comprises a sequence at least 80% identical to SEQ ID NO:334; the oligonucleotide comprises a sequence at least 80% identical to SEQ ID NO:71 and the polynucleotide comprises a sequence at least 80% identical to SEQ ID NO:335; the oligonucleotide comprises a sequence at least 80% identical to SEQ ID NO:72 and the polynucleotide comprises a sequence at least 80% identical to SEQ ID NO:336; the oligonucleotide comprises a sequence at least 80% identical to SEQ ID NO:73 and the polynucleotide comprises a sequence at least 80% identical to SEQ ID NO:337; the oligonucleotide comprises a sequence at least 80% identical to SEQ ID NO:74 and the polynucleotide comprises a sequence at least 80% identical to SEQ ID NO:338; the oligonucleotide comprises a sequence at least 80% identical to SEQ ID NO:75 and the polynucleotide comprises a sequence at least 80% identical to SEQ ID NO:339; the oligonucleotide comprises a sequence at least 80% identical to SEQ ID NO:76 and the polynucleotide comprises a sequence at least 80% identical to SEQ ID NO:340; the oligonucleotide comprises a sequence at least 80% identical to SEQ ID NO:77 and the polynucleotide comprises a sequence at least 80% identical to SEQ ID NO:341; the oligonucleotide comprises a sequence at least 80% identical to SEQ ID NO:78 and the polynucleotide comprises a sequence at least 80% identical to SEQ ID NO:342; the oligonucleotide comprises a sequence at least 80% identical to SEQ ID NO:79 and the polynucleotide comprises a sequence at least 80% identical to SEQ ID NO:343; the oligonucleotide comprises a sequence at least 80% identical to SEQ ID NO:80 and the polynucleotide comprises a sequence at least 80% identical to SEQ ID NO:344; the oligonucleotide comprises a sequence at least 80% identical to SEQ ID NO:81 and the polynucleotide comprises a sequence at least 80% identical to SEQ ID NO:345; the oligonucleotide comprises a sequence at least 80% identical to SEQ ID NO:82 and the polynucleotide comprises a sequence at least 80% identical to SEQ ID NO:346; the oligonucleotide comprises a sequence at least 80% identical to SEQ ID NO:83 and the polynucleotide comprises a sequence at least 80% identical to SEQ ID NO:347; the oligonucleotide comprises a sequence at least 80% identical to SEQ ID NO:84 and the polynucleotide comprises a sequence at least 80% identical to SEQ ID NO:348; the oligonucleotide comprises a sequence at least 80% identical to SEQ ID NO:85 and the polynucleotide comprises a sequence at least 80% identical to SEQ ID NO:349; the oligonucleotide comprises a sequence at least 80% identical to SEQ ID NO:86 and the polynucleotide comprises a sequence at least 80% identical to SEQ ID NO:350; the oligonucleotide comprises a sequence at least 80% identical to SEQ ID NO:87 and the polynucleotide comprises a sequence at least 80% identical to SEQ ID NO:351; the oligonucleotide comprises a sequence at least 80% identical to SEQ ID NO:88 and the polynucleotide comprises a sequence at least 80% identical to SEQ ID NO:352; the oligonucleotide comprises a sequence at least 80% identical to SEQ ID NO:89 and the polynucleotide comprises a sequence at least 80% identical to SEQ ID NO:353; the oligonucleotide comprises a sequence at least 80% identical to SEQ ID NO:90 and the polynucleotide comprises a sequence at least 80% identical to SEQ ID NO:354; the oligonucleotide comprises a sequence at least 80% identical to SEQ ID NO:91 and the polynucleotide comprises a sequence at least 80% identical to SEQ ID NO:355; the oligonucleotide comprises a sequence at least 80% identical to SEQ ID NO:92 and the polynucleotide comprises a sequence at least 80% identical to SEQ ID NO:356; the oligonucleotide comprises a sequence at least 80% identical to SEQ ID NO:93 and the polynucleotide comprises a sequence at least 80% identical to SEQ ID NO:357; the oligonucleotide comprises a sequence at least 80% identical to SEQ ID NO:94 and the polynucleotide comprises a sequence at least 80% identical to SEQ ID NO:358; the oligonucleotide comprises a sequence at least 80% identical to SEQ ID NO:95 and the polynucleotide comprises a sequence at least 80% identical to SEQ ID NO:359; the oligonucleotide comprises a sequence at least 80% identical to SEQ ID NO:96 and the polynucleotide comprises a sequence at least 80% identical to SEQ ID NO:360; the oligonucleotide comprises a sequence at least 80% identical to SEQ ID NO:97 and the polynucleotide comprises a sequence at least 80% identical to SEQ ID NO:361; the oligonucleotide comprises a sequence at least 80% identical to SEQ ID NO:98 and the polynucleotide comprises a sequence at least 80% identical to SEQ ID NO:362; the oligonucleotide comprises a sequence at least 80% identical to SEQ ID NO:99 and the polynucleotide comprises a sequence at least 80% identical to SEQ ID NO:363; the oligonucleotide comprises a sequence at least 80% identical to SEQ ID NO:100 and the polynucleotide comprises a sequence at least 80% identical to SEQ ID NO:364; the oligonucleotide comprises a sequence at least 80% identical to SEQ ID NO:101 and the polynucleotide comprises a sequence at least 80% identical to SEQ ID NO:365; the oligonucleotide comprises a sequence at least 80% identical to SEQ ID NO:102 and the polynucleotide comprises a sequence at least 80% identical to SEQ ID NO:366; the oligonucleotide comprises a sequence at least 80% identical to SEQ ID NO:103 and the polynucleotide comprises a sequence at least 80% identical to SEQ ID NO:367; the oligonucleotide comprises a sequence at least 80% identical to SEQ ID NO:104 and the polynucleotide comprises a sequence at least 80% identical to SEQ ID NO:368; the oligonucleotide comprises a sequence at least 80% identical to SEQ ID NO:105 and the polynucleotide comprises a sequence at least 80% identical to SEQ ID NO:369; the oligonucleotide comprises a sequence at least 80% identical to SEQ ID NO:106 and the polynucleotide comprises a sequence at least 80% identical to SEQ ID NO:370; the oligonucleotide comprises a sequence at least 80% identical to SEQ ID NO:107 and the polynucleotide comprises a sequence at least 80% identical to SEQ ID NO:371; the oligonucleotide comprises a sequence at least 80% identical to SEQ ID NO: 108 and the polynucleotide comprises a sequence at least 80% identical to SEQ ID NO:372; the oligonucleotide comprises a sequence at least 80% identical to SEQ ID NO:109 and the polynucleotide comprises a sequence at least 80% identical to SEQ ID NO:373; the oligonucleotide comprises a sequence at least 80% identical to SEQ ID NO:110 and the polynucleotide comprises a sequence at least 80% identical to SEQ ID NO:374; the oligonucleotide comprises a sequence at least 80% identical to SEQ ID NO:111 and the polynucleotide comprises a sequence at least 80% identical to SEQ ID NO:375; the oligonucleotide comprises a sequence at least 80% identical to SEQ ID NO: 112 and the polynucleotide comprises a sequence at least 80% identical to SEQ ID NO:376; the oligonucleotide comprises a sequence at least 80% identical to SEQ ID NO:113 and the polynucleotide comprises a sequence at least 80% identical to SEQ ID NO:377; the oligonucleotide comprises a sequence at least 80% identical to SEQ ID NO:114 and the polynucleotide comprises a sequence at least 80% identical to SEQ ID NO:378; the oligonucleotide comprises a sequence at least 80% identical to SEQ ID NO:115 and the polynucleotide comprises a sequence at least 80% identical to SEQ ID NO:379; the oligonucleotide comprises a sequence at least 80% identical to SEQ ID NO:116 and the polynucleotide comprises a sequence at least 80% identical to SEQ ID NO:380; the oligonucleotide comprises a sequence at least 80% identical to SEQ ID NO:117 and the polynucleotide comprises a sequence at least 80% identical to SEQ ID NO:381; the oligonucleotide comprises a sequence at least 80% identical to SEQ ID NO:118 and the polynucleotide comprises a sequence at least 80% identical to SEQ ID NO:382; the oligonucleotide comprises a sequence at least 80% identical to SEQ ID NO:119 and the polynucleotide comprises a sequence at least 80% identical to SEQ ID NO:383; the oligonucleotide comprises a sequence at least 80% identical to SEQ ID NO:120 and the polynucleotide comprises a sequence at least 80% identical to SEQ ID NO:384; the oligonucleotide comprises a sequence at least 80% identical to SEQ ID NO:121 and the polynucleotide comprises a sequence at least 80% identical to SEQ ID NO:385; the oligonucleotide comprises a sequence at least 80% identical to SEQ ID NO:122 and the polynucleotide comprises a sequence at least 80% identical to SEQ ID NO:386; the oligonucleotide comprises a sequence at least 80% identical to SEQ ID NO:123 and the polynucleotide comprises a sequence at least 80% identical to SEQ ID NO:387; the oligonucleotide comprises a sequence at least 80% identical to SEQ ID NO:124 and the polynucleotide comprises a sequence at least 80% identical to SEQ ID NO:388; the oligonucleotide comprises a sequence at least 80% identical to SEQ ID NO: 125 and the polynucleotide comprises a sequence at least 80% identical to SEQ ID NO:389; the oligonucleotide comprises a sequence at least 80% identical to SEQ ID NO:126 and the polynucleotide comprises a sequence at least 80% identical to SEQ ID NO:390; the oligonucleotide comprises a sequence at least 80% identical to SEQ ID NO: 127 and the polynucleotide comprises a sequence at least 80% identical to SEQ ID NO:391; the oligonucleotide comprises a sequence at least 80% identical to SEQ ID NO:128 and the polynucleotide comprises a sequence at least 80% identical to SEQ ID NO:392; the oligonucleotide comprises a sequence at least 80% identical to SEQ ID NO:129 and the polynucleotide comprises a sequence at least 80% identical to SEQ ID NO:393; the oligonucleotide comprises a sequence at least 80% identical to SEQ ID NO:130 and the polynucleotide comprises a sequence at least 80% identical to SEQ ID NO:394; the oligonucleotide comprises a sequence at least 80% identical to SEQ ID NO:131 and the polynucleotide comprises a sequence at least 80% identical to SEQ ID NO:395; or the oligonucleotide comprises a sequence at least 80% identical to SEQ ID NO:132 and the polynucleotide comprises a sequence at least 80% identical to SEQ ID NO:396.

Embodiment P19. A cell or tissue comprising a composition of any one of Embodiments P1 to P18.

Embodiment P20. A target molecule bound to a composition of any one of Embodiments P1 to P18.

Embodiment P21. The target molecule of Embodiment P20, wherein the target molecule is selected from: PD-L1, CD8, CD3, PD-1, CD45, CD4, CD68, CD11c, FoxP3, α-SMA, CD20, Ki67, CD56, CD31, CTLA-4, PanCK, CD45RO, CD45RA, ATPase, Pan-Cadherin, Vimentin, Beta-2-microglobulin, and HLA-DR.

Embodiment P22. A composition comprising: a specific binding agent covalently attached to an oligonucleotide, wherein the oligonucleotide comprises a sequence at least 80% identical to a sequence selected from SEQ ID NO:397 to SEQ ID NO:529.

Embodiment P23. A kit comprising the composition of any one of Embodiments P1 to P18.

Embodiment P24. A method of detecting a target molecule in or on a cell or tissue, said method comprising binding a polynucleotide probe comprising a first binding sequence and a second binding sequence to an oligonucleotide, wherein the oligonucleotide comprises a sequence selected from SEQ ID NO:1 to SEQ ID NO:132 and is attached to the target molecule; amplifying the polynucleotide probe to form an amplification product comprising one or more copies of the first binding sequence and the second binding sequence; and hybridizing a primer to the amplification product and incorporating a labeled nucleotide into the primer, thereby detecting the target molecule.

Embodiment P25. The method of Embodiment P24, wherein prior to binding the polynucleotide probe, the method comprises binding a specific binding agent to a target molecule in or on a cell or tissue.

Embodiment P26. The method of Embodiment P25, wherein the oligonucleotide is at least partially double-stranded.

Embodiment P27. The method of Embodiment P25, wherein the oligonucleotide comprises a blocking polynucleotide hybridized to the sequence, and wherein the blocking polynucleotide is removed prior to binding the polynucleotide probe.

Embodiment P28. The method of Embodiment P24, wherein the target molecule is CD11c, CD20, CD3e, CD31, CD4, CD45RA, CD56, CD8, HLA-DR, Ki-67, PanCK, PD-1, and/or PD-L1.

What is claimed is:

1. A method of detecting a target molecule in or on a cell or tissue, said method comprising
binding a specific binding agent comprising an oligonucleotide to the target molecule in or on a cell or tissue, wherein said oligonucleotide comprises a first blocking oligonucleotide hybridized to a first sequence of the oligonucleotide, and a second blocking oligonucleotide hybridized to a second sequence of the oligonucleotide;
removing said blocking oligonucleotides and binding a polynucleotide probe comprising a first binding sequence and a second binding sequence to the oligonucleotide;
amplifying the polynucleotide probe to form an amplification product comprising one or more copies of the first binding sequence and the second binding sequence; and
binding a fluorescent moiety to the amplification product and detecting the fluorescent moiety, thereby detecting the target molecule.

2. The method of claim 1, wherein the specific binding agent is an antibody, single-chain Fv fragment (scFv), antibody fragment-antigen binding (Fab), affimer, or an aptamer.

3. The method of claim 1, wherein the specific binding agent is an antibody, single-chain Fv fragment (scFv), or antibody fragment-antigen binding (Fab).

4. The method of claim 1, wherein the specific binding agent is an enzyme, enzyme mutant, peptide, Molecular Imprinted Polymer (MIP), DARPin (Designed Ankyrin Repeat Protein), peptoid, lectin, siRNA, or miRNA molecule.

5. The method of claim 1, wherein binding the specific binding agent comprises incubation in a buffer at 30° C. to 40° C.

6. The method of claim 5, wherein binding the polynucleotide probe comprises incubation in a buffer at 40° C. to 50° C.

7. The method of claim 6, wherein amplifying comprises incubation in a buffer at 30° C. to 40° C.

8. The method of claim 7, wherein amplifying comprises rolling circle amplification for 15 minutes to 16 hours.

9. The method of claim 7, wherein amplifying comprises rolling circle amplification for 15 minutes to 1 hour.

10. The method of claim 1, wherein the fluorescent moiety comprises a fluorescently labeled oligonucleotide.

11. The method of claim 1, wherein the fluorescent moiety is a fluorescently labeled nucleotide, and the method further comprises binding a sequencing primer to the amplification product and binding the fluorescently labeled nucleotide to the sequencing primer.

12. The method of claim 1, wherein the oligonucleotide is 30 to 40 nucleotides.

13. The method of claim 1, wherein the target molecule is selected from: PD-L1, CD8, CD3, PD-1, CD45, CD4, CD68, CD11c, FoxP3, α-SMA, CD20, Ki67, CD56, CD31, CTLA-4, PanCK, CD45RO, CD45RA, ATPase, Pan-Cadherin, Vimentin, Beta-2-microglobulin, and HLA-DR.

14. A computer-implemented method for designing oligonucleotide sequences, said method comprising:
generating a plurality of oligonucleotide sequences comprising 20 to 40 nucleotides;
removing oligonucleotide sequences comprising a GC percentage greater than 60% over a portion of the oligonucleotide sequence;
removing oligonucleotide sequences comprising five consecutive strong bases and or five consecutive weak bases;
removing oligonucleotide sequences comprising a secondary structure; and.
removing oligonucleotide sequences comprising complementarity to the transcriptome.

15. The computer-implemented method of claim 14, further comprising removing oligonucleotide sequences comprising complementarity to the genome.

16. The computer-implemented method of claim 14, further comprising removing oligonucleotide sequences with a homopolymer sequence greater than 4 nucleotides.

17. An oligonucleotide comprising a sequence formed according to the method of claim 14.

18. The oligonucleotide of claim 17, wherein said oligonucleotide is covalently attached to a specific binding agent, wherein the specific binding agent is an antibody, single-chain Fv fragment (scFv), antibody fragment-antigen binding (Fab), affimer, aptamer, enzyme, peptide, Molecular Imprinted Polymer (MIP), DARPin (Designed Ankyrin Repeat Protein), peptoid, or lectin.

19. A cell or tissue comprising the oligonucleotide of claim 18.

20. A composition comprising:
a specific binding agent covalently attached to an oligonucleotide, wherein
the oligonucleotide is hybridized to a first blocking oligonucleotide and a second blocking oligonucleotide,
the oligonucleotide is 30 to 40 nucleotides
the oligonucleotide does not include five consecutive weak bases and/or five consecutive strong bases; and
the oligonucleotide does not comprise secondary structure.
